(19) United States
(12) Patent Application Publication
(10) Pub. No.: US 2021/0213010 A1
(43) Pub. Date: Jul. 15, 2021

(54) TLR7/8 AGONISTS AND LIPOSOME COMPOSITIONS

(71) Applicant: Torque Therapeutics, Inc., Cambridge, MA (US)

(72) Inventors: Thomas Lars Andresen, Vanløse (DK); Jonas Rosager Henriksen, Allerød (DK); Martin Kisha Kraemer, København (DK); Kira Røpke Jørgensen, København (DK)

(21) Appl. No.: 17/262,623

(22) PCT Filed: Jul. 24, 2019
(86) PCT No.: PCT/US2019/043319

§ 371 Date: Jan. 22, 2021

Related U.S. Application Data
(60) Provisional application No. 62/863,352, filed on Jun. 19, 2019.
(60) Provisional application No. 62/702,425, filed on Jul. 24, 2018.

(51) Int. Cl. A61K 31/4745; A61K 9/127; A61K 35/14; A61K 47/69; A61K 47/68; A61P 35/00; C07D 473/18; C07D 471/04

(52) U.S. Cl. CPC A61K 31/4745; A61K 9/1278; A61K 9/1271; A61K 35/14; A61K 47/6915; C07D 471/04; A61K 47/6871; A61P 35/00; A61K 9/1275; C07D 473/18; A61K 47/6913

(57) ABSTRACT

The present disclosure relates to a method of loading a toll like receptor (TLR)7/8 agonist into a liposome using remote loading and a kit of parts suitable for the loading of a TLR7/8 agonist into a liposome by said method. The present disclosure further relates to a liposome comprising a salt of a TLR7/8 agonist in the liposome interior and to the use of said liposome for stimulation of an immune response and/or treatment of a clinical condition. Finally, the present disclosure relates to a TLR7/8 agonist which is suitable for being remotely loaded into a liposome.

### CROSS-REFERENCE TO RELATED APPLICATIONS

This application claims priority to and the benefit of U.S. Provisional Application Nos. 62/702,425 filed Jul. 24, 2018 and 62/863,352 filed Jun. 19, 2019, the entire disclosures of both of which are incorporated herein by reference.

### FIELD

The present disclosure relates to a method of loading a toll like receptor (TLR) 7/8 agonist into a liposome using remote loading and a kit of components suitable for the loading of a TLR7/8 agonist into a liposome by said method. The present disclosure further relates to a liposome comprising a salt of a TLR7/8 agonist in the liposome interior and to the use of said liposome for stimulation of an immune response and/or treatment of a clinical condition. Finally, the present disclosure relates to a TLR7/8 agonist which is suitable for being remotely loaded into a liposome.

### BACKGROUND

Liposomes are characterized as nano-scale vesicles consisting of an interior core separated from the outer environment by a membrane of one or more bilayers. Liposomes have shown advantages as vesicles for delivery of a wide range of encapsulated and/or membrane-incorporated therapeutic or diagnostic entities. Liposomes can entrap both lipophilic and hydrophilic compounds enabling a diverse range of drugs to be encapsulated by these vesicles. Hydrophobic molecules are inserted into the bilayer membrane, and hydrophilic molecules can be entrapped in the aqueous interior. Upon delivery, the lipid bilayer of the liposome can fuse with other bilayers such as the cell membrane, thus delivering the liposome contents. The purpose of encapsulating a pharmaceutical drug into a liposome includes protection of the drug from the destructive environment and rapid excretion in vivo, targeting of the drug containing liposome to specific sites and minimizing systemic toxicity of the drug.

The technology of using liposomes for drug delivery is currently used in the clinic. Examples include the antifungal drug amphotericin B, marketed as Abelcet™, AMBisome™ and Amphicil™, and the anti-cancer drug doxorubicin, marketed as Doxil™ and DuanoXome™.

Different methods are used for the loading of the drug into liposomes. Passive loading of a drug may be achieved by co-dispersing the lipid and the drug in an aqueous buffer, thus achieving entrapment while the liposomes are being formed. Different passive loading techniques include mechanical dispersion methods, such as lipid film hydration or sonication, solvent dispersion methods, such as ethanol injection, or detergent removal techniques, such as detergent removal by dialysis. Passive loading often offers low loading efficiency with less than 50% of the intended drug being loaded into the liposome. Furthermore, the entrapment stability of the drug is often low.

For drug delivery in liposomal formulations, it is advantageous that the drug is entrapped inside the liposome in a therapeutic dose, such that the drug-to-lipid ratio is high. Otherwise the amount of lipids and/or other constituents of the liposomes can become toxic or the pharmacokinetics of enclosed drugs can be negatively affected. Active loading or remote loading of therapeutic or diagnostic entities into liposomes has proven to provide better loading efficiency than passive loading. In active loading, the drug is loaded into the liposome after the liposomes have been formed. The method may utilize a gradient, such as a salt gradient or a pH gradient across the liposome membrane for loading a compound. Alternatively, a carrier molecule may be utilized for transport across the liposome membrane.

TLR7/8 agonists are known to be immunostimulating and have antitumor activity. TLR7/8 agonists bind to and activate the TLR7 and/or TLR8 receptors, thereby mediating an immune response by the production of proinflammatory cytokines. Examples of TLR7/8 agonists include Imiquimod, Gardiquimod and Resiquimod. TLR7/8 agonists bearing a fatty acid tail have been incorporated in liposomes for use as adjuvants in vaccines with lowered systemic cytokine induction [Smirnov, D. et al., 2011], as anticancer treatment [Klauber, T. C. B. et al. 2017], or as anti-inflammatory treatment [Johansen, P. T. et al., 2015]. TLR7/8 agonists are known to dimerize prior to binding to the receptor. Hence, modifications to the structure of the TLR7/8 agonists have a large impact on the potency of the agonist. Attachment of large fatty acid tails to the TLR7/8 agonist for loading into liposomal structures is not considered optimal. An unmodified TLR7 agonist has also been co-formulated with a TLR4 agonist in liposomes using passive loading [Fox, C. et al., 2014]. However, as discussed above, the loading efficiency and entrapment stability using passive loading is not optimal, and new methods for loading of TLR7/8 agonists are warranted.

One example of loading of Resiquimod (R848), a TLR7/8 agonist, using remote loading has been reported [Duong, A. D. et al., 2016]. An ammonium sulfate gradient was used for the loading, with high ammonium sulfate concentration in the liposome interior. The document describes that “Resiquimod is a good candidate for remote loading because it has a primary amine that can act as proton acceptor”. The amine of Resiquimod is, however, in conjugation with the aromatic system and thus has a pKa of the conjugate acid of approximately 4. Resiquimod therefore comprises no primary amine which can be protonated by ammonium (pKa of 9.2), and hence, remote loading of Resiquimod using the described method is not plausible.

Thus, a need exists for improved methods and compositions for the remote loading of TLR7/8 agonists into liposomes.

### SUMMARY

The present disclosure provides a method for remote loading of TLR7/8 agonists comprising a carboxylic acid or an aliphatic amine into liposomes. The TLR7/8 agonists are designed to have acid/base properties suitable for enabling remote loading into liposomes. Similarly, the liposomal suspension is designed to provide optimal loading as well as stability of the liposomes and TLR7/8 agonist before, during and after loading. Provided herein is a class of compounds which have the desired properties for remote loading while retaining the biological activity as TLR7/8 agonists. Prior to the present disclosure, this has been challenging due to the mechanism of binding to the TLR7 and/or TLR8 receptors as described above.

The method as described in the present disclosure provides improved loading efficiency of the TLR7/8 agonist comprising a carboxylic acid or an aliphatic amine. Improved loading efficiency is highly desired as seen from both a cost perspective with less TLR7/8 agonist lost in the loading procedure and due to the ease of purification of the loaded liposome. Furthermore, a high drug-to-lipid ratio can be obtained, providing better efficacy and less toxicity of the liposome formulation.

In addition, the method as described herein provides improved entrapment stability of the loaded TLR7/8 agonist. The stability is increased by the low membrane permeability of the TLR7/8 agonist salt formed in the liposome interior. Furthermore, the salt can be designed to precipitate within the liposome, further improving the entrapment stability.

As described above, TLR7/8 agonists are known to dimerize prior to binding to the TLR7 and/or TLR8 receptors. Modification of the structure of TLR7/8 agonists while maintaining the biological activity has therefore proven challenging due to the modification affecting both the dimerization ability and the binding affinity to the receptor. The present disclosure provides weakly acidic/basic TLR7/8 agonists suitable for remote loading into liposomes with maintained biological activity.

In one embodiment, a pH or salt gradient across the liposome membrane drives the loading of the TLR7/8 agonist comprising a carboxylic acid or an aliphatic amine into the liposome. Upon entry in the liposome, a salt of the TLR7/8 agonist is formed, providing low membrane permeability of the TLR7/8 agonist and thereby high entrapment stability.

Thus, the present disclosure provides a method for loading of a TLR7/8 agonist comprising a carboxylic acid or an aliphatic amine, the method providing improved loading efficiency, improved drug-to-lipid ratio and improved entrapment stability of the loaded liposome.

The present disclosure further provides liposomes comprising a salt of a TLR7/8 agonist comprising a carboxylic acid or an aliphatic amine, the liposome having improved entrapment stability.

In one aspect, the present disclosure provides a method of loading a toll like receptor (TLR) 7/8 agonist comprising a carboxylic acid or an aliphatic amine into a liposome, the method comprising the steps of
a. providing a liposome in an exterior buffer solution, wherein a pH gradient exists across the liposome membrane,
b. providing a TLR7/8 agonist comprising a carboxylic acid or an aliphatic amine, and
c. combining said liposome in an exterior buffer solution with said TLR7/8 agonist comprising a carboxylic acid or an aliphatic amine, whereby at least part of said TLR7/8 agonist is loaded into the liposome.

In one aspect, the present disclosure provides a kit of components comprising
a. a suspension comprising a liposome in an exterior buffer solution, wherein a pH or salt gradient exists across the liposome membrane, and
b. a TLR7/8 agonist comprising a carboxylic acid or an aliphatic amine.

In another aspect, provided herein is a compound or a pharmaceutically acceptable salt thereof, wherein the compound or salt thereof is substantially purified and wherein the compound has formula (I):

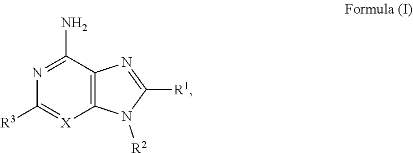

wherein:
X is selected from C or N;
R¹ is selected from the group consisting of OH and R^{A}, wherein R^{A} is optionally substituted with one or more Y;
R² is R^{A} optionally substituted with one or more of: —C₆-C₁₀-aryl (such as phenyl) optionally substituted with one or more R⁴, —C₄-C₁₀-heteroaryl optionally substituted with one or more R⁴, and/or Y;
R³ is (i) R^{A}, or (ii) together with X and the carbon to which it is attached, forms a C₄-C₁₀, preferably —C₅-C₆, aryl or heteroaryl, wherein R³ is optionally substituted with one or more Y;
R⁴ is selected from the group consisting of —C(O)—NH—R^{A}—R⁵, —R^{A}—NH—R^{A}—R⁵, and —R^{A}—NH—R⁵;
R⁵ is selected from the group consisting of H, —C₅-C₁₂-cycloalkyl, —C₄-C₁₂-heterocycloalkyl, —C₆-C₁₀-aryl, and —C₄-C₁₀-heteroaryl, wherein R⁵ is optionally substituted with one or more C₀-C₆-alkyl-Y;
Y is —COOH or —N(R^{B})(R^{C}), wherein (i) R^{B} and R^{C} are each independently selected from the group consisting of H, R^{A}, and (amino acid)ₙ wherein n is an integer selected from 1-6, or (ii) R^{B} and R^{C} together with N form a 3-6-membered ring; and
R^{A}, at each occurrence, is independently selected from the group consisting of —C₁-C₁₂-alkyl, —C₂-C₁₂-alkenyl, —C₂-C₁₂-alkynyl, —C₁-C₁₂-heteroalkyl, —C₂-C₁₂-heteroalkenyl, and —C₂-C₁₂-heteroalkynyl; and
wherein the compound has at least one carboxylic acid group or at least one amine group;
wherein when the compound has at least one carboxylic acid group, the compound has a logD above 0 in the pH range of about 6-10 and/or a logD below 0 in the pH range of about 4-6; and
wherein when the compound has at least one amine group, the compound has a logD above 0 in the pH range of about 2-6 and/or a logD below 0 in the pH range of about 6-9.

In some embodiments, in formula (I):
R¹ is OH or —C₁-C₄-heteroalkyl;
R² is selected from the group consisting of (a) —C₁-C₆-alkyl-phenyl, wherein the phenyl ring is optionally substituted with R⁴, (b) —C₁-C₆-alkyl-NH₂ and (c) —C₁-C₆-alkyl-NH-amino acid;
R³ is —C₁-C₆-heteroalkyl or when taken together with X (which is C) and the carbon to which it is attached, forms a C₅-C₆ aryl or heteroaryl; and
R⁴ is —C(O)—NH—C₁-C₄-alkyl-R⁵, —CH₂—NH—C₁-C₄-alkyl-R⁵, or —CH₂NHR⁵.

In some embodiments, in formula (I):
R¹ is selected from the group consisting of OH, —CH₂—O—CH₂—CH₃ and —CH₂—NH—CH₂—CH₃;
R² is selected from the group consisting of benzyl wherein the phenyl ring is substituted with R⁴, —CH₂—CH₂—NH₂ and —CH₂—CH₂—NH—C(O)—C(R⁶R⁷)—NH₂;
R³ is —O—CH₂—CH₂—O—CH₃ or when taken together with X (which is C) and the carbon to which it is attached, forms a benzene ring;
R⁴ is selected from the group consisting of —C(O)—NH—CH₂—CH₂—R⁵, —C(O)—NH—CH₂—R⁵, —CH₂—NH—CH₂—CH₂—R⁵, —CH₂—NH—CH₂—R⁵, and CH₂NHR⁵;
R⁵ is selected from the group consisting of H, N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl;
R⁶ is H or —CH₃; and R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

In some embodiments, in formula (I):
X is N;
R¹ is OH;
R² is benzyl, wherein the phenyl ring is substituted with R⁴;
R³ is —O—CH₂—CH₂—O—CH₃;
R⁴ is selected from the group consisting of —C(O)—NH—CH₂—CH₂—R⁵, —C(O)—NH—CH₂—R⁵, —CH₂—NH—CH₂—CH₂—R⁵ or —CH₂—NH—CH₂—R⁵; and
R⁵ is selected from the group consisting of H, N-methyl-N′-piperazinyl, N-pyrrolidinyl,
N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl.

In some embodiments, in formula (I):
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

In some embodiments, in formula (I):
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is —CH₃ or —CH(CH₃)₂.

In some embodiments, in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

In some embodiments, in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is selected from the group consisting of H, —CH₃, —CH(CH₃)₂ and —CH₂—CH(CH₃)₂.

In some embodiments, in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is —CH₃ or —CH(CH₃)₂.

In some embodiments, the compound is

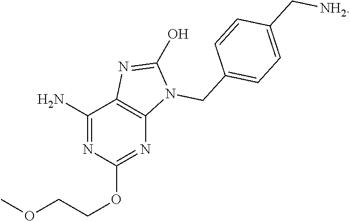 [SMILES: COCCOc1nc(N)c2nc(O)n(Cc3ccc(CN)cc3)c2n1]

In some embodiments, the compound of formula (I) is a TLR7/8 agonist and/or induces expression of one or more cytokines such as IL-6, IL-12p40 and/or IFNα.

In one aspect, the present disclosure provides a liposome comprising a salt of a TLR7/8 agonist, wherein the TLR7/8 agonist comprises a carboxylic acid or an aliphatic amine.

In one aspect, the present disclosure provides a TLR7/8 agonist. In some embodiments, the TLR7/8 agonist can be loaded into a liposome using the method of the present disclosure. In some embodiments, the TLR7/8 agonist has formula (I):

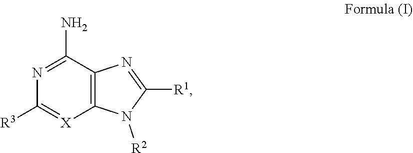

wherein:
X is selected from C or N;
R¹ is OH or —C₁-C₄-heteroalkyl;
R² is selected from the group consisting of —C₁-C₆-alkyl-phenyl, —C₁-C₆-alkyl-NH₂ and —C₁-C₆-alkyl-NH-amino acid, wherein the phenyl ring is substituted with R⁴;
R³ is —C₁-C₆-heteroalkyl or when taken together with X, wherein X is C and to which it is attached, forms an aromatic ring;
R⁴ is —C(O)—NH—C₁-C₄-alkyl-R⁵ or —CH₂—NH—C₁-C₄-alkyl-R⁵; and
R⁵ comprises at least one N and is an optionally substituted heterocycle or aryl, or a pharmaceutically acceptable salt thereof.

Also provided herein is a liposome composition comprising a liposome and a salt of Gardiquimod and/or one or more of the compounds disclosed herein, wherein the salt is entrapped inside the liposome, wherein the liposome comprises an interior buffer solution. In some embodiments, the compound comprises an aliphatic amine group and the interior buffer solution comprises an acidic component, such that inside the liposome the compound reacts with the acidic component to form the salt. In certain embodiments, the compound comprises a carboxylic acid group and the interior buffer solution comprises a basic component, such that inside the liposome the compound reacts with the basic component to form the salt.

The liposome can include in its membrane one or more of: DSPC (1,2-distearoyl-sn-glycero-3-phosphocholine), HSPC (hydrogenated soybean phosphatidylcholine), CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy (polyethylene glycol)]). In some embodiments, the liposome comprises in its membrane about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

A further aspect relates to a method of liposome loading, comprising: providing a liposome in an exterior buffer solution, wherein the liposome comprises an interior buffer solution and wherein a pH gradient exists between the exterior buffer solution and the interior buffer solution across liposome membrane, wherein preferably the pH gradient is at least about 2.0; providing a TLR7/8 agonist comprising a carboxylic acid and/or an aliphatic amine group, wherein optionally the TLR7/8 agonist is Gardiquimod and/or one or more of the compounds disclosed herein, and combining said liposome in said exterior buffer solution with said compound, thereby loading at least a portion of Gardiquimod and/or said compound into the liposome to form a loaded liposome. In some embodiments, the method has a loading efficiency of at least 50%, such as at least 70%, at least 80%, or at least 90%.

In some embodiments, the compound comprises an aliphatic amine group and the interior buffer solution comprises an acidic component, such that inside the liposome the compound reacts with the acidic component to form a salt. In some embodiments, the acidic component is selected from the group consisting of ammonium sulphate, ammonium phosphate, ammonium citrate, ammonium acetate, citric acid, acetic acid, oxalic acid, tartronic acid, dihydroxymalonic acid, fumaric acid, malic acid, tartaric acid, glutaric acid, phosphoric acid, sodium phosphonate, potassium phosphonate, sulfonic acid, sucrose octasulfonic acid, or the basic component is selected from the group consisting of ammonium acetate, potassium acetate, sodium acetate, calcium acetate, ammonium benzoate, potassium benzoate, sodium benzoate and calcium benzoate.

In some embodiments, the compound comprises a carboxylic acid group and the interior buffer solution comprises a basic component, such that inside the liposome the compound reacts with the basic component to form a salt. In some embodiments, the basic component is selected from the group consisting of ammonium acetate, potassium acetate, sodium acetate, calcium acetate, ammonium benzoate, potassium benzoate, sodium benzoate and calcium benzoate.

In some embodiments, the salt is a precipitate.

In some embodiments, in the loaded liposome, less than about 20%, preferably less than about 10%, more preferably less than about 5% of the compound is released from the liposome after 1 month or longer at 5° C., such as after 1 week or longer at 5° C. In some embodiments, in the loaded liposome, a drug-to-lipid ratio is at least 0.2, for example at least 0.25, such as at least 0.3.

In some embodiments, the compound has a logD above 0 in the exterior buffer solution and/or a logD below 0 in the interior buffer solution.

In some embodiments, the compound contains an aliphatic amine group. In some embodiments, the interior buffer solution has a pH in the range of about 4-6.5, such as in the range of 4-6 or in the range of 5-6. In some embodiments, the exterior buffer solution has a pH in the range of 7-9.5, such as in the range of 7-9, in the range of 7-8.5, or in the range of 7-8. In some embodiments, the exterior buffer solution comprises a buffering component selected from the group consisting of HEPES, TAPS, phosphate, histidine, citrate, Bicine, TRIS, TAPSO, TES, Bis-tris, ADA, ACES, PIPES, MOPSO, BES, TES, DIPSO, MOBS, TAPSO, Trizma, HEPPSO, POPSO, TEA, EPPS, Tricine, Gly-Gly, HEPBS, AMPD, TABS, AMPSO, CHES, CAPSO, AMP and MOPS. In some embodiments, a conjugate acid of the compound has a pKa in the range of about 5.5-10, such as in the range of 6-9, for example in the range of 6.5-9, such as in the range of 6.5-8.5, for example in the range of 6.5-8. In some embodiments, the compound has a logD above 0 in the pH range of 6-10 and/or a logD below 0 in the pH range of 4-6.

In some embodiments, the compound comprises a carboxylic acid group. In some embodiments, the interior buffer solution has a pH in the range of 7-9, such as in the range of 7.5-9 or in the range of 8-9. In some embodiments, the interior buffer solution comprises a basic component selected from the group consisting of ammonium acetate, potassium acetate, sodium acetate, calcium acetate, ammonium benzoate, potassium benzoate, sodium benzoate and calcium benzoate. In some embodiments, the exterior buffer solution has a pH in the range of 2.5-6, such as in the range of 2.5-5, in the range of 2.5-4, or in the range of 2.5-3. In some embodiments, the exterior buffer solution comprises a buffering component selected from the group consisting of citric acid, acetic acid, phosphate, histidine, MES, Bis-Tris and ADA. In some embodiments, the compound has a pKa in the range of 2-6, for example in the range of 2-5, in the range of 2-4, or in the range of 2-3. In some embodiments, the compound has a logD above 0 in the pH range of 2-6 and/or a logD below 0 in the pH range of 6-9.

In some embodiments, the liposome comprises in its membrane one or more of: DSPC (1,2-distearoyl-sn-glycero-3-phosphocholine), HSPC (hydrogenated soybean phosphatidylcholine), CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy(polyethylene glycol)]). In some embodiments, the liposome comprises in its membrane about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

Also provided herein is a liposome composition prepared by the method disclosed herein.

Another aspect relates to a liposome composition comprising a membrane and a TLR7/8 agonist entrapped inside the membrane, wherein the membrane comprises one or more of: DSPC (1,2-distearoyl-sn-glycero-3-phosphocholine), HSPC (hydrogenated soybean phosphatidylcholine), CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy(polyethylene glycol)]); wherein the TLR7/8 agonist is Gardiquimod and/or one or more of the compounds disclosed herein. In some embodiments, the membrane comprises about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

Also provided herein is a pharmaceutical composition comprising the liposome composition disclosed herein and a pharmaceutically acceptable carrier.

Also provided herein is a cell composition comprising the liposome composition disclosed herein and a nucleated cell such as an immune cell.

Also provided herein is a composition comprising the compound or salt disclosed herein, and further comprising a pharmaceutically acceptable salt, a liposome, and/or a nucleated cell such as an immune cell.

Also provided herein is a composition comprising Gardiquimod, and further comprising a pharmaceutically acceptable salt, a liposome, and/or a nucleated cell such as an immune cell.

Also provided herein is use of any one or more of the liposome composition disclosed herein, the pharmaceutical composition disclosed herein, the cell composition disclosed herein, or the composition disclosed herein, in the manufacture of a medicament for stimulating an immune response in an individual in need thereof, such as for the treatment of cancer, an infectious disease, an inflammatory condition or disease and autoimmune disease or allergy.

### BRIEF DESCRIPTION OF THE DRAWINGS

FIG. 1A: Synthesis of a TLR7 agonist compound library. Reagents and conditions: a) 4-(Bromomethyl)benzonitrile, Na₂CO₃, DMF, room temperature, 18 hr; b) NH₃, MeOH, 60° C., 18 hr; c) Na, 2-methoxyethanol, 115° C., 21 hr; d) NBS, DCM, room temperature, 20 hr; e) NaOMe, MeOH, reflux, 18 hr; f) NaOH, H₂O, reflux; g) PyAOP, R—NH₂, DCM, DMF; h) DIBAL-H, THF, −78° C. to RT then NaI, ClSiMe₃, MeCN; i) NaCNBH₃, R—NH₂, DCM or DMF.

FIG. 1B: Potency of TLR7 agonist compound library (EC₅₀=half maximal effective concentration). Benchmarks (Gdq and R848) indicated with dashed lines.

FIGS. 1C-1E: B and T cell activation of R848 and compounds 1j (Formula I, A7) and 2 (KRJ2-110, Formula I, B11) from FIG. 1A.

FIG. 2A: Synthesis of MK079-D from 2,4-quinolinediol.

FIG. 2B: General procedure for amino acid derivatization of MK079-D.

FIG. 3: A visualization of the general procedure for small molecule remote loading.

FIG. 4: Remote loading of imidazoquinolines in liposomes that are dialyzed against HEPES.

FIG. 5: Remote loading of MK079-D in formulation F1 dialyzed against HEPES at varying drug/lipid concentrations.

FIG. 6: Remote loading of MK079-D in formulation F2 dialyzed against HEPES at varying drug/lipid concentrations.

FIG. 7: A comparison of remote loading of MK079-D in formulations F1 and F2 drug/lipid ratio of 0.1 when liposomes were dialyzed against HEPES compared to dialysis against TAPS.

FIG. 8: Loading efficiencies of Gardiquimod, MK087, MK088, and MK089 in the formulation F1 dialyzed against HEPES at a drug/lipid ratio of 0.1.

FIG. 9: Loading efficiencies of Gardiquimod, MK087, MK088, and MK089 in the formulation F2 dialyzed against HEPES at a drug/lipid ratio of 0.1.

FIG. 10: Cryo-TEM image of MK079-D remote loaded into formulation F1 at drug/lipid ratio of 0.25 showed precipitation in the liposomes. The insert displays four liposomes at 50% increased magnification level.

FIG. 11: Cryo-TEM image of MK079-D remote loaded into formulation F1 at drug/lipid ratio of 0.50 showed precipitation in the liposomes. The insert displays five liposomes at 50% increased magnification level.

FIG. 12: Cryo-TEM image of MK079-D remote loaded into formulation F2 at drug/lipid ratio of 0.25 showed precipitation in the liposomes. The insert displays two liposomes at 75% increased magnification level.

FIG. 13: Cryo-TEM image of MK079-D remote loaded into formulation F2 at drug/lipid ratio of 0.50 showed precipitation in the liposomes. The insert displays liposomes at 100% increased magnification level.

FIG. 14: Expressed levels of IL-6 upon stimulation of whole human blood with Resiquimod (R848) and MK079-D. While stimulation with R848 showed dose dependent expression of IL-12p40 with similar expression levels for both donors at 10 μM and 1 μM, stimulation with MK079-D gave an expression of IL-12p40 at 10 μM in both donors.

FIG. 15: Expressed levels of IL-12p40 upon stimulation of whole human blood with R848 and MK079-D. While stimulation with R848 showed dose dependent expression of IL-12p40 with similar expression levels for both donors at 10 μM and 1 μM, stimulation with MK079-D gave an expression of IL-12p40 at 10 μM in both donors.

FIG. 16: Expressed levels of IL-6 upon stimulation of whole human blood with MK079-D remote loaded in formulations F1 or F2 resulted in expression of cytokine IL-6. Formulation F2 induced a higher expression of IL-6 compared to formulation F1 when dialyzed against both HEPES and TAPS.

FIG. 17: Expressed levels of IL-12p40 upon stimulation of whole human blood with MK079-D remote loaded in formulations F1 or F2 dialyzed against HEPES or TAPS. Stimulation with MK079-D in formulation F1 did not lead to any expression of IL-12p40 while stimulation of MK079-D in formulation F2 lead to a larger expression of IL-12p40 when dialyzed against HEPES compared to TAPS.

FIG. 18: Expressed levels of IL-6 upon stimulation of whole human blood with Gardiquimod, MK088, MK089, MK090, MK091, MK093, and MK094. Gardiquimod, MK088, MK089, MK090, MK091, and MK093 all induced expression of IL-6 at 10 μM in donor 2, while only MK090 was able to induce expression of IL-6 in both donors.

FIG. 19: Expressed levels of IL-12p40 upon stimulation of whole human blood with Gardiquimod, MK088, MK089, MK090, MK091, MK093, and MK094. MK088, MK090, MK091, and MK093 all induced expression of IL-12p40 at 10 μM in donor 2, while none of the molecules were able to induce expression of IL-12p40 in donor 1 in the concentrations tested.

FIG. 20: Expressed levels of IL-6 upon stimulation of whole human blood with Gardiquimod, MK087, MK088, and MK089 which were remote loaded in formulation F1 dialyzed against HEPES and remote loaded with a drug/lipid ratio of 0.1. In donor 2, MK087 and MK088 were capable of inducing expression of IL-6 at 10 μM when remote loaded into formulation F1. No induction of IL-6 expression was seen with Gardiquimod or MK089 when remote loaded into formulation F1 in the concentrations tested.

FIG. 21: Expressed levels of IL-12p40 upon stimulation of whole human blood with Gardiquimod, MK087, MK088, and MK089 which were remote loaded in formulation F1 dialyzed against HEPES and remote loaded with a drug/lipid ratio of 0.1. In both donors, none of the small molecules Gardiquimod, MK087, MK088, and MK089 were capable of inducing expression of IL-12p40 when remote loaded into formulation F1 in the concentrations tested.

FIG. 22: Expressed levels of IL-6 upon stimulation of whole human blood with Gardiquimod, MK087, MK088, and MK089 which were remote loaded in formulation F2 dialyzed against HEPES and remote loaded with a drug/lipid ratio of 0.1. Gardiquimod, MK087, MK088, and MK089 were all capable of inducing expression of IL-6 at 10 μM when remote loaded into formulation F2. Gardiquimod and MK089 were capable of inducing expression of IL-6 in both donors, while MK087 and MK088 were not capable of inducing expression of IL-6 in donor 1 in the concentrations tested.

FIG. 23: Expressed levels of IL-12p40 upon stimulation of whole human blood with with Gardiquimod, MK087, MK088, and MK089 which were remote loaded in formulation F2 dialyzed against HEPES and remote loaded with a drug/lipid ratio of 0.1. Gardiquimod, MK088, and MK089 were all capable of inducing expression of IL-12p40 in one donor at 10 μM when remote loaded into formulation F2, while MK087 was not capable of inducing expression of IL-12p40 when remote loaded into formulation F2 in the concentrations tested.

FIG. 24: Synthesis of KRJ1-068.

FIG. 25: Synthesis of KRJ1-085.

FIG. 26: Synthesis of KRJ1-092.

FIG. 27: Synthesis of KRJ1-101.

FIG. 28: Synthesis of KRJ2-006.

FIG. 29: Synthesis of KRJ2-002.

FIG. 30: Synthesis of KRJ2-014.

FIG. 31: Synthesis of KRJ2-015.

FIG. 32: Synthesis of KRJ1-098.

FIG. 33: Synthesis of MK130. Reagents and conditions: (a) 1-(N-Boc-aminomethyl)-4-(aminomethyl)benzene, Et₃N, DCM, 20° C., 3 h; (b) H₂ (5 bar), 10 wt % Pd/C, ethyl acetate, rt, 8 h; (c) Valeroyl chloride, Et₃N, THF, rt, o.n.; (d) i) Ethanol/5M NaOH 37:1 (v/v), 20° C., 4 h; ii) NaN₃, DMSO, 160° C., 12 h; (e) i) PPh₃, 160° C., 30 min; ii) acetonitrile/H₂O/TFA 10:3:3 (v/v), 80° C., 24 h.

FIG. 34: Remote loading of MK130 and derivatives thereof. Encapsulation efficiencies (Loading %) measured at 1 h and 3 h imidazoquinolines remote loaded in formulation F3 (150 mM ammonium sulfate/25 mM HEPES gradient) at a drug to lipid ration of 0.1. Results represent mean±SD (n=2).

FIG. 35: Remote loading of MK130 and MK135 at varying drug/lipid ratios. Remote loading of MK130 (left) and MK135 (right) at drug/lipid ratios 0.10, 0.25, and 0.50 in formulation F3 (150 mM ammonium sulfate/25 mM HEPES gradient). Encapsulation efficiencies (Loading %) were measured at 1 h and 3 h and results represent mean±SD (n=2).

FIG. 36: Remote loading of MK138 at varying drug/lipid ratios in F3 and F4. Remote loading of MK138 at drug/lipid ratios 0.10 and 0.25 in formulations F3 (150 mM ammonium sulfate/25 mM HEPES gradient) and F4 (250 mM ammonium sulfate/25 mM HEPES gradient). Encapsulation efficiencies (Loading %) were measured at 1 h and 3 h and results represent mean±SD (n=2).

FIG. 37: Cryo-TEM imaging of liposomal MK130 in formulation F3. MK130 was remote loaded into formulation F3 at a drug to lipid ratio of 0.1 as described in example 65. Scale bar depicts 100 nm.

FIG. 38: Cryo-TEM imaging of liposomal MK138 in formulation F3. MK138 was remote loaded into formulation F3 at a drug to lipid ratio of 0.1 as described in example 65. Scale bar depicts 100 nm.

FIG. 39: Cryo-TEM imaging of liposomal MK138 in formulation F4. MK138 was remote loaded into formulation F4 at a drug to lipid ratio of 0.1 as described in example 65. Scale bar depicts 100 nm.

FIG. 40: TLR7 agonist activity of MK130 and derivatives in TLR7 reporter cell assay. Compounds are plotted according to their NF-κB induction at 0.32 μM (HEK-Blue TLR7 stimulation). Top: Compounds that exhibited an activity lower than or equal to R848 (grey). Bottom: Compounds that exhibited an activity higher than or equal to R848 (grey). 10 mM DMSO solutions of the compounds were diluted in PBS. Concentrations represent the final concentration in each well. Absorbances were normalized to DMSO dilutions (blank). Results are given as mean±SD (n=3).

FIG. 41: TLR7 agonist activity of liposomal MK130 and MK138. HEK Blue mTLR7 assay with non-formulated and liposomal MK130 (top) and MK138 (bottom) respectively (HEK-Blue TLR7 stimulation). Free compounds were diluted from 10 mM DMSO solutions in PBS. Liposomal formulations were diluted in PBS. Concentrations represent the final compound concentration in each well. Absorbances were normalized to DMSO dilutions (blank). Results are given as mean±SD (n=3).

FIG. 42: Stimulation of RAW 264.7 cells with Resiquimod (R848), Gardiquimod (Gdq), and MK130 and derivatives thereof. NF-κB induction in RAW 264.7 cells incubated with compounds diluted from DMSO solutions in PBS. Concentrations represent final concentrations in each well. Absorbances were normalized to background. Results are given as mean±SD (n=2).

FIG. 43: Synthesis of KRJ2-110.

FIG. 44: Cryo-TEM image of KRJ2-110 remote loaded.

FIG. 45: Cryo-TEM image of KRJ2-110 remote loaded.

FIG. 46: Synthesis of Cholesterol-R3.

FIG. 47: % Recovery of liposome loaded cells relative to mock loaded cells measured by flow cytometry by counting live cells.

FIG. 48: Amount of Gardiquimod loaded per 1×10⁶ PMEL CD8 T cells immediately after 60 minutes incubation and washing of cells. Saturated formulations load more GDQ than unsaturated formulations.

FIG. 49: % of Gardiquimod release from PMEL CD8 T cells with time as cells grow in culture with 100 IU/mL IL-2.

FIG. 50: Tumor volume (B6D2F1 SQ) on treatment day of adoptive cell transfer (ACT, i.e., day 0).

FIG. 51: B16-F10 SubQ tumor growth days after adoptive cell transfer (ACT).

FIG. 52: Weight of B16-F10 SubQ tumor bearing mice with time before and after treatment of adoptive cell transfer (ACT, day 0).

FIGS. 53A-53B: Cytokine/chemokine release kinetics in blood in B6D2F1 mice post treatment with PMEL cells, PMEL loaded with liposomes or systemically delivered TLR agonist.

FIG. 54: Amount of Gardiquimod loaded (measured by HPLC) onto 1×10⁶ human CD3 or mouse PMEL CD8 T cells for 2 different formulations (antibody mediated versus Arg3 peptide mediated loading).

FIG. 55: Amount of KRJ2-110 loaded (measured by HPLC) onto 1×10⁶ human CD3 or mouse PMEL CD8 T cells for 2 different formulations (antibody mediated versus Arg3 peptide mediated loading).

FIG. 56: Left panel: % Recovery of human CD3 T cells relative to mock loaded control after liposome loading and washing 0 and 4 hrs after culturing. Right panel: Amount of KRJ2-110 per 1×10⁶ T cells after loading and washing measured by HPLC.

FIG. 57: Amount of Gardiquimod or KRJ2-110 loaded onto human CEF stimulated muti-targeted T cells with load times of 1, 2 and 3 hrs, washed and measured by HPLC.

FIG. 58: Top panel: % of drug released from cells immediately after thaw versus 1 hr after thaw for cells that were loaded for 1 hr prior to freezing. Bottom panel: % of drug released from cells immediately after thaw versus 1 hr after thaw for cells that were loaded for 2 hr prior to freezing.

FIG. 59: B16-F10 tumor volume on day adoptive cell therapy (ACT) with PMEL cells (day 0).

FIG. 60: Left panel: B16-F10 tumor volume in B6D2F1 mice versus days post treatment of adoptive cell therapy (PMEL). Right panel: Survival of B6D2F1 mice versus days post treatment of adoptive cell therapy (PMEL).

FIG. 61: Amount of Gardiquimod or KRJ2-110 loaded onto 1×10⁶ TAA specific multi-targeted T cells (MTCs) after 1-2 hr of loading.

FIG. 62: % of Gardiquimod (Gdq) or KRJ2-110 (KRJ) release from cells immediately after thaw or after 1 hr post thaw.

FIG. 63: Markers and cell subsets characterized by flow cytometry in various tissues and organs.

FIG. 64: Number of convention type 1 Dendritic Cells (cDC1) and plasmacytoid Dendritic Cells (pDC1) per gram of tumor.

FIG. 65: % of proliferating (Ki-67+) PMEL CD8 T cells and endogenous CD8+ T cells in blood, spleen and tumor 4 days after treatment by adoptive cell transfer (ACT).

FIG. 66: % of degranulated (CD107a+) activated (CD25+) CD8 PMEL T cells and endogenous CD8 T cells (non-PMEL) in tumor draining lymphnodes and nondraining lymphnodes 4 and 7 days after treatment by adoptive cell transfer (ACT).

FIG. 67: % CD8 PMEL T cells that are exhausted by PD1-1+LAG3+ markers 4 and 7 days after treatment by adoptive cell transfer (ACT).

FIG. 68: Number of Monocytic and Granulocytic Myeloid Derived Suppressor Cells (MDSC) per gram of tumor 7 days after treatment by adoptive cell transfer (ACT).

FIG. 69: MHCII expression on CD45+ cells in the spleen and tumor 4 and 7 days after adoptive cell transfer (ACT).

FIG. 70: Amount of liposome loaded onto PMEL cells measured by Median Fluorescence Intensity (MFI) using flow cytometry and gating on live cells via 7AAD staining.

FIG. 71: Correlation between liposome loading onto CD8 PMEL cells and receptor density on the PMEL cell surface measured by the BD Quantibrite kit.

FIG. 72: Amount of liposome loaded onto PMEL CD8 and human CD3 T cells measured by Median Fluorescence Intensity (MFI) using flow cytometry and gating on live cells via 7AAD staining.

FIG. 73: Persistence of liposome Median Fluorescence Intensity (MFI) on the surface of human T cells after liposome loading and washing, measured by flow cytometry.

FIG. 74: Persistence of liposome Median Fluorescence Intensity (MFI) on the surface of human T cells after liposome loading and washing, measured by flow cytometry.

FIG. 75: Amount of Gardiquimod measured by HPLC on 1×10⁶ human CD3 T cells after loading and washing.

FIG. 76: KRJ2-110 loading per 1×10⁶ human CD3 T cells measured by HPLC after loading and washing.

FIG. 77: Amount of MK-138 (TLR7/8 agonist) and KRJ2-110 (TLR7 agonist) loaded onto human CD3 T cells via liposome carriers. Measurements made by HPLC after liposomes incubation with cells and washing.

### DETAILED DESCRIPTION

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992), Oxford Dictionary of Biochemistry and Molecular Biology, Smith et al. (Eds.), Oxford University Press (revised ed., 2000); Encyclopedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (Pt ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

A “liposome” refers to a vesicle or a microscopic particle formed by at least one lipid bilayer. The liposomes may be artificially prepared. In some embodiments, the liposomes can have an average diameter of about 50-900 nm, about 50-500 nm, about 60-480 nm, about 80-450 nm, about 100-400 nm, about 50-300 nm, about 80-250 nm, or about 100-200 nm. Liposomes may enclose an aqueous compartment and are capable of entrapping or housing a drug, antigen., vaccine, enzyme, adjuvant or another substance capable of being targeted to cells.

As used herein, the term “lipids” refers to any of a group of organic compounds, including the fats, oils, waxes, sterols, and triglycerides, that are insoluble in water but soluble in nonpolar organic solvents, are oily to the touch, and together with carbohydrates and proteins constitute the principal structural material of living cells. The lipid can be modified to have a peptide or antibody conjugated thereto.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, hydroxyproline, gamma-carboxy glutarnate, and O-phospho serine. Amino acid analogs refer to compounds that have the same basic chemical structure as a naturally occurring amino acid, i.e. a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetic's refers to chemical compounds that have a structure that is different from the general chemical structure of an amino acid, but that functions in a manner similar to a naturally occurring amino acid.

“Antigen” (Ag) as used herein refers to a macromolecule, including all proteins or peptides. In some embodiments, an antigen is a molecule that can provoke activation of certain immune cells (including immune regulatory cells) and/or antibody generation. Any macromolecule, including almost all proteins or peptides, can be an antigen. Antigens can also be derived from genomic or recombinant DNA or RNA. For example, any DNA comprising a nucleotide sequence or a partial nucleotide sequence that encodes a protein capable of eliciting an immune response encodes an antigen. In embodiments, an antigen does not need to be encoded solely by a full-length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full-length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

As used herein, a “cytokine” or “cytokine molecule” refers to full length, a fragment or a variant of a naturally-occurring, wild type cytokine (including fragments and functional variants thereof having at least 10% of the activity of the naturally-occurring cytokine molecule). In embodiments, the cytokine molecule has at least 30, 50, or 80% of the activity, e.g., the immunomodulatory activity, of the naturally-occurring molecule. In embodiments, the cytokine molecule further comprises a receptor domain, e.g., a cytokine receptor domain, optionally, coupled to an immunoglobulin Fc region. In other embodiments, the cytokine molecule is coupled to an immunoglobulin Fc region. In other embodiments, the cytokine molecule is coupled to an antibody molecule (e.g., an immunoglobulin Fab or scFv fragment, a Fab fragment, a FAB₂ fragment, or an affibody fragment or derivative, e.g., a sdAb (nanobody) fragment, a heavy chain antibody fragment, single-domain antibody, a bi-specific or multispecific antibody), or non-antibody scaffolds and antibody mimetics (e.g., lipocalins (e.g., anticalins), affibodies, fibronectin (e.g., monobodies or Adnectins), knottins, ankyrin repeats (e.g., DARPins), and A domains (e.g., avimers)).

“Nucleated cells” are cells which contain nucleus. In some embodiments, the nucleated cells can be immune cells.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Immune cells include immune regulatory cells (e.g., Tregs) and immune effector cells described herein. Immune cell may include modified versions of cells involved in an immune response, e.g. modified NK cells, including NK cell line NK-92 (ATCC cat. No. CRL-2407), haNK (an NK-92 variant that expresses the high-affinity Fc receptor FcγRIIIa (158V)) and taNK (targeted NK-92 cells transfected with a gene that expresses a CAR for a given tumor antigen). Immune cells include immune effector cells.

“Immune effector cell,” as that term is used herein, refers to a cell that is involved in an immune response, e.g., in the promotion of an immune effector response. Examples of immune effector cells include, but are not limited to, T cells, e.g., CD4+ T cells, CD8+ T cells, alpha T cells, beta T cells, gamma T cells, and delta T cells; B cells; natural killer (NK) cells; natural killer T (NKT) cells; dendritic cells; and mast cells. In some embodiments, the immune cell is an immune cell (e.g., T cell or NK cell) that comprises, e.g., expresses, a Chimeric Antigen Receptor (CAR), e.g., a CAR that binds to a cancer antigen. In other embodiments, the immune cell expresses an exogenous high affinity Fc receptor. In some embodiments, the immune cell comprises, e.g., expresses, an engineered T-cell receptor. In some embodiments, the immune cell is a tumor infiltrating lymphocyte. In some embodiments the immune cells comprise a population of immune cells and comprise T cells that have been enriched for specificity for a tumor-associated antigen (TAA), e.g. enriched by sorting for T cells with specificity towards MI-ICs displaying a TAA of interest, e.g. MART-1. In some embodiments immune cells comprise a population of immune cells and comprise T cells that have been “trained” to possess specificity against a TAA by an antigen presenting cell (APC), e.g. a dendritic cell, displaying TAA peptides of interest. In some embodiments, the T cells are trained against a TAA chosen from one or more of MART-1, MAGE-A4, NY-ESO-1, SSX2, Survivin, or others. In some embodiments the immune cells comprise a population of T cells that have been “trained” to possess specificity against a multiple TAAs by an APC, e.g. a dendritic cell, displaying multiple TAA peptides of interest. In some embodiments, the immune cell is a cytotoxic T cell (e.g., a CD8+ T cell). In some embodiments, the immune cell is a helper T cell, e.g., a CD4+ T cell.

The term “mol %”, as used herein, is defined as the molar amount of a constituent, divided by the total molar amount of all constituents in a mixture, multiplied by 100.

The term “saturated” or “unsaturated”, when referring to a lipid or liposome, means that the lipid or lipid components of the liposome is a saturated or unsaturated compound. A saturated compound has only single bonds between carbon atoms and resists the addition reactions, such as hydrogenation, oxidative addition, and binding of a Lewis base. An unsaturated compound has at least one double bond. A saturated lipid in general has a higher melting temperature than comparable, unsaturated lipid. In some embodiments, saturated lipids are preferred for liposome formulations, to increase entrapment stability of compounds.

The term “PEG”, as used herein, refers to the polyether compound polyethylene glycol. PEG is currently available in several sizes and may e.g. be selected from PEG350, PEG550, PEG750, PEG1000, PEG2000, PEG3000, PEG5000, PEG10000, PEG20000 and PEG30000. The number refers to the molecular weight of the polyethylene glycol.

The term “subject” includes living organisms in which an immune response can be elicited (e.g., mammals, human). In one embodiment, the subject is a patient, e.g., a patient in need of immune cell therapy. In another embodiment, the subject is a donor, e.g. an allogenic donor of immune cells, e.g., intended for allogenic transplantation.

The term “treatment”, as used herein, refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof. In some embodiments, the compositions disclosed herein can be used for prophalactic treatment—i.e, to prevent a disease or condition, or to prevent activating a latent or dormant disease or condition (shingles for example), or to prevent the manifestation of physiological symptoms.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

The term “salt”, as used herein, refers to an ionic compound that is formed by the neutralization reaction of an acid and a base. A salt may be composed of a cation and an anion so that the product is electrically neutral. In one embodiment, the salt is formed by a protonated amine and an anionic counter ion. In one embodiment, the salt is formed by a deprotonated carboxylic acid and a cationic counter ion.

The term “precipitate” refers to a compound or salt thereof that is not completely solved in a solution and instead, forms a solid inside a liposome. In one embodiment, the precipitate is a salt of any one of the compounds disclosed herein and ammonium sulphate in the interior buffer solution within the liposome. In some embodiments, the precipitate can be visualized by electron microscopy as crystalline structures.

The term “aliphatic amine”, as used herein, refers to an amine containing only hydrogen atom and/or alkyl substituents. Thus, in one embodiment, the term “aliphatic amine” refers to an amine which contains no aromatic substituents. The aliphatic amine may contain one, two or three alkyl substituents and thus may be a primary, secondary or tertiary amine.

The term “loading”, as used herein, refers to the incorporation of a TLR7/8 agonist into the interior of the liposome by passage of the TLR7/8 agonist from the exterior buffer solution, over the membrane of the liposome and into the interior of the liposome.

As used herein, a liposome can be “loaded” with active pharmaceutical ingredients such as the TLR agonists disclosed herein. Such a “loaded liposome” can be used as a delivery vehicle to “load” cells with TLR 7/8 agonists. Thus, a “loaded cell” is one that has effectively received, or taken up, a loaded liposome or a TLR 7/8 agonist. In some embodiments, the term “Deep TLR” is used to refer to the loaded liposome.

The term “loading efficiency”, as used herein, refers to the fraction of incorporation of a TLR7/8 agonist into the interior of liposome expressed as a percentage of the total amount of TLR7/8 agonist used in the preparation.

The term “drug-to-lipid ratio”, as used herein, refers to the molar ratio of drug entrapped in the liposome and the lipids forming the liposome. A drug-to-lipid ratio of 0.2 refers to the presence of one mol drug for each 5 moles of lipids, i.e. 1 mol/5 mol=0.2.

The terms “entrapment,” “encapsulation,” “entrapped” and “encapsulated” are used interchangeably herein and refer to the state of a TLR7/8 agonist being loaded into a liposome and retained therein.

The term “entrapment stability”, as used herein, refers to the stability of the loaded TLR7/8 agonist in the liposome in terms of the ability of the TLR7/8 agonist to be retained in the liposome during storage and/or under in vivo conditions.

The term “pKa”, as used herein, refers to the negative logarithm of the disassociation constant for the first ionization of a compound or of the conjugate acid of a compound.

The term “conjugate acid”, as used herein, refers to the protonated form of a given compound. In one embodiment, the conjugate acid is a protonated aliphatic amine, forming an aminium ion.

The term “logD”, as used herein, refers to the partitioning coefficient of a given compound between a water phase and a 1-octanol phase. LogD is given as the logarithm of the ratio of concentrations of the given compound in the water and the 1-octanol phase. LogD is a measure of the difference in solubility of the compound in these two phases. Positive logD values are generally characteristic of hydrophobic compounds, whereas negative logD values indicate a hydrophilic compound.

The term “logD above/below X in the pH range of Y—C”, as used herein, refers to the given compound having a logD above or below the given value X at at least one given pH within the range Y—Z. Therefore, the logD is not necessarily above/below X in the full pH range given. For example, the term “logD above 0 in the pH range of 8-9” refers to the given compound having a logD above 0 at e.g. pH 8, pH 8.5 and/or pH 9. Therefore, the logD is not necessarily above 0 in the full pH range of 8-9.

The term “Cₙ-Cₘ-heteroalkyl”, as used herein, refers to alkyl substituents comprising between n and m carbon atoms and further comprising at least one heteroatom, such as at least one oxygen, nitrogen or sulphur. Thus, “C₁-C₄-heteroalkyl” refers to alkyl substituents comprising between 1 and 4 carbon atoms and further comprising at least one heteroatom, such as at least one oxygen, nitrogen or sulphur. The heteroatom may be positioned within the alkyl-chain, i.e. not being present as an alcohol, primary amine or sulfhydryl group.

The term “Cₙ-Cₘ-alkyl”, as used herein, refers to an alkyl substituent comprising between n and m carbon atoms, Thus, “C₁-C₆-alkyl” refers to an alkyl substituent comprising between 1 and 6 carbon atoms.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The terms “carbocyclyl,” as used herein, refer to a non-aromatic C₃₋₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “substituent” refers to a group “substituted” on, e.g., an alkyl, haloalkyl, cycloalkyl, heterocyclyl, heterocycloalkenyl, cycloalkenyl, aryl, or heteroaryl group at any atom of that group. In one aspect, the substituents) on a group are independently any one single, or any combination of two or more of the permissible atoms or groups of atoms delineated for that substituent, in another aspect, a substituent may itself be substituted with any one of the above substituents. Further, as used herein, the phrase “optionally substituted” means unsubstituted (e.g., substituted with an H) or substituted. As used herein, the term “substituted” means that a hydrogen atom is removed and replaced by a substituent. It is understood that substitution at a given atom is limited by valency.

Compounds of the present disclosure can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

The term “EC₅₀ value”, as used herein, refers to the half maximum effective concentration of a given compound. Thus, the EC₅₀ value refers to the concentration of a given compound providing 50% of the maximal effect.

The term “buffer” or “buffering component”, as used herein, refers to a chemical compound which in an aqueous solution is present as an equilibrium between the acid and the conjugate base and which is capable of keeping the pH of the given solution nearly constant around the pKa of the compound.

The term “basic component”, as used herein, refers to a chemical compound which is capable of accepting a proton. Thus, “basic component” refers to a Brønsted base.

The term “acidic component”, as used herein, refers to a chemical compound which is capable of donating a proton. Thus, “acidic component” refers to a Brønsted acid.

The term “pH or salt gradient” across the liposome membrane, as used herein, refers to a difference in pH or salt concentration between the solution on one side of the liposome membrane and the solution on the other side of the liposome membrane. Thus, the pH or salt concentration of the solution surrounding the liposome and the pH or salt concentration of the solution in the interior of the liposome may be different. In some embodiments, the pH gradient can be about 2-5, about 2-4 or about 2.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions etc.

As used herein, the term “pharmaceutically acceptable salt” means any pharmaceutically acceptable salt of the compounds disclosed herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valences, etc., and to give compounds which are not inherently unstable. For example, any carbon atom will be bonded to two, three, or four other atoms, consistent with the four valence electrons of carbon.

Various aspects of the disclosure are described in further detail below. Additional definitions are set out throughout the specification.

Method of Loading TLR7/8 Agonist into Liposome

In one aspect, the present disclosure provides a method of loading a toll like receptor (TLR)7/8 (TLR7 and/or TLR8) agonist comprising a carboxylic acid or an aliphatic amine into a liposome, the method comprising the steps of
a. providing a liposome in an exterior buffer solution, wherein a pH or salt gradient exists across the liposome membrane,
b. providing a TLR7/8 agonist comprising a carboxylic acid or an aliphatic amine, and
c. combining said liposome in an exterior buffer solution with said TLR7/8 agonist comprising a carboxylic acid or an aliphatic amine, whereby said TLR7/8 agonist is loaded into the liposome.

In one embodiment, a salt of the TLR7/8 agonist is formed in the interior of the liposome upon loading. The salt of the TLR7/8 agonist may precipitate in the interior of the liposome.

In one embodiment, the TLR7/8 agonist comprises an aliphatic amine. The salt may then be formed by protonation of the aliphatic amine of the TLR7/8 agonist by an acidic component in the liposome interior, thus forming a salt comprising the protonated amine and the deprotonated acidic component.

In one embodiment, the acidic component is selected from the group consisting of ammonium sulphate, ammonium phosphate, ammonium citrate, ammonium acetate, citric acid, acetic acid, oxalic acid, tartronic acid, dihydroxymalonic acid, fumaric acid, malic acid, tartaric acid, glutaric acid, phosphoric acid, sodium phosphonate, potassium phosphonate, sulfonic acid, sucrose octasulfonic acid.

Thus, in one embodiment, the interior of the liposome comprises a buffer selected from the group consisting of ammonium sulphate, ammonium phosphate, ammonium citrate, ammonium acetate, sodium citrate, potassium citrate, sodium acetate, potassium acetate, oxalic acid, tartronic acid, dihydroxymalonic acid, fumaric acid, malic acid, tartaric acid, glutaric acid, sodium phosphate, potassium phosphate, Sodium sulfate, potassium sulfate, sucrose octasulfate ammonium salt, sucrose octasulfate sodium salt, sucrose octasulfate potassium salt and sucrose octasulfate aluminium salt. In one embodiment, the pH of said buffer has been adjusted to provide the conjugate acid of the buffering component.

In one embodiment, the TLR7/8 agonist comprises a carboxylic acid. The salt may then be formed by deprotonation of the carboxylic acid of the TLR7/8 agonist by the basic component in the liposome interior, thus forming a salt comprising the deprotonated carboxylic acid and the protonated basic component.

In one embodiment, the basic component is selected from the group consisting of ammonium acetate, potassium acetate, sodium acetate, calcium acetate, ammonium benzoate, potassium benzoate, sodium benzoate and calcium benzoate. The phenyl ring of the benzoate may be substituted with electron donating groups to destabilize the benzoate anion and make the benzoate more basic.

In order for the TLR7/8 agonist to be loaded into the liposome, the properties of the TLR7/8 agonist in the exterior buffer solution comprising the liposome must be such that the TLR7/8 agonist is capable of permeabilizing the membrane of the liposome. Similarly, in order for the TLR7/8 agonist to be retained in the liposome, the properties of the TLR7/8 agonist in the liposome interior must be such that the TLR7/8 agonist has a low membrane permeability. Thus, in one embodiment, the TLR7/8 agonist has a logD above 0 in the exterior buffer solution and a logD below 0 in the interior of the liposome.

In an alternative embodiment, the TLR7/8 agonist has a logD above 0 in the interior of the liposome but forms a precipitate, thereby obtaining a low membrane permeability.

The method of loading a TLR7/8 agonist as described herein may provide improved loading efficiency as compared to passive loading methods known in the art. Thus, in one embodiment, the method provides a loading efficiency of the TLR7/8 agonist of at least 30%, for example at least 40%, such as at least 50%, for example at least 60% such as at least 70%, for example at least 75%, such as at least 80%, for example at least 85%, such as at least 90%, for example at least 95%.

The loading efficiency may be determined as the percentage of TLR7/8 agonist provided in step (b) of the method as described herein which is comprised in the loaded liposome.

In one embodiment, the method provides loading of more than 30%, for example more than 40%, such as more than 50%, for example more than 60% such as more than 70%, for example more than 75%, such as more than 80%, for example more than 85%, such as more than 90%, for example more than 95% of the TLR7/8 agonist provided in step b.

In one embodiment, the present disclosure provides a liposome comprising a TLR7/8 agonist obtainable by the method as described herein.

The pH or salt gradient across the liposome membrane may be as described herein below.

In one embodiment, the present disclosure provides a liposome comprising a carboxylic acid or an aliphatic amine obtainable by the method as described herein.

In one aspect, the present disclosure provides a kit of components suitable for preparation of a liposome according to the present disclosure and/or for performing the method of loading a TLR7/8 agonist into a liposome as described herein.

Thus, in one embodiment, a kit of components is provided, comprising
a. a suspension comprising a liposome in an exterior buffer solution, wherein a pH or salt gradient exist across the liposome membrane, and
b. a TLR7/8 agonist comprising a carboxylic acid or an aliphatic amine.

The pH or salt gradient across the liposome membrane may be as described herein below.

### Liposome Comprising Salt of TLR7/8 Agonist

In one aspect, the present disclosure provides a liposome comprising a salt of a TLR7/8 agonist, wherein the TLR7/8 agonist comprises a carboxylic acid or an aliphatic amine.

In one embodiment, the salt of the TLR7/8 agonist is formed between the TLR7/8 agonist and an acidic component or a basic component in the liposome interior.

In one embodiment, the salt of the TLR7/8 agonist is a precipitate.

In one embodiment, the TLR7/8 agonist comprises an aliphatic amine. The salt may then be formed by protonation of the aliphatic amine of the TLR7/8 agonist by an acidic component in the liposome interior, thus forming a salt comprising the protonated amine and the deprotonated acidic component.

In one embodiment, the acidic component is selected from the group consisting of ammonium sulphate, ammonium phosphate, ammonium citrate, ammonium acetate, citric acid, acetic acid, oxalic acid, tartronic acid, dihydroxymalonic acid, fumaric acid, malic acid, tartaric acid, glutaric acid, phosphoric acid, sodium phosphonate, potassium phosphonate, sulfonic acid, sucrose octasulfonic acid.

In one embodiment, the TLR7/8 agonist comprises a carboxylic acid. The salt may then be formed by deprotonation of the carboxylic acid of the TLR7/8 agonist by the basic component in the liposome interior, thus forming a salt comprising the deprotonated carboxylic acid and the protonated basic component.

In one embodiment, the basic component is selected from the group consisting of ammonium acetate, potassium acetate, sodium acetate, calcium acetate, ammonium benzoate, potassium benzoate, sodium benzoate and calcium benzoate. The phenyl ring of the benzoate may be substituted with electron donating groups to destabilize the benzoate anion and make the benzoate more basic.

In one embodiment, the liposome is comprised in an exterior buffer solution.

In one embodiment, the exterior buffer solution is selected from the list consisting of citric acid, acetic acid, MES, HEPES, TAPS, phosphate, histidine, citrate, Bicine, TRIS, TAPSO, TES, Bis-tris, ADA, ACES, PIPES, MOPSO, BES, TES, DIPSO, MOBS, TAPSO, Trizma, HEPPSO, POPSO, TEA, EPPS, Tricine, Gly-Gly, HEPBS, AMPD, TABS, AMPSO, CHES, CAPSO, AMP and MOPS.

In order for the TLR7/8 agonist to be comprised in the liposome, the properties of the TLR7/8 agonist in the liposome interior must be such that the TLR7/8 agonist has a low membrane permeability. Thus, in one embodiment, the TLR7/8 agonist has a logD above 0 in the exterior buffer solution and a logD below 0 in the interior of the liposome.

In an alternative embodiment, the TLR7/8 agonist has a logD above 0 in the interior of the liposome but forms a precipitate, thereby obtaining low membrane permeability.

### TLR7/8 Agonist Comprising Aliphatic Amine

The method of loading a TLR7/8 agonist as described herein may be performed with a TLR7/8 agonist comprising an aliphatic amine. Also, the kit of components as described herein may comprise a TLR7/8 agonist comprising an aliphatic amine.

Thus, in one embodiment, the present disclosure provides a method of loading a toll like receptor (TLR)7/8 agonist comprising an aliphatic amine into a liposome, the method comprising the steps of
a. providing a liposome in an exterior buffer solution, wherein a pH gradient exist across the liposome membrane,
b. providing a TLR7/8 agonist comprising an aliphatic amine, and
c. combining said liposome in an exterior buffer solution with said TLR7/8 agonist comprising an aliphatic amine,

whereby said TLR7/8 agonist is loaded into the liposome.

In a separate embodiment, a kit of components is provided, comprising
a. a suspension comprising a liposome in an exterior buffer solution, wherein a pH or salt gradient exists across the liposome membrane, and
b. a TLR7/8 agonist comprising an aliphatic amine.

The desired properties of the TLR7/8 agonist comprising an aliphatic amine for loading into a liposome using the method as described herein and for being retained in a liposome formed by said method may be obtained at the below described conditions. Such conditions are also preferred in order for the kit of components being suitable for preparation of a liposome according to the present disclosure and/or for performing the method of loading a TLR7/8 agonist into a liposome as described herein.

In one embodiment, a method or a kit of components or a composition as described herein is provided wherein the interior of the liposome, before loading, has a pH in the range of 4-6.5, such as in the range of 4-6, for example in the range of 4-5.

In one embodiment, a method or a kit of components or a composition as described herein is provided wherein the interior of the liposome, before loading, has a pH in the range of 4-6.5, such as in the range of 4.5-6.5, for example in the range of 5-6.5.

In one embodiment, a method or a kit of components or a composition as described herein is provided wherein the interior of the liposome, before loading, has a pH in the range of 4-6.5, such as in the range of 4.5-6, for example in the range of 5-6.

In one embodiment, a method or a kit of components or a composition as described herein is provided wherein the liposome comprises an acidic component selected from the group consisting of ammonium sulphate, ammonium phosphate, ammonium citrate, ammonium acetate, citric acid, acetic acid, oxalic acid, tartronic acid, dihydroxymalonic acid, fumaric acid, malic acid, tartaric acid, glutaric acid, phosphoric acid, sodium phosphonate, potassium phosphonate, sulfonic acid, sucrose octasulfonic acid.

In one embodiment, a method or a kit of components or a composition as described herein is provided wherein the interior of the liposome comprises ammonium in a non-acidic environment. Such conditions may provide loading of the TLR7/8 agonist comprising an aliphatic amine by protonation of the aliphatic amine once inside the liposome, whereby ammonia is formed. The gaseous ammonia diffuses out of the liposome, thereby driving the equilibrium towards loading of the TLR7/8 agonist.

In one embodiment, the pH gradient across the liposome membrane is generated by addition of an ionophore to a liposome suspension comprising a transmembrane salt gradient. The ionophore will facilitate an outward movement of cations coupled with an inward movement of protons, thereby generating a pH gradient across the liposome membrane.

In one embodiment, a method or a kit of components or a composition as described herein is provided wherein the liposome is comprised in an exterior buffer solution having a pH in the range of 7-9.5, such as in the range of 7.5-9.5, for example in the range of 8-9.5, such as in the range of 8.5-9.5.

In one embodiment, a method or a kit of components or a composition as described herein is provided wherein the liposome is comprised in an exterior buffer solution having a pH in the range of 7-9.5, such as in the range of 7-9, for example in the range of 7-8.5, such as in the range of 7-8.

In one embodiment, a method or a kit of components or a composition as described herein is provided wherein the liposome is comprised in an exterior buffer solution having a pH in the range of 7-9.5, such as in the range of 7.5-9, for example in the range of 7.5-8.5, such as in the range of 7.5-8.

In one embodiment, a method or a kit of components or a composition as described herein is provided wherein the exterior buffer solution comprises a buffering component selected from the group consisting of HEPES, TAPS, phosphate, histidine, citrate, Bicine, TRIS, TAPSO, TES, Bis-tris, ADA, ACES, PIPES, MOPSO, BES, TES, DIPSO, MOBS, TAPSO, Trizma, HEPPSO, POPSO, TEA, EPPS, Tricine, Gly-Gly, HEPBS, AMPD, TABS, AMPSO, CHES, CAPSO, AMP and MOPS. The exterior buffer solution can also include components for obtaining iso-tonicity, i.e. sucrose, glucose, salts or other sugars.

In one embodiment, a method or a kit of components or a composition as described herein is provided wherein the exterior buffer solution comprises a buffering component selected from the group consisting of HEPES, TAPS, phosphate, histidine and citrate.

In yet another embodiment, the liposome as described herein may comprise a salt of a TLR7/8 agonist comprising an aliphatic amine. Thus, in one embodiment, the present disclosure provides a liposome comprising a salt of a TLR7/8 agonist, wherein the TLR7/8 agonist comprises an aliphatic amine.

In one embodiment, a method, a kit of components, a composition or a liposome as described herein is provided wherein the TLR7/8 agonist comprising an aliphatic amine has a logD above 0 in the exterior buffer solution and a logD below 0 in the interior of the liposome.

In one embodiment, a method, a kit of components, a composition or a liposome as described herein is provided wherein the TLR7/8 agonist comprising an aliphatic amine has a logD below 0 in the interior of the liposome, such as below −1, for example below −2, such as below −3, for example below −4 in the interior of the liposome.

In one embodiment, a method, a kit of components, a composition or a liposome as described herein is provided wherein the TLR7/8 agonist comprising an aliphatic amine has a logD above 0 in the exterior buffer solution comprising the liposome, such as above 1, for example above 2 in the exterior buffer solution comprising the liposome.

In one embodiment, a method, a kit of components, a composition or a liposome as described herein is provided wherein the TLR7/8 agonist comprising an aliphatic amine has a pKa of the conjugate acid in the range of about 5.5-10, such as in the range of about 6-10, about 7-10, about 8-10 or about 9-10.

In one embodiment, a method, a kit of components, a composition or a liposome as described herein is provided wherein the TLR7/8 agonist comprising an aliphatic amine has a pKa of the conjugate acid in the range of about 5.5-10, about 5.5-9, about 5.5-8, about 5.5-7.5, or about 5.5-7.

In one embodiment, a method, a kit of components, a composition or a liposome as described herein is provided wherein the TLR7/8 agonist comprising an aliphatic amine has a pKa of the conjugate acid in the range of about 5.5-10, about 6-9, about 6.5-9, about 6.5-8.5, or about 6.5-8.

In one embodiment, a method, a kit of components, a composition or a liposome as described herein is provided wherein the TLR7/8 agonist comprising an aliphatic amine has a logD above 0 in the pH range of about 6-10, such as above about 0.5, above about 1, above about 1.5 or above about 2 in the pH range of about 6-10.

In one embodiment, a method, a kit of components, a composition or a liposome as described herein is provided wherein the TLR7/8 agonist comprising an aliphatic amine has a logD above 0 in the pH range of about 6-10, such as above 0 in the pH range of about 7-10, or above 0 in the pH range of about 8-10.

In one embodiment, a method, a kit of components, a composition or a liposome as described herein is provided wherein the TLR7/8 agonist comprising an aliphatic amine has a logD below 0 in the pH range of about 4-6, such as below about −1, below about −2, below about −3, or below about −4 in the pH range of about 4-6.

In one embodiment, a method, a kit of components, a composition or a liposome as described herein is provided wherein the TLR7/8 agonist comprising an aliphatic amine has a logD below 0 in the pH range of about 4-6, such as below 0 in the pH range of about 4-5.5, or below 0 in the pH range of about 4-5.

In one embodiment, a method, a kit of components, a composition or a liposome as described herein is provided wherein the TLR7/8 agonist comprising an aliphatic amine has a logD below 0 in the pH range of about 4-6, such as below 0 in the pH range of about 4.5-6, or below 0 in the pH range of about 5-6.

In one embodiment, a method, a kit of components, a composition or a liposome as described herein is provided wherein the TLR7/8 agonist comprising an aliphatic amine has a logD below 0 in the pH range of about 4-6, such as below 0 in the pH range of about 4.5-5.5, or below 0 in the pH range of about 5-5.5.

In one embodiment, a method, a kit of components, a composition or a liposome as described herein is provided wherein the TLR7/8 agonist comprising an aliphatic amine has a structure according to formula (I):

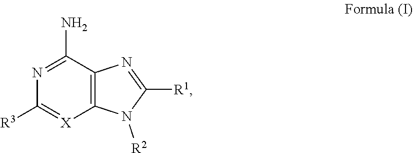

wherein:
X is selected from C or N;
R¹ is OH or —C₁-C₄-heteroalkyl;
R² is selected from the group consisting of —C₁-C₆-alkyl-OH, —C₁-C₆-alkyl-phenyl, —C₁-C₆-alkyl-NH₂ and —C₁-C₆-alkyl-NH-amino acid, wherein the phenyl is optionally substituted with R⁴;
R³ is —C₁-C₆-heteroalkyl or when taken together with X, wherein X is C and to which it is attached, forms an aromatic ring;
R⁴ is —C(O)—NH—C₁-C₄-alkyl-R⁵ or —CH₂—NH—C₁-C₄-alkyl-R⁵; and
R⁵ comprises at least one N and is an optionally substituted heterocycle or aryl,
or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is selected from C or N;
R¹ is selected from the group consisting of OH, —CH₂—O—CH₂—CH₃ and —CH₂—NH—CH₂—CH₃;
R² is selected from the group consisting of —C(CH₃)₂—OH, benzyl, —CH₂—CH₂—NH₂ and —CH₂—CH₂—NH—C(O)—C(R⁶R⁷)—NH₂, wherein the phenyl ring in benzyl is optionally substituted with R⁴;
R³ is —O—CH₂—CH₂—O—CH₃ or when taken together with X, wherein X is C and to which it is attached, forms a benzene ring;
R⁴ is selected from the group consisting of —C(O)—NH—CH₂—CH₂—R⁵, —C(O)—NH—CH₂—R⁵, —CH₂—NH—CH₂—CH₂—R⁵, and —CH₂—NH—CH₂—R⁵;
R⁵ is selected from the group consisting of N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl;
R⁶ is H or —CH₃; and
R⁷ is selected from the group consisting of H, —CH₃, —CH(CH₃)₂, —CH₂—CH(CH₃)₂,
—CH₂—CH₂—S—CH₃ and —CH₂—CH₂—CH₂—CH₂—N(CH₃)₂, or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is N;
R¹ is OH;
R² is benzyl, wherein the phenyl ring is substituted with R⁴;
R³ is —O—CH₂—CH₂—O—CH₃;
R⁴ is selected from the group consisting of —C(O)—NH—CH₂—CH₂—R⁵, —C(O)—NH—CH₂—R⁵, —CH₂—NH—CH₂—CH₂—R⁵ and —CH₂—NH—CH₂—R⁵; and
R⁵ is selected from the group consisting of N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl,
or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is C;
R¹ is —CH₂—O—CH₂—CH₃ or —CH₂—NH—CH₂—CH₃;
R² is selected from the group consisting of —C(CH₃)₂—OH, —CH₂—NH₂ and —CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂,
or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is C;
R¹ is —CH₂—O—CH₂—CH₃ or —CH₂—NH—CH₂—CH₃;
R² is selected from the group consisting of —C(CH₃)₂—OH, —CH₂—NH₂ and —CH₂—NH—C(O)—C(R⁶R⁷)—NH₂;
R³ is taken together with X, to which it is attached, to form a benzene ring;
R⁶ is H or —CH₃; and
R⁷ is —CH₃ or —CH(CH₃)₂,
or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is C;
R¹ is —CH₂—O—CH₂—CH₃;
R² is —CH₂—NH₂ or —CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂,
or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is C;
R¹ is —CH₂—O—CH₂—CH₃;
R² is —CH₂—NH₂ or —CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂ and —CH₂—CH(CH₃)₂, or a pharmaceutically acceptable salt thereof.

In one embodiment, a method, a kit of components, a composition or a liposome as described herein is provided wherein the TLR7/8 agonist comprising an aliphatic amine has a structure according to formula (I), wherein:
X is C;
R¹ is —CH₂—O—CH₂—CH₃;
R² is —CH₂—NH₂ or —CH₂—NH—C(O)—C(R⁶R⁷)—NH₂;
R³ is taken together with X, to which it is attached, to form a benzene ring;
R⁶ is H or —CH₃; and
R⁷ is —CH₃ or —CH(CH₃)₂,
or a pharmaceutically acceptable salt thereof.

In one embodiment, if R⁷ is attached to a stereogenic C, R⁷ has a configuration to form the S-enantiomer.

Thus, in one embodiment, the amino acid comprised in e.g. Formulas (I, C3-4 and C6-8) is an L-amino acid.

In one embodiment, a method, a kit of components, a composition or a liposome as described herein is provided wherein the TLR7/8 agonist comprising an aliphatic amine is selected from the group consisting of:

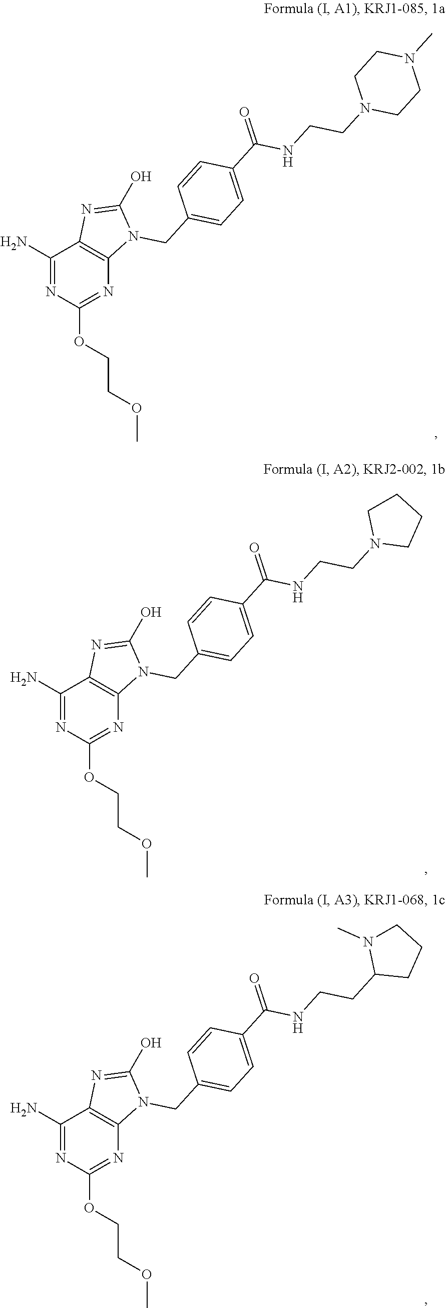 [SMILES: COCCOc1nc(N)c2nc(O)n(Cc3ccc(C(=O)NCCN4CCN(C)CC4)cc3)c2n1 ; COCCOc1nc(N)c2nc(O)n(Cc3ccc(C(=O)NCCN4CCCC4)cc3)c2n1 ; COCCOc1nc(N)c2nc(O)n(Cc3ccc(C(=O)NCCC4CCCN4C)cc3)c2n1]

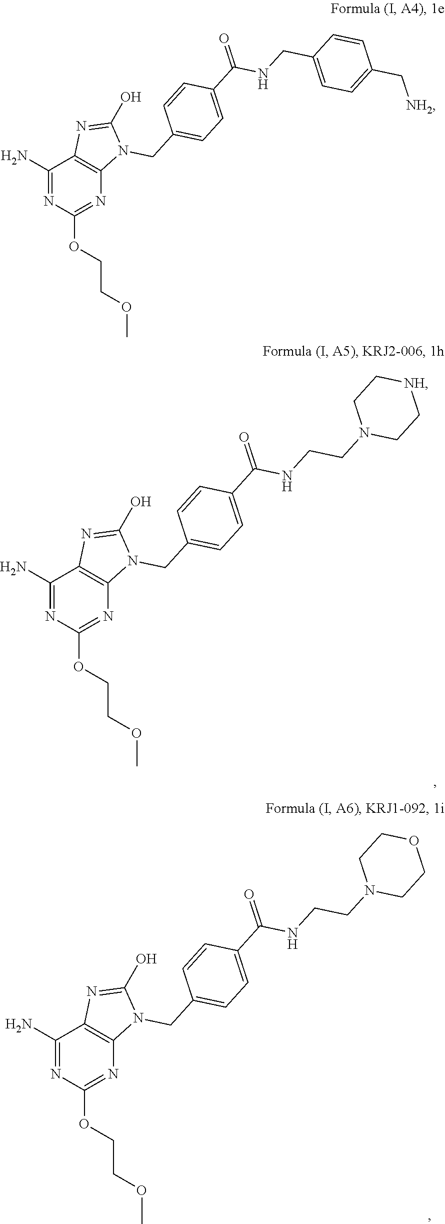 [SMILES: COCCOc1nc(N)c2nc(O)n(Cc3ccc(C(=O)NCc4ccc(CN)cc4)cc3)c2n1 ; COCCOc1nc(N)c2nc(O)n(Cc3ccc(C(=O)NCCN4CCNCC4)cc3)c2n1 ; COCCOc1nc(N)c2nc(O)n(Cc3ccc(C(=O)NCCN4CCOCC4)cc3)c2n1]

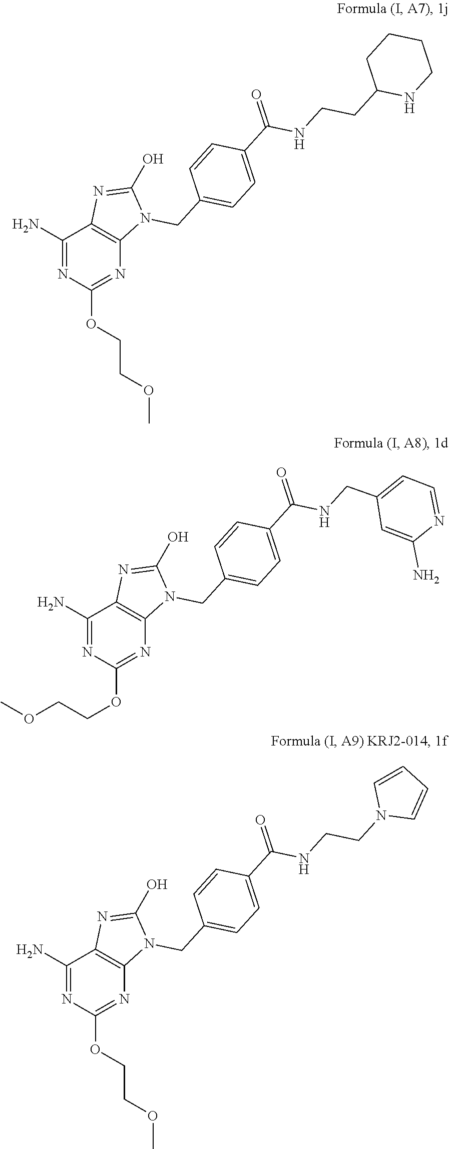 [SMILES: COCCOc1nc(N)c2nc(O)n(Cc3ccc(C(=O)NCCC4CCCCN4)cc3)c2n1 ; COCCOc1nc(N)c2nc(O)n(Cc3ccc(C(=O)NCc4ccnc(N)c4)cc3)c2n1 ; COCCOc1nc(N)c2nc(O)n(Cc3ccc(C(=O)NCCn4cccc4)cc3)c2n1]

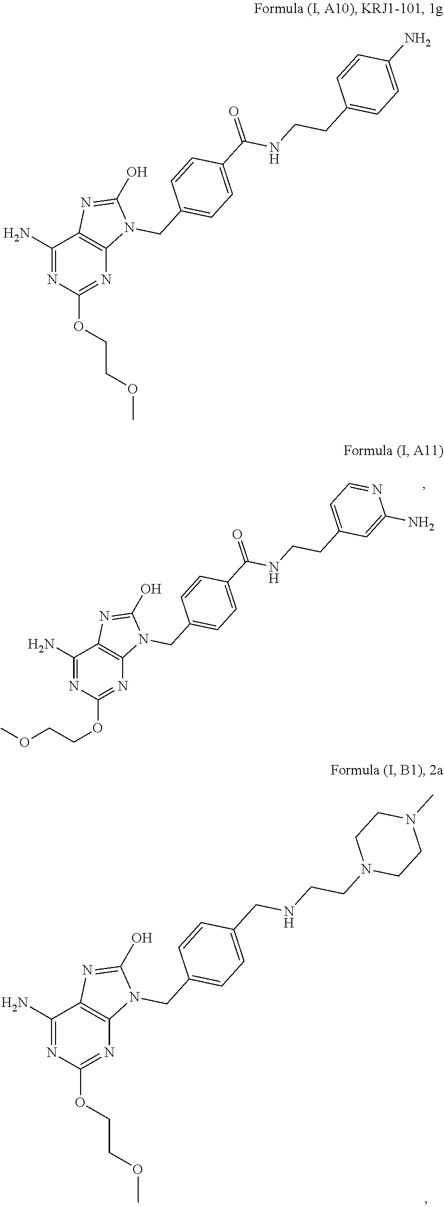 [SMILES: COCCOc1nc(N)c2nc(O)n(Cc3ccc(C(=O)NCCc4ccc(N)cc4)cc3)c2n1 ; COCCOc1nc(N)c2nc(O)n(Cc3ccc(C(=O)NCCc4ccnc(N)c4)cc3)c2n1 ; COCCOc1nc(N)c2nc(O)n(Cc3ccc(CNCCN4CCN(C)CC4)cc3)c2n1]

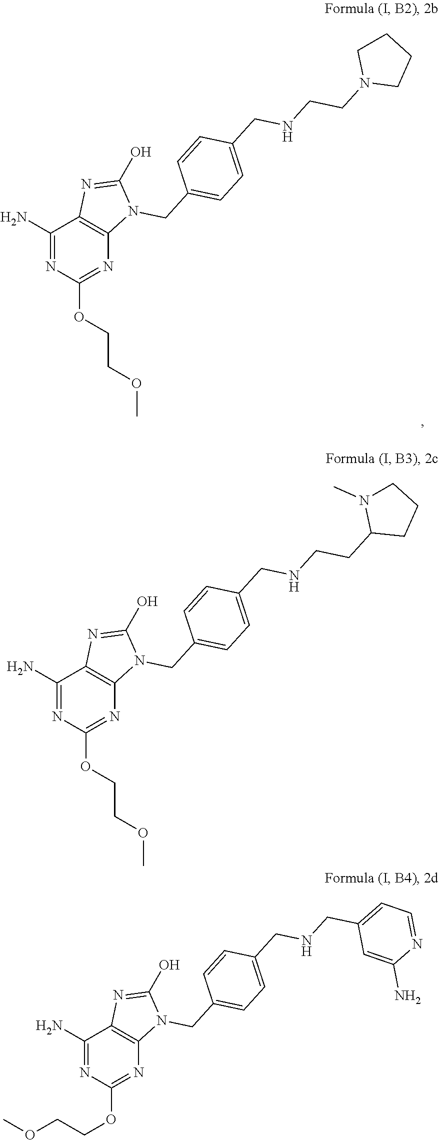 [SMILES: COCCOc1nc(N)c2nc(O)n(Cc3ccc(CNCCN4CCCC4)cc3)c2n1 ; COCCOc1nc(N)c2nc(O)n(Cc3ccc(CNCCC4CCCN4C)cc3)c2n1 ; COCCOc1nc(N)c2nc(O)n(Cc3ccc(CNCc4ccnc(N)c4)cc3)c2n1]

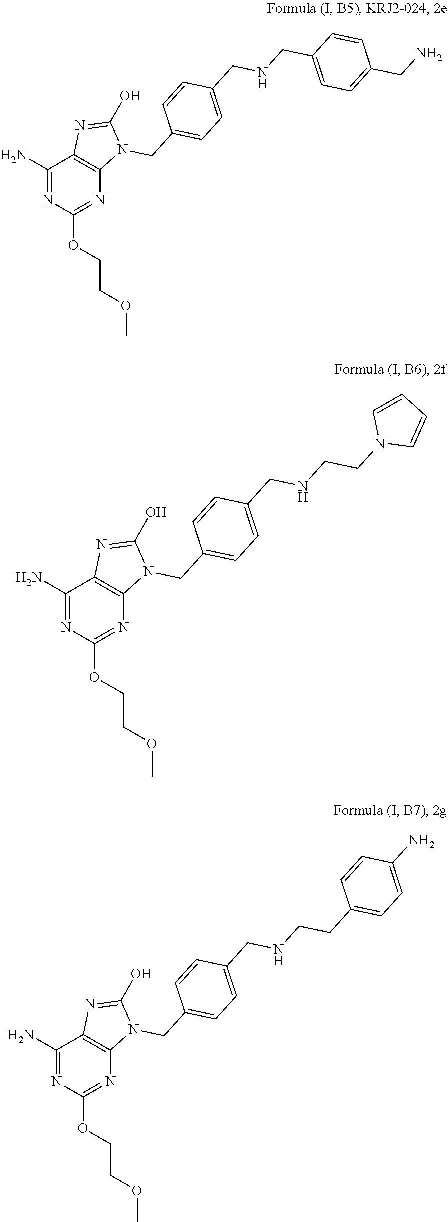 [SMILES: COCCOc1nc(N)c2nc(O)n(Cc3ccc(CNCc4ccc(CN)cc4)cc3)c2n1 ; COCCOc1nc(N)c2nc(O)n(Cc3ccc(CNCCn4cccc4)cc3)c2n1 ; COCCOc1nc(N)c2nc(O)n(Cc3ccc(CNCCc4ccc(N)cc4)cc3)c2n1]

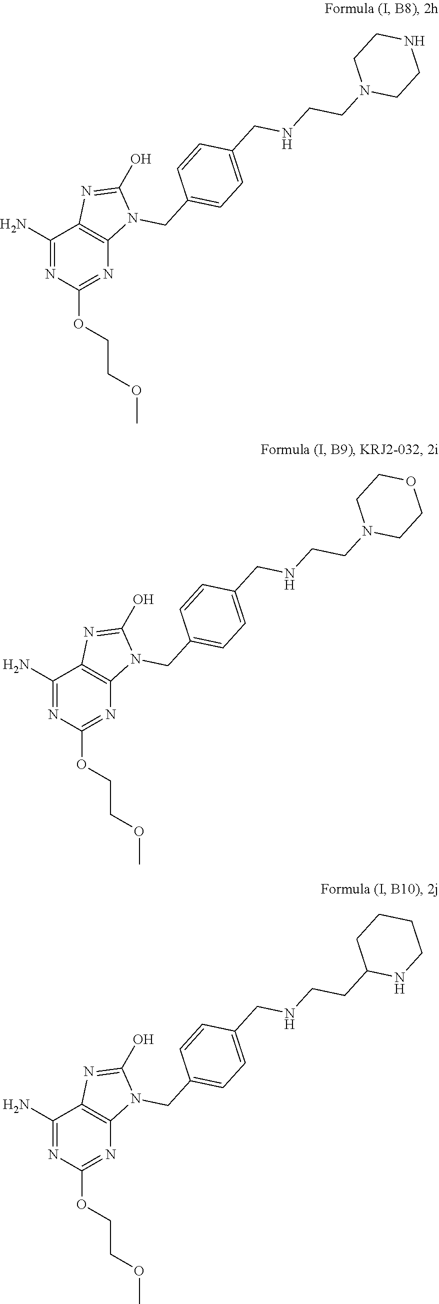 [SMILES: COCCOc1nc(N)c2nc(O)n(Cc3ccc(CNCCN4CCNCC4)cc3)c2n1 ; COCCOc1nc(N)c2nc(O)n(Cc3ccc(CNCCN4CCOCC4)cc3)c2n1 ; COCCOc1nc(N)c2nc(O)n(Cc3ccc(CNCCC4CCCCN4)cc3)c2n1]

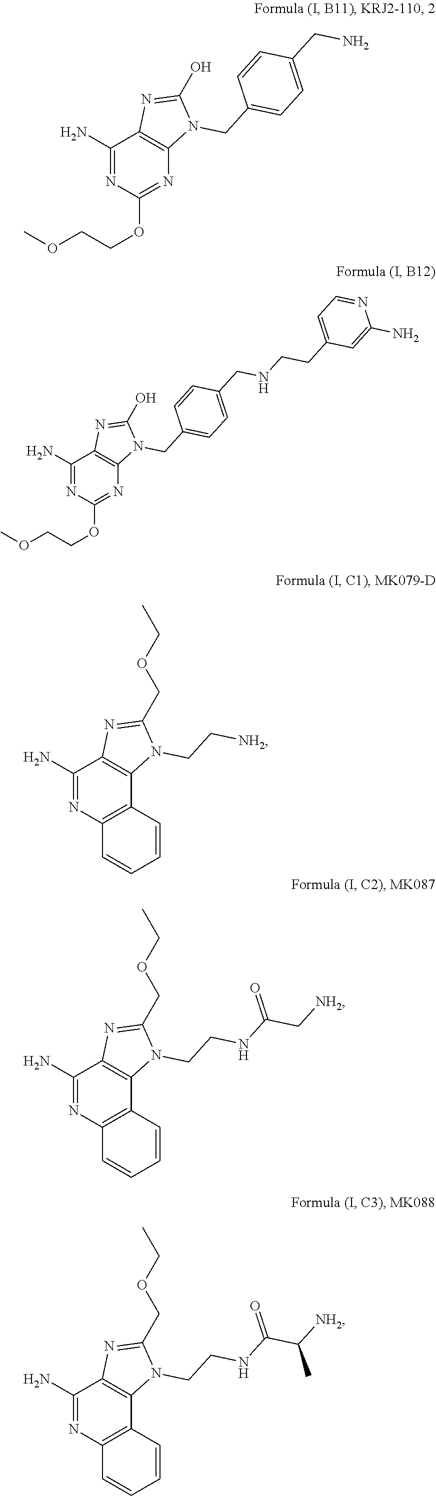

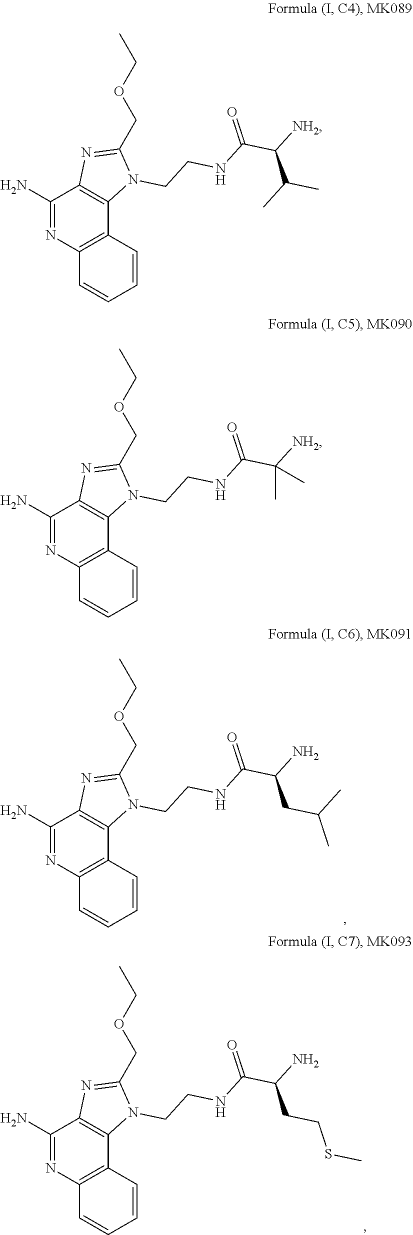

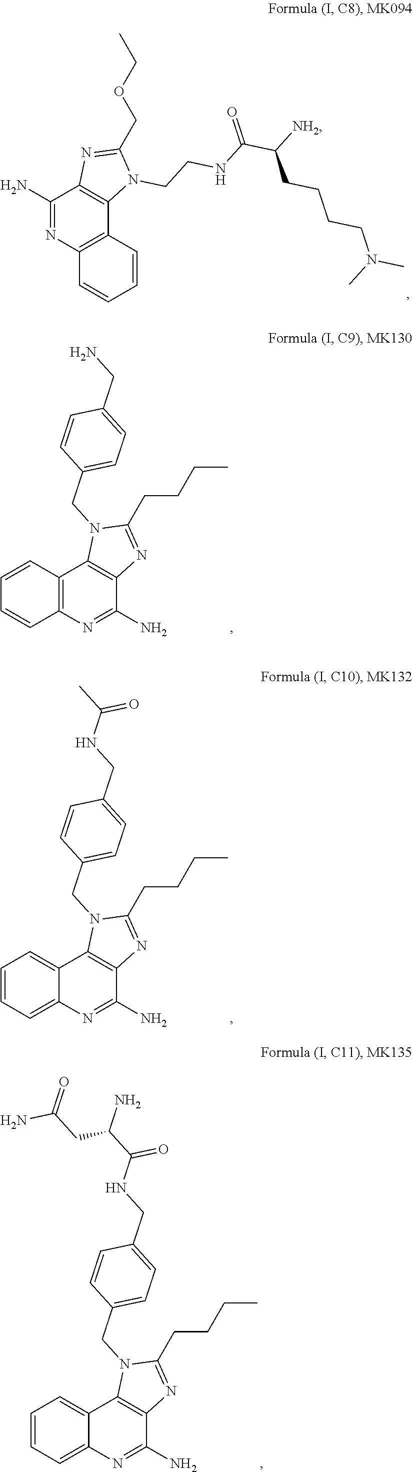 [SMILES: CCOCc1nc2c(N)nc3ccccc3c2n1CCNC(=O)[C@@H](N)CCCCN(C)C ; CCCCc1nc2c(N)nc3ccccc3c2n1Cc1ccc(CN)cc1 ; CCCCc1nc2c(N)nc3ccccc3c2n1Cc1ccc(CNC(C)=O)cc1 ; CCCCc1nc2c(N)nc3ccccc3c2n1Cc1ccc(CNC(=O)[C@@H](N)CC(N)=O)cc1]

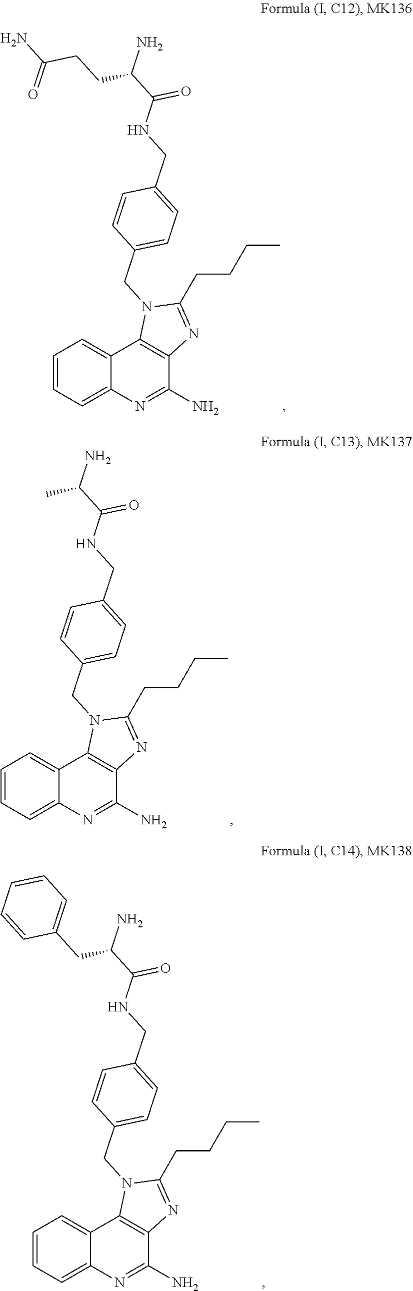 [SMILES: CCCCc1nc2c(N)nc3ccccc3c2n1Cc1ccc(CNC(=O)[C@@H](N)CCC(N)=O)cc1 ; CCCCc1nc2c(N)nc3ccccc3c2n1Cc1ccc(CNC(=O)[C@H](C)N)cc1 ; CCCCc1nc2c(N)nc3ccccc3c2n1Cc1ccc(CNC(=O)[C@@H](N)Cc2ccccc2)cc1]

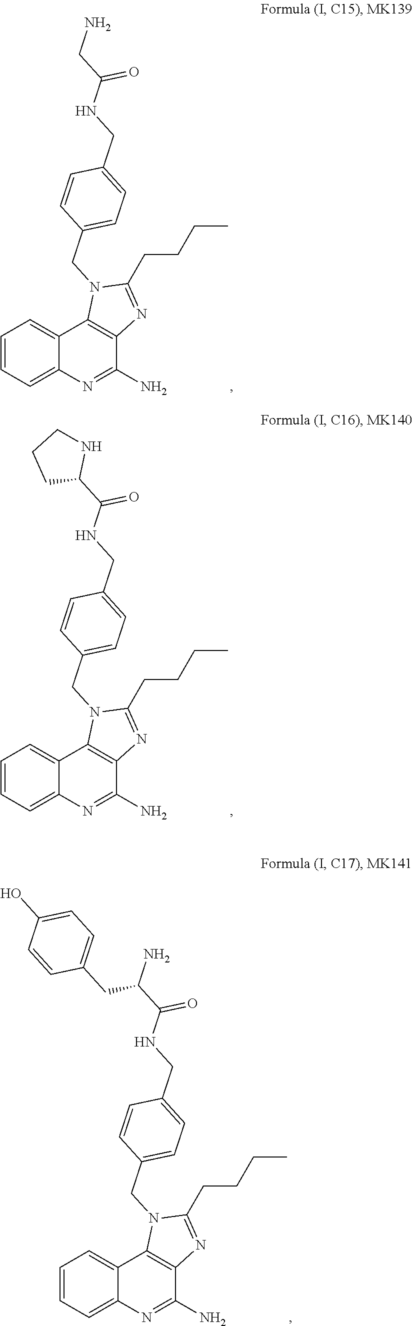 [SMILES: CCCCc1nc2c(N)nc3ccccc3c2n1Cc1ccc(CNC(=O)CN)cc1 ; CCCCc1nc2c(N)nc3ccccc3c2n1Cc1ccc(CNC(=O)[C@@H]2CCCN2)cc1 ; CCCCc1nc2c(N)nc3ccccc3c2n1Cc1ccc(CNC(=O)[C@@H](N)Cc2ccc(O)cc2)cc1]

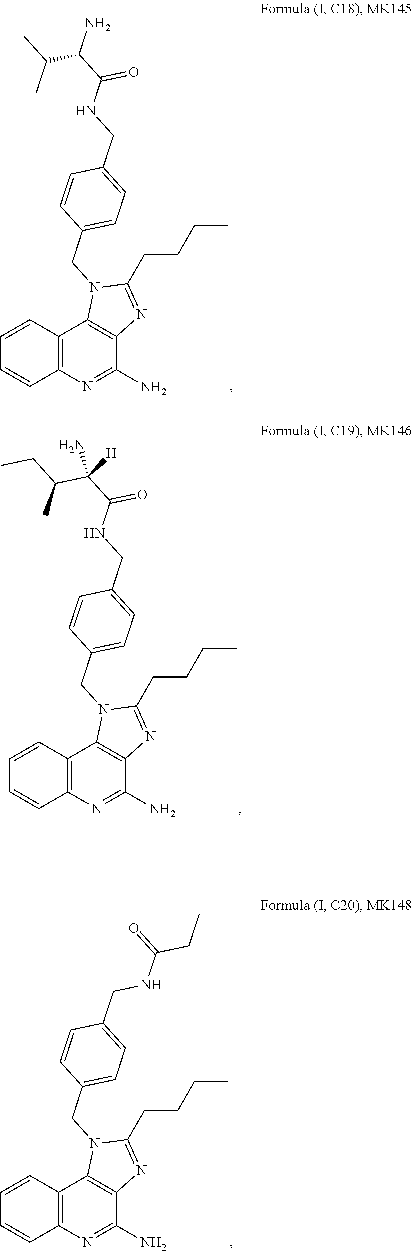

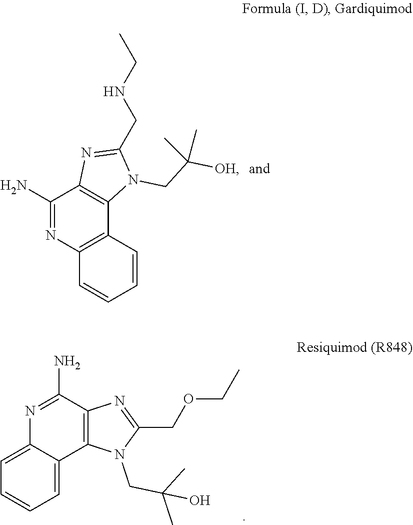 [SMILES: CCNCc1nc2c(N)nc3ccccc3c2n1CC(C)(C)O ; CCOCc1nc2c(N)nc3ccccc3c2n1CC(C)(C)O]

In one embodiment, a method, a kit of components, a composition or a liposome as described herein is provided wherein the TLR7/8 agonist comprising an aliphatic amine is selected from the group consisting of: Formula (I, A1), Formula (I, A2), Formula (I, A3), Formula (I, A4), Formula (I, A5), Formula (I, A6), Formula (I, A7), Formula (I, B1), Formula (I, B2), Formula (I, B3), Formula (I, B4), Formula (I, B5), Formula (I, B6), Formula (I, B7), Formula (I, B8), Formula (I, B9), Formula (I, B10), and Formula (I, B11).

In one embodiment, a method, a kit of components, a composition or a liposome as described herein is provided wherein the TLR7/8 agonist comprising an aliphatic amine is selected from the group consisting of: Formula (I, C1), Formula (I, C2), Formula (I, C3), Formula (I, C4), Formula (I, C5), Formula (I, C6), Formula (I, C7) and Formula (I, C8).

In one embodiment, a method, a kit of components, a composition or a liposome as described herein is provided wherein the TLR7/8 agonist comprising an aliphatic amine is selected from the group consisting of: Formula (I, C1), Formula (I, C2), Formula (I, C3), Formula (I, C4), Formula (I, C5), Formula (I, C6) and Formula (I, D).

In one embodiment, a method, a kit of components, a composition or a liposome as described herein is provided wherein the TLR7/8 agonist comprising an aliphatic amine is selected from the group consisting of: Formula (I, C1), Formula (I, C2), Formula (I, C3), Formula (I, C4), Formula (I, C5) and Formula (I, C6).

In one embodiment, a method, a kit of components, a composition or a liposome as described herein is provided wherein the TLR7/8 agonist comprising an aliphatic amine is selected from the group consisting of: Formula (I, C1), Formula (I, C3), Formula (I, C4), Formula (I, C5), and Formula (I, D).

In one embodiment, a method, a kit of components, a composition or a liposome as described herein is provided wherein the TLR7/8 agonist comprising an aliphatic amine is selected from the group consisting of: Formula (I, C1), Formula (I, C3), Formula (I, C4) and Formula (I, C5).

In one embodiment, a method, a kit of components, a composition or a liposome as described herein is provided wherein the TLR7/8 agonist comprising an aliphatic amine has a structure according to formula (I, D). Compound according to Formula (I, D) is also known in the literature as gardiquimod.

### TLR7/8 Agonist Comprising a Carboxylic Acid

The method of loading a TLR7/8 agonist as described herein may be performed with a TLR7/8 agonist comprising a carboxylic acid. Also, the kit of components as described herein may comprise a salt of a TLR7/8 agonist comprising a carboxylic acid.

Thus, in one embodiment, the present disclosure provides a method of loading a TLR7/8 agonist comprising a carboxylic acid into a liposome, the method comprising the steps of
a. providing a liposome in an exterior buffer solution, wherein a pH or salt gradient exist across the liposome membrane,
b. providing a TLR7/8 agonist comprising a carboxylic acid, and
c. combining said liposome in an exterior buffer solution with said TLR7/8 agonist comprising a carboxylic acid,

whereby said TLR7/8 agonist is loaded into the liposome.

In a separate embodiment, a kit of components is provided, comprising
a. a suspension comprising a liposome in an exterior buffer solution, wherein a pH or salt gradient exist across the liposome membrane, and
b. a TLR7/8 agonist comprising a carboxylic acid.

The desired properties of the TLR7/8 agonist comprising a carboxylic acid for loading into a liposome using the method as described herein and for being retained in a liposome formed by said method may be obtained at the below described conditions. Such conditions are also preferred in order for the kit of components being suitable for preparation of a liposome according to the present disclosure and/or for performing the method of loading a TLR7/8 agonist into a liposome as described herein.

In one embodiment, a method or a kit of components or a composition as described herein is provided wherein the interior of the liposome, before loading, has a pH in the range of 6-9, such as in the range of 7-9, or in the range of 7.5-9, for example in the range of 8-9.

In one embodiment, a method or a kit of components or a composition as described herein is provided wherein the interior of the liposome has a pH in the range of 6-9, such as in the range of 7-8.5, for example in the range of 7-8.

In one embodiment, a method or a kit of components or a composition as described herein is provided wherein the interior of the liposome has a pH in the range of 6-9, such as in the range of 7.5-8.5, for example in the range of 7.5-8.

In one embodiment, a method or a kit of components or a composition as described herein is provided wherein the interior of the liposome comprises a basic component selected from the group consisting of ammonium acetate, potassium acetate, sodium acetate, calcium acetate, ammonium benzoate, potassium benzoate, sodium benzoate and calcium benzoate. The phenyl ring of the benzoate may be substituted with electron donating groups to destabilize the benzoate anion and make the benzoate more basic.

In one embodiment, a method or a kit of components or a composition as described herein is provided wherein the interior of the liposome comprises acetate or benzoate in a non-basic environment. Such conditions may provide loading of the TLR7/8 agonist comprising carboxylic acid by deprotonation of the carboxylic acid once inside the liposome, whereby acetic acid or benzoic acid is formed. Acetic acid and benzoic acid are membrane permeable and diffuses out of the liposome, thereby driving the equilibrium towards loading of the TLR7/8 agonist.

In one embodiment, a method or a kit of components or a composition as described herein is provided wherein the exterior buffer solution has a pH in the range of 2.5-7.5, such as in the range of 2.5-6 or 2.5-5, for example in the range of 2.5-4, such as in the range of 2.5-3.

In one embodiment, a method or a kit of components or a composition as described herein is provided wherein the exterior buffer solution has a pH in the range of 2.5-7.5, such as in the range of 3-6, for example in the range of 4-6.

In one embodiment, a method or a kit of components or a composition as described herein is provided wherein the exterior buffer solution has a pH in the range of 2.5-7.4, such as in the range of 3-5, for example in the range of 3-4.

In one embodiment, a method or a kit of components or a composition as described herein is provided wherein the exterior buffer solution comprises a buffering component selected from the group consisting of citric acid, acetic acid, phosphate, MES, Bis-Tris and ADA.

In yet another embodiment, the liposome as described herein may comprise a salt of a TLR7/8 agonist comprising a carboxylic acid. Thus, in one embodiment, the present disclosure provides a liposome comprising a salt of a TLR7/8 agonist, wherein the TLR7/8 agonist comprises a carboxylic acid.

In one embodiment, a method, a kit of components, a composition or a liposome as described herein is provided wherein the TLR7/8 agonist comprising a carboxylic acid has a logD above 0 in the exterior buffer solution and a logD below 0 in the interior of the liposome.

In one embodiment, a method, a kit of components, a composition or a liposome as described herein is provided wherein the TLR7/8 agonist comprising a carboxylic acid has a logD below 0 in the interior of the liposome, such as below −1, for example below −2, such as below −3, for example below −4 in the interior of the liposome.

In one embodiment, a method, a kit of components, a composition or a liposome as described herein is provided wherein the TLR7/8 agonist comprising a carboxylic acid has a logD above 0 in the exterior buffer solution comprising the liposome, such as above 1, for example above 2 in the exterior buffer solution comprising the liposome.

In one embodiment, a method, a kit of components, a composition or a liposome as described herein is provided wherein the TLR7/8 agonist comprising a carboxylic acid has a pKa in the range of 2-6, for example in the range of 2-5, such as in the range of 2-4, for example in the range of 2-3.

In one embodiment, a method, a kit of components, a composition or a liposome as described herein is provided wherein the TLR7/8 agonist comprising a carboxylic acid has a pKa in the range of 2-6, for example in the range of 3-6, such as in the range of 4-6, for example in the range of 5-6.

In one embodiment, a method, a kit of components, a composition or a liposome as described herein is provided wherein the TLR7/8 agonist comprising a carboxylic acid has a pKa in the range of 2-6, for example in the range of 2.5-5.5, such as in the range of 3.5-4.5, for example in the range of 3-4.

In one embodiment, a method, a kit of components, a composition or a liposome as described herein is provided wherein the TLR7/8 agonist comprising a carboxylic acid has a logD above 0 in the pH range of 2-6, such as above 0.5, for example above 1, such as above 1.5 for example above 2 at pH in the range of 2-6.

In one embodiment, a method, a kit of components, a composition or a liposome as described herein is provided wherein the TLR7/8 agonist comprising a carboxylic acid has a logD above 0 in the pH range of 2-6, such as above 0 at pH in the range of 2-5, for example above 0 at pH in the range of 2-4, such as at a pH in the range of 2-3.

In one embodiment, a method, a kit of components, a composition or a liposome as described herein is provided wherein the TLR7/8 agonist comprising a carboxylic acid has a logD above 0 in the pH range of 2-6, such as above 0 at pH in the range of 3-6, for example above 0 at pH in the range of 4-6, such as at a pH in the range of 5-6.

In one embodiment, a method, a kit of components, a composition or a liposome as described herein is provided wherein the TLR7/8 agonist comprising a carboxylic acid has a logD above 0 in the pH range of 2-6, such as above 0 at pH in the range of 2.5-5.5, for example above 0 at pH in the range of 3.5-4.5, such as at a pH in the range of 3-4. In one embodiment, a method, a kit of components, a composition or a liposome as described herein is provided wherein the TLR7/8 agonist comprising a carboxylic acid has a logD below 0 in the pH range of 6-9, such as below −1, for example below −2, such as below −3, for example below −4 in the pH range of 6-9.

In one embodiment, a method, a kit of components, a composition or a liposome as described herein is provided wherein the TLR7/8 agonist comprising a carboxylic acid has a logD below 0 in the pH range of 6-9, such as below 0 at pH in the range of 7-9, for example below 0 in the pH range of 8-9.

In one embodiment, a method, a kit of components, a composition or a liposome as described herein is provided wherein the TLR7/8 agonist comprising a carboxylic acid has a logD below 0 in the pH range of 6-9, such as below 0 at pH in the range of 6-8, for example below 0 in the pH range of 6-7.

In one embodiment, a method, a kit of components, a composition or a liposome as described herein is provided wherein the TLR7/8 agonist comprising a carboxylic acid has a logD below 0 in the pH range of 6-9, such as below 0 at pH in the range of 6.5-8.5, for example below 0 in the pH range of 7-8.

In some embodiments, the TLR7/8 agonist comprising a carboxylic acid can have formula (I) as disclosed herein.

In one embodiment, a method, a kit of components, a composition or a liposome as described herein is provided wherein the TLR7/8 agonist comprising a carboxylic acid is selected from the group consisting of (m is an integer selected from 0-12, such as 1-12 or 1-6):

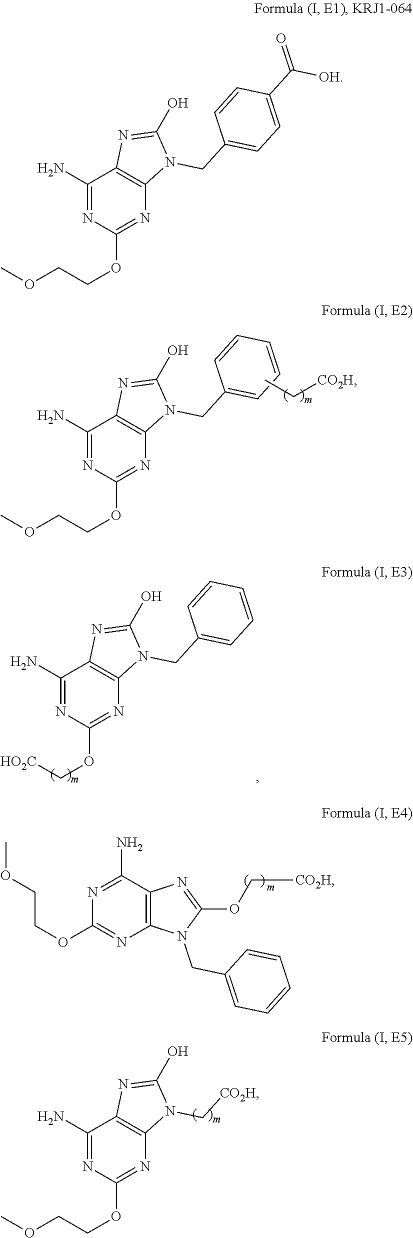

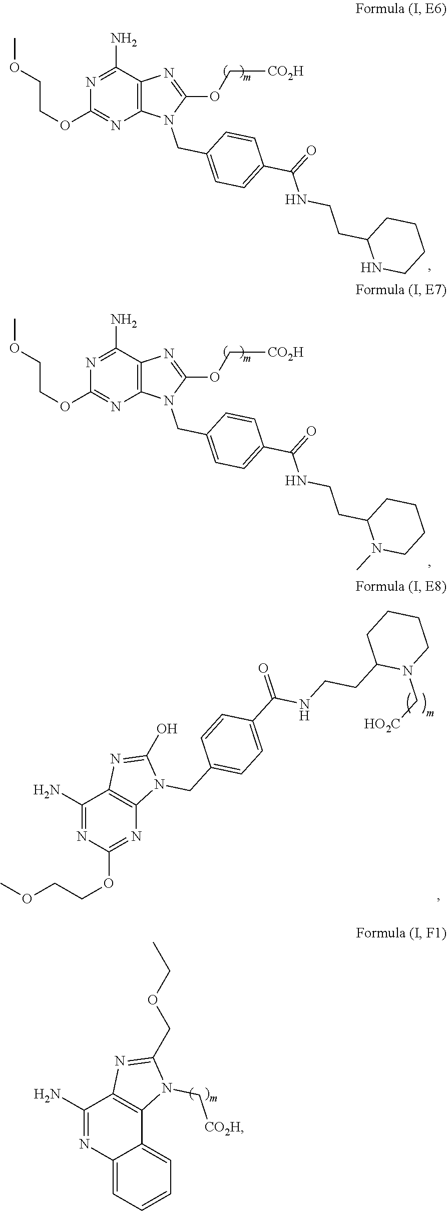

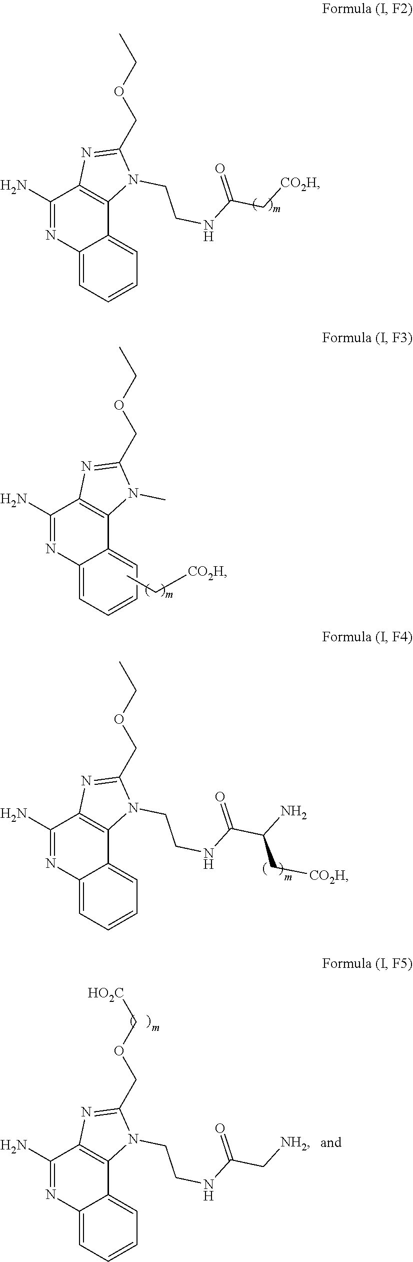

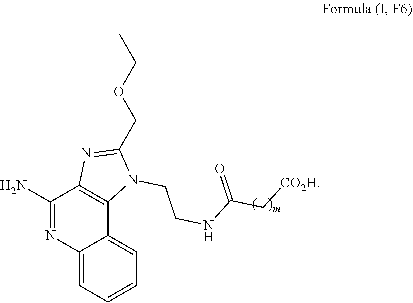

### Biological Activity of TLR7/8 Agonist

The method of the present disclosure, the kit of components of the present disclosure and the liposome of the present disclosure comprise a TLR7/8 agonist which comprises a carboxylic acid or an aliphatic amine.

The liposomes obtainable by the method of the present disclosure and the liposome of the present disclosure may comprise a TLR7/8 agonist comprising a carboxylic acid or an aliphatic amine. The liposomes obtainable by the method of the present disclosure and the liposome of the present disclosure may be suitable for delivery of said TLR7/8 agonists to a desired target.

Toll like receptor 7 (TLR7) and toll like receptor 8 (TLR8) are receptors which plays an important role in pathogen recognition and activation of the innate immune response. TLR7 and TLR8 detect single stranded RNA viruses such as influenza, HIV and HCV. Upon recognition, TLR7 and TLR8 activates transcription factor NF-κB and mediates production of cytokines and chemokines necessary for the development of an immune response.

TLR7/8 agonists are capable of activating the TLR7 and/or TLR8 receptor and are thus immunostimulatory compounds. Thus, the TLR7/8 agonists of the present disclosure may be capable of inducing an immune response. The induced immune response may be evaluated as increased expression of cytokines, such as increased expression of IL-6, IL-12p40 and/or IFNα.

Thus, in one embodiment, the TLR7/8 agonist of the present disclosure induces cytokine expression, such as IL-6, IL-12p40 and/or IFNα expression in a whole blood sample at a concentration of TLR7/8 agonist of about 2 μM, such as at about 3 μM, for example at about 5 μM, such as at about 6 μM, for example at about 7 μM, such as at about 8 μM, for example at about 9 μM, such as at about 10 μM.

In one embodiment, the TLR7/8 agonist of the present disclosure provides increased cytokine levels, such as IL-6, IL-12p40 and/or IFNα levels in a whole blood sample after 24 h treatment with the TLR7/8 agonist at a concentration of about 2 μM, such as at about 3 μM, for example at about 5 μM, such as at about 6 μM, for example at about 7 μM, such as at about 8 μM, for example at about 9 μM, such as at about 10 μM, when compared to an untreated control whole blood sample.

In one embodiment, the TLR7/8 agonist of the present disclosure induces cytokine expression, such as IL-6, IL-12p40 and/or IFNα expression in a PBMC sample at a concentration of TLR7/8 agonist of about 2 μM, such as at about 3 μM, for example at about 5 μM, such as at about 6 μM, for example at about 7 μM, such as at about 8 μM, for example at about 9 μM, such as at about 10 μM.

In one embodiment, the TLR7/8 agonist of the present disclosure provides increased cytokine levels, such as IL-6, IL-12p40 and/or IFNα levels in a PBMC sample after 24 h treatment with the TLR7/8 agonist at a concentration of about 2 μM, such as at about 3 μM, for example at about 5 μM, such as at about 6 μM, for example at about 7 μM, such as at about 8 μM, for example at about 9 μM, such as at about 10 μM, when compared to an untreated control PBMC sample.

The induction of an immune response by the TLR7/8 agonist of the present disclosure may be performed in vivo, in vitro or ex vivo.

Thus, in one embodiment, the induction of an immune response by the TLR7/8 agonist of the present disclosure may be performed in vivo by administering a compound or a liposome as described herein to an individual in need thereof.

In one embodiment, the induction of an immune response by the TLR7/8 agonist of the present disclosure may be performed in vitro by treatment of a whole blood sample with a compound or a liposome as described herein.

In one embodiment, the induction of an immune response by the TLR7/8 agonist of the present disclosure may be performed ex vivo by first treating of a whole blood sample with a compound or a liposome as described herein, and subsequently reintroducing said whole blood sample into an individual in need thereof.

### Liposomes

The liposome of the present disclosure may be any liposome known in the art, such as those disclosed in PCT International Application No. PCT/US2019/032315, incorporated herein by reference in its entirety.

For example, the liposome can include one or more peptide-lipid conjugates. One example of the peptide portion is polyArginine such as (Arginine)₃₋₁₂ (used interchangeably with Arg₃₋₁₂ and R₃₋₁₂), for example R₃ and R₈. The lipid portion of the conjugate can be cholesterol (chol). One example of the conjugate is Cholesterol-R3 (used interchangeably with Arg3-CHOL). Cholesterol-R3 can be used in liposomes at about 0.1-10 mol % or about 1.5-2 mol %. Without wishing to be bound by theory, it is believed that polyArginine increases cell internalization of drugs loaded in liposomes containing polyArginine-lipid conjugates. In addition, the positive charges on polyarginine helps binding of liposomes to cell membrane which is negatively charged. Other cell-penetrating peptides known in the art can also be used.

In some embodiments, the liposome can include one or more antibody-lipid conjugates. The antibody or antigen-binding fragment thereof can be selected to bind to an immune cell surface receptor, such as CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR. In some embodiments, the antibody or antigen-binding fragment thereof can be selected to bind to PD-1, PD-L1, LAG-3, TIM-3, or CTLA-4. The antibody can be conjugated to a lipid such as DSPE-PEG via a maleimide reaction with free thiols after reduction with either TCEP or DTT.

The peptide-lipid conjugates or antibody-lipid conjugates can be co-formulated with other lipids to prepare the liposomes disclosed herein. Alternatively, the peptide-lipid conjugates or antibody-lipid conjugates can be post-inserted into a pre-formed liposome.

The liposome may be composed of synthetic or naturally-occurring amphiphatic compounds which comprises a hydrophilic part and a hydrophobic part. The liposome may be composed of for example, fatty acids, neutral fats, phosphatides, glycolipids, aliphatic alcohols, and steroids.

The liposome of the present disclosure may comprise a hydrophilic polymer such as for example a polyethylene glycol (PEG) component or a derivate thereof, or a polysaccharide. In such a case the liposome is said to be derivatized with the hydrophilic polymer (e.g. PEG) or the polysaccharide.

In one embodiment, the attachment of the polymer (e.g. PEG) to the liposome composition, allows for prolonged circulation time within the blood stream. Vesicles comprising PEG chains on their surface are capable of extravasating leaky blood vessels.

Examples of suitable liposome forming components used in the liposomes of the method, the kit or the liposome of the present disclosure include, but are not limited to: phosphatidylcholines such as 1,2-dioleoyl-phosphatidylcholine, 1,2-dipalmitoyl-phosphatidylcholine, 1,2-dimyristoylphosphatidylcholine, 1,2-distearoyl-phosphatidylcholine, 1-oleoyl-2-palmitoylphosphatidylcholine, 1-oleoyl-2-stearoyl-phosphatidylcholine, 1-palmitoyl-2-oleoyl, phosphatidylcholine and 1-stearoyl-2-oleoyl-phosphatidylcholine; phosphatidylethanolamines such as 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl phosphatidylethanolamine, 1-stearoyl-2-oleoyl-phosphatidylethanolamine and succinyl-dioleoyl-phosphatidylethanolamine; phosphatidylserines such as 1,2-dioleoylphosphatidylserine, 1,2-dipalmitoyl-phosphatidylserine, 1,2-dimyristoylphosphatidylserine, 1,2-distearoyl-phosphatidylserine, 1-oleoyl-2-palmitoylphosphatidylserine, 1-oleoyl-2-stearoyl-phosphatidylserine, 1-palmitoyl-2-oleoylphosphatidylserine and 1-stearoyl-2-oleoyl-phosphatidylserine; phosphatidylglycerol such as 1,2-dioleoyl-phosphatidylglycerol, 1,2-dipalmitoyl-phosphatidylglycerol, 1,2-dimyristoyl-phosphatidylglycerol, 1,2-distearoyl-phosphatidylglycerol, 1-oleoyl-2-palmitoyl-phosphatidylglycerol, 1-oleoyl-2-stearoyl-phosphatidylglycerol, 1-palmitoyl-2-oleoyl-phosphatidylglycerol and 1-stearoyl-2-oleoyl-phosphatidylglycerol; pegylated lipids; pegylated phospoholipids such as phophatidylethanolamine-N-[methoxy(polyethyleneglycol)-1000], phophatidylethanolamine-N-[methoxy(polyethyleneglycol)-2000], phophatidylethanolamine-N-[methoxy(polyethyleneglycol)-3000], phophatidylethanolamine-N-[methoxy(polyethyleneglycol)-5000]; pegylated ceramides such as N-octanoylsphingosine-1-{succinyl[methoxy(polyethyleneglycol)1000]}, N-octanoyl-sphingosine-1-{succinyl[methoxy(polyethyleneglycol)2000]}, N-octanoyl-sphingosine-1-{succinyl[methoxy(polyethyleneglycol)3000]}, N-octanoyl-sphingosine-1-{succinyl[methoxy(polyethyleneglycol)5000]}; lyso-phosphatidylcholines, lysophosphatidylethanolamines, lyso-phosphatidylglycerols, lyso-phosphatidylserines, ceramides; sphingolipids; glycolipids such as ganglioside GM1; glucolipids; sulphatides; phosphatidic acid, such as di-palmitoyl-glycerophosphatidic acid; palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isostearic fatty acids; sterol and sterol derivatives such as cholesterol, cholesterol hemisuccinate, cholesterol sulphate, and cholesteryl-(4-trimethylammonio)-butanoate, ergosterol, lanosterol; polyoxyethylene fatty acids esters and polyoxyethylene fatty acids alcohols; polyoxyethylene fatty acids alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropylene fatty acid polymers; polyoxyethylene fatty acid stearates; d i-oleoyl-sn-glycerol; dipalmitoyl-succinylglycerol; 1,3-dipalmitoyl-2-succinylglycerol; 1-alkyl-2-acyl-phosphatidylcholines such as 1-hexadecyl-2-palmitoylphosphatidylcholine; 1-alkyl-2-acyl-phosphatidylethanolamines such as 1-hexadecyl-2-palmitoyl-phosphatidylethanolamine; 1-alkyl-2-acyl-phosphatidylserines such as 1-hexadecyl-2-palmitoyl-phosphatidylserine; 1-alkyl-2-acyl-phosphatidylglycerols such as 1-hexadecyl-2-palmitoyl-phosphatidylglycerol; 1-alkyl-2-alkyl-phosphatidylcholines such as 1-hexadecyl-2-hexadecyl-phosphatidylcholine; 1-alkyl-2-alkylphosphatidylethanolamines such as 1-hexadecyl-2-hexadecylphosphatidylethanolamine; 1-5 alkyl-2-alkyl-phosphatidylserines such as 1-hexadecyl-2-hexadecyl-phosphatidylserine; 1-alkyl-2-alkyl-phosphatidylglycerols such as 1-hexadecyl-2-hexadecyl-phosphatidylglycerol; N-Succinyl-dioctadecylamine; palmitoylhomocysteine; lauryltrimethylammonium bromide; cetyltrimethyl-ammonium bromide; myristyltrimethylammonium bromide; N-[1,2,3-dioleoyloxy)-propyl]-N,N,Ntrimethylammoniumchloride (DOTMA); 1,2-dioleoyloxy-3 (trimethylammonium)propane (DOTAP); and 1,2-dioleoyl-c-(4′-trimethylammonium)-butanoyl-snglycerol (DOTB).

In one embodiment the liposome forming component include compounds selected from the group consisting of DSPC (1,2-distearoyl-sn-glycero-3-phosphocholine), HSPC (hydrogenated soybean phosphatidylcholine), CHOL (Cholesterol), DSPE-PEG-2000 (1,2-distearoyl-sn-glycero-3-phosphoethanolamine-N-[methoxy(polyethyleneglycol)-2000]), POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG2000 (1,2-Dioleoyl-sn-glycero-3-phosphoethanolamine-N-[methoxy(polyethylene glycol)-2000]).

In one embodiment, the liposome comprises HSPC (hydrogenated soybean phosphatidylcholine), Cholesterol and DSPE-PEG2000 (1,2-distearoyl-sn-glycero-3-phosphoethanolamine-N-[methoxy(polyethylene glycol)-2000]).

In one embodiment, the liposome comprises amphiphatic compounds selected from the group consisting of HSPC “A”, cholesterol “B”, and DSPE-PEG-2000 “C” in the molar ratio of A:B:C, wherein A is selected from the interval 45 to 65, B is selected from the interval 35 to 45, and C is selected from the interval 2 to 20 and wherein A+B+C=100.

In one embodiment the liposome comprises HSPC, Cholesterol, DSPE-PEG-2000 in a molar ratio of about 58:38:5.

In one embodiment, the liposome comprises DSPC (1,2-distearoyl-sn-glycero-3-phosphocholine), Cholesterol and DSPE-PEG2000 (1,2-distearoyl-sn-glycero-3-phosphoethanolamine-N-[methoxy(polyethylene glycol)-2000]).

In one embodiment, the liposome comprises amphiphatic compounds selected from the group consisting of DSPC “A”, cholesterol “B”, and DSPE-PEG-2000 “C” in the molar ratio of A:B:C, wherein A is selected from the interval 55 to 75, B is selected from the interval 25 to 45, and C is selected from the interval 0 to 5 and wherein A+B+C=100.

In one embodiment the liposome comprises DSPC, Cholesterol, DSPE-PEG-2000 in a molar ratio of about 64.5:35:0.5.

In one embodiment, the liposome comprises POPC (1-palmitoyl-2-oleoyl-sn-glycero-3-phosphocholine), Cholesterol and DSPE-PEG2000 (1,2-distearoyl-sn-glycero-3-phosphoethanolamine-N-[methoxy(polyethylene glycol)-2000]).

In one embodiment, the liposome comprises amphiphatic compounds selected from the group consisting of POPC “A”, cholesterol “B”, and DSPE-PEG-2000 “C” in the molar ratio of A:B:C, wherein A is selected from the interval 55 to 75, B is selected from the interval 25 to 45, and C is selected from the interval 0 to 5 and wherein A+B+C=100.

In one embodiment the liposome comprises POPC, Cholesterol, DSPE-PEG-2000 in a molar ratio of about 64.5:35:0.5.

In one embodiment, the liposome is a cationic liposome.

In one embodiment, the liposome comprises a cationic lipid selected from the group consisting of N-[1-(2,3-Dioleoyloxy)propyl]-N,N,N-trimethylammonium (DOTAP), 1,2-di-O-octadecenyl-3-trimethylammonium propane (DOTMA), 1,2-dilauroyl-sn-glycero-3-ethylphosphocholine (Ethyl PC), Dimethyldioctadecylammonium (DDAB), 1,2-dioleyloxy-3-dimethylaminopropane (DODMA) and 1,2-Distearoyl-3-trimethylammonium-propane (DSTAP).

In one embodiment, the liposome comprises POPC (1-palmitoyl-2-oleoyl-sn-glycero-3-phosphocholine), DOTAP (N-[1-(2,3-Dioleoyloxy)propyl]-N,N,N-trimethylammonium), Cholesterol and DOPE-PEG2000 (1,2-Dioleoyl-sn-glycero-3-phosphoethanolamine-N-[methoxy (polyethylene glycol)-2000]).

In one embodiment, the liposome comprises amphiphatic compounds selected from the group consisting of POPC “A”, DOTAP “B”, cholesterol “C” and DOPE-PEG-2000 “D” in the molar ratio of A:B:C:D, wherein A is selected from the interval 30 to 50, B is selected from the interval 20 to 30, C is selected from the interval 15 to 35 and D is selected in the interval 2 to 20 and wherein A+B+C+D=100.

In another preferred embodiment the liposome comprises POPC, DOTAP, Cholesterol and DOPE-PEG2000 in a molar ratio of about 40:30:25:5.

In one embodiment, the liposome comprises POPC (1-palmitoyl-2-oleoyl-sn-glycero-3-phosphocholine), DOTAP (N-[1-(2,3-Dioleoyloxy)propyl]-N,N,N-trimethylammonium), Cholesterol and DSPE-PEG2000 (1,2-distearoyl-sn-glycero-3-phospho ethanolamine-N-[methoxy (polyethylene glycol)-2000]).

In one embodiment, the liposome comprises amphiphatic compounds selected from the group consisting of POPC “A”, DOTAP “B”, cholesterol “C” and DSPE-PEG-2000 “D” in the molar ratio of A:B:C:D, wherein A is selected from the interval 45 to 65, B is selected from the interval 5 to 15, C is selected from the interval 25 to 45 and D is selected in the interval 0 to 5 and wherein A+B+C+D=100.

In another preferred embodiment the liposome comprises POPC, DOTAP, Cholesterol and DSPE-PEG2000 in a molar ratio of about 54.5:10:35:0.5.

In one embodiment, the liposome comprises DSPC (1,2-distearoyl-sn-glycero-3-phosphocholine), DSTAP (1,2-Distearoyl-3-trimethylammonium-propane), Cholesterol and DSPE-PEG2000 (1,2-distearoyl-sn-glycero-3-phosphoethanolamine-N-[methoxy (polyethylene glycol)-2000]).

In one embodiment, the liposome comprises amphiphatic compounds selected from the group consisting of DSPC “A”, DSTAP “B”, cholesterol “C” and DSPE-PEG-2000 “D” in the molar ratio of A:B:C:D, wherein A is selected from the interval 45 to 65, B is selected from the interval 5 to 15, C is selected from the interval 25 to 45 and D is selected in the interval 0 to 5 and wherein A+B+C+D=100.

In another preferred embodiment the liposome comprises DSPC, DSTAP, Cholesterol and DSPE-PEG2000 in a molar ratio of about 54.5:10:35:0.5.

In one embodiment, the size of the liposome is in the range of 100-300 nm, such as in the range of 100-200 nm.

In one embodiment, the seta potential of the liposome is in the range of −10 to 10, such as in the range of −10 to 0, such as in the range of 0 to 10.

### Loading Efficiency and Entrapment Stability

The present disclosure provides a method for loading of a TLR7/8 agonist comprising a carboxylic acid or an aliphatic amine, the method providing improved loading efficiency and improved entrapment stability of the loaded liposome as compared to passive loading methods known in the art. The present disclosure furthermore provides liposomes comprising a salt of a TLR7/8 agonist comprising a carboxylic acid or an aliphatic amine, the liposome having improved stability over liposomes comprising the TLR7/8 agonist not in the form of a salt.

The pH or salt gradient across the liposome membrane drives the loading of the TLR7/8 agonist comprising a carboxylic acid or an aliphatic amine into the liposome. In some embodiments, the TLR7/8 agonist can exist in a neutral or uncharged form in the exterior buffer solution; upon entry in the liposome, an ionized or charged form of the TLR7/8 agonist can be formed due to the different pH inside the liposome than the pH of the exterior buffer solution.

Also provided herein is a liposome composition comprising a liposome and a salt of Gardiquimod and/or one or more of the compounds disclosed herein, wherein the salt is entrapped inside the liposome, wherein the liposome comprises an interior buffer solution. In some embodiments, the compound comprises an aliphatic amine group and the interior buffer solution comprises an acidic component, such that inside the liposome the compound reacts with the acidic component to form the salt. In certain embodiments, the compound comprises a carboxylic acid group and the interior buffer solution comprises a basic component, such that inside the liposome the compound reacts with the basic component to form the salt. Without wishing to be bound by theory, it is believed that the salt provides low membrane permeability of the TLR7/8 agonist and thereby high entrapment stability.

The loading efficiency and/or entrapment stability of the liposome comprising a salt of a TLR7/8 agonist comprising a carboxylic acid or an aliphatic amine may be even higher when the salt of the TLR7/8 agonist is a precipitate. Precipitation of the salt of the TLR7/8 agonist may be obtained by formation of a salt comprising a counterion with good precipitation properties according to the Hofmeister series, such as the counterions selected from the group consisting of: fluoride, sulphate, hydrogen phosphate, acetate, chloride, ammonium, potassium and sodium.

Thus, in one embodiment, a method, a kit of components, a composition or a liposome as described herein is provided wherein the salt of the TLR7/8 agonist comprising a carboxylic acid or an aliphatic amine in the liposome is a precipitate.

The loading efficiency and/or entrapment stability of the liposome comprising a salt of a TLR7/8 agonist comprising a carboxylic acid or an aliphatic amine may be even higher when the ionic strength is higher in the liposome interior than in the exterior buffer solution. Such high ionic strength inside the liposome and low ionic strength outside the liposome promote stability of charged species inside the liposome and non-charged species outside the liposome, which overall facilitate loading. A high ionic strength may be obtained by addition of salts such as sodium chloride, calcium acetate, ammonium sulfate and other salts known in the art to the liposome interior. Low ionic strength buffers may comprise buffered solutions of sugars, such as sucrose.

Thus, in one embodiment, a method, a kit of components, a composition or a liposome as described herein is provided wherein the TLR7/8 agonist is provided wherein the ionic strength is higher in the liposome interior than in the exterior buffer solution.

In one embodiment, the method of the present disclosure provides a loading efficiency of the TLR7/8 agonist of at least 30%, for example at least 40%, such as at least 50%, for example at least 60% such as at least 70%, for example at least 75%, such as at least 80%, for example at least 85%, such as at least 90%, for example at least 95%.

The loading efficiency may be determined as the percentage of TLR7/8 agonist provided in step b. of the method as described herein which is comprised in the loaded liposome.

In one embodiment, the method of the present disclosure provides loading of more than 30%, for example more than 40%, such as more than 50%, for example more than 60% such as more than 70%, for example more than 75%, such as more than 80%, for example more than 85%, such as more than 90%, for example more than 95% of the TLR7/8 agonist provided in step b.

In one embodiment, the present disclosure provides a method or a liposome as described herein, wherein the drug-to-lipid ratio is at least 0.1, for example at least 0.15, such as at least 0.2, for example at least 0.25, such as at least 0.26, for example at least 0.27, such as at least 0.28, for example at least 0.29, such as at least 0.3.

In one embodiment, the present disclosure provides a method or a liposome as described herein, wherein the drug-to-lipid ratio is at least 0.2, for example at least 0.25, such as at least 0.3.

In one embodiment, less than 20%, for example less than 10%, such as less than 5% of the TLR7/8 agonist is released from the liposome after 1 week at 5° C.

In one embodiment, less than 20%, for example less than 10%, such as less than 5% of the TLR7/8 agonist is released from the liposome after 1 month at 5° C.

In one embodiment, a method, a kit of components, a composition or a liposome as described herein is provided wherein the TLR7/8 agonist is provided in and/or combined with the liposome suspension in an organic phase. This may be beneficial for loading of TLR7/8 agonists having low water solubility. The organic phase may be selected from the group consisting of dimethylsulfoxide, dioxane, tetrahydrofuran, dimethylformamide, acetonitrile, dimethylacetamide, sulfolane, gamma butyrolactone, pyrrolidones, 1-methyl-2-pyrrolidinone, methylpyrroline, ethylene glycol monomethyl ether, diethylene glycol monomethyl ether, poly ethylene glycol.

### Compounds According to Formula (I)

Provided herein, in one aspect, is a compound or a pharmaceutically acceptable salt thereof, wherein the compound or salt thereof is substantially purified and wherein the compound has formula (I):

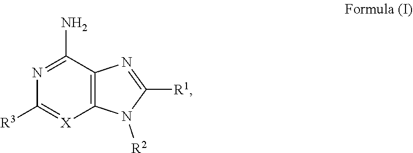

wherein:
X is selected from C or N;
R¹ is selected from the group consisting of OH and R^{A}, wherein R^{A} is optionally substituted with one or more Y;
R² is R^{A} optionally substituted with one or more of: —C₆-C₁₀-aryl (such as phenyl) optionally substituted with one or more R⁴, —C₄-C₁₀-heteroaryl optionally substituted with one or more R⁴, and/or Y;
R³ is (i) R^{A}, or (ii) together with X and the carbon to which it is attached, forms a C₄-C₁₀, preferably —C₅-C₆, aryl or heteroaryl, wherein R³ is optionally substituted with one or more Y;
R⁴ is selected from the group consisting of —C(O)—NH—R^{A}—R⁵, —R^{A}—NH—R^{A}—R⁵, and —R^{A}—NH—R⁵;
R⁵ is selected from the group consisting of H, —C₅-C₁₂-cycloalkyl, —C₄-C₁₂-heterocycloalkyl, —C₆-C₁₀-aryl, and —C₄-C₁₀-heteroaryl, wherein R⁵ is optionally substituted with one or more C₀-C₆-alkyl-Y;
Y is —COOH or —N(R^{B})(R^{C}), wherein (i) R^{B} and R^{C} are each independently selected from the group consisting of H, R^{A}, and (amino acid)ₙ wherein n is an integer selected from 1-6, or (ii) R^{B} and R^{C} together with N form a 3-6-membered ring; and
R^{A}, at each occurrence, is independently selected from the group consisting of —C₁-C₁₂-alkyl, —C₂-C₁₂-alkenyl, —C₂-C₁₂-alkynyl, —C₁-C₁₂-heteroalkyl, —C₂-C₁₂-heteroalkenyl, and —C₂-C₁₂-heteroalkynyl; and
wherein the compound has at least one carboxylic acid group or at least one amine group;
wherein when the compound has at least one carboxylic acid group, the compound has a logD above 0 in the pH range of about 6-10 and/or a logD below 0 in the pH range of about 4-6; and
wherein when the compound has at least one amine group, the compound has a logD above 0 in the pH range of about 2-6 and/or a logD below 0 in the pH range of about 6-9.

In another aspect, the present disclosure provides a compound according to formula (I),

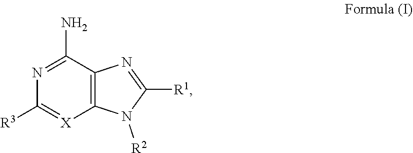

wherein:
X is selected from C or N;
R¹ is OH or —C₁-C₄-heteroalkyl;
R² is selected from the group consisting of —C₁-C₄-alkyl-phenyl, wherein the phenyl ring is substituted with R⁴, —C₁-C₆-alkyl-NH₂ and —C₁-C₆-alkyl-NH-amino acid;
R³ is —C₁-C₆-heteroalkyl or when taken together with X, wherein X is C and to which it is attached, forms an aromatic ring;
R⁴ is —C(O)—NH—C₁-C₄-alkyl-R⁵ or —CH₂—NH—C₁-C₄-alkyl —R⁵; and
R⁵ comprises at least one N and is an optionally substituted heterocycle or aryl, or a pharmaceutically acceptable salt thereof.

In one embodiment, the present disclosure provides a compound according to formula (I), wherein:
X is selected from C or N;
R¹ is selected from the group consisting of OH, —CH₂—O—CH₂—CH₃ and —CH₂—NH—CH₂—CH₃;
R² is selected from the group consisting of benzyl, —CH₂—CH₂—NH₂ and —CH₂—CH₂—NH—C(O)—C(R⁶R⁷)—NH₂, wherein the phenyl ring in benzyl is substituted with R⁴;
R³ is —O—CH₂—CH₂—O—CH₃ or when taken together with X, wherein X is C and to which it is attached, forms a benzene ring;
R⁴ is selected from the group consisting of —C(O)—NH—CH₂—CH₂—R⁵, —C(O)—NH—CH₂—R⁵, —CH₂—NH—CH₂—CH₂—R⁵ and —CH₂—NH—CH₂—R⁵;
R⁵ is selected from the group consisting of N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂, or a pharmaceutically acceptable salt thereof.

In one embodiment, the present disclosure provides a compound according to formula (I), wherein:
X is N;
R¹ is OH;
R² is benzyl, wherein the phenyl ring is substituted with R⁴;
R³ is —O—CH₂—CH₂—O—CH₃;
R⁴ is selected from the group consisting of —C(O)—NH—CH₂—CH₂—R⁵, —C(O)—NH—CH₂—R⁵, —CH₂—NH—CH₂—CH₂—R⁵ or —CH₂—NH—CH₂—R⁵; and
R⁵ is selected from the group consisting of N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl,
or a pharmaceutically acceptable salt thereof.

In one embodiment, the present disclosure provides a compound according to formula (I), wherein:
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂, or a pharmaceutically acceptable salt thereof.

In one embodiment, the present disclosure provides a compound according to formula (I), wherein:
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is —CH₃ or —CH(CH₃)₂,
or a pharmaceutically acceptable salt thereof.

In one embodiment, the present disclosure provides a compound according to formula (I), wherein:
X is C;
R¹ is —CH₂—O—CH₂—CH₃;
R² is —CH₂—NH₂ or —CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂, or a pharmaceutically acceptable salt thereof.

In one embodiment, the present disclosure provides a compound according to formula (I), wherein:
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂ and —CH₂—CH(CH₃)₂,
   or a pharmaceutically acceptable salt thereof.

In one embodiment, the present disclosure provides a compound according to formula (I), wherein:
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is —CH₃ or —CH(CH₃)₂,
   or a pharmaceutically acceptable salt thereof.

In one embodiment, the R⁷ substituent has a configuration to form the S-enantiomer.

Thus, in one embodiment, the amino acid comprised in e.g. Formulas (I, C3-4 and C6-8) is an L-amino acid.

In one embodiment, the present disclosure provides a compound selected from the group consisting of: Formula (I, A1), Formula (I, A2), Formula (I, A3), Formula (I, A4), Formula (I, A5), Formula (I, A6), Formula (I, A7), Formula (I, B1), Formula (I, B2), Formula (I, B3), Formula (I, B4), Formula (I, B5), Formula (I, B6), Formula (I, B7), Formula (I, B8), Formula (I, B9), Formula (I, B10), Formula (I, B11), Formula (I, C1), Formula (I, C2), Formula (I, C3), Formula (I, C4), Formula (I, C5), Formula (I, C6), Formula (I, C7) and Formula (I, C8).

In one embodiment, the present disclosure provides a compound selected from the group consisting of: Formula (I, A1), Formula (I, A2), Formula (I, A3), Formula (I, A4), Formula (I, A5), Formula (I, A6), Formula (I, A7), Formula (I, B1), Formula (I, B2), Formula (I, B3), Formula (I, B4), Formula (I, B5), Formula (I, B6), Formula (I, B7), Formula (I, B8), Formula (I, B9), Formula (I, B10), and Formula (I, B11).

In one embodiment, the present disclosure provides a compound selected from the group consisting of: Formula (I, C1), Formula (I, C2), Formula (I, C3), Formula (I, C4), Formula (I, C5), Formula (I, C6), Formula (I, C7) and Formula (I, C8).

In one embodiment, the present disclosure provides a compound selected from the group consisting of: Formula (I, C1), Formula (I, C2), Formula (I, C3), Formula (I, C4), Formula (I, C5) and Formula (I, C6).

In one embodiment, the present disclosure provides a compound selected from the group consisting of: Formula (I, C1), Formula (I, C3), Formula (I, C4) and Formula (I, C5).

In one embodiment, the compound as described herein is a TLR7/8 agonist. In one embodiment, the compound as described herein is a TLR7 agonist. In one embodiment, the compound as described herein is a TLR8 agonist.

The biological activity of the compound may be as described herein above.

Thus, in one embodiment, the present disclosure provides a TLR7/8 agonist which comprises an aliphatic amine. Said TLR7/8 agonist may be suitable for remote loading into a liposome, such as for example by using the method of the present disclosure as described herein above.

### Pharmaceutical Composition

The compounds as described herein and the liposome of the present disclosure are useful as constituents of a pharmaceutical formulation. Thus, in one embodiment, the present disclosure provides a pharmaceutical composition comprising the compound as described herein and/or the liposome as described herein.

Any form of such formulation which is suitable for administration to a mammal is contemplated.

The pharmaceutical formulation according to the present disclosure is preferably in the form of a solution, dispersion, suspension, lyophilisate, or frozen form.

In one embodiment, the administration route may be intravenous, oral, subcutaneous, intradermal, intramuscular, nasal, intraperitoneal, pulmonary or renal administration.

Compositions, including pharmaceutical compositions, comprising the compounds and liposomes are provided herein. A composition can be formulated in pharmaceutically-acceptable amounts and in pharmaceutically-acceptable compositions. The term “pharmaceutically acceptable” means a non-toxic material that does not interfere with the effectiveness of the biological activity of the active ingredients. Such compositions may, in some embodiments, contain salts, buffering agents, preservatives, and optionally other therapeutic agents. Pharmaceutical compositions also may contain, in some embodiments, suitable preservatives. Pharmaceutical compositions may, in some embodiments, be presented in unit dosage form and may be prepared by any of the methods well-known in the art of pharmacy. Pharmaceutical compositions suitable for parenteral administration, in some embodiments, comprise a sterile preparation of the liposomes and/or cell therapies, which is, in some embodiments, isotonic with the blood of the recipient subject. This preparation may be formulated according to known methods. A sterile injectable preparation also may be a sterile injectable solution or suspension in a non-toxic parenterally-acceptable diluent or solvent.

Additional compositions include modified cells, such as modified immune cells further comprising one or more liposomes on their cell surface. This can be useful for ex vivo preparation of a cell therapy such as an adoptive cell therapy, CAR-T cell therapy, engineered TCR T cell therapy, a tumor infiltrating lymphocyte therapy, an antigen-trained T cell therapy, an enriched antigen-specific T cell therapy, or an NK cell therapy.

In some embodiments, the compounds and liposomes of the present disclosure can be administered directly to a patient in need thereof. Such direct administration can be systemic (e.g., parenteral such as intravenous injection or infusion) or local (e.g., intratumoral, e.g., injection into the tumor microenvironment). The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

In some embodiments, the liposomes of the present disclosure can be used as ex vivo agents to (1) induce maturation of APCs such as dendritic cells; and/or (2) induce activation and expansion of isolated autologous and allogenic cells (e.g., T cells) prior to administration or reintroduction to a patient, via systemic or local administration. For example, the expanded cells can be used in T cell therapies including ACT (adoptive cell transfer) and also with other important immune cell types, including for example, B cells, tumor infiltrating lymphocytes, NK cells, antigen-specific CD8 T cells, T cells genetically engineered to express chimeric antigen receptors (CARs) or CAR-T cells, T cells genetically engineered to express T-cell receptors specific to an tumor antigen, tumor infiltrating lymphocytes (TILs), and/or antigen-trained T cells (e.g., T cells that have been “trained” by antigen presenting cells (APCs) displaying antigens of interest, e.g., tumor associated antigens (TAA)).

Uses and Method of treatment

The compound according to formula (I) as described herein and/or the liposome of the present disclosure may be used in treatment of cancer, an infectious disease, an inflammatory condition or disease, an autoimmune disease or allergy.

In one embodiment, the compound according to formula (I) as described herein and/or the liposome as described herein is used in treatment of cancer.

In one embodiment, the compound according to formula (I) as described herein and/or the liposome as described herein is used in treatment of an infectious disease.

In one embodiment, the present disclosure provides a compound according to formula (I) as described herein and/or the liposome as described and/or the pharmaceutical composition as described herein for use as a medicament.

In one embodiment, the present disclosure provides a compound according to formula (I) as described herein and/or the liposome as described and/or the pharmaceutical composition as described herein for use in the treatment of cancer, an infectious disease, an inflammatory condition or disease and autoimmune disease or allergy.

In one embodiment, the present disclosure relates to the use of a compound according to formula (I) as described herein and/or the liposome as described and/or the pharmaceutical composition as described herein in the manufacture of a medicament for treatment of cancer, an infectious disease, an inflammatory condition or disease and autoimmune disease or allergy.

In one embodiment, the present disclosure provides a method of treatment of cancer, an infectious disease, an inflammatory condition or disease and autoimmune disease or allergy, the method comprising administering a compound according to formula (I) as described herein and/or the liposome as described and/or the pharmaceutical composition as described herein to an individual in need thereof.

In one embodiment, the present disclosure provides a method for stimulating an immune response in an individual in need thereof, the method comprising administering a compound according to formula (I) as described herein and/or the liposome as described and/or the pharmaceutical composition as described herein to said individual in need thereof.

In some embodiments, the liposomes disclosed herein can be used in combination with one or more compositions to treat various conditions such as cancer, infectious disease, an inflammatory condition or disease and autoimmune disease or allergy. For example, two different liposomes comprising two different active pharmaceutical agents (e.g., a TLR agonist and a Shp2 inhibitor) can be used. In another embodiment, a TLR agonist loaded liposome can be used in combination with an antibody such as an anti-IL-12 antibody. In a further embodiment, a TLR agonist loaded liposome can be used in combination with a tethered fusion protein (e.g., IL-15 tethered fusion and IL-12 tethered fusion) such as those disclosed in PCT International Application Nos. PCT/US2018/040777, PCT/US2018/040783 and PCT/US2018/040786, all incorporated herein by reference.

### EXAMPLES

### Materials

Lipids (POPC, DSPC, Cholesterol, DOTAP Chloride, DSTAP Sulfate, DOPE-Peg2000, HSPC, and DSPE-Peg2000) for liposomal formulation were bought from Avanti Polar Lipids, Inc (Alabaster, Ala., USA). All solvents were bought in HPLC quality from Sigma-Aldrich Co. (St. Louis, Mo., USA) and dry solvents were bought with crimped bottle caps with septums (Sure/Seal™). Resiquimod (R848), Gardiquimod, and all other chemicals for syntheses were bought from Sigma-Aldrich Co. as well. IL-6 and IL-12 ELISA kits were bought from RnDSystems (Minneapolis, Minn., USA). Nunc™ 96-Well polystyrene round bottom and conical bottom Microwell™ plates for incubation with whole human blood were bought from Thermo Scientific (Waltham, Mass., USA).

### Methods

HPLC analyses were performed on Gilson HPLC (Gilson Valvemate, UV/VIS-155, 321 Pump, 234 Autoinjector) with Waters XBridge® C₁₈ 5 μm (4.6×150 mm) column at 30° C. Eluent: (A) 5% acetonitrile, 0.1% trifluoroacetic acid (TFA) in water, (B) 0.1% TFA in acetonitrile. Gradient profile; linear gradient from 0% B to 25%, 50%, or 100% B over 15 min indicated by dotted lines in the chromatograms. Flow rate; 1 mL/min. UV wavelengths: 220 nm and 280 nm.

Examples 36-40: HPLC was recorded on an Shimadzu Nexera X2 UHPLC with a Waters XTerra 5 μm C18 column (4.6×150 mm) with UV detection at 254 and 280 nm. MP A: 0.1% TFA, 5% MeCN in H₂O (v/v/v), MP B: 0.1% TFA in MeCN (v/v/v). Flow rate: 1 mL/min. Method A: Gradient: 0-45% B over 10 min, gradient starting at 2 min. Method B: Gradient: 0-60% B over 10 min, gradient starting at 2 min.

UPLCMS analyses were performed on a Waters AQUITY UPLC system with AQUITY UPLC BEH C₈ (1.7 μm, 2.1×50 mm) or C₁₈ (1.7 μm, 2.1×50 mm) column as noted at 40° C. Eluent: (A) 0-1% HCO₂H in water, (B) 0.1% HCO₂H in MeCN. Flow rate; 0.4 mL/min. Gradient profile (LC1): Linear gradient from 5% B to 100% B over 3 min. Gradient profile (LC2): Linear gradient from 5% B to 100% B over 6 min. The instrument was equipped with a QDa electrospray MS detector.

Semi-preparative HPLC was performed on a Waters Semi-Preparative HPLC (Waters Corporation, Milford, Mass.) which was equipped with a Waters 600 Controller & 52 Pump, and a Waters 2489 UV/Visible Detector, and carried out with a Knauer Eurospher 100-5 C₁₈ (250×20 mm) column or a Waters Xterra C₈ (150×10 mm) at room temperature with the same eluent systems as for analytical HPLC.

NMR spectroscopy was carried out on a Bruker Ascend 400 (operating at 400 MHz for proton and 101 MHz for carbon). This spectrometer was used for the recording of 1H-, 13C-, COSY-, HSQC-, and HMBC-NMR spectra. The chemical shifts (δ) are reported in parts per million (ppm) and the coupling constants (J) in Hz. Solvent chemical shifts were used as internal standards (δ¹H/¹³C for DMSO-d6=2.50/39.52 and for chloroform (CDCl₃)=7.26/77.16).

MALDI-TOF MS was performed on Bruker Autoflex TOF/TOF™ (Bruker Daltonics GmbH, Leipzig, Germany) in reflector mode using 19.0 kV/16.7 kV ion acceleration. The spectrum was recorded at a detector voltage of at least 1.872 kV (detector gain 6.0), expressed as the mean of 4000 shots with a frequency of 500 shots/sec. Matrix: 2,5-dihydroxy benzoic acid (DHB) (60 mg/mL), sodium trifluoroacetate (1 mg/mL) in methanol.

Thin layer chromatography (TLC) was carried out on TLC Silica gel 60 F254 coated aluminium sheet by Merck Millipore Corporation, visualized by UV or stained with a cerium-ammonium-molybdate solution (cemol stain).

Size exclusion was performed on disposable desalting PD-10 columns (GE Healthcare Europe GmbH, Brondby) in 25 mM HEPES, 10 vol % sucrose buffer, pH 7.4 with 10×1 mL fraction collection.

Liposomal size, polydispersity, and zeta potential were analyzed by light scattering using a ZetaPals system (Brookhaven Instruments Corporation, NY, USA). Samples were diluted 200-fold to a final concentration of 200 μM (25 mM HEPES, 10 vol % sucrose buffer, pH 7.4), and particle size distribution was determined by five sub runs of 30 s each, and zeta potential was determined by 10 sub runs with a target residual of 0.04.

CryoTEM samples of 3 μL of liposome solution was placed on a lacy carbon 300 mesh copper TEM grid, blotted and plunge frozen in liquid ethane using a FEI Vitrobot Mark IV. Samples were imaged using a FEI Tecnai G2 20 TWIN transmission electron microscope operated at 200 keV in low dose mode with a FEI High-Sensitive (HS) 4 k×4 k Eagle camera.

Phosphor lipid content was determined by ICP-MS (iCAP Q, Thermo Scientific). Samples and standards (25, 50 and 100 ppb P) were prepared in 2% Hydrochloric acid (HCl) containing 10 ppb Ga for internal standard.

LogD calculations were calculated using MarvinSketch 17.27.0 by ChemAxon Ltd. The LogP values were calculated by the ChemAxon method [https://docs.chemaxon.com/display/docs/LogP+and+logD+calculations, Viswanadhan et al. 1989] with the electrolyte concentrations of chloride, sodium, and potassium set to zero. Four distinct pH values 5.5, 7.4, 8.5 and 9.0 were chosen as output.

Compounds prepared and used in the following examples include: Formula (I, A1) (KRJ1-085), Formula (I, A2) (KRJ2-002), Formula (I, A3) (KRJ1-068), Formula (I, A5) (KRJ2-006), Formula (I, A6) (KRJ1-092), Formula (I, B11) (KRJ2-110), Formula (I, C1) (MK079-D), Formula (I, C2) (MK087), Formula (I, C3) (MK088), Formula (I, C4) (MK089), Formula (I, C5) (MK090), Formula (I, C6) (MK091), Formula (I, C7) (MK093), Formula (I, C8) (MK094), Formula (I, C9) (MK130), Formula (I, C10) (MK132), Formula (I, C11) (MK135), Formula (I, C12) (MK136), Formula (I, C13) (MK137), Formula (I, C14) (MK138), Formula (I, C15) (MK139), Formula (I, C16) (MK140), Formula (I, C17) (MK141), Formula (I, C18) (MK145), Formula (I, C19) (MK146), Formula (I, C8) (MK094), Formula (I, C8) (MK094), Formula (I, C8) (MK094), Formula (I, D) Gardiquimod, and Resiquimod (R848).

### Example 1: General Procedure for the Preparation of the Compounds of Set 1 and Set 2

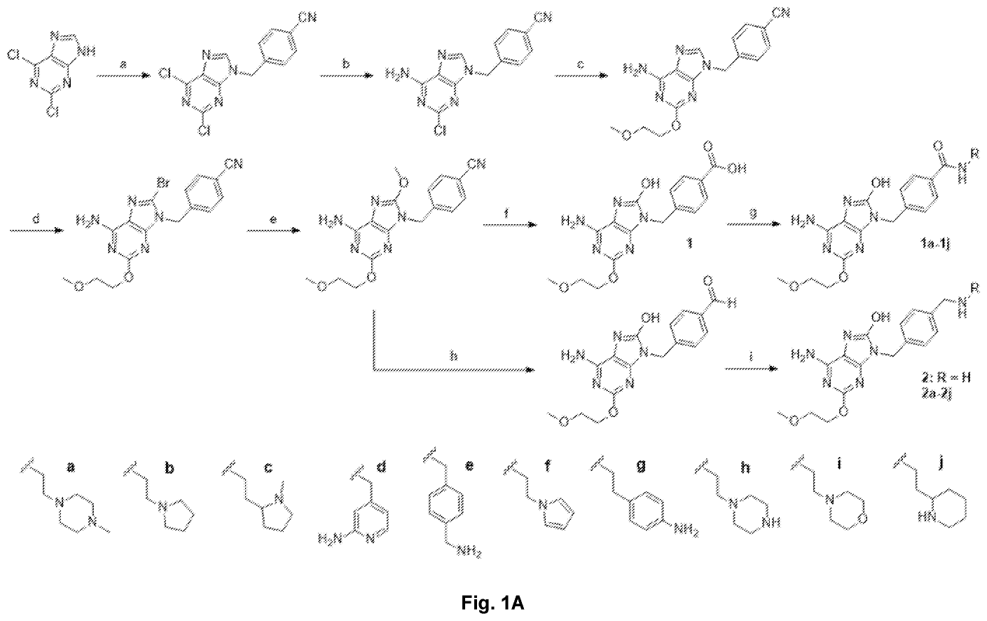

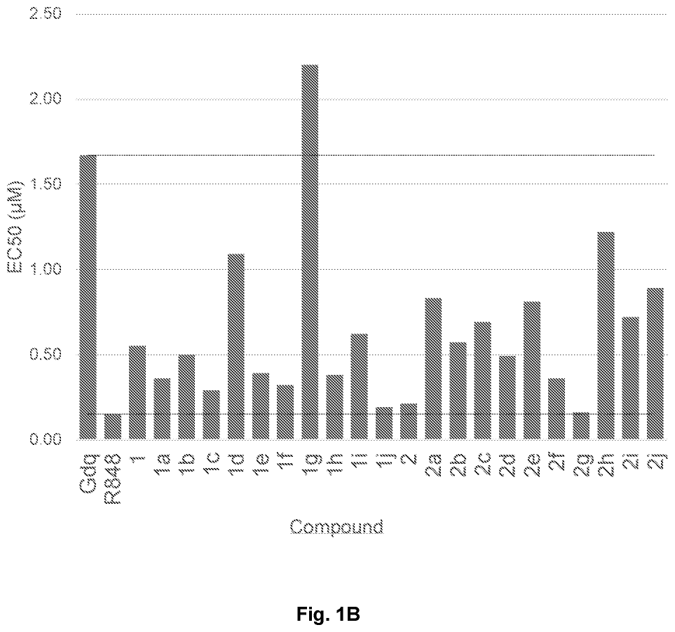

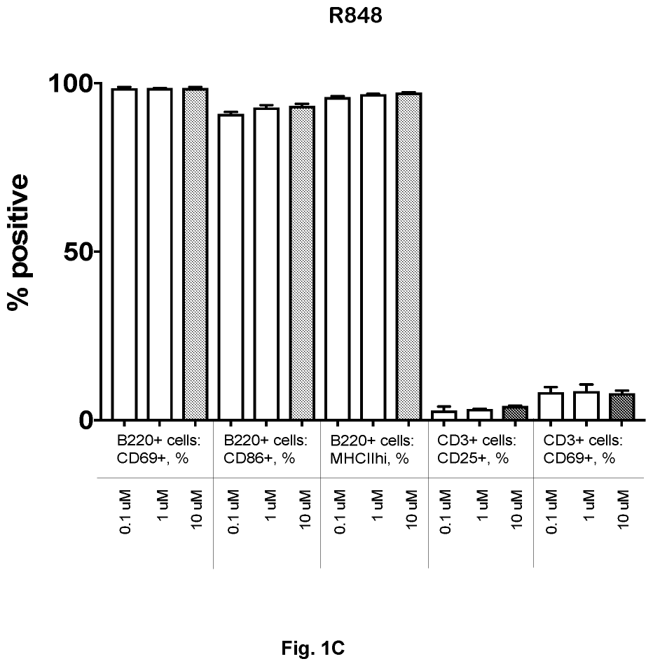

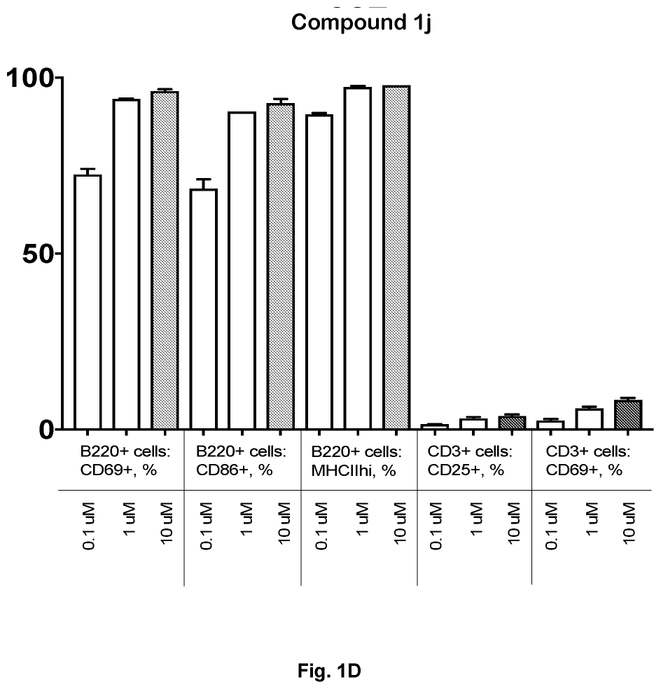

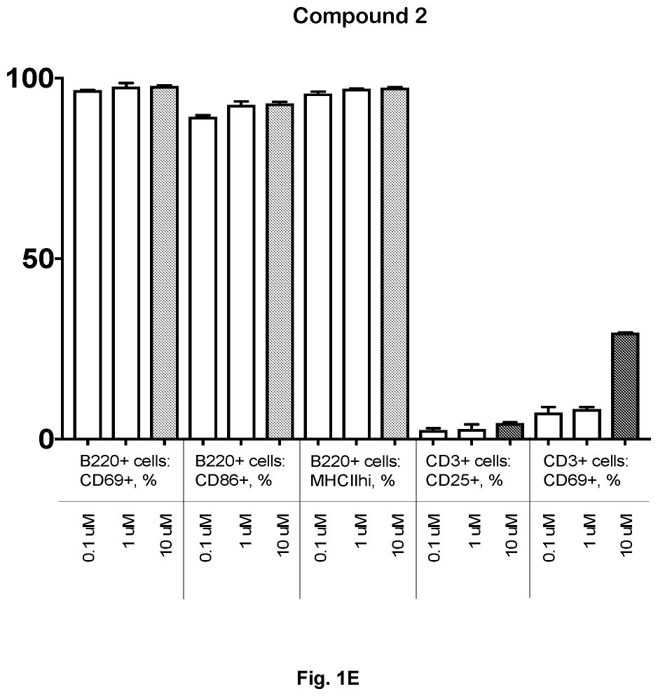

A general synthesis scheme is shown in FIG. 1A according to the procedure by Wu et al. Immunotherapeutic activity of a conjugate of a Toll-like receptor 7 ligand. Proc. Natl. Acad. Sci. 104, 3990-3995 (2007), with some modifications and in good yields. Final derivatization using standard amide bond formation or reductive amination conditions, gave the desired library of 22 compounds divided into two sets (set 1, 1a-1j and set 2, 2a-2j). Next it was evaluated if the synthesized compounds had retained their activity upon derivatization. As benchmarks, the known TLR agonists Gardiquimod (Gdq, TLR7) and Resiquimod (R848, TLR7/8) were used. This was done by determining their potency using RAW Blue cells as TLR reporter cells as shown in FIG. 1B. Cellular screenings showed that most compounds retained their function upon derivatization, being within the benchmark area of Gdq and R848. Furthermore, activation of B and T cells were investigated using mouse splenocytes and human PBMCs as shown in FIGS. 1C-1E. The compounds were found to effectively activate B cells, induce the production of INFγ and maintain the cell viability.

### Example 2: Procedure for the preparation of tert-butyl (2-(4-chloro-2-(ethoxymethyl)-1H-imidazo[4,5-c]quinolin-1-yl)ethyl)carbamate (MK078-C)

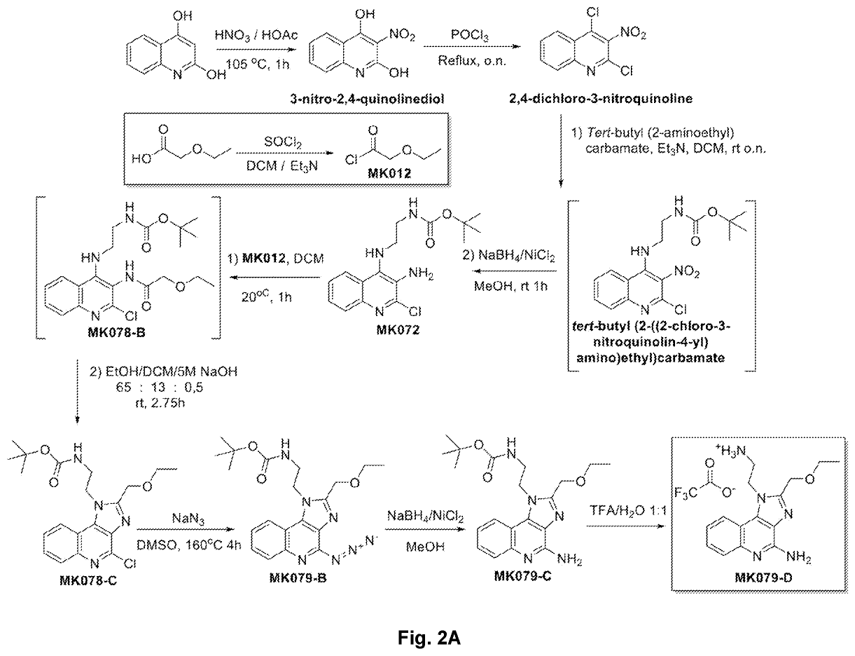

MK078-C was synthesized in the following steps from 2,4-quinolinediol as seen in FIG. 2a with modified procedures from previous reports [Gerster et al. 2005, Shukla et al. 2010, Fujita et al. 2016].

1) 3-Nitro-2,4-quinolinediol

A 25 mL round bottomed flask was fitted with a stir bar and 2,4-quinolinediol (2287 mg, 14.20 mmol), which was dispersed in acetic acid (AcOH) (45 mL, >98%) before nitric acid (HNO₃) (70%, 5.4 mL, 84.78 mmol) was added at room temperature (rt) before the flask was fitted with a condenser. This mixture was then stirred at 105° C. for 1 hour (h), and subsequently cooled in an ice-bath before the precipitate was filtered and dried on oil pump overnight (o.n), which yielded 3-nitro-2,4-quinolinediol as an orange powder, 2434 mg (83%). Rf=0.11 (EtOAc/MeOH 10:1). UPLCMS (LC1, C₁₈): Retention time=0.94 min: Calculated mass for C₉H₆N₂O₄=206.03; Observed m/z [M+H]+=207.1. ¹H-NMR (400 MHz, DMSO-d6) δ 11.97 (s, 1H), 8.03 (dd, J=8.1, 0.9 Hz, 1H), 7.67-7.61 (m, 1H), 7.33 (d, J=8.2 Hz, 1H), 7.30-7.23 (m, 1H); ¹³C-NMR (101 MHz, DMSO-d6) δ 156.39, 155.83, 138.14, 133.14, 27.26, 124.50, 122.34, 115.87, 114.14.

2) 2,4-Dichloro-3-nitroquinoline

A dry 25 mL round bottomed flask fitted with a magnetic stir bar was filled with 3-nitro-2,4-quinolinediol (608 mg, 2.95 mmol) and blown over with nitrogen (N₂) before phosphorus oxychloride (POCl₃) (10 mL, 107.30 mmol) was added. The flask was equipped with a condenser, and this mixture was stirred at 100° C. o.n. Now, the mixture was cooled in an ice-bath before the POCl₃ was quenched in 400 mL ice water. The quenched mixture was stirred in the ice water for 10 min before the precipitate was filtered and dried on oil-pump o.n., which yielded in a brown powder, 669 mg (93%). Rf=0.70 (Dichloromethane (DCM)/Hexane 5:1). UPLCMS (LC1, C₁₈): Retention time=2.04 min: Calculated mass for C₉H₄Cl₂N₂O₂=241.97; Observed m/z [M+H; M+H+2; M+H+4]⁺=243.0; 245.0; 247.1 in approx. ratios 9:6:1. ¹H NMR (400 MHz, DMSO) δ 8.36 (dd, J=8.4, 0.7 Hz, 1H), 8.19 (dd, J=8.4, 0.6 Hz, 1H), 8.13 (ddd, J=8.4, 6.9, 1.3 Hz, 1H), 7.99 (ddd, J=8.3, 6.9, 1.3 Hz, 1H). ¹³C NMR (101 MHz, DMSO) δ 146.16, 138.30, 135.56, 134.40, 130.65, 128.93, 125.39, 124.0.

3) Tert-butyl (2-((3-amino-2-chloroquinolin-4-yl)amino)ethyl)carbamate (MK072) [WO2006/29115]

Modified procedure from Izumi et al. 2003:

a) 2,4-dichloro-3-nitroquinoline (2190 mg, 9.01 mmol) was dissolved in DCM (45 mL) and added triethylamine Et₃N (1900 μL, 13.52 mmol) before tert-butyl (2-aminoethyl)carbamate (1400 μL, 9.01 mmol) was added to the mixture. This mixture was stirred under N₂ for 17 h where TLC (Hexane/Ethyl acetate (EtOAc) 6:1) showed full conversion of 2,4-dichloro-3-nitroquinoline. The mixture was therefore concentrated at reduced pressure, dissolved in EtOAc and water (60 mL each), and the aqueous phase was extracted with 2×60 mL EtOAc before the combined organic phases were dried with Na₂SO₄, filtered, and concentrated at reduced pressure to a yellow/brown solid containing intermediate tert-butyl (2-((2-chloro-3-nitroquinolin-4-yl)amino)ethyl)carbamate. Rf=0.34 (Toluene/EtOAc 10:1). UPLCMS (LC2, C₈): Retention time=3.80 min: Calculated mass for C₁₆H₁₉ClN₄O₄=366.11; Observed m/z [M+H; M+H+2]⁺=367.2; 369.3 in approx. ratios 3:1.

b) Sodium borohydride (NaBH₄) (460 mg, 12.27 mmol) was dissolved in methanol (MeOH) (100 mL) then added nickel chloride (NiCl₂) (561 mg, 4.33 mmol) slowly. This mixture was stirred at rt for 20 min under N₂ before a methanol solution (45 mL) containing intermediate tert-butyl (2-((2-chloro-3-nitroquinolin-4-yl)amino)ethyl)carbamate was added to the mixture. An additional portion of NaBH₄ (440 mg, 11.63 mmol) was added to the mixture after 5 min, TLC showed full conversion of starting material after 30 min stirring at rt. Now water (70 mL) was added to the mixture, which was subsequently filtered through a pad of celite. The methanol was then removed at reduced pressure, and the resulting slurry was added saturated aqueous NaHCO₃ (30 mL) to pH=9, before EtOAc (100 mL) was added to the aqueous solution. The phases were separated, and the aqueous phase was extracted with 2×50 mL EtOAc before the combined organic phases were dried with Na₂SO₄, filtered, and concentrated at reduced pressure on to celite. The crude product was then purified by dry column vacuum chromatography [Pedersen et al. 2001] (Toluene/EtOAc 80:0→56:24 over 12 fractions), which yielded 1386 mg (49%) of tert-butyl (2-((3-amino-2-chloroquinolin-4-yl)amino)ethyl)carbamate (MK072) as a green oil. UPLCMS (LC2, C₈): Retention time=2.65 min: Calculated mass for C₁₆H₂₁ClN₄O₂=336.14; Observed m/z [M+H; M+H+2]⁺=337.3; 339.3 in approx. ratios 3:1. ¹H NMR (400 MHz, DMSO-d6) δ 8.03-7.95 (m, 1H), 7.73-7.64 (m, 1H), 7.46-7.37 (m, 2H), 6.89 (t, J=5.6 Hz, 1H), 5.13 (t, J=6.8 Hz, 1H), 5.05 (s, 2H), 3.26 (q, J=6.7 Hz, 2H), 3.11 (q, J=6.5 Hz, 2H), 1.35 (s, 9H). ¹³C NMR (101 MHz, DMSO) δ 155.7, 140.9, 140.9, 136.1, 128.8, 128.0, 125.7, 125.3, 123.2, 121.8, 77.7, 46.4, 40.8, 28.2.

4) 2-ethoxyacetyl chloride (MK012) was synthesized as previously [U.S. Pat. No. 6,908,939B2] with some modifications:

A flame dried 250 mL round bottomed flask with 120 mL anhydrous DCM was added ethoxyacetic acid (13.0 mL, 137.60 mmol), cooled to 0° C., added Et₃N (19.0 mL, 137.32 mmol), then cooled to −35° C. before SOCl₂ (12.0 mL, 165.13 mmol) was added dropwise with vivid stirring. This mixture was then allowed to reach rt o.n. under N₂ (balloon). The triethylamine hydrochloric salt was then precipitated and removed with filtration, by addition of 50 mL ice-cold Et₂O over four rounds. The combined filtrate was then concentrated at reduced pressure yielding 13335 mg (71%) of product as a brown liquid. ¹H NMR (400 MHz, DMSO) δ 3.96 (s, 2H), 3.48 (q, J=7.0 Hz, 2H), 1.11 (t, J=7.0 Hz, 3H). ¹³C NMR (101 MHz, DMSO) δ 171.7, 67.2, 65.8, 15.0.

5) Tert-butyl (2-(4-chloro-2-(ethoxymethyl)-1H-imidazo[4,5-c]quinolin-1-yl)ethyl)carbamate (MK078-C)

a) MK072 (1362 mg, 4.04 mmol) was dissolved in anhydrous DCM (17 mL) and stirred in a water-bath under N₂ at 20° C., when MK012 (735 uL, 6.07 mmol) was added dropwise. The mixture was then stirred at 20° C. for 1 h, then diluted with DCM (25 mL), washed with saturated aqueous NaHCO₃ (50 mL) that was extracted with 2×25 mL DCM before the organic phases were combined and dried with Na₂SO₄, filtered, and concentrated at reduced pressure yielding intermediate MK078B as a yellow solid. Rf=0.30 (Toluene/EtOAc 2:3). UPLCMS (LC2, C₈): Retention time=2.72 min: Calculated mass for C₂₀H₂₇ClN₄O₄=422.17; Observed m/z [M+H; M+H+2]⁺=423.3; 425.3 in approx. ratios 3:1.

b) The resulting yellow solid was dissolved in ethanol (EtOH (65 mL) and DCM (13 mL) then added 5M NaOH (0.5 mL) before this mixture was stirred at rt for 2.75 h, where TLC showed full conversion of the intermediate. The reaction mixture was then concentrated at reduced pressure to an approximate 10 mL residue that was added DCM (100 mL) and saturated aqueous NaHCO₃ (50 mL). The phases were then separated before the aq phase was extracted with DCM (100 mL). The combined organic phases were dried with Na₂SO₄, filtered, and concentrated at reduced pressure onto celite and was then purified by dry column vacuum chromatography (Tol/EtOAc 5:1→2:3 over 12 fractions). Yielded 1517 mg (93%) of MK078-C as pale solid. Rf=0.29 (Tol/EtOAc 3:2). UPLCMS (LC2, C₈): Retention time=3.16 min: Calculated mass for C₂₀H₂₅ClN₄O₃=404.16; Observed m/z [M+H; M+H+2]⁺=405.3; 407.3 in approx. ratios 3:1. ¹H NMR (400 MHz, DMSO-d6) δ 8.57-8.48 (m, 1H), 8.16-8.03 (m, 1H), 7.77 (ddd, J=8.3, 7.0, 1.5 Hz, 1H), 7.72 (ddd, J=8.4, 7.0, 1.6 Hz, 1H), 7.11 (t, J=6.0 Hz, 1H), 4.82 (s, 2H), 4.77 (t, J=6.2 Hz, 2H), 3.60 (q, J=7.0 Hz, 2H), 3.48 (q, J=6.2 Hz, 2H), 1.28 (s, 9H), 1.18 (t, J=7.0 Hz, 3H). ¹³C NMR (101 MHz, DMSO) δ 155.8, 155.6, 152.0, 143.4, 135.7, 132.6, 129.2, 128.2, 127.1, 121.3, 117.4, 78.1, 65.7, 64.1, 45.5, 39.4, 28.0, 28.0, 28.0, 14.9.

### Example 3: Procedure for the preparation of tert-butyl (2-(4-azido-2-(ethoxymethyl)-1H-imidazo[4,5-c]quinolin-1-yl)ethyl)carbamate (MK079-B)

MK079-B was synthesized from MK078-C as seen in FIG. 2A using the following procedure: Sodium azide (323 mg, 4.97 mmol) dissolved in DMSO (10 mL) was transferred to a flask containing MK078-C (805 mg, 1.99 mmol) before this mixture was stirred at 100° C. for 20 h then at 160° C. for 4 h under a stream of nitrogen. The mixture was then allowed to reach rt before DCM and saturated aqueous NaHCO₃ (50 mL each) were added to the mixture. The aqueous phase was extracted with DCM (50 mL) before the combined organic phases were dried with Na₂SO₄, filtered, and concentrated to a crude residue solid that was purified by column chromatography on silica gel (DCM/MeOH 40:1→30:1). Yielded 751 mg (92%) of MK079-B as a white solid. Rf=0.36 (toluene/EtOAc 1:2). UPLCMS (LC2, C₈): Retention time=3.04 min. Calculated mass for C₂₀H₂₅N₇O₃=411.20; Observed m/z [M+H]⁺=412.4, and [2M+H]⁺=845.5. ¹H NMR (400 MHz, Chloroform-d) δ 8.66 (dt, J=7.4, 3.6 Hz, 1H), 8.35 (dd, J=6.3, 3.4 Hz, 1H), 7.86-7.75 (m, 2H), 5.99 (t, J=5.8 Hz, 1H), 4.87 (s, 2H), 4.81 (t, J=6.1 Hz, 2H), 3.87 (q, J=6.0 Hz, 2H), 3.63 (q, J=7.0 Hz, 2H), 1.47 (s, 9H), 1.24 (t, J=7.0 Hz, 3H). ¹³C NMR (101 MHz, CDCl3) δ 156.4, 152.0, 143.2, 130.0, 129.0, 128.6, 128.4, 125.3, 122.0, 118.4, 115.0, 80.2, 66.9, 64.9, 46.2, 40.5, 28.6, 28.6, 28.6, 15.2. Conclusion: MK079-B was synthesized from MK078-C in a 92% yield.

### Example 4: Procedure for the preparation of tert-butyl (2-(4-amino-2-(ethoxymethyl)-1H-imidazo[4,5-c]quinolin-1-yl)ethyl)carbamate (MK079-C)

MK079-C was synthesized from MK079-B as seen in FIG. 2A using the following procedure: NaBH₄ (244 mg, 6.44 mmol) was dissolved in MeOH (40 mL), then added NiCl₂ (262 mg, 2.02 mmol) slowly. This mixture was then stirred under N₂ for 25 min at rt, then added a solution of MK079-B (751 mg, 1.83 mmol) in MeOH (40 mL). This mixture was then added NaBH₄ (252 mg, 6.66 mmol) and was subsequently allowed to stir at rt o.n. The solution was then filtered through celite, redissolved in EtOAc, and filtered through a 2 cm pad of sand. The filtrate was then concentrated to a crude residue that was purified by column chromatography on silica gel (EtOAc/MeOH 40:1→30:1). Yielded 361 mg (51%) of MKK079-C as a pale white solid. Rf=0.30 (EtOAc/MeOH 60:1). UPLCMS (LC2, C₈): Retention time=1.81 min. Calculated mass for C₂₀H₂₇N₅O₃=385.21; Observed m/z [M+H]⁺=386.3. ¹H NMR (400 MHz, DMSO-d6) δ 9.09 (s, 2H), 8.39 (d, J=8.2 Hz, 1H), 7.82 (dd, J=8.4, 1.2 Hz, 1H), 7.74 (ddd, J=8.3, 7.1, 1.1 Hz, 1H), 7.56 (ddd, J=8.3, 7.1, 1.3 Hz, 1H), 7.13 (t, J=6.0 Hz, 1H), 4.79 (s, 2H), 4.72 (t, J=6.1 Hz, 2H), 3.60 (q, J=7.0 Hz, 2H), 3.47 (q, J=6.1 Hz, 2H), 1.27 (s, 9H), 1.19 (t, J=7.0 Hz, 3H). ¹³C NMR (101 MHz, DMSO) δ 155.7, 152.3, 149.3, 135.9, 134.3, 129.8, 125.0, 124.4, 122.1, 118.6, 112.8, 78.1, 65.8, 63.8, 45.7, 39.3, 28.0, 28.0, 28.0, 14.9. Conclusion: MK079-C was synthesized from MK079-B in a 51% yield.

### Example 5: Procedure for the preparation of 1-(2-aminoethyl)-2-(ethoxymethyl)-1H-imidazo[4,5-c]quinolin-4-amine (MK079-D)

MK079-D was synthesized from MK079-C as seen in FIG. 2A using the following procedure:

MK079-C (361 mg, 0.94 mmol) was dissolved in 60 mL H₂O/TFA 1:1 and was stirred for 1 h before the TFA was removed at reduced pressure. The product was then purified by prep-HPLC on the Knauer EurospherC18 column. Yielded 338 mg (90%) of MK079-C as a white solid trifluoroacetate salt after lyophilization. UPLCMS (LC2, C₈): Retention time=0.55 min. Calculated mass for C₁₅H₁₉N₅O=285.16; Observed m/z [M+H]⁺=286.2. ¹H NMR (400 MHz, DMSO-d₆) δ 14.23 (s, 1H), 9.24 (s, 2H), 8.33 (d, J=8.3 Hz, 1H), 8.25 (s, 3H), 7.84 (d, J=8.3 Hz, 1H), 7.77 (t, J=7.7 Hz, 1H), 7.58 (t, J=7.7 Hz, 1H), 4.93 (t, J=7.5 Hz, 2H), 4.83 (s, 2H), 3.61 (q, J=6.9 Hz, 2H), 1.18 (t, J=6.9 Hz, 3H). ¹³C NMR (101 MHz, DMSO) δ 152.2, 149.5, 135.7, 134.3, 130.1, 125.1, 124.5, 121.9, 118.6, 112.4, 65.8, 63.8, 43.0, 37.9, 14.9. Conclusion: MK079-D was synthesized from MK079-C in a 90% yield.

### Example 6: General Procedure for the Amino Acid Derivatization of MK079-D

A solution of fluorenylmethyloxycarbonyl (Fmoc)-protected L-amino acid (0.1M in DMF, 1.2 equivalents) was mixed with a solution of 1-[bis(dimethylamino)methylene]-1H-1,2,3-triazolo[4,5-b]pyridinium 3-oxide hexafluorophosphate (HATU) (0.1M in DMF, 1.1 equivalents) before a solution of 2,4,6-trimethylpyridine (2M in DMF, 2.1 equivalents) was then added. This mixture was stirred at 20° C. for 15 minutes before a solution of MK079-D (0.1M in DMF, 1.0 equivalent) was added. This mixture was then stirred at 20° C. for 30 minutes before the crude Fmoc-protected intermediate was purified by semi prep-HPLC on the Knauer EurospherC₁₈ column. The fractions containing the Fmoc-protected intermediate were lyophilized to a fluffy white powder that was added an acetonitrile/water/piperidine 40:40:20 solution and was stirred at rt for 45 minutes before the solution was made acidic by slowly adding neat TFA. This mixture was subsequently diluted with 40 mL water and the crude product was purified by semi prep-HPLC on the Knauer EurospherC18 column unless otherwise stated. Fractions with >95% purity (HPLC) were pooled and lyophilized to yield the product as the trifluoroacetic acid salt. The general procedure for the preparation of amino acid derivatization of MK079-D is summarized in FIG. 2B.

This general procedure for the amino acid derivatization of MK079-D was used to generate examples 7-13.

### Example 7: Procedure for the preparation of 2-amino-N-(2-(4-amino-2-(ethoxymethyl)-1H-imidazo[4,5-c]quinolin-1-yl)ethyl)acetamide (MK087)

MK087 was synthesized according to the general procedure: Fmoc-glycine (Fmoc-Gly-OH) (0.045 mmol), HATU (0.041 mmol), 2,4,6-trimethylpyridine (0.079 mmol), and MK079-D (0.0375 mmol). Purification of the Fmoc-protected intermediate was carried out with a linear gradient over 20 min (A/B 50:50→25:75). UPLCMS of intermediate (LC1): Retention time=1.42 min. Calculated mass for C₃₂H₃₂N₆O₄=564.25; Observed m/z [M+H]⁺=564.9. Purification of the product was carried out with a linear gradient over 15 min (A/B 85:15→65:35). Yielded 6.3 mg (37%) as a fluffy white solid. HPLC >95%. ¹H NMR (400 MHz, DMSO-d6) δ 14.05 (s, 1H), 9.08 (s, 2H), 8.77 (t, J=6.0 Hz, 1H), 8.49 (dd, J=8.3, 1.3 Hz, 1H), 8.04 (s, 2H), 7.83 (dd, J=8.4, 1.3 Hz, 1H), 7.80-7.71 (m, 1H), 7.60-7.51 (m, 1H), 4.82 (s, 2H), 4.74 (t, J=7.1 Hz, 2H), 3.69 (q, J=6.7 Hz, 2H), 3.60 (q, J=7.0 Hz, 2H), 3.48 (s, 2H), 1.19 (t, J=7.0 Hz, 3H). ¹³C NMR (101 MHz, DMSO-d6) δ 166.9, 152.1, 149.4, 135.6, 134.9, 129.9, 124.9, 124.5, 121.9, 119.0, 112.7, 65.8, 63.9, 44.9, 38.9, 38.3, 15.0. Conclusion: MK087 was synthesized as a glycine derivatization of MK079-D in a 37% yield over two steps.

### Example 8: Procedure for the preparation of (S)-2-amino-N-(2-(4-amino-2-(ethoxymethyl)-1H-imidazo[4,5-c]quinolin-1-yl)ethyl)propanamide (MK088)

MK088 was synthesized according to the general procedure: Fmoc-L-alanine (Fmoc-Ala-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 100:0→40:60). UPLCMS of intermediate (LC1, C₁₈): Retention time=1.66 min. Calculated mass for C₃₃H₃₄N₆O₄=578.26; Observed m/z [M+H]⁺=578.9. Purification of the product was carried out with a linear gradient over 15 min (A/B 85:15→65:35). Yielded 15.3 mg (65%) as a fluffy white solid. HPLC >95%. ¹H NMR (400 MHz, DMSO-d6) δ 14.21 (s, 1H), 9.18 (s, 2H), 8.81 (t, J=6.0 Hz, 1H), 8.50 (dd, J=8.4, 1.2 Hz, 1H), 8.09 (s, 2H), 7.82 (dd, J=8.4, 1.2 Hz, 1H), 7.75 (ddd, J=8.3, 7.0, 1.1 Hz, 1H), 7.56 (ddd, J=8.3, 7.0, 1.3 Hz, 1H), 4.87-4.78 (m, 2H), 4.77-4.68 (m, 2H), 3.85-3.72 (m, 2H), 3.67-3.55 (m, 3H), 1.22-1.13 (m, 6H). ¹³C NMR (101 MHz, DMSO-d6) δ 170.3, 152.2, 149.4, 135.8, 134.5, 129.9, 124.9, 124.5, 122.1, 118.8, 112.7, 65.8, 63.9, 48.2, 44.8, 38.2, 16.7, 14.9. Conclusion: MK088 was synthesized as an alanine derivatization of MK079-D in a 65% yield over two steps.

### Example 9: Procedure for the preparation of (S)-2-amino-N-(2-(4-amino-2-(ethoxymethyl)-1H-imidazo[4,5-c]quinolin-1-yl)ethyl)-3-methylbutanamide (MK089)

MK089 was synthesized according to the general procedure: Fmoc-L-valine (Fmoc-Val-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 90:10→30:70). UPLCMS of intermediate (LC1, C₁₈): Retention time=1.85 min. Calculated mass C₃₅H₃₈N₆O₄=606.30; Observed m/z [M+H]⁺=607.0. Purification of the product was carried out with a linear gradient over 15 min (A/B 100:0→80:20) on a Waters XterraC18 column. Yielded 17.9 mg (72%) as a fluffy white solid. HPLC >95%. ¹H NMR (400 MHz, DMSO-d6) δ 14.15 (s, 1H), 9.15 (s, 2H), 8.92 (t, J=5.9 Hz, 1H), 8.53 (dd, J=8.4, 1.2 Hz, 1H), 8.13 (s, 2H), 7.83 (dd, J=8.4, 1.2 Hz, 1H), 7.76 (ddd, J=8.4, 7.1, 1.1 Hz, 1H), 7.56 (ddd, J=8.3, 7.1, 1.3 Hz, 1H), 4.91-4.78 (m, 2H), 4.72 (dq, J=15.0, 7.2, 6.6 Hz, 2H), 3.86-3.73 (m, 1H), 3.68-3.56 (m, 3H), 2.06-1.94 (m, 1H), 1.19 (t, J=7.0 Hz, 3H), 0.91 (d, J=6.9 Hz, 3H), 0.83 (d, J=6.8 Hz, 3H). ¹³C NMR (101 MHz, DMSO) δ 169.0, 152.1, 149.4, 135.7, 134.6, 130.0, 125.0, 124.4, 122.0, 118.9, 112.7, 65.8, 63.9, 57.6, 44.6, 38.1, 29.6, 18.4, 17.3, 14.9. Conclusion: MK089 was synthesized as a valine derivatization of MK079-D in a 72% yield over two steps.

### Example 10: Procedure for the preparation of 2-amino-N-(2-(4-amino-2-(ethoxymethyl)-1H-imidazo[4,5-c]quinolin-1-yl)ethyl)-2-methylpropanamide (MK090)

MK090 was synthesized according to the general procedure: N-α-Fmoc-α-aminoisobutyric acid (Fmoc-Aib-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 80:20→10:90). UPLCMS of intermediate (LC1, C₁₈): Retention time=1.77 min. Calculated mass C₃₄H₃₆N₆O₄=592.28; Observed m/z [M+H]⁺=593.0. Purification of the product was carried out with a linear gradient over 15 min (A/B 100:0→80:20) on a Waters XterraC18 column. Yielded 14.1 mg (58%) as a fluffy white solid. HPLC >95%. ¹H NMR (400 MHz, DMSO-d6) δ 14.31 (s, 1H), 9.21 (s, 2H), 8.66 (t, J=6.0 Hz, 1H), 8.49 (dd, J=8.3, 1.3 Hz, 1H), 8.14 (s, 2H), 7.82 (dd, J=8.4, 1.3 Hz, 1H), 7.76 (ddd, J=8.3, 7.0, 1.1 Hz, 1H), 7.57 (ddd, J=8.4, 7.1, 1.3 Hz, 1H), 4.82 (s, 2H), 4.77 (t, J=6.7 Hz, 2H), 3.72 (q, J=6.5 Hz, 2H), 3.60 (q, J=7.0 Hz, 2H), 1.31 (s, 6H), 1.19 (t, J=7.0 Hz, 3H). ¹³C NMR (101 MHz, DMSO) δ 172.3, 152.2, 149.5, 135.7, 134.6, 129.9, 124.9, 124.5, 122.1, 118.8, 112.7, 65.7, 63.8, 56.2, 44.7, 38.6, 23.4, 14.9. Conclusion: MK090 was synthesized as an a-aminoisobutyric acid derivatization of MK079-D in a 58% yield over two steps.

### Example 11: Procedure for the preparation of (S)-2-amino-N-(2-(4-amino-2-(ethoxymethyl)-1H-imidazo[4,5-c]quinolin-1-yl)ethyl)-4-methylpentanamide (MK091)

MK091 was synthesized according to the general procedure: Fmoc-L-leucine (Fmoc-Leu-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 80:20→10:90). UPLCMS of intermediate (LC1, C₁₈): Retention time=1.95 min. Calculated mass C₃₆H₄₀N₆O₄=620.31; Observed m/z [M+H]⁺=621.2. Purification of the product was carried out with a linear gradient over 15 min (A/B 100:0→65:35). Yielded 20.1 mg (78%) as a fluffy white solid. HPLC >95%. ¹H NMR (400 MHz, DMSO-d₆) δ 14.21 (s, 1H), 9.17 (s, 2H), 8.94 (t, J=6.0 Hz, 1H), 8.51 (dd, J=8.4, 1.2 Hz, 1H), 8.13 (s, 2H), 7.82 (dd, J=8.4, 1.3 Hz, 1H), 7.75 (ddd, J=8.4, 7.1, 1.1 Hz, 1H), 7.56 (ddd, J=8.4, 7.0, 1.3 Hz, 1H), 4.91-4.77 (m, 2H), 4.79-4.67 (m, 2H), 3.83 (dq, J=14.1, 7.2 Hz, 1H), 3.68 (t, J=7.2 Hz, 1H), 3.64-3.54 (m, 3H), 1.61-1.44 (m, 1H), 1.35 (ddd, J=14.2, 8.7, 5.7 Hz, 1H), 1.25 (ddd, J=14.1, 8.5, 5.7 Hz, 1H), 1.19 (t, J=7.0 Hz, 3H), 0.82 (d, J=2.2 Hz, 3H), 0.81 (d, J=2.1 Hz, 3H). ¹³C NMR (101 MHz, DMSO) δ 170.1, 152.2, 149.4, 135.8, 134.6, 129.9, 124.9, 124.4, 122.1, 118.9, 112.7, 65.8, 63.9, 50.8, 44.6, 39.9, 38.2, 23.4, 22.6, 22.6, 14.9. Conclusion: MK091 was synthesized as a leucine derivatization of MK079-D in a 78% yield over two steps

### Example 12: Procedure for the preparation of (S)-2-amino-N-(2-(4-amino-2-(ethoxymethyl)-1H-imidazo[4,5-c]quinolin-1-yl)ethyl)-4-(methylthio)butanamide (MK093)

MK093 was synthesized according to the general procedure: Fmoc-L-methionine (Fmoc-Met-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 80:20-10:90). UPLCMS of intermediate (LC1, C₁₈): Retention time=1.88 min. Calculated mass C₃₅H₃₈N₆O₄S=638.27; Observed m/z [M+H]⁺=639.1. Purification of the product was carried out with a linear gradient over 15 min (A/B 100:0→60:40). Yielded 22.5 mg (85%) as a fluffy white solid. HPLC >95%. ¹H NMR (400 MHz, DMSO-d₆) δ 14.26 (s, 1H), 9.19 (s, 2H), 8.96 (t, J=5.9 Hz, 1H), 8.51 (dd, J=8.3, 1.2 Hz, 1H), 8.23 (s, 2H), 7.82 (dd, J=8.4, 1.3 Hz, 1H), 7.75 (ddd, J=8.3, 7.1, 1.1 Hz, 1H), 7.56 (ddd, J=8.3, 7.0, 1.3 Hz, 1H), 4.90-4.76 (m, 2H), 4.83-4.68 (m, 2H), 3.79 (dq, J=21.1, 6.9 Hz, 2H), 3.60 (q, J=7.0 Hz, 3H), 2.41 (ddd, J=9.3, 6.6, 4.2 Hz, 2H), 2.03 (s, 3H), 1.90-1.78 (m, 2H), 1.19 (t, J=7.0 Hz, 3H). ¹³C NMR (101 MHz, DMSO) δ 169.2, 152.2, 149.5, 135.7, 130.0, 124.9, 124.5, 122.0, 112.7, 65.8, 63.9, 51.6, 44.6, 38.3, 30.5, 28.2, 14.9, 14.4. Conclusion: MK093 was synthesized as a methionine derivatization of MK079-D in a 85% yield over two steps.

### Example 13: Procedure for the preparation of (S)-2-amino-N-(2-(4-amino-2-(ethoxymethyl)-1H-imidazo[4,5-c]quinolin-1-yl)ethyl)-6-(dimethylamino)hexanamide (MK094)

MK094 was synthesized according to the general procedure: (2S)-6-(Dimethylamino)-2-{[(9H-fluoren-9-ylmethoxy)carbonyl] amino}hexanoic acid (Fmoc-Lys(Me)₂-OH) (0.060 mmol), HATU (0.055 mmol), 2,4,6-trimethylpyridine (0.105 mmol), and MK079-D (0.050 mmol). Purification of the Fmoc-protected intermediate was carried out with a linear gradient over 15 min (A/B 90:10-20:80). UPLCMS of intermediate (LC1, C₁₈): Retention time=1.39 min. Calculated mass C₃₈H₄₅N₇O₄=663.35; Observed m/z [M+H]⁺=664.1 and [M+2H]⁺=332.7. Purification of the product was carried out with a linear gradient over 20 min (A/B 100:0→50:50). Yielded 20.9 mg (75%) as a fluffy white solid. HPLC >95%. ¹H NMR (400 MHz, DMSO-d₆) δ 14.33 (s, 1H), 9.22 (s, 2H), 9.04 (t, J=6.0 Hz, 1H), 8.53 (dd, J=8.4, 1.2 Hz, 1H), 8.22 (s, 2H), 7.83 (dd, J=8.4, 1.3 Hz, 1H), 7.76 (ddd, J=8.3, 7.0, 1.1 Hz, 1H), 7.56 (ddd, J=8.4, 7.0, 1.3 Hz, 1H), 4.88-4.79 (m, 2H), 4.79-4.66 (m, 2H), 3.81-3.55 (m, 5H), 3.06-2.89 (m, 2H), 2.76 (s, 6H), 1.70-1.48 (m, 2H), 1.19 (t, J=7.0 Hz, 5H). ¹³C NMR (101 MHz, DMSO) δ 169.5, 152.2, 149.5, 135.7, 130.0, 124.9, 124.4, 122.0, 118.6, 112.7, 65.8, 63.9, 56.1, 52.0, 44.6, 42.1, 42.1, 38.2, 23.3, 21.6, 21.2, 14.9. Conclusion: MK094 was synthesized as an N^{ε},N^{ε}-dimethyl lysine derivatization of MK079-D in a 75% yield over two steps.

### Example 14: General Procedure for Small Molecule Remote Loading

A solution of the small molecule (modified TLR agonist) was lyophilized in a vial equipped with a magnetic stir bar over night to form a fluffy white powder. This powder was added either formulation F1 or F2 to the wanted drug/lipid ratio. The resulting mixture was stirred at 55° C. or 41° C. for formulation F1 and F2 respectively for 3h, then allowed to cool to rt before the solutions were refrigerated and stored at 4° C.

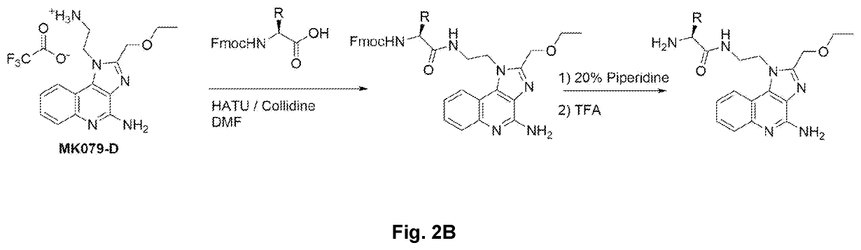

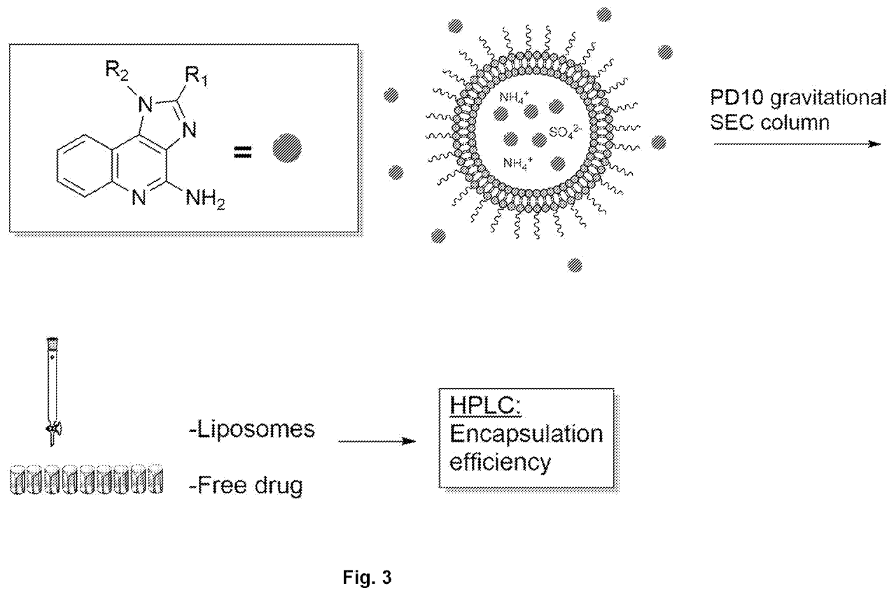

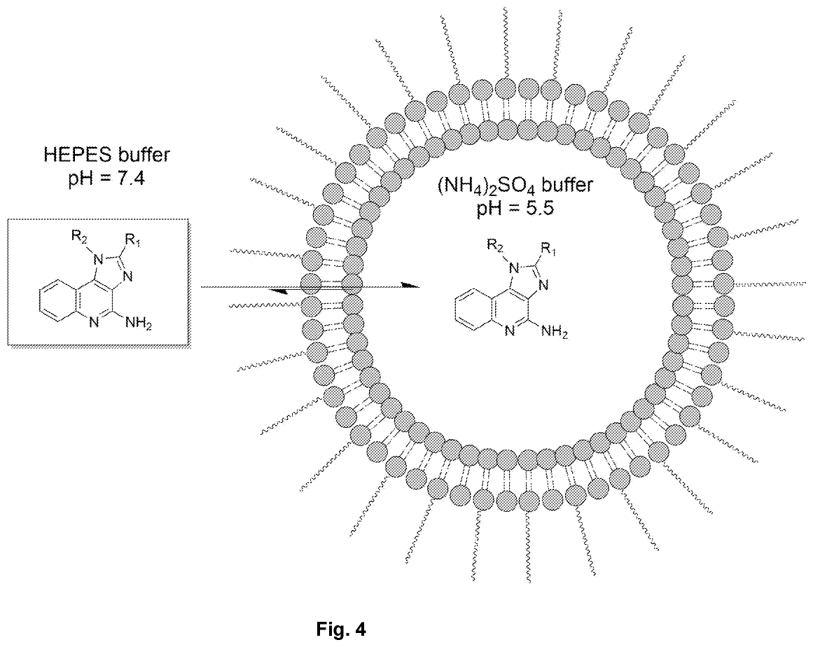

Remote loading efficiency was determined at 1 h and 3 h by taking out 100 μL of the mixture and applying this and 900 μL buffer (25 mM HEPES, 10 vol % sucrose buffer, pH 7.4) to a PD-10 column size exclusion column. Following, 10×1 mL fractions were collected, and the small molecule compound concentrations in the loading mixture and in the liposome containing fractions were determined on analytical HPLC. Loading efficiency was calculated as percentage of molecules found in liposome containing fractions out of the applied amount. The general procedure for the small molecule remote loading is visualized in FIG. 3. The general concept of remote loading imidazoquinolines in liposomes that are dialyzed against HEPES is visualized in FIG. 4.

This general procedure for small molecule remote loading was used in examples below.

### Example 15: General Procedure for Liposome Preparation

This example describes the general procedure for the preparation of the liposomes which were used in the examples 16-18. Specifically, liposomes were prepared by lyophilizing tert-butanol/water (9:1) mixtures of lipids followed by rehydration at 65° C. for formulation F1 and 55° C. for formulation F2 with vortexing every 10 minutes in 150 mM ammonium sulfate to a lipid concentration of 40 mM. The multilamellar vesicles were subsequently downsized by extrusion through 2×100 nm polycarbonate filters at 70° C. or 55° C. for formulations F1 and F2 respectively on a thermobarrel pressure extruder with 6 repetitions. The formulations were subsequently dialyzed against HEPES buffer (25 mM HEPES, 10 vol % sucrose, pH 7.4) or TAPS buffer (25 mM TAPS, 10 vol % sucrose, pH 8.5) in at least 100x formulation volume, with two buffer changes every 12h.

Formulation F1: HSPC/Chol/DSPE-PEG2k-56:38:5 (mol/mol)
Formulation F2: POPC/Chol/DOTAP Cl/DOPE-PEG2k-40:30:25:5 (mol/mol)

TABLE 1
Characterization of formulations F1 and F2

| Formulation | Size (nm) | PDI | Zeta (mV) |
|---|---|---|---|
| F1 | 149 ± 2 | 0.064 | −10 ± 1 |
| F2 | 157 ± 1 | 0.051 | 6 ± 1 |

The liposomes had a similar average size of 153 nm and a low polydispersity with zeta potentials reflecting their composition.

### Example 16: Remote Loading of MK079-D in Formulations F1 and F2 with Varying Drug/Lipid Ratios

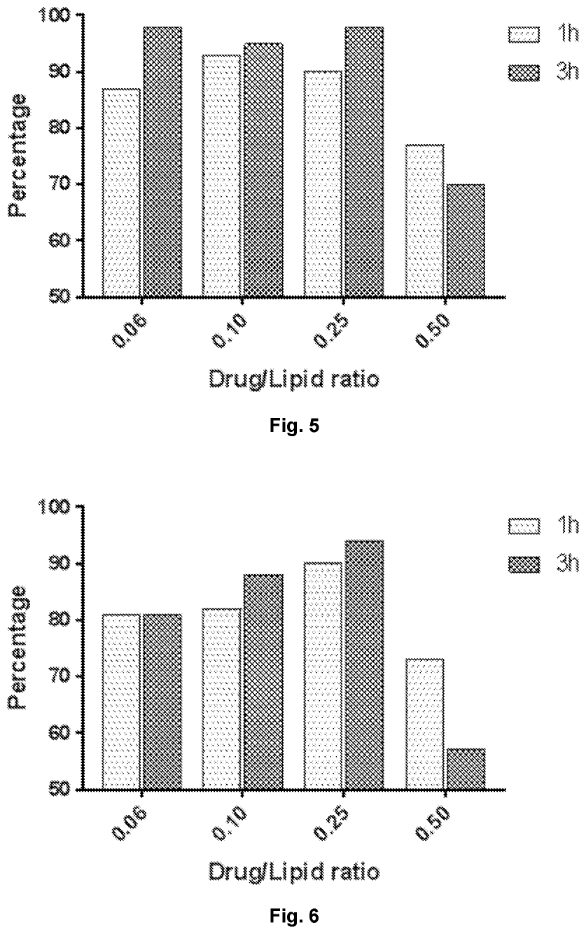

In this example we investigated the loading efficiency of MK079-D at varying drug/lipid ratios in both formulations F1 and F2. Remote loading of MK079-D was carried out as described in example 14 with a solution of MK079-D and formulations F1 or F2 dialyzed against HEPES buffer. Remote loading efficiencies measured by HPLC are summarized in table 2 below and in FIG. 5 and FIG. 6.

TABLE 2
Overview of remote loading efficiency
of MK079-D in formulations F1 and F2

| Formulation | Drug/Lipid ratio | 1 h (%) | 3 h (%) |
|---|---|---|---|
| F1 | 0.06 | 87 | 98 |
| | 0.10 | 93 | 95 |
| | 0.25 | 90 | 98 |
| | 0.50 | 77 | 70 |
| F2 | 0.06 | 81 | 81 |
| | 0.10 | 82 | 88 |
| | 0.25 | 90 | 94 |
| | 0.50 | 73 | 57 |

In formulation F1, the loading efficiency of MK079-D increased over time for drug/lipid ratios 0.06, 0.10, and 0.25, but decreased over time at the highest drug/lipid ratio of 0.5. In formulation F1, similar loading efficiency was obtained at drug/lipid 0.006, 0.10 and 0.25, while the loading efficiency was reduced at drug/lipid ratio 0.50.

In formulation F2, the loading efficiency of MK079-D increased over time for drug/lipid ratios 0.10, and 0.25, but decreased over time at the highest drug/lipid ratio of 0.5. In formulation F2, the maximal loading efficiency was obtained at drug/lipid 0.25.

For both formulation F1 and F2, the loading decreased at the drug/lipid ratio 0.50 indicating that the pH gradient was exhausted.

### Example 17: Remote Loading of MK079-D in HEPES Buffer Vs TAPS Buffer

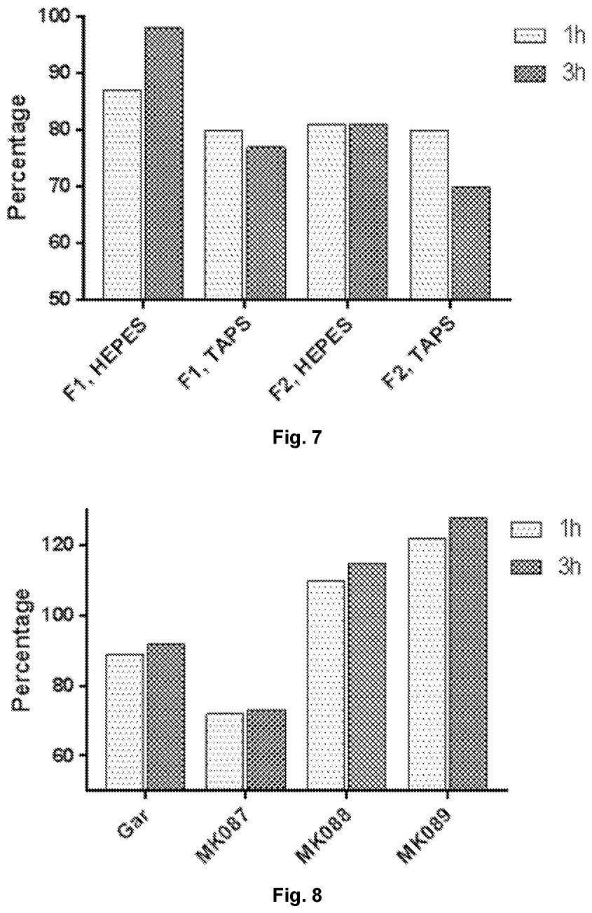

In this example we investigated the loading efficiency of MK079-D with formulations F1 and F2 when dialyzed against the two different buffers HEPES (pH 7.4) or TAPS (pH 8.5). Remote loading of MK079-D was carried out as described in example 14 with a drug/lipid ratio of 0.06 in HEPES or TAPS buffer. Remote loading efficiencies measured by HPLC are summarized in table 3 below and in FIG. 7.

TABLE 3
Overview of remote loading efficiency of
MK079-D in formulations F1 and F2 in
HEPES and TAPS buffer at drug/lipid ratio 0.06

| Formulation | 1 h (%) | 3 h (%) |
|---|---|---|
| F1, HEPES | 87 | 98 |
| F1, TAPS | 80 | 77 |
| F2, HEPES | 81 | 81 |
| F2, TAPS | 80 | 70 |

Both formulations had higher loading efficiencies in HEPES at pH 7.4 compared to TAPS at pH 8.5, at a drug/lipid ratio of 0.06. Furthermore, the loading efficiency in HEPES increased over time or remained at the same level, while the loading efficiencies in TAPS decreased over time at a drug/lipid ratio of 0.06.

### Example 18: Remote Loading of Gardiquimod, MK087, MK088, and MK089 in Formulations F1 and F2

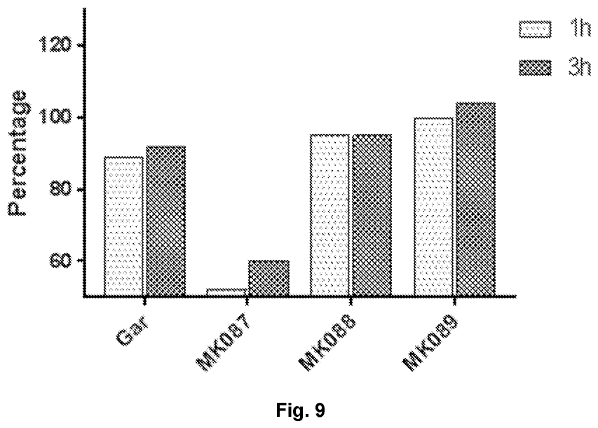

In this example we investigated the loading efficiency of Gardiquimod, MK087, MK088, and MK089 with formulations F1 and F2 when dialyzed against HEPES buffer. Remote loading of Gardiquimod, MK087, MK088, and MK089 was carried out as described in example 14 with a drug/lipid ratio of 0.10 in HEPES buffer. Remote loading efficiencies measured by HPLC are summarized in Table 4 below and in FIG. 8 and FIG. 9.

TABLE 4
Overview of remote loading efficiencies of Gardiquimod,
MK087, MK088, and MK089 in formulations F1 and F2

| Formulation | Small molecule | 1 h (%) | 3 h (%) |
|---|---|---|---|
| F1 | Gardiquimod | 89 | 92 |
| | MK087 | 72 | 73 |
| | MK088 | 110 | 115 |
| | MK089 | 122 | 128 |
| F2 | Gardiquimod | 89 | 92 |
| | MK087 | 52 | 60 |
| | MK088 | 95 | 95 |
| | MK089 | 100 | 104 |

The loading efficiency was found to follow the sequence MK078<Gardiquimod<MK088<MK089 for both formulation F1 and F2.

### Example 19: Remote Loaded MK079-D Precipitated in Formulation F1 a Drug/Lipid Ratio of 0.25

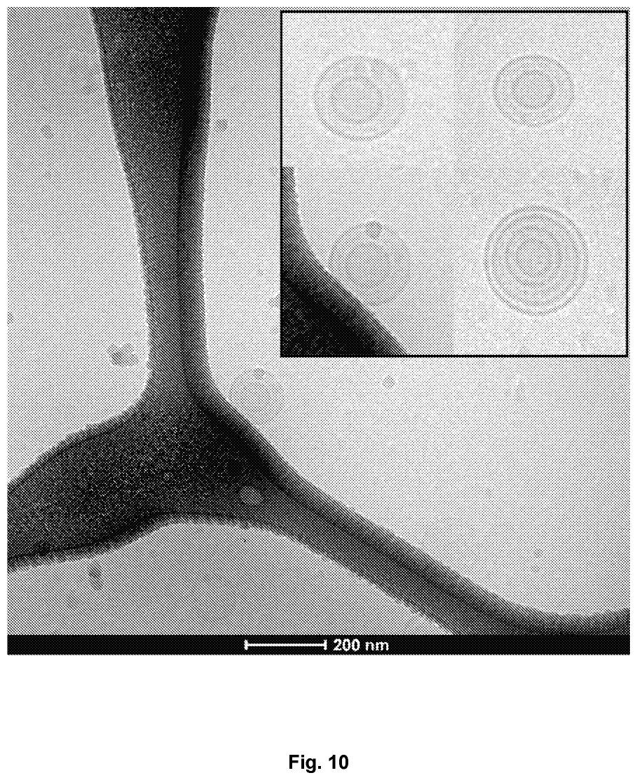

In this example we investigated whether the small molecules would precipitate upon protonation in the liposomes when remote loaded at a drug/lipid ratio of 0.25 in formulation F1. Remote loading of MK079-D was carried out as described in example 14 with a solution of MK079-D and formulation F1 dialyzed against HEPES buffer at a drug/lipid ratio of 0.25. Cryo-TEM of the liposomes then showed precipitation of the small molecule inside the aqueous core of the liposomes in formulation F1 at a drug/lipid ratio of 0.25, as seen in FIG. 10.

### Example 20: Remote Loaded MK079-D Precipitated in Formulation F1 a Drug/Lipid Ratio of 0.50

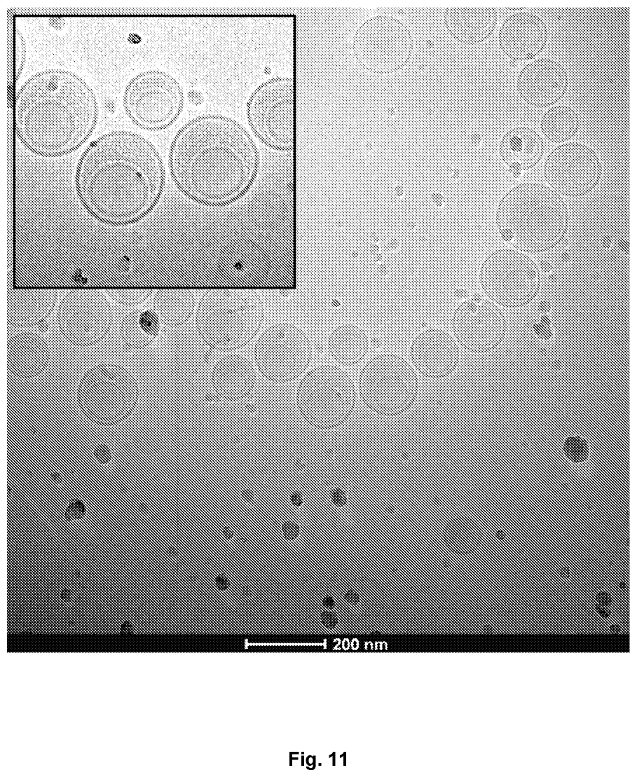

In this example we investigated whether the small molecules would precipitate upon protonation in the liposomes when remote loaded at a drug/lipid ratio of 0.50 in formulation F1. Remote loading of MK079-D was carried out as described in example 14 with a solution of MK079-D and formulation F1 dialyzed against HEPES buffer at a drug/lipid ratio of 0.50. Cryo-TEM of the liposomes showed precipitation of the small molecule in the aqueous core of the liposomes in formulation F1 at a drug/lipid ratio of 0.50, as seen in FIG. 11.

### Example 21: Remote Loaded MK079D Precipitated in Formulation F2 a Drug/Lipid Ratio of 0.25

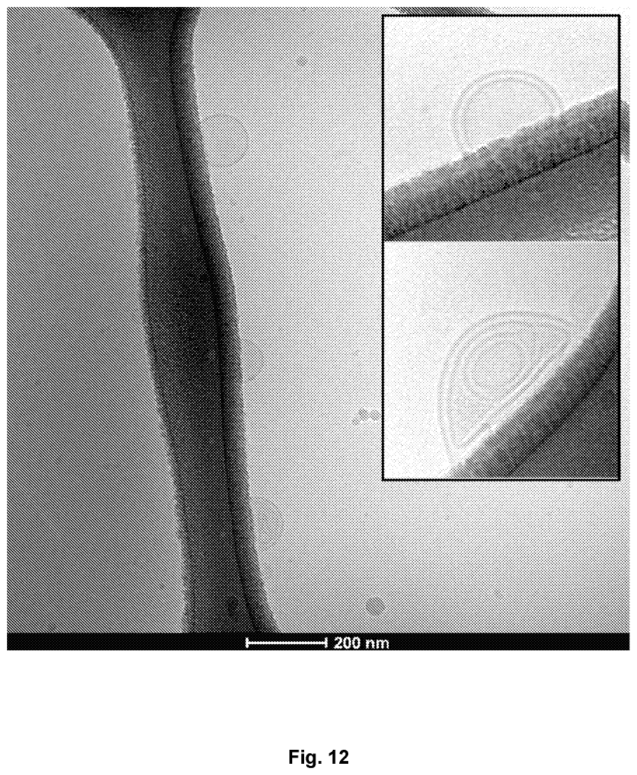

In this example we investigated whether the small molecules would precipitate upon protonation in the liposomes when remote loaded at a drug/lipid ratio of 0.25 in formulation F2. Remote loading of MK079-D was carried out as described in example 14 with a solution of MK079-D and formulation F1 dialyzed against HEPES buffer at a drug/lipid ratio of 0.25. Cryo-TEM of the liposomes then showed precipitation of the small molecule in the aqueous core of the liposomes in formulation F2 at a drug/lipid ratio of 0.25, as seen in FIG. 12.

### Example 22: Remote Loaded MK079D Precipitated in Formulation F2 a Drug/Lipid Ratio of 0.50

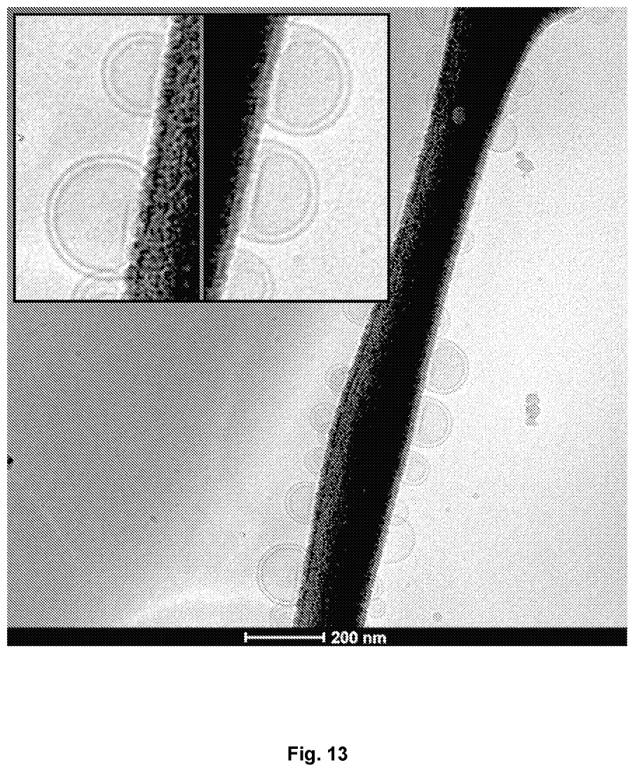

In this example we investigated whether the small molecules would precipitate upon protonation in the liposomes when remote loaded at a drug/lipid ratio of 0.50 in formulation F2. Remote loading of MK079-D was carried out as described in example 14 with a solution of MK079-D and formulation F1 dialyzed against HEPES buffer at a drug/lipid ratio of 0.50. Cryo-TEM of the liposomes then showed precipitation of the small molecule in the aqueous core of the liposomes in formulation F2 at a drug/lipid ratio of 0.50, as seen in FIG. 13.

### Example 23: General Procedure for the Measurement of IL-6 and IL-12p40 Cytokine Expression in Whole Human Blood Stimulated with Small Molecules or Small Molecules in Formulations F1 and F2

This example describes the general procedure used to assess the level of cytokine IL-6 and IL-12p40 expressions upon stimuli of whole human blood with small molecules or small molecules in liposomal formulations F1 and F2. 10 mM DMSO solutions of the small molecules were diluted in RPMI media (RPMI-1640, Sigma Aldrich) with 1% penicillin/streptomycin (p/s). 204, of each dilution was transferred to Nunc™ 96-Well Polystyrene Conical Bottom MicroWell™ Plates in duplicates. Blood was drawn from two human donors, diluted in RPMI with 1% p/s and added to each well to reach final concentrations of 10 μM, 1 μM, and 0.1 μM of the small molecules. The plates were incubated at 37° C. in a cell incubator for 24h. The plates were then spun down in a plate centrifuge with 3200 revolutions per minute (rpm) for 10 min before 50 uL of the supernatant was transferred to new 96-well plates. The supernatant samples were subsequently stored at −80° C. until the day of cytokine measurements.

The level of cytokine expression was measured using either an IL-6 ELISA Kit (RnDSystems, cat #DY206) for IL-6, or an IL-12p40 ELISA Kit (RnDSystems, cat #DY1240) according to the manufacturer's protocols. This general procedure was used to generate the examples 24-33.

### Example 24: Stimulation of Whole Human Blood with MK079-D Resulted in Expression of Cytokine IL-6

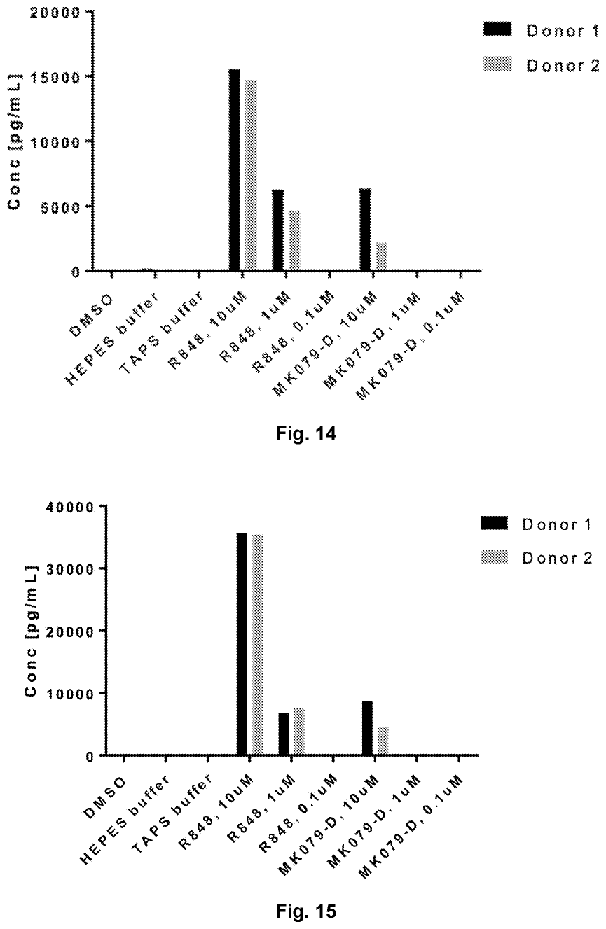

This experiment describes the expression of IL-6 upon stimulation of whole human blood with MK079-D and R848. Stimulation of whole human blood and measurement of IL-6 expression was carried out as described in example 23 with MK079-D and R848 diluted in PBS from 10 mM DMSO stocks. While stimulation with R848 showed dose dependent expression of IL-6 with similar expression levels for both donors at 10 μM and 1 μM, stimulation with MK079-D gave an expression of IL-6 at 10 μM in both donors. Results are illustrated in FIG. 14. Stimulation of human whole blood with MK079-D at 10 μM resulted in expression of IL-6, as seen in FIG. 14.

### Example 25: Stimulation of Whole Human Blood with MK079-D Resulted in Expression of Cytokine IL-12p40

This experiment describes the expression of IL-12p40 upon stimulation of whole human blood with MK079-D and R848. Stimulation of whole human blood and measurement of IL-12p40 expression was carried out as described in example 23 with MK079-D and R848 diluted in PBS from 10 mM DMSO stocks. While stimulation with R848 showed dose dependent expression of IL-12p40 with similar expression levels for both donors at 10 μM and 1 μM, stimulation with MK079-D gave an expression of IL-12p40 at 10 μM in both donors. Results are illustrated in FIG. 15. Stimulation of whole human blood with MK079-D at 10 μM resulted in expression of IL-12p40, as seen in FIG. 15.

### Example 26: Stimulation of Whole Human Blood with MK079-D Remote Loaded in Formulations F1 or F2 Resulted in Expression of Cytokine IL-6

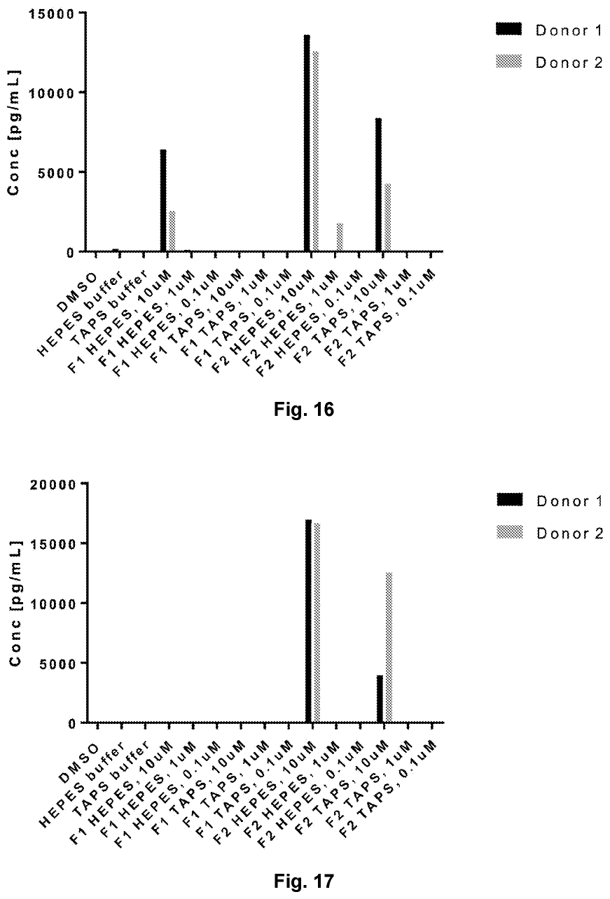

This experiment describes the expression of IL-6 upon stimulation of whole human blood with MK079-D remote loaded in formulations F1 or F2 in HEPES or TAPS buffers at a drug/lipid ratio of 0.06. Stimulation of whole human blood and measurement of IL-6 expression was carried out as described in example 23 with a solution of MK079-D remote loaded in formulations F1 or F2 at a drug/lipid ratio of 0.06 and dialyzed against HEPES buffer or TAPS buffer as described in example 14. As seen in FIG. 16, stimulation with MK079-D in formulation F1 dialyzed against HEPES resulted in an expression of IL-6 at 10 μM which was lower than the expression of IL-6 upon stimulation of MK079-D in formulation F2 dialyzed against HEPES. Furthermore, stimulation with MK079-D in formulation F1 dialyzed against TAPS resulted in no expression of IL-6 at 10 μM while stimulation with MK079-D in formulation F2 dialyzed against TAPS resulted in an expression of IL-6, as seen in FIG. 16. Formulation F2 induced a higher expression of IL-6 compared to formulation F1 when dialyzed against both HEPES and TAPS, as seen in FIG. 16. Both formulations F1 and F2 were however able to induce expression of IL-6 when dialyzed against HEPES. A larger response in IL-6 induction was observed for liposomes dialyzed against HEPES compared to TAPS buffer.

### Example 27: Stimulation of Whole Human Blood with MK079-D Remote Loaded in Formulation F2 Resulted in Expression of Cytokine IL-12p40

This experiment describes the expression of IL-12p40 upon stimulation of whole human blood with MK079-D remote loaded in formulations F1 or F2 in HEPES or TAPS buffers at a drug/lipid ratio of 0.06. Stimulation of whole human blood and measurement of IL-12p40 expression was carried out as described in example 23 with a solution of MK079-D remote loaded in formulations F1 or F2 at a drug/lipid ratio of 0.06 and dialyzed against HEPES buffer or TAPS buffer as described in example 14. As seen in FIG. 17, stimulation with MK079-D in formulation F1 did not lead to any expression of IL-12p40 while stimulation of MK079-D in formulation F2 lead to a larger expression of IL-12p40 when dialyzed against HEPES compared to TAPS. Induction of IL-12p40 expression was observed upon stimulation with MK079-D remote loaded into formulation F2, but not when remote loaded into formulation F1. A larger response in IL-12p40 induction was observed for liposomes dialyzed against HEPES compared to TAPS buffer.

### Example 28: Stimulation of Whole Human Blood with Small Molecules Gardiquimod, MK088, MK089, MK090, MK091, MK093, and MK094 Resulted in Expression of Cytokine IL-6

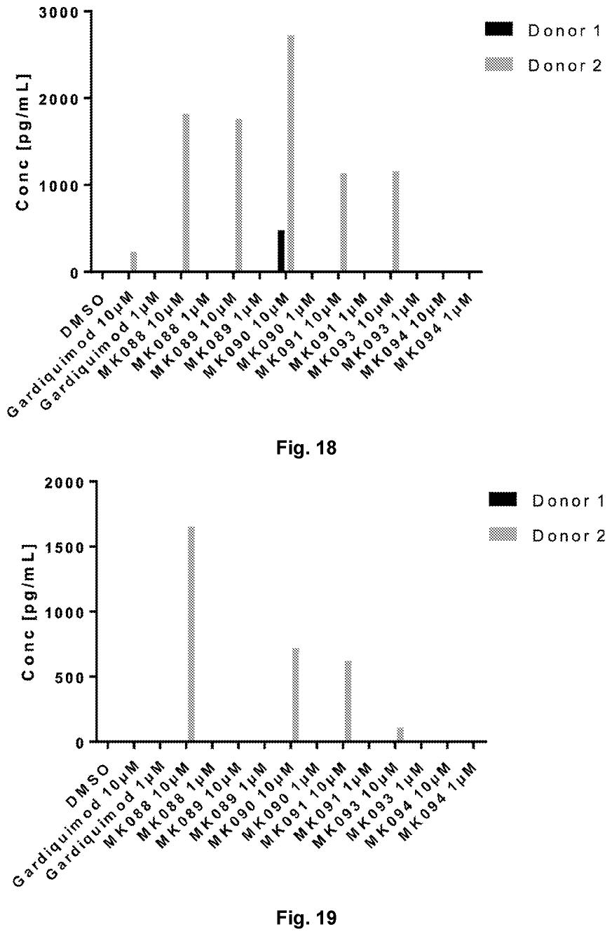

This experiment describes the expression of IL-6 upon stimulation of whole human blood with the small molecules Gardiquimod, MK088, MK089, MK090, MK091, MK093, and MK094. Stimulation of whole human blood and measurement of IL-6 expression was carried out as described in example 23 with Gardiquimod, MK088, MK089, MK090, MK091, MK093, and MK094 diluted in PBS from 10 mM DMSO stocks. Gardiquimod, MK088, MK089, MK090, MK091, and MK093 all induced expression of IL-6 at 10 μM in donor 2, while only MK090 was able to induce expression of IL-6 in both donors, as seen in FIG. 18. MK090 was furthermore able to induce a higher level of IL-6 expression. Gardiquimod, MK088, MK089, MK090, MK091, and MK093 were all capable of inducing expression of IL-6 in a donor. MK090 was the most potent inducer of IL-6.

### Example 29: Stimulation of Whole Human Blood with Small Molecules Gardiquimod, MK088, MK089, MK090, MK091, MK093, and MK094 Resulted in Expression of Cytokine IL-12p40

This experiment describes the expression of IL-12p40 upon stimulation of whole human blood with the small molecules Gardiquimod, MK088, MK089, MK090, MK091, MK093, and MK094. Stimulation of whole human blood and measurement of IL-12p40 expression was carried out as described in example 23 with Gardiquimod, MK088, MK089, MK090, MK091, MK093, and MK094 diluted in PBS from 10 mM DMSO stocks. MK088, MK090, MK091, and MK093 all induced expression of IL-12p40 at 10 μM in donor 2, while none of the molecules were able to induce expression of IL-12p40 in donor 1 in the concentrations tested, as seen in FIG. 19. MK088 was furthermore able to induce the highest level of IL-12p40 expression. MK088, MK090, MK091, and MK093 were all capable of inducing expression of IL-12p40 in a donor in the concentrations tested. MK088 was the most potent inducer of IL-12p40.

### Example 30: Stimulation of Whole Human Blood with Small Molecules Gardiquimod, MK087, MK088, and MK089 Remote Loaded in Formulation F1 Resulted in Expression of Cytokine IL-6

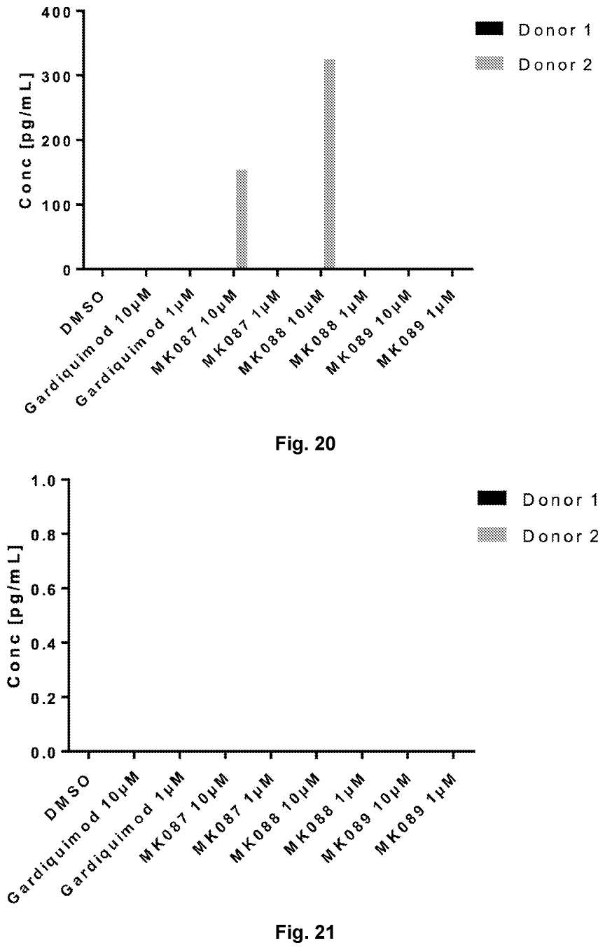

This experiment describes the expression of IL-6 upon stimulation of whole human blood with small molecules Gardiquimod, MK087, MK088, and MK089 when remote loaded in formulation F1. Stimulation of whole human blood and measurement of IL-6 expression was carried out as described in example 23 with Gardiquimod, MK087, MK088, and MK089, which were remote loaded in formulation F1 dialyzed against HEPES and remote loaded with a drug/lipid ratio of 0.1 using the method described in example 14. In donor 2, MK087 and MK088 were capable of inducing expression of IL-6 at 10 μM when remote loaded into formulation F1, as seen in FIG. 20. No induction of IL-6 expression was seen with Gardiquimod or MK089 when remote loaded into formulation F1 in the concentrations tested. Upon stimulation of whole human blood with small molecules Gardiquimod, MK087, MK088, and MK089 remote loaded in formulation F1, only MK087 and MK088 were capable of inducing expression of IL-6 at 10 μM.

### Example 31: Stimulation of Whole Human Blood with Small Molecules Gardiquimod and MK087-89 Remote Loaded in Formulation F1 Resulted in Expression of Cytokine IL-12p40

This experiment describes the expression of IL-12p40 upon stimulation of whole human blood with small molecules Gardiquimod, MK087, MK088, and MK089 when remote loaded in formulation F1. Stimulation of whole human blood and measurement of IL-12p40 expression was carried out as described in example 23 with Gardiquimod, MK087, MK088, and MK089 which were remote loaded in formulation F1 dialyzed against HEPES and remote loaded with a drug/lipid ratio of 0.1 using the method described in example 14. In both donors, none of the small molecules Gardiquimod, MK087, MK088, and MK089 were capable of inducing expression of IL-12p40 when remote loaded into formulation F1 in the concentrations tested, as seen in FIG. 21. None of the small molecules Gardiquimod, MK087, MK088, and MK089 were capable of inducing expression of IL-12p40 when remote loaded into formulation F1 in the concentrations tested.

### Example 32: Stimulation of Whole Human Blood with Small Molecules Gardiquimod and MK087, MK088, and MK089 Remote Loaded in Formulation F2 Resulted in Expression of Cytokine IL-6

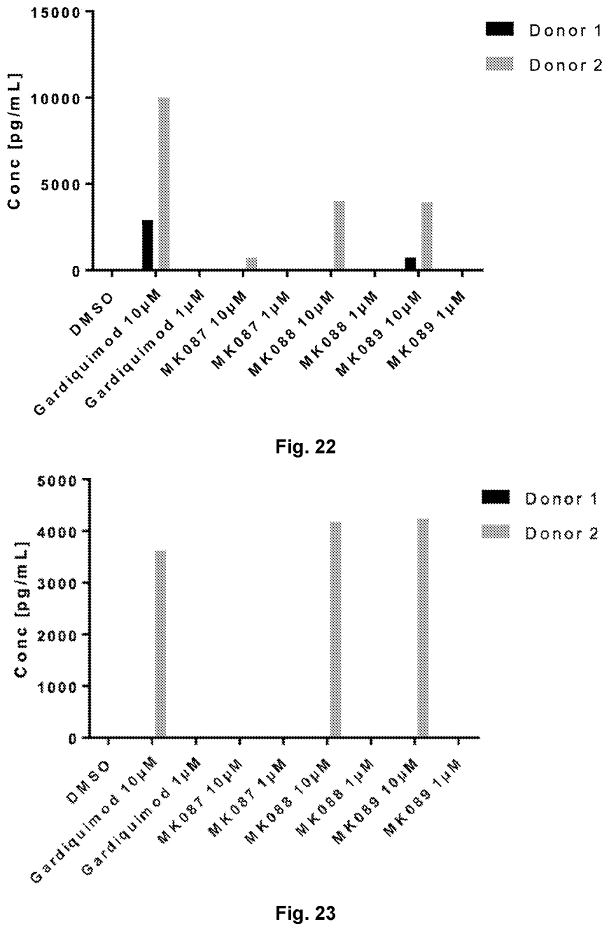

This experiment describes the expression of IL-6 upon stimulation of whole human blood with small molecules Gardiquimod, MK087, MK088, and MK089 when remote loaded in formulation F2. Stimulation of whole human blood and measurement of IL-6 expression was carried out as described in example 23 with Gardiquimod, MK087, MK088, and MK089 which were remote loaded in formulation F2 dialyzed against HEPES and remote loaded with a drug/lipid ratio of 0.1 using the method described in example 14. Gardiquimod, MK087, MK088, and MK089 were all capable of inducing expression of IL-6 at 10 μM when remote loaded into formulation F2, as seen in FIG. 22. Gardiquimod and MK089 were capable of inducing expression of IL-6 in both donors, while MK087 and MK088 were not capable of inducing expression of IL-6 in donor 1 in the concentrations tested. Furthermore, Gardiquimod was the most potent of these molecules when remote loaded into formulation F2, as seen in FIG. 22. When remote loaded into formulation F2, Gardiquimod, MK087, MK088, and MK089 were all capable of inducing expression of IL-6, where Gardiquimod was the most potent.

### Example 33: Stimulation of Whole Human Blood with Small Molecules Gardiquimod and MK087, MK088, and MK089 Remote Loaded in Formulation F2 Resulted in Expression of Cytokine IL-12p40

This experiment describes the expression of IL-12p40 upon stimulation of whole human blood with small molecules Gardiquimod, MK087, MK088, and MK089 when remote loaded in formulation F2. Stimulation of whole human blood and measurement of IL-12p40 expression was carried out as described in example 23 with Gardiquimod, MK087, MK088, and MK089, which were remote loaded in formulation F2 dialyzed against HEPES and remote loaded with a drug/lipid ratio of 0.1 using the method described in example 14. Gardiquimod, MK088, and MK089 were all capable of inducing expression of IL-12p40 in one donor at 10 μM when remote loaded into formulation F2, as seen in FIG. 23, while MK087 was not capable of inducing expression of IL-12p40 when remote loaded into formulation F2 in the concentrations tested. Gardiquimod, MK088, and MK089 were all capable of inducing expression of IL-12p40 when remote loaded into formulation F2 in the concentrations tested.

### Example 34: cLogD Example for Remote Loading of Small Molecule Imidazoquinoline Derivatives

This example lists the calculated LogD (cLogD) values at pH values 5.5, 7.4, 8.5 and 9.0 of the imidazoquinolines in the examples above. The cLogD profile is important for the compounds ability to be remote loaded into liposomes. For remote loading of compounds using the ammonium sulfate gradient, the compound should preferably be hydrophobic (cLogD>0) outside the liposome as this aids the membrane permeation of the compound. Once the compound has crossed the membrane barrier, the compound should preferably change physicochemical characteristics from hydrophobic to hydrophilic (cLogP<0) thereby hindering the compound in escaping the aqueous core of the liposome. Such change in physicochemical property can be realized by: i) chemical reactions modifying the compound, ii) chelation to ions that changes the overall charge of the compound, or iii) change in charge of the compound by deprotonation or protonation as in the current case with the ammonium sulfate gradient liposomes. The cLogD values were calculated using MarvinSketch 17.27.0 by ChemAxon Ltd. The cLogD values were calculated by the ChemAxon method with the electrolyte concentrations of chloride, sodium, and potassium were set to zero. Four distinct pH values 5.5, 7.4, 8.5 and 9.0 were chosen as output. The cLogD values of all imidazoquinolines in this example were increasing from pH=5.5 to pH=9.0 as seen from the table below. Furthermore, most imidazoquinolines had a change from a negative to a positive cLogD value when increasing the pH from 5.5 to 9.0.

TABLE 4
cLogD values at pH values 5.5, 7.4, 8.5 and 9.0
of the imidazoquinolines in the examples above.

| Molecule | cLogD_{5.5} | cLogD_{7.4} | cLogD_{8.5} | cLogD_{9.0} |
|---|---|---|---|---|
| Gardiquimod | −1.55 | 0.38 | 1.15 | 1.29 |
| MK079-D | −3.22 | −1.23 | −0.16 | 0.29 |
| MK087 | −2.86 | −0.84 | −0.18 | −0.08 |
| MK088 | −2.57 | −0.52 | 0.27 | 0.42 |
| MK089 | −1.83 | 0.24 | 1.08 | 1.26 |
| MK090 | −2.63 | −0.55 | 0.34 | 0.56 |
| MK091 | −1.42 | 0.64 | 1.44 | 1.61 |
| MK093 | −2.08 | −0.02 | 0.78 | 0.94 |
| MK094 | −6.38 | −2.42 | −0.53 | 0.09 |

The cLogD values of all imidazoquinolines in this example increased from pH=5.5 to pH=9.0, while the cLogD values of Gardiquimod, MK079-D, MK088, MK089, MK090, MK091, MK093, and MK094 changed from a negative to a positive cLogD value when increasing the pH from 5.5 to 9.0. The crossover from positive to negative cLogD value for MK087, MK088 and MK089 occurs at pH>9, pH>7.4 and pH>5.5 respectively. This change in cLogD may impact the loading efficiency, which is observed to follow the sequence MK087<MK088<MK089 in example 18.

### Example 35: Remote Loading of Gardiquimod into Neutral or Cationic Liposomes Composed of Saturated or Unsaturated Lipids

In the current example, the loading efficiency for remote loading of Gardiquimod is investigated for ammonium sulfate gradient liposomes with either a DiStearoyl (DS) or Palmitoyl-Oleoyl (PO) fatty acid-based lipid composition. The following compositions were investigated:

RGNeu_DS: DSPC:Cholesterol:DSPE-PEG2k (64.5:35:0.5)

RGNeu_PO: POPC:Cholesterol:DSPE-PEG2k (64.5:35:0.5)

RGCatDS: DSPC:DSTAP:Cholesterol:DSPE-PEG2k (54.5:10:35:0.5)

RGCatPO: POPC:DOTAP:Cholesterol:DSPE-PEG2k (54.5:10:35:0.5)

Liposomes were prepared as described in example 15. Briefly, liposomes were prepared by lyophilizing tert-butanol/water (9:1) mixtures of lipids followed by rehydration at 65° C. for all formulations with vortexing every 10 minutes in 150 mM ammonium sulfate to a lipid concentration of 50 mM. The multilamellar vesicles were subsequently downsized by extrusion through 2×100 nm polycarbonate filters at 70° C. on a thermobarrel pressure extruder with 10 repetitions. The formulations were subsequently dialyzed against HEPES buffer (25 mM HEPES, 10 vol % sucrose, pH 7.4) in at least 100× formulation volume, with two buffer changes every 12h. The size of the liposomes was determined as described previously in the method section. Remote loading of Gardiquimod was carried out at a drug/lipid ratio of 0.25 by adding liposomes directly to dry Gardiquimod. Following, the samples were magnetically stirred and heated to 55° C. (DS type formulations) or 41° C. (PO type formulations) for 3 h.

Remote loading efficiency was determined at 3h by taking out 100 μL of the mixture and applying this and 900 μL buffer (25 mM HEPES, 10 vol % sucrose buffer, pH 7.4) to a PD-10 column size exclusion column. Following, 10×1 mL fractions were collected, and the small molecule compound concentrations and phosphor lipid content in the loading mixture and in the main liposome fraction (3&4) were determined on analytical HPLC and ICP-MS respectively. The remote loading efficiency was determined as the ratio of drug to lipid in the PD-10 column fraction measured relative to the ratio of drug to lipid in the loading mixture. Liposomes with reduced amount of PEG2k were prepared with sizes in the range 107-137 nm and low PDI. Gardiquimod was remote loaded into both cationic and neutral DS or PO based liposomes with equal loading efficiency (RE (%)) as given in table 5. Gardiquimod could be remote loaded into ammonium sulfate gradient liposomes with or without cationic surface charge at reduced amount of DSPE-PEG2k. Hence, the cationic surface charge of RGCat_DS/RGCat_PO did not affect the pH transmembrane gradient, which would have impaired the loading efficiency.

TABLE 5
Results for remote loading of Gardiquimod into neutral or cationic liposomes.
RE = Remote loading efficiency, D = Gardiquimod concentration, L = Lipid concentration.

| | | | RE | | | |
|---|---|---|---|---|---|---|
| Formulation | Size (nm) | PDI | (%) | D (mM) | L (mM) | D/L |
| RGCat_PO | 107 | 0.06 | 63 | 17.4 | 54.4 | 0.32 |
| RGCat_DS | 137 | 0.10 | 70 | 15.7 | 53.9 | 0.29 |
| RGNeu_PO | 112 | 0.07 | 70 | 18.0 | 55.6 | 0.32 |
| RGNeu_DS | 108 | 0.02 | 69 | 13.1 | 43.0 | 0.30 |

### Example 36: Procedure for preparation of 4-((6-amino-8-hydroxy-2-(2-methoxyethoxy)-9H-purin-9-yl)methyl)-N-(2-(1-methylpyrrolidin-2-yl)ethyl)benzamide (KRJ1-068)

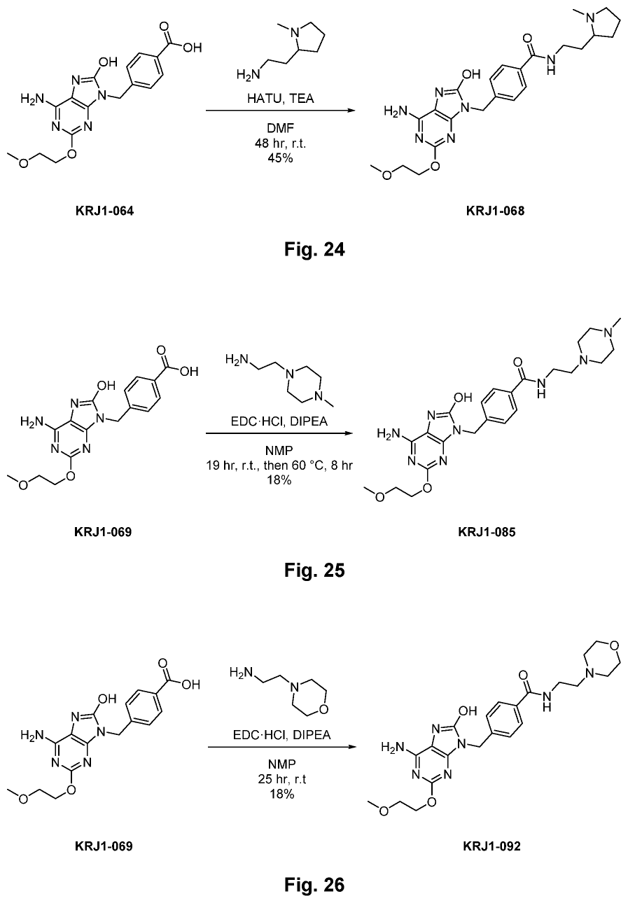

KRJ1-068 was synthesized from KRJ1-064 as seen in FIG. 24 using the following procedure. KRJ1-064 (58.0 mg, 0.161 mmol) was dissolved in anhydrous DMF (8 mL). HATU (76.0 mg, 0.199 mmol), triethylamine (50 mL, 0.359 mmol) and 2-(2-aminoethyl)-1-methylpyrrolidine (30 mL, 0.207 mmol) was added. The reaction mixture was stirred at room temperature for 24 hr, before additional 2-(2-aminoethyl)-1-methylpyrrolidine (20 mL, 0.138 mmol) was added. Stirring was continued for 24 hr at room temperature. The mixture was concentrated in vacuo. The crude product was purified by preparative HPLC to give the product KRJ1-068 as an orange solid (32.5 mg, 45%). R_{f}=0.60 (2% TEA, 5% MeOH in DCM, v/v/v). HPLC RT: 6.79 min (method A). LC-MS: found: 470.2 [M+H]⁺, C₂₃H₃₂N₇O₄⁺ requires M, 470.25. ¹H NMR (400 MHz, DMSO-d₆) δ 10.15 (s, 1H), 8.54 (t, J=5.7 Hz, 1H), 7.79 (d, J=8.3 Hz, 2H), 7.37 (d, J=8.3 Hz, 2H), 6.56 (s, 2H), 4.91 (s, 2H), 4.29-4.21 (m, 2H), 3.64-3.49 (m, 3H), 3.35 (qd, J=6.2, 2.8 Hz, 2H), 3.26 (s, 3H), 3.24-3.15 (m, 1H), 3.04 (dq, J=11.5, 8.1 Hz, 1H), 2.80 (d, J=4.8 Hz, 3H), 2.30 (ddd, J=16.1, 8.1, 4.7 Hz, 1H), 2.13 (dtd, J=14.2, 7.3, 4.0 Hz, 1H), 2.05-1.80 (m, 2H), 1.76-1.56 (m, 2H). ¹³C NMR (101 MHz, DMSO-d₆) δ 166.15, 159.80, 152.28, 149.16, 147.76, 140.37, 133.46, 127.45, 127.28, 98.40, 70.22, 66.33, 65.33, 58.08, 55.15, 42.14, 38.59, 36.28, 29.90, 29.06, 20.98. Conclusion: KRJ1-068 was synthesized from KRJ1-064 in 45% yield.

### Example 37: Procedure for preparation of 4-((6-amino-8-hydroxy-2-(2-methoxyethoxy)-9H-purin-9-yl)methyl)-N-(2-(4-methylpiperazin-1-yl)ethyl)benzamide (KRJ1-085) KRJ1-085 was synthesized from KRJ1-069 as seen in FIG. 25 using the following procedure

KRJ1-069 (50.8 mg, 0.141 mmol) and EDC hydrochloride (27.1 mg, 0.141 mmol) were dissolved in anh. NMP (2.5 mL). 2-(4-Methyl-piperazin-1-yl)-ethylamine (29.5 mg, 0.206 mmol) and DIPEA (50 mL, 0.283 mmol) were dissolved in anh. NMP (2.5 mL). Both mixtures were stirred for 12 hr at room temperature, then combined and stirred for additional 7 hr at room temperature. 2-(4-Methyl-piperazin-1-yl)-ethylamine (20.1 mg, 0.140 mmol) and DIPEA (10 mL, 0.057 mmol) were dissolved in anh. NMP (0.5 mL) and stirred for 1 hr at room temperature, before added to the reaction mixture. The mixture was stirred for 12 hr at room temperature before heated to 60° C. for 8 hr. The mixture was concentrated in vacuo. The crude product was purified by preparative HPLC to give the product KRJ1-085 as a white solid (12.0 mg, 18%). Contaminated fractions were collected and will be purified again. HPLC RT: 5.478 min (method B). LC-MS: found: 485.2 [M+H]⁺, C₂₃H₃₃N₈O₄⁺ requires M, 485.26. ¹H NMR (400 MHz, DMSO-d₆) δ 10.12 (s, 1H), 8.52 (t, J=5.5 Hz, 1H), 7.79 (d, J=8.3 Hz, 2H), 7.37 (d, J=8.3 Hz, 2H), 6.55 (brs, 2H), 4.91 (s, 2H), 4.61-3.90 (brm and m, 10H), 3.61-3.54 (m, 2H), 3.49 (q, J=6.1, 5.3 Hz, 2H), 3.26 (s, 3H), 2.93 (s, 2H), 2.78 (s, 3H). ¹³C NMR (101 MHz, DMSO-d₆) δ 166.30, 159.77, 152.26, 149.15, 147.72, 140.47, 133.25, 127.49, 127.27, 98.39, 70.21, 65.34, 58.07, 55.18, 51.00, 48.85, 42.14 (2×C), 35.37. Conclusion: KRJ1-085 was synthesized from KRJ1-069 in 18% yield.

### Example 38: Procedure for preparation of 4-((6-amino-8-hydroxy-2-(2-methoxyethoxy)-9H-purin-9-yl)methyl)-N-(2-(4-methylpiperazin-1-yl)ethyl)benzamide (KRJ1-092)

KRJ1-092 was synthesized from KRJ1-069 as seen in FIG. 26 using the following procedure. KRJ1-069 (50.5 mg, 0.141 mmol) was dissolved in anh. NMP (0.7 mL). EDC hydrochloride (28.0 mg, 0.146 mmol) and DIPEA (50 mL, 0.283 mmol) were added. The reaction mixture was stirred for 30 min at room temperature. 4-(2-Aminoethyl)morpholine (39.8 mg, 0.306 mmol) was added and the mixture was stirred for 19 hr at room temperature. Additional EDC hydrochloride (19.1 mg, 0.100 mmol) and 4-(2-aminoethyl)morpholine (20.0 mg, 0.154 mmol) were added and the reaction mixture was stirred for 6 hr at room temperature. DCM (5 mL) and water (5 mL) were added to the reaction mixture and the phases were separated. The aq. phase was extracted with DCM (5 mL) and a mixture of CHCl₃ and EtOH (2:1, v/v)(2×5 mL). The combined organic phases were dried over MgSO₄ and evaporated. The crude product was purified by preparative HPLC to give the product KRJ1-092 as a white solid (12.0 mg, 18%). Contaminated fractions were collected and will be purified again. HPLC RT: 6.102 min (method B). LC-MS: found: 472.3 [M+H]⁺, C₂₂H₃₀N₇O₅⁺ requires M, 472.23. ¹H NMR (800 MHz, DMSO-d₆) δ 10.23 (s, 1H), 8.72 (t, J=5.7 Hz, 1H), 7.81 (d, J=8.4 Hz, 2H), 7.38 (d, J=8.3 Hz, 2H), 6.60-6.57 (m, 2H), 4.91 (s, 2H), 4.26-4.22 (m, 2H), 3.98 (s, 2H), 3.72-3.64 (m, 2H), 3.63-3.60 (m, 2H), 3.58-3.54 (m, 2H), 3.52-3.50 (m, 2H), 3.30 (t, J=6.3 Hz, 2H), 3.26 (s, 3H), 3.16-3.11 (m, 2H). ¹³C NMR (201 MHz, DMSO-d₆) δ 166.56, 159.81, 152.30, 149.16, 147.80, 140.69, 132.93, 127.57, 127.28, 98.42, 70.22, 65.30, 63.31, 58.06, 55.30, 51.29, 41.80, 33.88. Conclusion: KRJ1-092 was synthesized from KRJ1-069 in 18% yield.

### Example 39: Procedure for preparation of 4-((6-amino-8-hydroxy-2-(2-methoxyethoxy)-9H-purin-9-yl)methyl)-N-(4-aminophenethyl)benzamide (KRJ1-101)

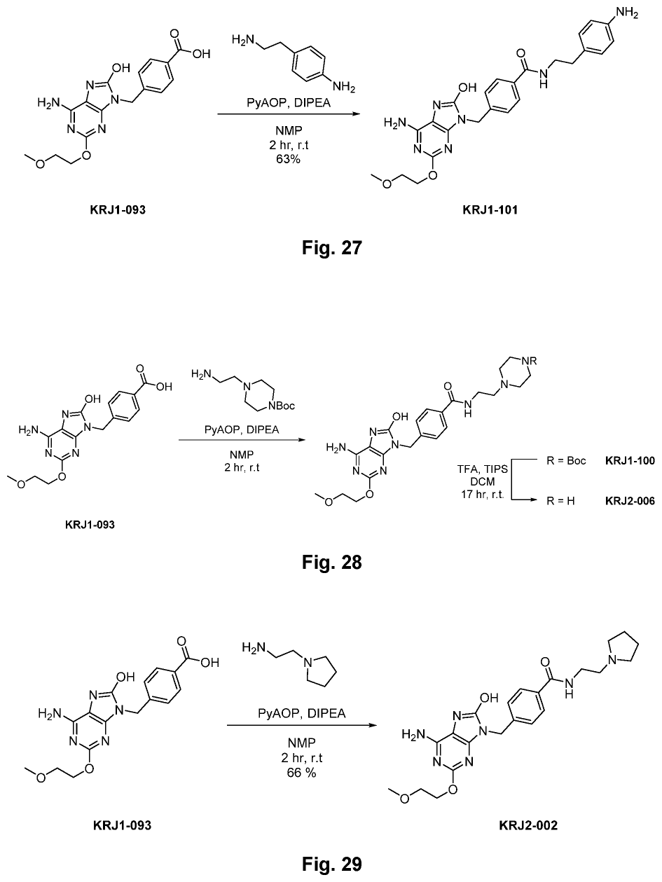

KRJ1-101 was synthesized from KRJ1-093 as seen in FIG. 27 using the following procedure. KRJ1-093 (59.9 mg, 0.167 mmol) was dissolved in anh. NMP (0.7 mL). PyAOP (90.8 mg, 0.174 mmol) and DIPEA (90 mL, 0.517 mmol) were added and the reaction mixture was stirred for 2 hr at room temperature. 4-(2-Aminoethyl)aniline (97%, 48.8 mg, 0.348 mmol) was added and the reaction mixture was stirred for 2 hr at room temperature. The mixture was concentrated in vacuo. The crude product was purified by preparative HPLC to give the product KRJ1-101 as a white solid (50.0 mg, 63%). R_{f}=0.60 (2% TEA, 5% MeOH in DCM, v/v/v). HPLC R_{T}: 7.168 min (method A). LC-MS: found: 478.2 [M+H]⁺, C₂₄H₂₈N₇O₄⁺ requires M, 478.22. ¹H NMR (400 MHz, DMSO-d₆) δ 10.15 (s, 1H), 8.49 (t, J=5.6 Hz, 1H), 7.74 (d, J=8.3 Hz, 2H), 7.34 (d, J=8.4 Hz, 2H), 7.29 (d, J=8.4 Hz, 2H), 7.17 (d, J=8.4 Hz, 2H), 6.56 (s, 2H), 4.90 (s, 2H), 4.29-4.21 (m, 2H), 3.60-3.54 (m, 2H), 3.50-3.42 (m, 2H), 3.26 (s, 3H), 2.83 (t, J=7.2 Hz, 2H). ¹³C NMR (101 MHz, DMSO-d₆) δ 166.30, 160.22, 152.71, 149.59, 148.18, 140.59, 134.16, 130.40, 127.80, 127.66, 122.49, 98.83, 72.95, 70.65, 65.79, 63.54, 58.52, 42.57, 41.05, 34.89. Conclusion: KRJ1-101 was synthesized from KRJ1-093 in 63% yield.

### Example 40: Procedure for preparation of 4-((6-amino-8-hydroxy-2-(2-methoxyethoxy)-9H-purin-9-yl)methyl)-N-(2-(piperazin-1-yl)ethyl)benzamide (KRJ2-006)

KRJ2-006 was synthesized from KRJ1-093 as seen in FIG. 28 using the following procedure.

KRJ1-093 (62.9 mg, 0.175 mmol) was dissolved in anh. NMP (0.7 mL). PyAOP (90.4 mg, 0.173 mmol) and DIPEA (90 mL, 0.517 mmol) were added and the reaction mixture was stirred for 2 hr at room temperature 4-(2-Aminoethyl)-1-boc-piperazine (97%, 88.0 mg, 0.372 mmol) was added and the reaction mixture was stirred for 2 hr at room temperature The mixture was concentrated in vacuo. The crude product was purified by preparative HPLC to give the intermediate product KRJ1-100 as a white solid (89.0 mg, >90% purity by HPLC). HPLC RT: 7.894 min (method B). LC-MS: found: 571.2 [M+H]⁺, C₂₇H₃₉N₈O₆⁺ requires M, 571.29. ¹H NMR (400 MHz, DMSO-d₆) δ 10.15 (s, 1H), 8.70 (t, J=5.6 Hz, 1H), 7.81 (d, J=8.3 Hz, 2H), 7.38 (d, J=8.2 Hz, 2H), 6.56 (s, 2H), 4.92 (s, 2H), 4.28-4.22 (m, 2H), 4.10-3.92 (m, 2H), 3.67-3.48 (m, 6H), 3.30 (t, J=6.2 Hz, 2H), 3.26 (s, 3H), 3.19-2.96 (m, 4H), 1.41 (s, 9H). ¹³C NMR (101 MHz, DMSO-d₆) δ 166.58, 159.76, 153.26, 152.27, 149.15, 147.71, 140.69, 132.92, 127.59, 127.29, 98.40, 79.93, 70.21, 65.35, 58.07, 54.88, 50.82 (2×C), 42.15, 34.08, 27.94.

KRJ1-100 (76 mg, 0.133 mmol) was dissolved in a mixture of DCM and DMF (10:1, v/v) (11 mL). A solution of TFA and TIPS [obtained by the addition of TFA (2 mL, 26.1 mmol) and TIPS (30 mL, 0.146 mmol) to DCM (5 mL) at 0° C.] was added dropwise to the reaction mixture, which was stirred for 17 hr at room temperature Water (20 mL) was added to the reaction mixture and the phases were separated. The aq. phase was extracted with DCM (2×30 mL) and a mixture of CHCl₃ and EtOH (2:1, v/v)(30 mL). The aq. phase was concentrated in vacuo giving the product KRJ2-006 as a white solid (72.0 mg, 87%) HPLC RT: 5.691 min (method B). LC-MS: found: 471.3 [M+H]⁺, C₂₂H₃₁N₈O₄⁺ requires M, 471.25. ¹H NMR (400 MHz, DMSO-d₆) δ 10.14 (s, 1H), 8.65 (t, J=5.6 Hz, 1H), 7.81 (d, J=8.3 Hz, 2H), 7.38 (d, J=8.3 Hz, 2H), 6.57 (s, 2H), 4.92 (s, 2H), 4.28-4.21 (m, 2H), 3.62-3.53 (m, 4H), 3.35 (brs, 8H), 3.26 (s, 5H). ¹³C NMR (101 MHz, DMSO-d₆) δ 166.51, 159.73, 152.25, 149.14, 147.67, 140.63, 132.98, 127.54, 127.28, 98.39, 70.20, 65.35, 58.06, 55.24, 48.47, 42.14, 40.60, 34.50.

Conclusion: KRJ2-006 was synthesized from KRJ1-093 in 87% yield.

### Example 41: Procedure for preparation of 4-((6-amino-8-hydroxy-2-(2-methoxyethoxy)-9H-purin-9-yl)methyl)-N-(2-(pyrrolidin-1-yl)ethyl)benzamide (KRJ2-002)

KRJ2-002 was synthesized from KRJ1-093 as seen in FIG. 29 using the following procedure.

KRJ1-093 (55.5 mg, 0.154 mmol) was dissolved in anh. NMP (0.7 mL). PyAOP (80.9 mg, 0.155 mmol) and DIPEA (90 mL, 0.517 mmol) were added and the reaction mixture was stirred for 2 hr at room temperature 1-(2-Aminoethyl)pyrrolidine (98%, 48.0 mg, 0.360 mmol) was added and the reaction mixture was stirred for 2 hr at room temperature The mixture was concentrated in vacuo. The crude product was purified by flash column chromatography (2% triethylamine, 8% MeOH in DCM, v/v/v) to give the product KRJ2-002 as a white solid (46.4 mg, 66%). R_{f}=0.25 (2% triethylamine, 8% MeOH in DCM, v/v/v). HPLC RT: 6.619 min (method A). LC-MS: found: 456.2 [M+H]⁺, C₂₂H₃₀N₇O₄⁺ requires M, 456.23. ¹H NMR (400 MHz, DMSO-d₆) δ 10.83 (s, 1H), 8.51 (t, J=5.7 Hz, 1H), 7.80 (d, J=8.3 Hz, 2H), 7.33 (d, J=8.2 Hz, 2H), 6.90 (s, 2H), 4.89 (s, 2H), 4.26-4.20 (m, 2H), 3.59-3.54 (m, 2H), 3.43-3.41 (m, 2H), 3.25 (s, 3H), 2.71-2.65 (m, 2H), 2.63-2.57 (m, 4H), 1.75-1.65 (m, 4H). ¹³C NMR (101 MHz, DMSO-d₆) δ 165.84, 159.80, 152.04, 148.92, 147.96, 140.27, 133.55, 127.43, 127.12, 98.46, 70.23, 65.21, 58.05, 54.57, 53.55, 42.04, 38.08, 23.03. Conclusion: KRJ2-002 was synthesized from KRJ1-093 in 66% yield.

### Example 42: Procedure for preparation of N-(2-(1H-pyrrol-1-yl)ethyl)-4-((6-amino-8-hydroxy-2-(2-methoxyethoxy)-9H-purin-9-yl)methyl)benzamide (KRJ2-014)

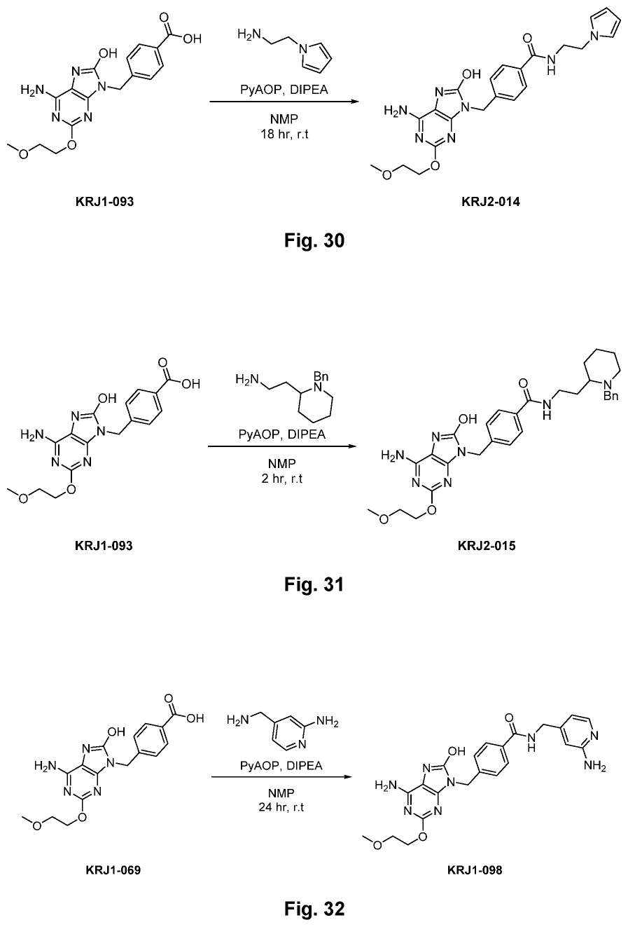

KRJ2-014 was synthesized from KRJ1-093 as seen in FIG. 30 using the following procedure.

KRJ1-093 (42.9 mg, 0.119 mmol) was dissolved in anh. NMP (0.4 mL). PyAOP (64.9 mg, 0.124 mmol) and DIPEA (90 mL, 0.517 mmol) were added and the reaction mixture was stirred for 2 hr at room temperature 2-(1H-Pyrrol-1-yl)ethanamine (95%, 36.2 mg, 0.312 mmol) and anh. NMP (0.3 mL) were added and the reaction mixture was stirred for 18 hr at room temperature The mixture was concentrated in vacuo. Purification is in process. HPLC RT: 8.304 min (method B). LC-MS: found: 560.5 [M+H]⁺, C₃₀H₃₈N₇O₄⁺ requires M, 560.29.

### Example 43: Procedure for preparation of 4-((6-amino-8-hydroxy-2-(2-methoxyethoxy)-9H-purin-9-yl)methyl)-N-(2-(1-benzylpiperidin-2-yl)ethyl)benzamide (KRJ2-015)

KRJ2-015 was synthesized from KRJ1-093 as seen in FIG. 31 using the following procedure. KRJ1-093 (56.6 mg, 0.158 mmol) was dissolved in anh. NMP (0.4 mL). PyAOP (83.5 mg, 0.160 mmol) and DIPEA (90 mL, 0.517 mmol) were added and the reaction mixture was stirred for 2 hr at room temperature. 2-(1-Benzylpiperidin-2-yl)ethan-1-amine (95%, 78.9 mg, 0.343 mmol) and anh. NMP (0.3 mL) were added and the reaction mixture was stirred for 2 hr at room temperature. The mixture was concentrated in vacuo. HPLC RT: 7.899 min (method B). LC-MS: found: 452.4 [M+H]⁺, C₂₂H₂₆N₇O₄⁺ requires M, 452.20.

### Example 44: Procedure for the Preparation of tert-butyl (4-(((2-chloro-3-pentanamidoquinolin-4-yl)amino)methyl)benzyl)carbamate (MK124)

MK124 was synthesized as a precursor of target molecule MK130 seen in FIG. 33.

MK124 was synthesized in the following steps from 2,4-dichloro-3-nitroquinoline as seen in FIG. 2A with a modified procedure from previous reports (Shukla et al, Bioorganic & Medicinal Chemistry Letters, 2010, 20, 6384-6386; Shukla et al, J. Med. Chem. 2010, 53, 4450-4465):

a) Tert-butyl (4-(((2-chloro-3-nitroquinolin-4-yl)amino)methyl)benzyl)carbamate:

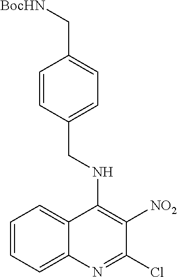 [SMILES: CC(C)(C)OC(=O)NCc1ccc(CNc2c([N+](=O)[O-])c(Cl)nc3ccccc23)cc1]

2,4-di chloro-3-nitro quinoline (150 mg, 0.617 mmol) was dispersed in anhydrous DCM (10 mL) and stirred at room temperature for 5 min before a solution of 1-(N-Boc-aminomethyl)-4-(aminomethyl)benzene (146 mg, 0.617 mmol) in DCM (10 mL) with Et₃N (90 μL) was added in dropwise. This mixture was then stirred at 20° C. for 3 h, then added a solution of 1-(N-Boc-aminomethyl)-4-(aminomethyl)benzene (90 mg) with Et₃N (90 uL) and was allowed to stir at 20° C. for 24 h, where LCMS saw full conversion of the dichloro-nitro-quinoline. The reaction mixture was diluted with DCM to 25 mL and was washed with brine, which was extracted with 2×50 mL DCM. The combined organic phases were dried with Na₂SO₄, filtered, and concentrated before the crude product was purified by flash column chromatography on silica (hexane/ethyl acetate 3:2). Product containing fractions with single spot by TLC were pooled and concentrated to an orange oil that was further concentrated to an orange solid at 0.2 mbar. Yielded 246.9 mg (90%).

R_{f}=0.57 (hexane/ethyl acetate 1:1 (v/v)). UPLCMS of product (LC2, C₁₈): Retention time=4.58 min. Calculated mass of C₂₂H₂₃ClN₄O₄=442.14; Observed [M+Na:M+Na+2]⁺ as m/z=465.1/467.2 in a 3:1 ratio.

¹H NMR (400 MHz, DMSO-d₆) δ 8.53 (d, J=8.5 Hz, 1H), 8.48 (t, J=6.3 Hz, 1H), 7.88-7.81 (m, 2H), 7.67 (ddd, J=8.4, 5.3, 2.9 Hz, 1H), 7.36 (t, J=6.2 Hz, 1H), 7.21 (d, J=8.3 Hz, 2H), 7.17 (d, J=8.4 Hz, 2H), 4.41 (d, J=6.1 Hz, 2H), 4.09 (d, J=6.2 Hz, 2H), 1.38 (s, 9H).

¹³C NMR (101 MHz, DMSO) δ 155.8, 145.3, 144.4, 141.1, 139.5, 136.1, 132.3, 128.6, 127.0, 126.9, 126.7, 126.5, 123.1, 119.7, 77.8, 46.6, 43.0, 28.2.

b) Tert-butyl (4-(((3-amino-2-chloroquinolin-4-yl)amino)methyl)benzyl)carbamate:

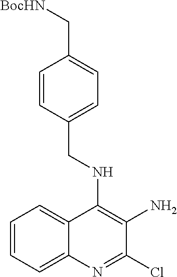 [SMILES: CC(C)(C)OC(=O)NCc1ccc(CNc2c(N)c(Cl)nc3ccccc23)cc1]

Tert-butyl (4-(((2-chloro-3-nitroquinolin-4-yl)amino)methyl)benzyl) carbamate (980 mg, 2.213 mmol) was dissolved in ethyl acetate (60 mL) was added Na₂SO₄ (314 mg, 2.213 mmol) and catalyst (10 wt % Pd/C, 118 mg, 0.111 mmol) before this dispersion was pipetted (pasteur pipette) into a 150 mL Swedgelok pressure vessel with 20 mL ethyl acetate. The vessel was sealed and was filled with H₂ to 5 bar. The vessel was tilted and shaken for 8 h before it was emptied. The liquid was filtered through a pad of Celite and concentrated at reduced pressure and the resulting solid was dissolved in acetonitrile/water 1:1 (v/v) (5 mL) and lyophilized o.n. The resulting fluffy green solid was purified by semi-prep HPLC with a Phenomenex Gemini® C₁₈ (250×21.2 mm) column using a linear gradient over 20 min (A/B 60:40 4 35:65). Yielded 205.4 mg (22%) as a pale green solid. UPLCMS: Calculated mass for C₂₂H₂₅ClN₄O₂=412.17. Observed [M+H:M+H+2]⁺ as m/z=413.2; 415.1 in a 3:1 ratio.
c) Tert-butyl (4-(((2-chloro-3-pentanamidoquinolin-4-yl)amino)methyl)benzyl)carbamate

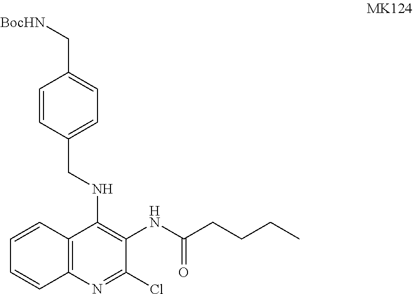 [SMILES: CCCCC(=O)Nc1c(Cl)nc2ccccc2c1NCc1ccc(CNC(=O)OC(C)(C)C)cc1]

Tert-butyl (4-(((2-chloro-3-nitroquinolin-4-yl)amino)methyl)benzyl) carbamate (195 mg, 0.472 mmol) was dissolved in tetrahydrofuran (9.5 mL) and stirred at room temperature, then added Et₃N (99 μL, 0.708 mmol) and valeroyl chloride (67 μL, 0.567 mmol). This reaction mixture was stirred at room temperature o.n. The reaction mixture was concentrated at reduced pressure and re-dissolved in 4 mL acetonitrile before the product was purified by semi-prep HPLC with a Phenomenex Gemini® C₁₈ (250×21.2 mm) with a linear gradient over 20 min (A/B 60:40→30:70). Yielded 71.8 mg (31%) as a fluffy pale green solid. UPLCMS of product (LC2, C₁₈): Retention time=3.98 min. Calculated mass of C₂₇H₃₃ClN₄O₃=496.22; Observed [M+H:M+H+2]⁺ as m/z=497.3; 499.1 in a 3:1 ratio.

¹H NMR (400 MHz, DMSO-d₆) δ 9.29 (s, 1H), 8.28 (d, J=8.6 Hz, 1H), 7.75-7.64 (m, 2H), 7.48 (dt, J=8.6, 4.2 Hz, 1H), 7.42-7.28 (m, 2H), 7.24 (d, J=7.9 Hz, 2H), 7.17 (d, J=8.2 Hz, 2H), 4.72 (s, 2H), 4.08 (d, J=6.2 Hz, 2H), 2.10 (t, J=7.5 Hz, 2H), 1.56-1.40 (m, 2H), 1.38 (s, 9H), 1.28 (h, J=7.4 Hz, 2H), 0.86 (t, J=7.3 Hz, 3H).

¹³C NMR (101 MHz, DMSO) δ 172.7, 155.8, 151.7, 150.5, 145.3, 138.7, 138.3, 130.2, 127.6, 126.9, 126.5, 125.1, 122.8, 120.0, 109.7, 77.7, 48.2, 43.1, 34.8, 28.2, 26.8, 21.9, 13.7. Conclusion: MK124 was synthesized from 2,4-quinolinediol in three steps in a 6% isolated yield.

### Example 45: Procedure for the Preparation of tert-butyl (4-((4-azido-2-butyl-1H-imidazo[4,5-c]quinolin-1-yl)methyl)benzyl)carbamate (MK125)

MK125 was synthesized as a precursor of target molecule seen in FIG. 33.

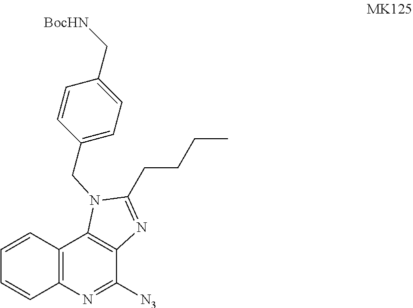 [SMILES: CCCCc1nc2c(N=[N+]=[N-])nc3ccccc3c2n1Cc1ccc(CNC(=O)OC(C)(C)C)cc1]

Tert-butyl (4-(((2-chloro-3-pentanamidoquinolin-4-yl)amino)methyl)benzyl) carbamate (MK124) (67 mg, 0.135 mmol) was dissolved in ethanol (37 mL) and 5M NaOH (1 mL). This mixture was stirred at 20° C. for 4 h where LCMS saw full conversion to imidazole ring-closed intermediate. The reaction mixture was concentrated to 10 mL, added water (30 mL) and ethyl acetate (100 mL). The aqueous phase was extracted with ethyl acetate (50 mL) and the combined organic phases were dried with Na₂SO₄, filtered, and concentrated at reduced pressure. UPLCMS of product (LC2, C₁₈): Retention time=4.44 min. Calculated mass of C₂₇H₃₁ClN₄O₂=478.21; Observed [M+H:M+H+2]⁺ as m/z=479.3; 481.3 in a 3:1 ratio.

The intermediate was subsequently dissolved in DMSO (10 mL) and added NaN₃ (22 mg, 0.34 mmol) and was stirred at 160° C. under stream of N₂ for 12 h. The mixture was allowed to reach ambient temperature and was then added to a separatory funnel with ethyl acetate and aqueous saturated NaHCO₃ (50 mL each). The aqueous phase was extracted with ethyl acetate (2×50 mL) and the combined organic phases were dried with Na₂SO₄, filtered, and concentrated at reduced pressure. The resulting crude product was purified by semi-prep HPLC with a Phenomenex Gemini® Cis (250×21.2 mm) column using a linear gradient 20 min (A/B 60:40→30:70). Yielded 29.7 mg (45%) as a fluffy white solid. UPLCMS of product (LC2, C₁₈): Retention time=4.07 min. Calculated mass of C₂₇H₃₁N₇O₂=485.25; Observed [M+H]⁺ as m/z=486.3.

NMR (400 MHz, DMSO-d₆) δ 8.73 (d, J=7.6 Hz, 1H), 8.20 (d, J=8.3 Hz, 1H), 7.80 (t, J=7.6 Hz, 1H), 7.66 (t, J=7.6 Hz, 1H), 7.33 (t, J=6.1 Hz, 1H), 7.17 (d, J=8.1 Hz, 2H), 7.03 (d, J=8.2 Hz, 2H), 6.00 (s, 2H), 4.06 (d, J=5.9 Hz, 2H), 2.99 (t, J=7.6 Hz, 2H), 1.79 (p, J=7.6 Hz, 2H), 1.48-1.31 (m, 11H), 0.89 (t, J=7.3 Hz, 3H).

¹³C NMR (101 MHz, DMSO) δ 156.5, 155.8, 143.5, 139.7, 134.0, 129.7, 128.5, 128.0, 127.8, 127.5, 125.5, 125.0, 122.3, 117.6, 114.8, 77.8, 48.1, 42.9, 29.0, 28.2, 26.2, 21.7, 13.7.

Conclusion: MK125 was synthesized from MK124 in two steps in a 45% isolated yield.

### Example 46: Procedure for the preparation of 1-(4-(aminomethyl)benzyl)-2-butyl-1H-imidazo[4,5-c]quinolin-4-amine (MK130)

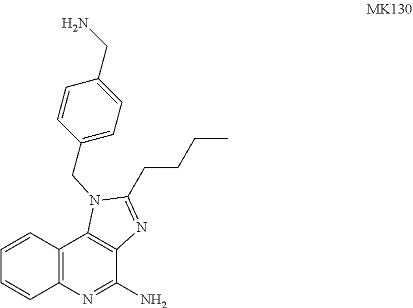 [SMILES: CCCCc1nc2c(N)nc3ccccc3c2n1Cc1ccc(CN)cc1]

Target molecule MK130 was synthesized as seen in FIG. 33. Tert-butyl (4-((4-azido-2-butyl-1H-imidazo[4,5-c]quinolin-1-yl)methyl) benzyl)carbamate (MK125) 125.0 mg, 0.257 mmol) was added triphenylphosphine (1000 mg, 3.861 mmol) and a stir bar. These solids were stirred at 160° C. under a stream of N₂ for 30 min, resulting in full conversion of the azide to the phosphazene intermediate. UPLCMS of intermediate (LC2, C₁₈): Retention time=4.10 min. Calculated mass of C45H46N5O2P=719.34. Observed [M+H]+ as m/z=720.3.

The triphenylphosphine solution was cooled to room temperature and added acetonitrile (5 mL), water (1.5 mL), and TFA (neat, 1.5 mL) and was subsequently stirred at 80° C. for 24 h before addition of water (2 mL) formed a milky solution that was filtered and added neat TFA (1 mL), stirred under a flow of N₂ for 1 h, and purified by semi-prep HPLC with a Phenomenex Gemini® C₁₈ (250×21.2 mm) column using a linear gradient over 15 min (A/B 90:10→75:25). Yielded 62.9 mg (68%) as a fluffy white solid. HPLC >95%. UPLCMS of product (LC1, C₁₈): Retention time=1.23 min. Calculated mass of C₂₂H₂₅N₅=359.21. Observed [M+H]⁺ as m/z=360.2.

¹H NMR (400 MHz, DMSO-d₆) δ 8.55 (s, 3H), 7.94 (dd, J=8.4, 1.2 Hz, 1H), 7.78 (dd, J=8.4, 1.1 Hz, 1H), 7.66-7.57 (m, 1H), 7.48 (d, J=8.0 Hz, 2H), 7.35 (ddd, J=8.3, 7.1, 1.2 Hz, 1H), 7.11 (d, J=8.1 Hz, 2H), 5.99 (s, 2H), 5.81 (s, 2H), 3.95 (q, J=5.8 Hz, 2H), 2.95 (t, J=7.7 Hz, 2H), 1.73 (p, J=7.5 Hz, 2H), 1.38 (h, J=7.3 Hz, 2H), 0.87 (t, J=7.3 Hz, 3H).

¹³C NMR (101 MHz, DMSO) δ 156.9, 149.0, 135.7, 135.3, 133.6, 133.5, 129.7, 129.5, 125.7, 124.7, 124.6, 121.6, 118.4, 112.3, 48.2, 41.6, 29.2, 26.2, 21.8, 13.7.

Conclusion: MK130 was synthesized from MK125 in two steps in a 68% isolated yield.

### Example 47: General Procedure for the Preparation of Amino Acid Derivatives of MK130

This example describes the general procedure for the amino acid derivatization of MK130 which forms examples 50-58.

A solution of (Fmoc)-protected L-amino acid (0.2M in DMF, 1.5 equivalents) was mixed with a solution of O-(6-chlorobenzotriazol-1-yl)-N,N,N′,N′-tetramethyluronium hexafluorophosphate (HCTU) (0.2M in DMF, 1.5 equivalents) before a solution of N,N-diisopropylethylamine (DIPEA) (2M in DMF, 3.0 equivalents) was added. This mixture was stirred at 20° C. for 15 minutes before a solution of imidazoquinoline MK130 (0.1M in DMF/DMSO 1:1 (v/v), 1.0 equivalent) was added. This mixture was then stirred at 20° C. for 30 minutes. Ethyl acetate (25 mL) and brine (25 mL) was then added, and the aqueous phase was extracted with ethyl acetate (25 mL). The combined organic phases were dried, filtered, and concentrated before addition of 20% piperidine in ethyl acetate (5 mL). The mixture was swirled for 15 minutes before the reaction mixture was concentrated at reduce pressure. Trityl or tert-butyl protected amino acid side chains were deprotected in TFA/triisopropylsilane/water 95:2.5:2.5 (v/v) for 15 minutes, then concentrated under a stream of nitrogen. The resulting oil was diluted with acidic water/acetonitrile 1:1 (4 mL), filtered, and purified by semi prep-HPLC with a Phenomenex Gemini® C₁₈ (250×21.2 mm) column using the indicated linear gradients to yield the products as fluffy white solids after lyophilization. The product-containing fractions with >95% purity (HPLC) were pooled and lyophilized to yield the product as the trifluoroacetic acid salt.

### Example 48: General Procedure for the Preparation of Acetylated Derivatives of MK130

This example describes the general procedure for the anhydride derivatization of MK130 which forms examples 49 and 59.

A solution of imidazoquinoline MK130 (0.1M in DMF/DMSO 1:1 (v/v), 1.0 equivalent) was added DIPEA neat (1.0 equivalent) followed by addition of the anhydride (1.5 equivalents) before this mixture was allowed to stir at 20° C. for 1 h. The solution was subsequently diluted to 5 mL in water/acetonitrile 1:1 (v/v)+0.1 vol % TFA, filtered, then purified and isolated as described in the general procedure for the preparation of amino acid derivatives of MK130 (example 47).

### Example 49: Procedure for the preparation of N-(4-((4-amino-2-butyl-1H-imidazo[4,5-c]quinolin-1-yl)methyl)benzyl)acetamide (MK132)

This example describes an acetylation of MK130.

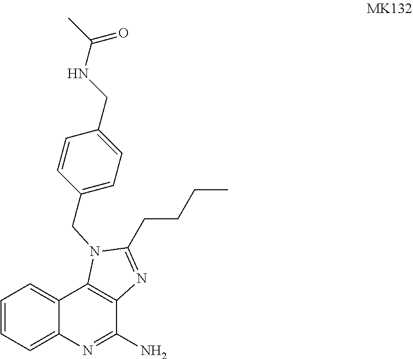 [SMILES: CCCCc1nc2c(N)nc3ccccc3c2n1Cc1ccc(CNC(C)=O)cc1]

MK132 was synthesized from MK130 according to the general procedure described in example 48:
MK130 (0.100 mmol), DIPEA (0.100 mmol), acetic anhydride (0.149 mmol). Purification of the product was carried out with a linear gradient over 15 min (A/B 85:15→70:30). Yielded 31.1 mg (61%) as a fluffy white solid. HPLC >95%. UPLCMS of product (LC2, C₁₈): Retention time=2.02 min. Calculated mass of C₂₄H₂₇N₅O=401.22; Observed [M+H]⁺ as m/z=402.2.

¹H NMR (400 MHz, DMSO-d₆) δ 9.03 (s, 2H), 8.31 (t, J=6.0 Hz, 1H), 7.95 (dd, J=8.4, 1.3 Hz, 1H), 7.78 (dd, J=8.4, 1.1 Hz, 1H), 7.62 (ddd, J=8.4, 7.2, 1.2 Hz, 1H), 7.36 (ddd, J=8.3, 7.2, 1.2 Hz, 1H), 7.20 (d, J=8.2 Hz, 2H), 7.02 (d, J=8.2 Hz, 2H), 5.94 (s, 2H), 4.19 (d, J=6.0 Hz, 2H), 3.00-2.91 (m, 2H), 1.82 (s, 3H), 1.73 (p, J=7.5 Hz, 2H), 1.38 (h, J=7.4 Hz, 2H), 0.87 (t, J=7.3 Hz, 3H).

¹³C NMR (101 MHz, DMSO) δ 169.1, 156.9, 149.0, 139.2, 135.3, 134.0, 133.9, 129.4, 127.8, 125.5, 124.7, 124.7, 121.6, 118.5, 112.4, 48.2, 41.6, 29.2, 26.2, 22.5, 21.8, 13.7.

Conclusion: MK132 was synthesized as from MK130 in a 61% yield.

### Example 50: Procedure for the preparation of (S)-2-amino-N-(4-((4-amino-2-butyl-1H-imidazo [4,5-c] quinolin-1-yl)methyl)benzyl)succinamide (MK135)

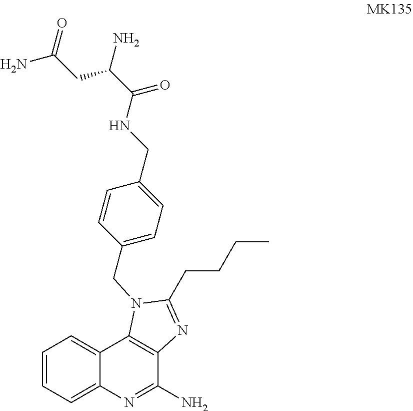 [SMILES: CCCCc1nc2c(N)nc3ccccc3c2n1Cc1ccc(CNC(=O)[C@@H](N)CC(N)=O)cc1]

This example describes an L-asparagine derivatization of MK130. MK135 was synthesized from MK130 according to the general procedure described in example 47: Fmoc-Asn(Trt)-OH (0.095 mmol), HCTU (0.095 mmol), DIPEA (0.190 mmol), and MK130 (0.063 mmol). UPLCMS of Fmoc-protected intermediate (LC1, C₁₈): Retention time=2.27 min. Calculated mass of C₆₀H₅₅N₇O₄=937.43; Observed [M+H]⁺ as m/z=938.5.

Purification of the product was carried out with a linear gradient over 15 min (A/B 90:10→75:25). Yielded 26.2 mg (70%) as a fluffy white solid. HPLC >95%. UPLCMS of product (LC1, C₁₈): Retention time=0.97 min. Calculated mass of C₂₆H₃₁N₇O₂=473.25; Observed [M+H]⁺ as m/z=474.3.

¹H NMR (400 MHz, DMSO-d₆) δ 9.18 (s, 2H), 8.83 (t, J=5.9 Hz, 1H), 8.11 (s, 3H), 7.95 (dd, J=8.4, 1.3 Hz, 1H), 7.77 (dd, J=8.4, 1.1 Hz, 1H), 7.66-7.58 (m, 2H), 7.36 (ddd, J=8.4, 7.1, 1.2 Hz, 1H), 7.23 (d, J=8.2 Hz, 2H), 7.23 (s, 1H), 7.03 (d, J=8.0 Hz, 2H), 5.95 (s, 2H), 4.30 (qd, J=15.6, 5.8 Hz, 2H), 4.04 (dd, J=8.0, 4.9 Hz, 1H), 2.96 (t, J=7.4 Hz, 2H), 2.68 (dd, J=16.8, 4.9 Hz, 1H), 2.59 (dd, J=16.8, 8.0 Hz, 1H), 1.74 (p, J=7.4 Hz, 2H), 1.39 (h, J=7.4 Hz, 2H), 0.88 (t, J=7.3 Hz, 3H).

¹³C NMR (101 MHz, DMSO) δ 170.5, 168.1, 156.9, 149.1, 138.0, 135.3, 134.2, 134.0, 129.5, 127.7, 125.5, 124.7, 124.7, 121.5, 118.4, 112.4, 49.3, 48.2, 41.9, 35.5, 29.2, 26.2, 21.8, 13.7.

Conclusion: MK135 was synthesized as from MK130 in a 70% yield.

### Example 51: Procedure for the preparation of (S)-2-amino-N-(4-((4-amino-2-butyl-1H-imidazo[4,5-c]quinolin-1-yl)methyl)benzyl)pentanediamide (MK136)

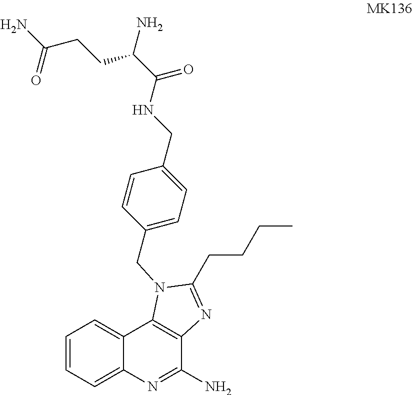 [SMILES: CCCCc1nc2c(N)nc3ccccc3c2n1Cc1ccc(CNC(=O)[C@@H](N)CCC(N)=O)cc1]

This example describes an L-glutamine derivatization of MK130. MK136 was synthesized from MK130 according to the general procedure described in example 47:

Fmoc-Gln(Trt)-OH (0.123 mmol), HCTU (0.123 mmol), DIPEA (0.246 mmol), and MK130 (0.082 mmol). UPLCMS of Fmoc-protected intermediate (LC1, C₁₈): Retention time=2.26 min. Calculated mass of C₆₁H₅₇N₇O₄=951.45; Observed [M+H]⁺ as m/z=952.4.

Purification of the product was carried out with a linear gradient over 15 min (A/B 85:15→70:30). Yielded 22.6 mg (46%) as a fluffy white solid. HPLC >95%. UPLCMS of product (LC1, C₁₈): Retention time=0.99 min. Calculated mass of C₂₇H₃₃N₇O₂=487.27; Observed [M+H]+ as m/z=488.4.

¹H NMR (400 MHz, DMSO-d₆) δ 9.21 (s, 2H), 8.92 (t, J=5.7 Hz, 1H), 8.22 (s, 3H), 7.95 (d, J=8.2 Hz, 1H), 7.76 (d, J=8.2 Hz, 1H), 7.66-7.57 (m, 1H), 7.42 (s, 1H), 7.38-7.32 (m, 1H), 7.26 (d, J=8.2 Hz, 2H), 7.05 (d, J=8.2 Hz, 2H), 6.93 (s, 1H), 5.95 (s, 2H), 4.31 (qd, J=15.4, 5.8 Hz, 2H), 3.84-3.75 (m, 1H), 2.99-2.92 (m, 2H), 2.16 (dt, J=9.0, 4.2 Hz, 3H), 1.91 (q, J=7.3 Hz, 2H), 1.73 (p, J=7.6 Hz, 2H), 1.39 (h, J=7.3 Hz, 2H), 0.87 (t, J=7.3 Hz, 3H).

¹³C NMR (101 MHz, DMSO) δ 173.1, 168.4, 156.9, 149.2, 138.0, 135.3, 134.3, 134.0, 129.5, 128.0, 125.6, 124.7, 124.7, 121.6, 121.5, 118.4, 112.4, 52.1, 48.2, 41.9, 40.1, 39.9, 39.7, 39.5, 39.3, 39.1, 38.9, 30.3, 29.2, 26.9, 26.2, 21.8, 13.7.

Conclusion: MK136 was synthesized as from MK130 in a 46% yield.

### Example 52: Procedure for the preparation of (S)-2-amino-N-(4-((4-amino-2-butyl-1H-imidazo[4,5-c]quinolin-1-yl)methyl)benzyl)propanamide (MK137)

This example describes an L-alanine derivatization of MK130.

MK137 was synthesized from MK130 according to the general procedure described in example 47:

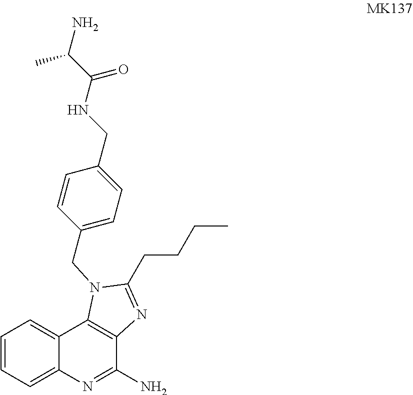 [SMILES: CCCCc1nc2c(N)nc3ccccc3c2n1Cc1ccc(CNC(=O)[C@H](C)N)cc1]

Fmoc-Ala-OH (0.139 mmol), HCTU (0.19 mmol), DIPEA (0.279 mmol), and MK130 (0.093 mmol). UPLCMS of Fmoc-protected intermediate (LC1, C₁₈): Retention time=1.81 min. Calculated mass of C₄₀H₄₀N₆O₃=652.32; Observed [M+H]⁺ as m/z=653.3.

Purification of the product was carried out by semi-prep HPLC with a linear gradient over 15 min (A/B 85:15→70:30). Yielded 36.1 mg (71%) as a fluffy white solid. HPLC >95%. UPLCMS of product (LC1, C₁₈): Retention

time=1.00 min. Calculated mass of C₂₅H₃₀N₆O=430.25; Observed [M+H]⁺ as m/z=431.2.

¹H NMR (400 MHz, DMSO-d₆) δ 9.25 (s, 1H), 8.88 (t, J=5.9 Hz, 1H), 8.15 (s, 2H), 7.94 (dd, J=8.5, 1.5 Hz, 1H), 7.76 (dd, J=8.4, 1.2 Hz, 1H), 7.66-7.58 (m, 1H), 7.35 (ddd, J=8.5, 7.2, 1.3 Hz, 1H), 7.23 (d, J=7.9 Hz, 2H), 7.04 (d, J=8.1 Hz, 2H), 5.95 (s, 2H), 4.30 (d, J=5.8 Hz, 2H), 3.88-3.78 (m, 1H), 2.99-2.92 (m, 2H), 1.73 (p, J=7.5 Hz, 2H), 1.44-1.35 (m, 2H), 1.34 (dd, J=7.0, 1.5 Hz, 3H), 0.87 (t, J=7.3 Hz, 3H).

¹³C NMR (101 MHz, DMSO) δ 169.5, 156.9, 149.2, 138.2, 135.3, 134.3, 134.0, 129.5, 127.8, 125.6, 124.7, 124.6, 121.5, 118.3, 112.4, 48.2, 48.2, 41.7, 29.2, 26.2, 21.8, 17.1, 13.7.

Conclusion: MK137 was synthesized as from MK130 in a 71% yield.

### Example 53: Procedure for the preparation of (S)-2-amino-N-(4-((4-amino-2-butyl-1H-imidazo[4,5-c]quinolin-1-yl)methyl)benzyl)-3-phenylpropanamide (MK138)

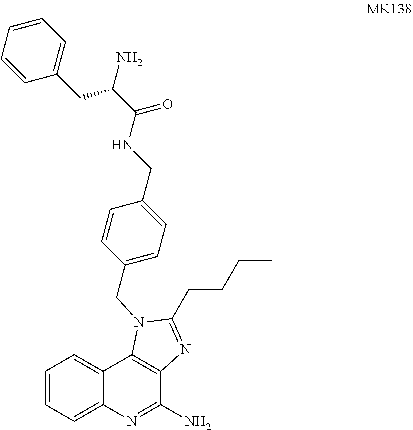 [SMILES: CCCCc1nc2c(N)nc3ccccc3c2n1Cc1ccc(CNC(=O)[C@@H](N)Cc2ccccc2)cc1]

This example describes an L-phenylalanine derivatization of MK130. MK138 was synthesized from MK130 according to the general procedure described in example 47:

Fmoc-Phe-OH (0.118 mmol), HCTU (0.118 mmol), DIPEA (0.237 mmol), and MK130 (0.079 mmol). UPLCMS of Fmoc-protected intermediate (LC1, C₁₈): Retention time=2.01 min. Calculated mass of C₄₆H₄₄N₆O₃=728.35; Observed [M+H]⁺ as m/z=729.4.

Purification of the product was carried out by semi-prep HPLC with a linear gradient over 15 min (A/B 75:25→60:40). Yielded 18.1 mg (37%) as a fluffy white solid. HPLC>95%. UPLCMS of product (LC1, C₁₈): Retention time=1.14 min. Calculated mass of C₃₁H₃₄N₆O=506.28; Observed [M+H]⁺ as m/z=507.4.

¹H NMR (400 MHz, DMSO-d₆) δ 9.20 (s, 2H), 8.84 (t, J=5.9 Hz, 1H), 8.28 (s, 3H), 7.95 (dd, J=8.4, 1.3 Hz, 1H), 7.77 (dd, J=8.4, 1.2 Hz, 1H), 7.63 (ddd, J=8.4, 7.1, 1.2 Hz, 1H), 7.35 (ddd, J=8.4, 7.2, 1.2 Hz, 1H), 7.23-7.12 (m, 5H), 7.02 (d, J=8.4 Hz, 2H), 6.97 (d, J=8.2 Hz, 2H), 5.95 (s, 2H), 4.31 (dd, J=15.4, 6.2 Hz, 1H), 4.15 (dd, J=15.4, 5.3 Hz, 1H), 3.99 (t, J=7.2 Hz, 1H), 3.02-2.93 (m, 4H), 1.74 (p, J=7.4 Hz, 2H), 1.39 (h, J=7.4 Hz, 2H), 0.87 (t, J=7.3 Hz, 3H).

¹³C NMR (101 MHz, DMSO) δ 167.8, 156.9, 149.2, 137.7, 135.3, 134.9, 134.2, 134.1, 129.4, 128.5, 127.9, 127.0, 125.5, 124.7, 124.6, 121.6, 118.4, 112.4, 53.6, 48.1, 41.7, 37.0, 29.2, 26.2, 21.8, 13.7

Conclusion: MK138 was synthesized as from MK130 in a 37% yield.

### Example 54: Procedure for the preparation of 2-amino-N-(4-((4-amino-2-butyl-1H-imidazo[4,5-c]quinolin-1-yl)methyl)benzyl)acetamide (MK139)

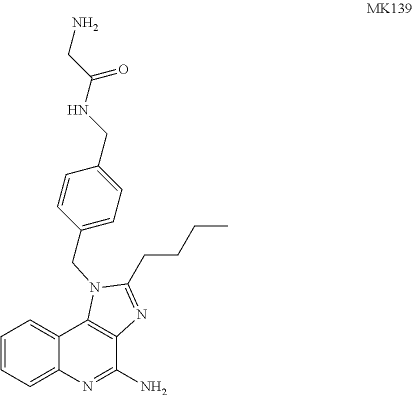 [SMILES: CCCCc1nc2c(N)nc3ccccc3c2n1Cc1ccc(CNC(=O)CN)cc1]

This example describes a glycine derivatization of MK130.

MK139 was synthesized from MK130 according to the general procedure described in example 47:

Fmoc-Gly-OH (0.144 mmol), HCTU (0.144 mmol), DIPEA (0.288 mmol), and MK130 (0.096 mmol). UPLCMS of Fmoc-protected intermediate (LC1, C₁₈): Retention time=1.76 min. Calculated mass of C₃₉H₃₈N₆O₃=638.30; Observed [M+H]⁺ as m/z=639.3.

Purification of the product was carried out by semi-prep HPLC with a linear gradient over 15 min (A/B 85:15→70:30). Yielded 31.6 mg (62%) as a fluffy white solid. HPLC>95%. UPLCMS of product (LC1, C₁₈): Retention time=0.80 min. Calculated mass of C₂₄H₂₈N₆O=416.23; Observed [M+H]⁺ as m/z=417.2.

¹H NMR (400 MHz, DMSO-d₆) δ 9.25 (s, 2H), 8.84 (t, J=5.9 Hz, 1H), 8.08 (s, 3H), 7.95 (dd, J=8.4, 1.3 Hz, 1H), 7.76 (dd, J=8.4, 1.2 Hz, 1H), 7.62 (ddd, J=8.4, 7.1, 1.2 Hz, 1H), 7.35 (ddd, J=8.4, 7.2, 1.2 Hz, 1H), 7.25 (d, J=8.3 Hz, 2H), 7.04 (d, J=8.0 Hz, 2H), 5.95 (s, 2H), 4.30 (d, J=5.8 Hz, 2H), 3.59-3.58 (m, 2H), 3.01-2.91 (m, 2H), 1.74 (p, J=7.4 Hz, 2H), 1.39 (h, J=7.4 Hz, 2H), 0.87 (t, J=7.3 Hz, 3H).

¹³C NMR (101 MHz, DMSO) δ 166.0, 156.9, 149.3, 138.2, 135.3, 134.3, 134.1, 129.5, 128.0, 125.6, 124.7, 124.6, 121.5, 118.3, 112.4, 48.2, 41.8, 40.1, 29.2, 26.2, 21.8, 13.7

Conclusion: MK139 was synthesized as from MK130 in a 62% yield.

### Example 55: Procedure for the preparation of (S)—N-(4-((4-amino-2-butyl-1H-imidazo[4,5-c]quinolin-1-yl)methyl)benzyl)pyrrolidine-2-carboxamide (MK140)

This example describes an L-proline derivatization of MK130.

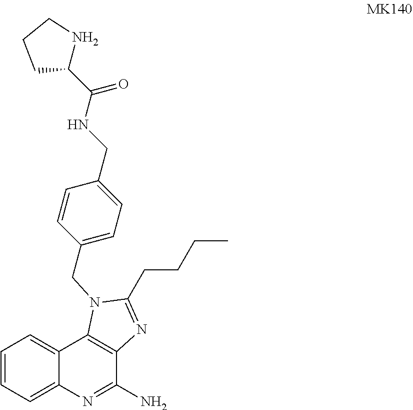 [SMILES: CCCCc1nc2c(N)nc3ccccc3c2n1Cc1ccc(CNC(=O)[C@@H]2CCCN2)cc1]

MK140 was synthesized from MK130 according to the general procedure described in example 47:

Fmoc-Pro-OH (0.131 mmol), HCTU (0.131 mmol), DIPEA (0.263 mmol), and MK130 (0.088 mmol). UPLCMS of Fmoc-protected intermediate (LC1, C₁₈): Retention time=1.81 min. Calculated mass of C₄₂H₄₂N₆O₃=678.33; Observed [M+H]⁺ as m/z=679.4.

Purification of the product was carried out by semi-prep HPLC with a linear gradient over 15 min (AB 80:20→65:35). Yielded 40.8 mg (82%) as a fluffy white solid. HPLC>95%. UPLCMS of product (LC1, C₁₈): Retention time=1.00 min. Calculated mass of C₂₇H₃₂N₆O=456.26; Observed [M+H]⁺ as m/z=457.3.

¹H NMR (400 MHz, DMSO-d₆) δ 9.61-9.59 (m, 1H), 9.27 (s, 2H), 9.03 (t, J=5.9 Hz, 1H), 8.56-8.55 (m, 1H), 7.94 (dd, J=8.4, 1.2 Hz, 1H), 7.75 (dd, J=8.3, 1.2 Hz, 1H), 7.62 (ddd, J=8.3, 7.2, 1.2 Hz, 1H), 7.35 (ddd, J=8.4, 7.2, 1.2 Hz, 1H), 7.24 (d, J=8.1 Hz, 2H), 7.05 (d, J=8.0 Hz, 2H), 5.95 (s, 2H), 4.32 (d, J=5.8 Hz, 2H), 4.21-4.16 (m, 1H), 3.21 (q, J=7.4 Hz, 2H), 2.96 (t, J=7.7 Hz, 2H), 2.33-2.22 (m, 1H), 1.93-1.78 (m, 3H), 1.73 (p, J=8.6, 8.0 Hz, 2H), 1.38 (h, J=7.4 Hz, 2H), 0.87 (t, J=7.3 Hz, 3H).

¹³C NMR (101 MHz, DMSO) δ 168.1, 156.9, 149.3, 138.1, 135.3, 134.3, 134.0, 129.4, 127.9, 125.7, 124.7, 124.6, 121.5, 118.3, 112.4, 59.1, 48.2, 45.6, 41.9, 29.5, 29.2, 26.2, 23.5, 21.8, 13.7.

Conclusion: MK140 was synthesized as from MK130 in an 82% yield.

### Example 56: Procedure for the preparation of (S)-2-amino-N-(4-((4-amino-2-butyl-1H-imidazo[4,5-c]quinolin-1-yl)methyl)benzyl)-3-(4-hydroxyphenyl)propanamide (MK141)

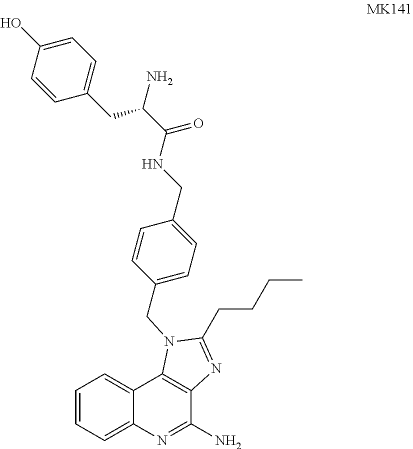 [SMILES: CCCCc1nc2c(N)nc3ccccc3c2n1Cc1ccc(CNC(=O)[C@@H](N)Cc2ccc(O)cc2)cc1]

This example describes an L-tyrosine derivatization of MK130. MK141 was synthesized from MK130 according to the general procedure described in example 47: Fmoc-Tyr(tBu)-OH (0.115 mmol), HCTU (0.115 mmol), DIPEA (0.230 mmol), and MK130 (0.077 mmol). UPLCMS of Fmoc-protected intermediate (LC1, C₁₈): Retention time=2.14 min. Calculated mass of C₅₀H₅₂N₆O₄=800.41; Observed [M+H]⁺ as m/z=801.3.

Purification of the product was carried out by semi-prep HPLC with a linear gradient over 15 min (A/B 80:20→65:35). Yielded 31.6 mg (65%) as a fluffy white solid. HPLC >95%. UPLCMS of product (LC1, C₁₈): Retention time=1.07 min. Calculated mass of C₃₁H₃₄N₆O₂=522.27; Observed [M+H]⁺ as m/z=523.3.

¹H NMR (400 MHz, DMSO-d₆) δ 9.42 (s, 1H), 9.26 (s, 2H), 8.84 (t, J=5.9 Hz, 1H), 8.23 (s, 3H), 7.92 (dd, J=8.4, 1.3 Hz, 1H), 7.75 (dd, J=8.4, 1.2 Hz, 1H), 7.61 (ddd, J=8.4, 7.1, 1.2 Hz, 1H), 7.34 (ddd, J=8.3, 7.1, 1.2 Hz, 1H), 7.07 (d, J=8.2 Hz, 2H), 7.00 (d, J=8.1 Hz, 2H), 6.95 (d, J=8.5 Hz, 2H), 6.63 (d, J=8.4 Hz, 2H), 5.94 (s, 2H), 4.29 (dd, J=15.5, 6.0 Hz, 1H), 4.19 (dd, J=15.5, 5.5 Hz, 1H), 3.91 (t, J=7.1 Hz, 1H), 3.00-2.91 (m, 2H), 2.88 (dd, J=7.1, 4.1 Hz, 2H), 1.73 (p, J=7.4 Hz, 2H), 1.39 (h, J=7.4 Hz, 2H), 0.87 (t, J=7.3 Hz, 3H). ¹³C NMR (101 MHz, DMSO) δ 168.0, 156.9, 156.6, 149.3, 137.8, 135.3, 134.2, 134.1, 130.4, 129.4, 128.0, 125.5, 124.8, 124.7, 124.6, 121.5, 118.5, 118.3, 112.4, 53.9, 48.2, 41.8, 36.3, 29.2, 26.2, 21.8, 13.7.

Conclusion: MK141 was synthesized as from MK130 in a 65% yield.

### Example 57: Procedure for the preparation of (S)-2-amino-N-(4-((4-amino-2-butyl-1H-imidazo[4,5-c]quinolin-1-yl)methyl)benzyl)-3-methylbutanamide (MK145)

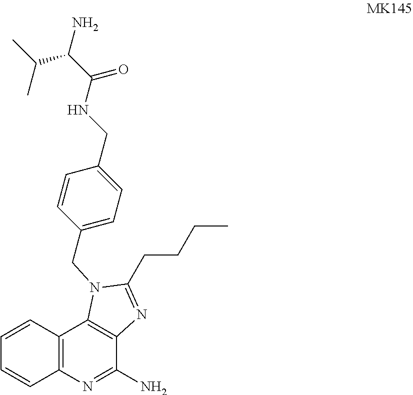 [SMILES: CCCCc1nc2c(N)nc3ccccc3c2n1Cc1ccc(CNC(=O)[C@@H](N)C(C)C)cc1]

This example describes an L-valine derivatization of MK130.

MK145 was synthesized from MK130 according to the general procedure described in example 47:

Fmoc-Val-OH (0.131 mmol), HCTU (0.131 mmol), DIPEA (0.262 mmol), and MK130 (0.087 mmol). UPLCMS of Fmoc-protected intermediate (LC2, C₁₈): Retention time=3.25 min. Calculated mass of C₄₂H₄₄N₆O₃=680.35; Observed [M+H]⁺ as m/z=681.3.

Purification of the product was carried out by semi-prep HPLC with a linear gradient over 15 min (A/B 80:20→65:35). Yielded 37.5 mg (75%) as a fluffy white solid. HPLC>99%. UPLCMS of product (LC1, C₁₈): Retention time=1.21 min. Calculated mass of C₂₇H₃₄N₆O=458.28; Observed [M+H]⁺ as m/z=459.3.

¹H NMR (400 MHz, DMSO-d₆) δ 9.15 (s, 2H), 8.89 (t, J=5.9 Hz, 1H), 8.12 (s, 3H), 7.94 (dd, J=8.4, 1.2 Hz, 1H), 7.78 (dd, J=8.4, 1.1 Hz, 1H), 7.63 (ddd, J=8.5, 7.2, 1.2 Hz, 1H), 7.34 (ddd, J=8.4, 7.1, 1.2 Hz, 1H), 7.27 (d, J=8.0 Hz, 2H), 7.05 (d, J=8.1 Hz, 2H), 5.96 (s, 2H), 4.36 (dd, J=15.2, 5.9 Hz, 1H), 4.27 (dd, J=15.2, 5.7 Hz, 1H), 3.56 (d, J=5.6 Hz, 1H), 3.01-2.92 (m, 2H), 2.04 (dq, J=13.5, 6.8 Hz, 1H), 1.73 (p, J=7.4 Hz, 2H), 1.39 (h, J=7.4 Hz, 2H), 0.90-0.85 (m, 9H).

¹³C NMR (101 MHz, DMSO) δ 167.8, 156.8, 149.1, 138.1, 135.3, 134.4, 134.1, 129.4, 128.2, 125.6, 124.7, 124.6, 121.5, 118.5, 112.4, 57.5, 48.2, 41.9, 29.7, 29.2, 26.2, 21.7, 18.3, 17.7, 13.6.

Conclusion: MK145 was synthesized as from MK130 in a 75% yield.

### Example 58: Procedure for the preparation of (2S,3S)-2-amino-N-(4-((4-amino-2-butyl-1H-imidazo[4,5-c]quinolin-1-yl)methyl)benzyl)-3-methylpentanamide (MK146)

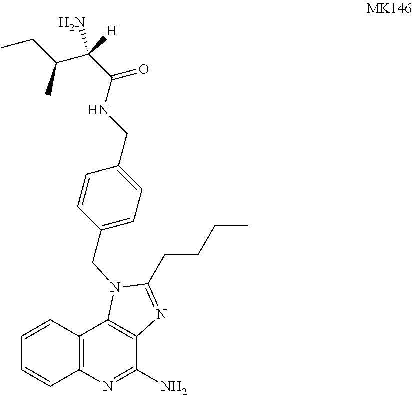 [SMILES: CCCCc1nc2c(N)nc3ccccc3c2n1Cc1ccc(CNC(=O)[C@@H](N)[C@@H](C)CC)cc1]

This example describes an L-isoleucine derivatization of MK130. MK146 was synthesized from MK130 according to the general procedure described in example 47:

Fmoc-Ile-OH (0.127 mmol), HCTU (0.127 mmol), DIPEA (0.254 mmol), and MK130 (0.085 mmol). UPLCMS of Fmoc-protected intermediate (LC2, C₁₈): Retention time=3.38 min. Calculated mass of C₄₃H₄₆N₆O₃=694.36; Observed [M+H]⁺ as m/z=695.4.

Purification of the product was carried out by semi-prep HPLC with a linear gradient over 15 min (A/B 80:20→65:35). Yielded 37.7 mg (76%) as a fluffy white solid. HPLC >95%. UPLCMS of product (LC1, C₁₈): Retention time=1.22 min. Calculated mass of C₂₈H₃₆N₆O=472.30; Observed [M+H]⁺ as m/z=473.4.

¹H NMR (400 MHz, DMSO-d₆) δ 9.15 (s, 2H), 8.87 (t, J=5.9 Hz, 1H), 8.16-8.06 (m, 3H), 7.93 (dd, J=8.4, 1.3 Hz, 1H), 7.77 (dd, J=8.4, 1.2 Hz, 1H), 7.62 (ddd, J=8.3, 7.1, 1.2 Hz, 1H), 7.33 (ddd, J=8.4, 7.2, 1.2 Hz, 1H), 7.26 (d, J=8.1 Hz, 2H), 7.04 (d, J=8.0 Hz, 2H), 5.95 (s, 2H), 4.34 (dd, J=15.2, 5.9 Hz, 1H), 4.26 (dd, J=15.2, 5.7 Hz, 1H), 3.59-3.58 (m, 1H), 3.01-2.91 (m, 2H), 1.80-1.68 (m, 3H), 1.46-1.32 (m, 3H), 1.04 (ddt, J=14.2, 9.5, 7.2 Hz, 1H), 0.87 (t, J=7.4 Hz, 3H), 0.82 (d, J=6.9 Hz, 3H), 0.79 (t, J=7.3 Hz, 3H).

¹³C NMR (101 MHz, DMSO) δ 167.8, 156.9, 149.1, 138.1, 135.3, 134.3, 134.0, 129.4, 128.2, 125.6, 124.7, 124.6, 121.5, 118.4, 112.4, 56.6, 48.2, 41.9, 36.1, 29.2, 26.2, 24.0, 21.7, 14.6, 13.6, 11.1.

Conclusion: MK146 was synthesized as from MK130 in a 76% yield.

### Example 59: Procedure for the preparation of N-(4-((4-amino-2-butyl-1H-imidazo[4,5-c]quinolin-1-yl)methyl)benzyl)propionamide (MK148)

This example describes a propionylation of MK130.

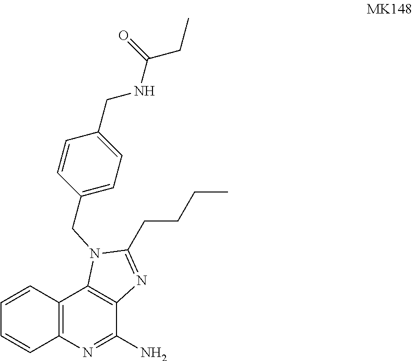 [SMILES: CCCCc1nc2c(N)nc3ccccc3c2n1Cc1ccc(CNC(=O)CC)cc1]

MK148 was synthesized from MK130 according to the general procedure described in example 48:
Propionic anhydride (0.116 mmol), DIPEA (0.096 mmol), and MK130 (0.096 mmol). Purification of the product was carried out by semi-prep HPLC with a linear gradient over 15 min (A/B 70:30→50:50). Yielded 38.5 mg (76%) as a fluffy white solid. HPLC >95%. UPLCMS of product (LC2, C₁₈): Retention time=2.15 min. Calculated mass of C₂₅H₂₉N₅O=415.24; Observed [M+H]⁺ as m/z=416.2.

¹H NMR (400 MHz, DMSO-d₆) δ 9.08 (s, 2H), 8.24 (t, J=6.0 Hz, 1H), 7.95 (dd, J=8.4, 1.3 Hz, 1H), 7.77 (dd, J=8.4, 1.1 Hz, 1H), 7.62 (ddd, J=8.3, 7.1, 1.2 Hz, 1H), 7.36 (ddd, J=8.3, 7.1, 1.2 Hz, 1H), 7.19 (d, J=8.2 Hz, 2H), 7.02 (d, J=8.0 Hz, 2H), 5.93 (s, 2H), 4.20 (d, J=5.9 Hz, 2H), 2.98-2.93 (m, 2H), 2.10 (q, J=7.6 Hz, 2H), 1.73 (p, J=7.5 Hz, 2H), 1.38 (h, J=7.4 Hz, 2H), 0.98 (t, J=7.6 Hz, 3H), 0.87 (t, J=7.4 Hz, 3H).

¹³C NMR (101 MHz, DMSO) δ 172.9, 156.9, 149.0, 139.3, 135.3, 134.0, 133.8, 129.4, 127.7, 125.5, 124.7, 124.7, 121.6, 118.4, 112.4, 48.2, 41.5, 29.2, 28.4, 26.2, 21.8, 13.7, 9.9.

Conclusion: MK148 was synthesized as from MK130 in a 76% yield.

### Example 60: cLogD Example for Remote Loading of MK130 and Derivatives Thereof

This example lists cLogD values at pH values 5.5, 7.4, 8.5 and 9.0 of MK130 and the derivatives thereof that contain aliphatic primary amines.

The cLogD values were calculated using MarvinSketch 17.27.0 by ChemAxon Ltd. The LogP values were calculated by the ChemAxon method with the electrolyte concentrations of chloride, sodium, and potassium were set to zero. Four distinct pH values 5.5, 7.4, 8.5 and 9.0 were chosen as output.

The cLogD values of all imidazoquinolines in this example were increasing from pH=5.5 to pH=9.0 as seen from the table below. Furthermore, most imidazoquinolines had a change from a negative to a positive cLogD value when increasing the pH from 5.5 to 9.0.

TABLE 4
cLogD values at pH values 5.5, 7.4, 8.5 and 9.0
of MK130 and derivatives thereof

| Molecule | clogD_{5.5} | clogD_{7.4} | clogD_{8.5} | clogD_{9.0} |
|---|---|---|---|---|
| MK130 | 0.20 | 2.18 | 3.22 | 3.61 |
| MK135 | 0.13 | 1.80 | 2.04 | 2.06 |
| MK136 | −0.49 | 1.44 | 2.14 | 2.26 |
| MK137 | 0.58 | 2.52 | 3.31 | 3.46 |
| MK138 | 2.64 | 4.54 | 5.12 | 5.20 |
| MK139 | 0.29 | 2.21 | 2.86 | 2.96 |
| MK140 | −0.45 | 1.54 | 2.62 | 3.08 |
| MK141 | 2.33 | 4.23 | 4.79 | 4.79 |
| MK145 | 1.32 | 3.28 | 4.12 | 4.30 |
| MK146 | 1.73 | 3.68 | 4.52 | 4.70 |

The cLogD values of all imidazoquinolines in this example increased from pH=5.5 to pH=9.0. While MK136 and MK140 have negative cLogD values at pH=5.5, all other cLogD values were positive at pH>5.5. Moreover, MK138 has the highest cLogD values of the examples given here, with cLogD>5 at pH>8.2.

### Example 61: Preparation of Liposomal Formulations F3 and F4

This example describes the preparation of the liposomes which were used in the examples 62-66 and 70.

Liposomes were prepared according to the general procedure for liposome preparation (example 15) similar to the preparation of formulation F1. HEPES buffer (25 mM HEPES, 10 vol % sucrose, pH 7.4) was used for dialysis after preparation of formulation F3 in 150 mM ammonium sulfate ((NH₄)₂SO₄) and formulation F4 in 250 mM ammonium sulfate.

Formulation F3: HSPC/Chol/DSPE-PEG2k-56:38:5 (mol/mol).

Formulation F4: HSPC/Chol/DSPE-PEG2k-56:38:5 (mol/mol).

TABLE 6
Characterization of formulations F3 and F4

| | | | | Intra- |
|---|---|---|---|---|
| Formu- | | | | liposomal |
| lation | Size | PDI | Zeta | buffer |
| F3 | 125.5 +/− 0.9 | 0.043 | −7.07 +/− 0.31 | 150 mM |
| | | | | (NH₄)₂SO₄ |
| F4 | 133.4 +/− 0.8 | 0.078 | −1.94 +/− 0.27 | 250 mM |
| | | | | (NH₄)₂SO₄ |

The liposomes generated in this example were used to generate examples 62-66 and 70.

### Example 62: Remote Loading of MK130 and Derivatives Thereof in Formulation F3 at a Drug/Lipid Ratio of 0.1

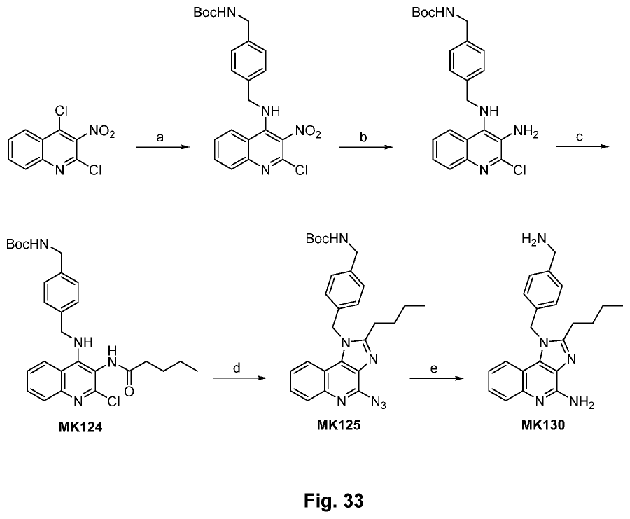

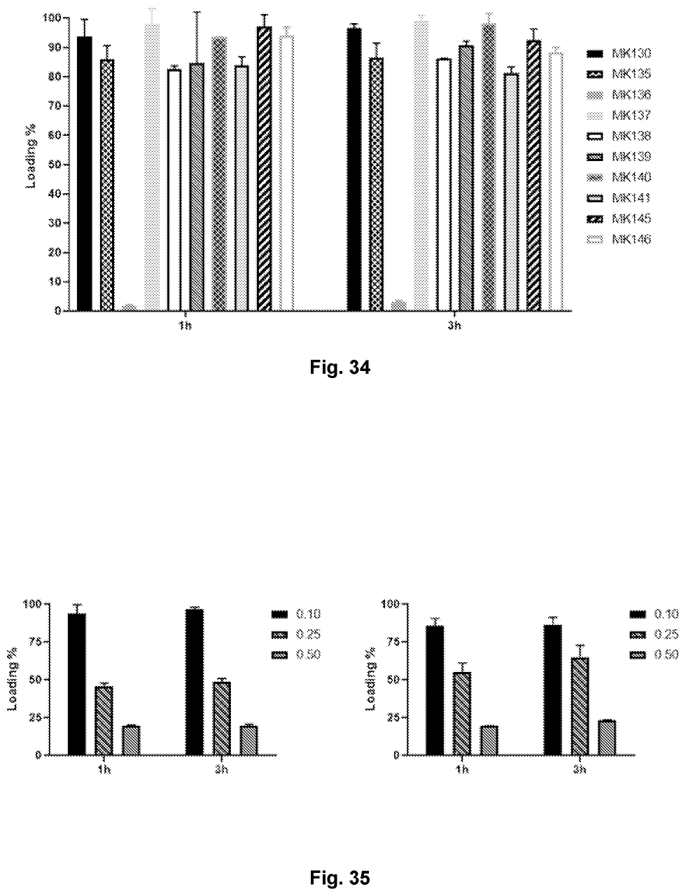

In this example we investigated the loading efficiency of MK130 and the derivatives thereof that contain aliphatic primary amines at a drug/lipid ratio of 0.1 in formulation F3. Remote loading of MK130 and the derivatives thereof that contain aliphatic primary amines was carried out as described in example 14 with solutions of the compounds and formulation F3 (example 61). Remote loading efficiencies measured by HPLC at 1 h and 3 h are summarized in table 7 below and in FIG. 34.

TABLE 7
Overview of remote loading efficiency of MK130 and
derivatives thereof in formulation F3

| Molecule | 1 h (%)^{a} | 3 h (%)^{a} |
|---|---|---|
| MK130 | 93.8 (5.8) | 96.7 (1.3) |
| MK135 | 85.9 (4.7) | 86.4 (5.1) |
| MK136 | 2.0 (0.2) | 3.1 (0.4) |
| MK137 | 98.1 (5.2) | 99.1 (1.8) |
| MK138 | 82.5 (1.2) | 86.2 (0.2) |
| MK139 | 84.7 (17.4) | 90.6 (1.6) |
| MK140 | 93.6 (0.1) | 98.3 (3.4) |
| MK141 | 83.8 (3.0) | 81.2 (2.1) |
| MK145 | 97.2 (4.0) | 92.4 (3.9) |
| MK146 | 94.1 (2.9) | 88.4 (1.5) |

^{a}Loading efficiencies are given as mean values with standard deviations given in parentheses (n = 2).

In formulation F3, the loading efficiency of MK130 and derivatives thereof were all above 80% at 1 h, except for MK136 which was formed insoluble particles in the liposomal formulation. Several of the compounds had loading efficiencies above 90%. Notably, under these conditions MK130, MK137, and MK140 were able to load into formulation F3 with loading efficiencies above 95%. Meanwhile, the most hydrophobic molecule (MK138) did not reach a loading efficiency of above 90% during the 3 h.

### Example 63: Remote Loading of MK130 and MK135 in Formulation F3 at Varying Drug/Lipid Ratios

In this example we investigated the loading efficiency of MK130 and MK135 at varying drug/lipid ratios in both formulations F3.

Remote loading of MK130 and MK135 was carried out as described in example 14 with imidazoquinoline solutions and formulation F3 (example 61). Remote loading efficiencies measured by HPLC are summarized in table 8 below and in FIG. 35.

TABLE 8
Overview of remote loading efficiency of MK130 and
MK135 in formulation F3 at varying drug/lipid ratios

| Molecule | Drug/lipid ratio | 1 h (%)^{a} | 3 h (%)^{a} |
|---|---|---|---|
| MK130 | 0.10 | 93.8 (5.8) | 96.7 (1.3) |
| | 0.25 | 45.9 (1.9) | 48.9 (2.1) |
| | 0.50 | 19.7 (0.4) | 19.5 (1.1) |
| MK135 | 0.10 | 85.9 (4.7) | 86.4 (5.1) |
| | 0.25 | 55.5 (5.7) | 65.0 (7.7) |
| | 0.50 | 19.5 (0.1) | 23.3 (0.3) |

^{a}Loading efficiencies are given as mean values with standard deviations given in parentheses (n = 2).

In formulation F3, the loading efficiencies of MK130 and MK135 gradually declined with increasing drug to lipid ratio. While loading efficiencies of both compounds were above 85% at a drug/lipid ratio of 0.1, this decreased to below 70% at a drug/lipid ratio of 0.25 and decreased further at a drug/lipid ratio of 0.5 to 20% and 23% at 3 h for MK130 and MK135 respectively.

### Example 64: Remote Loading of MK138 in formulations F3 and F4 at varying drug/lipid Ratios

In this example we investigated the loading efficiency of MK138 at varying intraliposomal buffer concentrations. The pH gradient between the intraliposomal and the extraliposomal buffers is important for the compounds ability to be remote loaded into liposomes. However, remote loading capacity can be limited by intraliposomal buffer concentration. Conversely, we investigated how an increase in the intraliposomal ammonium sulfate concentration affects loading efficiencies. MK138 is the most hydrophilic derivative of MK130 and could be improved regarding loading efficiency in F3 at a drug/lipid ratio of 0.1 (example 62). Accordingly, we investigated the loading efficiency of MK138 at drug to lipid ratio of 0.1 and 0.25 in both formulations F3 (150 mM ammonium sulfate/25 mM HEPES gradient) and F4 (250 mM ammonium sulfate/25 mM HEPES gradient).

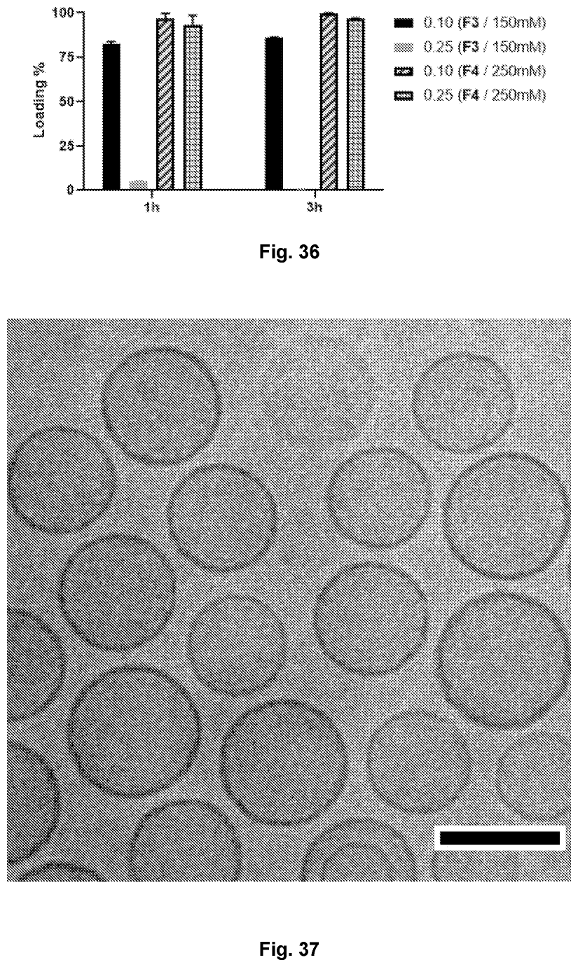

Remote loading of MK138 was carried out as described in example 14 with a solution of MK138 and formulations F3 and F4 (example 61). Remote loading efficiencies measured by HPLC are summarized in table 9 below and in FIG. 36.

TABLE 9
Overview of remote loading efficiency of MK138 in
formulations F3 and F4 at varying drug/lipid ratios

| Formulation | Drug/lipid ratio | 1 h (%)^{a} | 3 h (%)^{a} |
|---|---|---|---|
| F3 | 0.10 | 82.6 (1.2) | 86.2 (0.1) |
| | 0.25 | 5.1 (0.1) | 0.5 (0.1) |
| F4 | 0.10 | 96.5 (3.3) | 100.0 (0.1) |
| | 0.25 | 93.0 (5.7) | 96.5 (0.7) |

^{a}Loading efficiencies are given as mean values with standard deviations given in parentheses (n = 2).

At a drug/lipid ratio of 0.1, the loading efficiency of MK138 increased markedly from 86% at 3 h in formulation F3 (150 mM ammonium sulfate), to 100% in formulation F4 (250 mM ammonium sulfate). An even larger benefit of raising the intraliposomal buffer concentration was seen at a drug/lipid ratio of 0.25, where the loading efficiency of MK138 increased from 5% at 1 h in formulation F3 to 93% in formulation F4. These results clearly indicate the advantage in raising the intraliposomal buffer concentration with regards to loading efficiency.

### Example 65: Intraliposomal Precipitation of MK130 and MK138 in Formulations F3 and F4

In this example we examined the liposomes loaded with MK130 and MK138 to investigate whether these compounds would precipitate in liposomes after remote loading. Intraliposomal drug precipitation reduces drug leakage from the liposomes, which in turn can reduce in vivo drug toxicity.

Solutions of liposomal MK130 and MK138 in formulation F3 from example 62 and liposomal MK138 in formulation F4 from example 64 were examined by cryo-TEM. All liposomes were loaded at a drug to lipid ratio of 0.1.

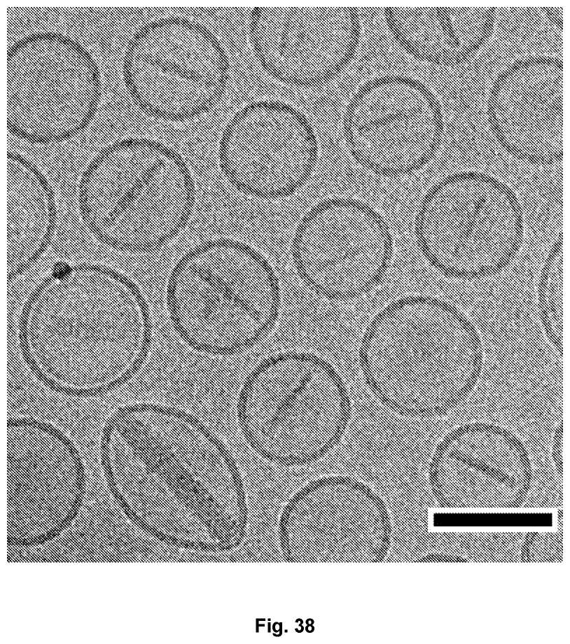

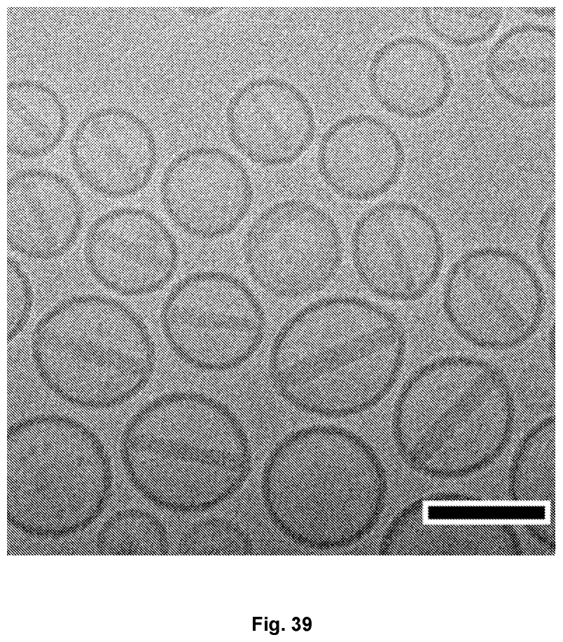

Cryo-TEM of the liposomes showed slight precipitation of MK130 in formulation F3 (FIG. 37), while MK138 precipitated to form rod-like crystals in formulation F3 (FIG. 38) which was even more pronounced when remote loaded into formulation F4 (FIG. 39).

These results taken together indicate an increased tendency to form rod-like crystals when increasing the hydrophobicity and π-stacking molecular properties in the compounds.

### Example 66: General Procedure for the Measurement of NF-κB Induction in HEK-Blue mTLR7 Cells Stimulated with Small Molecules or Small Molecules in Formulation F3

This example describes the general procedure used to assess the compounds' potency to induce NF-κB expression in human embryonic kidney (HEK) cells. The transcription factor NF-κB is expressed upon stimulation of TLR7/8 and is commonly used for quantifying TLR7/8 agonist potencies in reporter cell assays. Conversely, we used HEK-Blue mTLR-7 cells to evaluate the potencies of the synthesized derivatives, as previously reported (Shukla et al, J. Med. Chem. (2010), 53, 4450-4465).

HEK-Blue TLR7 assays were carried out using HEK-Blue™ mTLR7 cells (Invivogen, Toulouse, France, Cat. #hkb-mtlr7). The assays were carried out according to the protocol from Invivogen. TLR expressing cells were cultured in DMEM high glucose (4.5 g/L) supplemented with 10% (v/v) fetal bovine serum (FBS) and 1% (v/v) penicillin-streptomycin (P/S) to 50 U/ml penicillin and 50 mg/ml streptomycin. On the day of the study, the cells were seeded in a 96-well plate (ThermoFisher Scientific, Roskilde, Denmark) with 20 μL imidazoquinoline TLR7/8 agonist solutions per well at varying concentrations by adding 180 μL cell suspension, made by suspending cells to 0.22×10⁶ cells per mL in HEK-Blue™ Detection medium (Invivogen, Cat. #hb-det2) added Blasticidin (10 μg/ml) and Zeocin™ (100 μg/ml), leading to a final concentration of 40×10³ cells per well in triplicates. Cells were incubated with the imidazoquinoline TLR7/8 agonists, formulated in liposomes or dissolved in PBS, at 37° C. with 5% CO₂ for 16 h. The response was quantified spectrophotometrically at 655 nm and was normalized to the signal from a blank sample (DMSO dilution) run in parallel.

This general procedure was used to generate the examples 69 and 70.

### Example 67: General Procedure for the Measurement of NF-κB Induction in RAW-Blue Cells Stimulated with Small Molecules

This example describes the general procedure used to assess the compounds' potency to induce NF-κB expression in RAW 264.7 macrophages cells that stably express secreted embryonic alkaline phosphatase (SEAP) upon induction of NF-κB. RAW-Blue™ reporter cells are commonly used for quantifying TLR7/8 agonist potencies in reporter cell assays. Conversely, we used RAW-Blue™ cells to evaluate the potencies of the synthesized derivatives, as previously reported¹. ¹ Ryu et al, J. Am. Chem. Soc. (2014), 136, 10823-10825

RAW 264.7 assays were carried out using RAW-Blue™ cells and QUANTI-Blue™ detection solution (Invivogen, Toulouse, France, at. #raw-sp and rep-qbs respectively). The assays carried out according to the protocol from Invivogen. RAW 264.7 macrophages were cultured in DMEM high glucose (4.5 g/L) supplemented with 10% (v/v) FBS, 1% (v/v) P/S, and Normocin™ (100 μg/ml). On the day of the study, the cells were seeded in a 96-well plate (ThermoFisher Scientific, Roskilde, Denmark) with 20 μL TLR7/8 agonist solutions per well at varying concentrations by adding 180 μL cell suspension, made by suspending cells to 0.55×10⁶ cells per mL in the prepared cell medium added Zeocin™ (200 μg/ml), leading to a final concentration of 100×10³ cells per well in triplicates. Cells were incubated with the TLR7/8 agonists, diluted from 10 mM DMSO stocks in PBS, at 37° C. with 5% CO₂ for 24 h for SEAP production. 50 μL SEAP containing cell supernatant from each well was added 150 μL QUANTI-Blue™ detection solution and was incubated at 37° C. for 3-6 h. The resulting response was quantified spectrophotometrically at 640 nm and was normalized to the signal from a blank sample (DMSO dilution) run in parallel.

This general procedure was used to generate the example 70.

### Example 68: TLR7 Agonistic Activities of MK130 and Derivatives Thereof in HEK-Blue mTLR7 Cells

This example describes the TLR7 agonist activities of MK130, MK135, MK136, MK137, MK138, MK139, MK140, MK141, MK145, and MK146 evaluated in HEK-Blue™ mTLR7 cells.

The reporter cell assay was carried out according to the general procedure described in example 66.

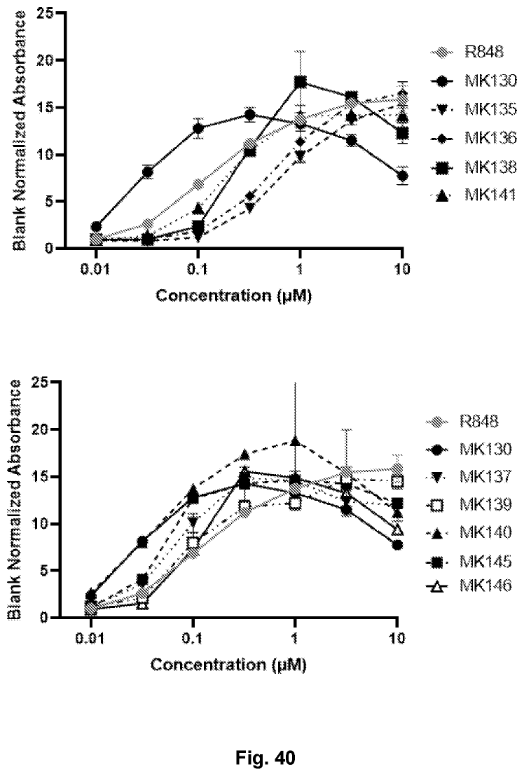

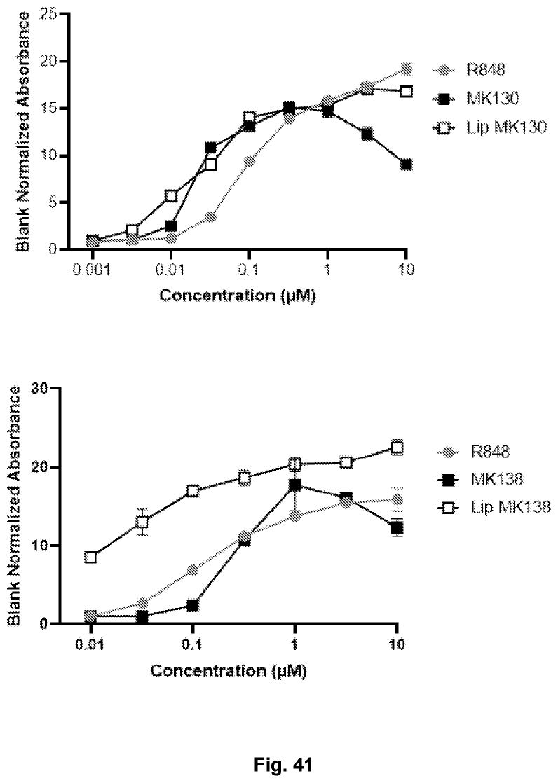

The results from this assay are summarized in FIG. 40. The derivatives of MK130 were compared to MK130 and R848 and were divided according to their potency at 0.32 μM.

All compounds showed typical decreasing dose-response curves below 0.32 μM. Cell death was observed for most compounds at concentrations >1 μM, except for R848, MK135, and MK136. Conversely, MK135 and MK136 showed the lowest potency of all derivatives regarding values. While MK138 did induce a higher response than both MK130 and R848 at 1 μM, only little activity was observed at concentrations <0.32 μM. Differently, compounds MK137, MK139, MK140, MK145, and MK146 showed potencies that were higher or comparable to that of R848. Notably MK140 gave a dose-response curve with similar potency and higher efficacy than that of MK130 in the HEK-Blue™ mTLR7 assay.

Conclusion: Several derivatives are as potent or more potent than R848 in the HEK-Blue™ mTLR7 assay.

### Example 69: TLR7 Agonistic Activities of MK130 and MK138 in Formulation F3 Evaluated in HEK-Blue TLR7 Cells

This example describes the TLR7 agonist activities of liposomal MK130 and MK138 in formulation F3.

The reporter cell assay was carried out according to the general procedure described in example 66 using solutions of MK130 and MK138 as well as liposomal formulations of MK130 and MK138 in formulation F3 prepared as in examples 62.

The results from this assay are summarized in FIG. 41. Both formulations stimulated a TLR7 response and none of the formulations exhibited the same cytotoxicity as the free drugs at concentrations ≥10 μM. Moreover, liposomal formulation of both MK130 and especially MK138 seemed to improve potency, which resulted in potencies much higher than R848.

Thus, liposomal MK130 and MK138 are able to induce TLR7/8 signaling at concentrations much lower than free R848 in the HEK-Blue™ mTLR7 assay.

### Example 70: TLR7 Agonistic Activities of MK130 and Derivatives Thereof in RAW-Blue™ Cells

This example describes the TLR7 agonist activities of MK130, MK132, MK135, MK136, MK137, MK138, MK139, MK140, MK141, MK145, and MK146 evaluated in RAW-Blue™ cells.

The reporter cell assay was carried out according to the general procedure described in example 67.

Half maximal effective concentration (EC₅₀) values were determined from sigmoidal 4PL fittings from the resulting dose-response curves in GraphPad Prism.

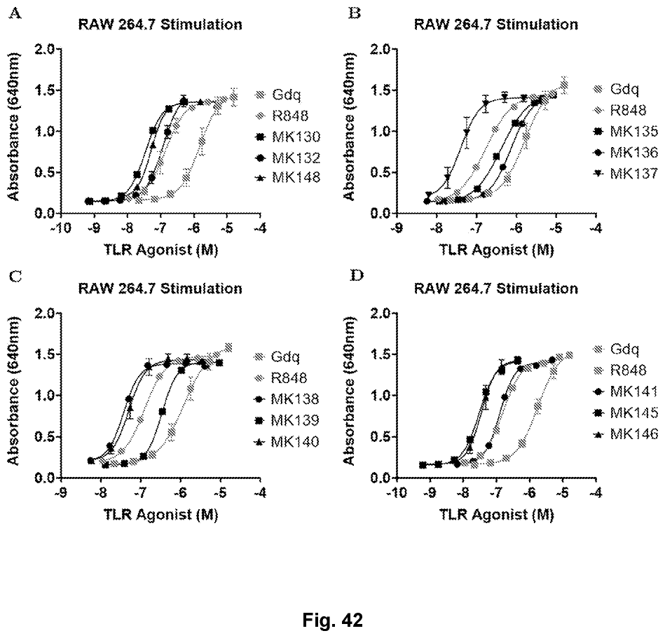

The results from this assay are summarized in FIG. 42 and EC₅₀ values are seen in Table 10 below.

TABLE 10
EC₅₀ values from RAW-Blue reporter cell
assay of MK130 and derivatives thereof

| Molecule | EC₅₀ [μM] | Subfigure^{a} |
|---|---|---|
| R848 | 0.15 | All |
| Gardiquimod | 1.67 | All |
| MK130 | 0.03 | A |
| MK132 | 0.12 | A |
| MK135 | 0.42 | B |
| MK136 | 0.74 | B |
| MK137 | 0.04 | B |
| MK138 | 0.04 | C |
| MK139 | 0.34 | C |
| MK140 | 0.05 | C |
| MK141 | 0.13 | D |
| MK145 | 0.03 | D |
| MK146 | 0.04 | D |
| MK148 | 0.05 | A |

^{a}Refers to placement of result in FIG. 42

MK130 and MK145 were the most potent compounds in the RAW-Blue™ cell assay with nearly similar potencies exhibited by MK137, MK138, MK140, MK146, and MK148, which all showed greater potencies than R848.

Conclusion: Stimulation of RAW-Blue™ cells with MK130 and several derivatives thereof presented here resulted in greater induction of NF-κB than when using R848.

### Example 71: Procedure for preparation of 4-((6-amino-8-hydroxy-2-(2-methoxyethoxy)-9H-purin-9-yl)methyl)-N-(2-(2-aminopyridin-4-yl)methyl)benzamide (KRJ1-098)

KRJ1-098 was synthesized from KRJ1-069 as seen in FIG. 32 using the following procedure:

KRJ1-069 (60.0 mg, 0.167 mmol) was dissolved in anh. NMP (0.7 mL). PyAOP (88.0 mg, 0.169 mmol) and DIPEA (90 mL, 0.517 mmol) were added and the reaction mixture was stirred for 2 hr at room temperature. 4-(2-Aminomethyl)pyridin-2-amine (95%, 48.1 mg, 0.371 mmol) and anh. NMP (0.7 mL) were added and the reaction mixture was stirred for 24 hr at room temperature. The mixture was concentrated in vacuo. LC-MS: found: 465.1 [M+H]⁺, C₂₂H₂₅N₈O₄⁺ requires M, 465.20.

### Example 72: Procedure for Preparation of KRJ2-110

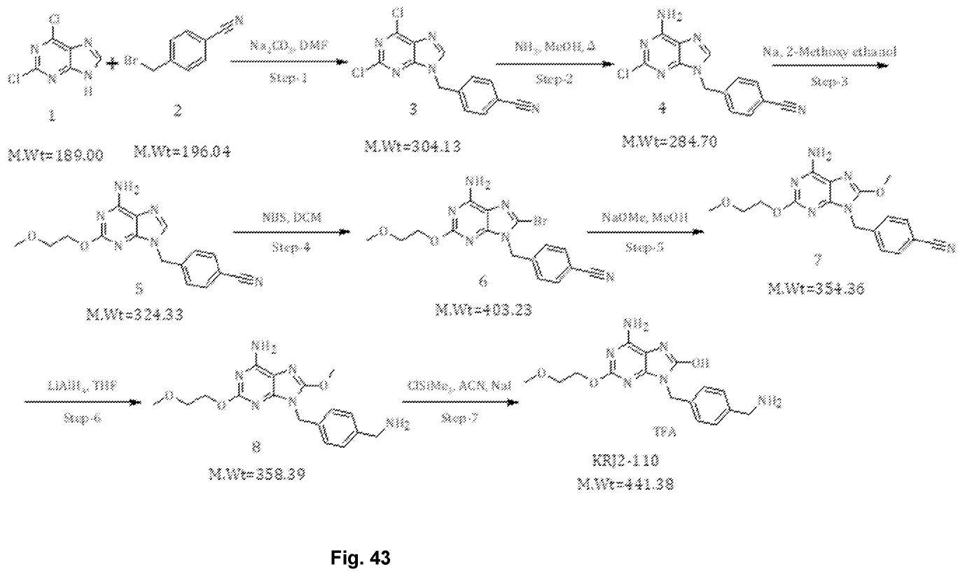

KRJ2-110 was synthesized as seen in FIG. 43 using the following procedure:

1) KRJ1-006: 4-((2,6-dichloro-9H-purin-9-yl)methyl)benzonitrile 2,6-Dichloro-9H-purine (10 g, 97%, 51.32 mmol) and Na₂CO₃ (16.32 g, 154 mmol) was dissolved in DMF (150 mL) and the reaction mixture was stirred for 15 min. 4-(Bromomethyl)benzonitrile (12.32 g, 98%, 61.59 mmol) was added and the reaction mixture was stirred at room temperature for 18 hr. The reaction mixture was filtered to remove insoluble salts and poured into water (400 mL). The aqueous phase was extracted with EtOAc (3×800 mL). The combined organic phases were dried over MgSO₄ and concentrated in vacuo. The crude product was purified by dry column chromatography (d: 5 cm, fractions: 200 mL, increments: 5%, from 0-70% n-hexane in EtOAc, v/v) to give the product KRJ1-006 as a white solid (9.41 g, 60%). Impure fractions were not purified further. TLC R_{f}=0.35 (30% n-hexane in EtOAC, v/v). LC-MS: found: 303.9 [M+H]⁺, C₁₃H₈N₅Cl₂⁺ requires M, 304.02. ¹H NMR (400 MHz, DMSO-d₆) δ 8.84 (s, 1H), 7.84 (d, J=8.3 Hz, 2H), 7.50 (d, J=8.4 Hz, 2H), 5.61 (s, 2H). ¹³C NMR (101 MHz, DMSO-d₆) δ 153.51, 151.18, 149.87, 148.45, 141.09, 132.73, 130.61, 128.40, 118.53, 110.87, 46.65.

2) KRJ1-024: 4-((6-amino-2-chloro-9H-purin-9-yl)methyl)benzonitrile

A steel reaction bomb (150 mL) was equipped with KRJ1-006 (1.90 g, 6.24 mmol) and NH₃ in MeOH (7M, 80 mL), sealed and heated to 60° C. for 18 hr. The steel reaction bomb was cooled on ice before opened, and the reaction mixture was concentrated in vacuo. This gave the crude product KRJ1-024 as a white solid (2.10 g), which was deemed to be of sufficient purity to be used in the next step with no further purification. TLC R_{f}=0.20 (20% EtOAC, 20% acetone in n-heptane, v/v/v). LC-MS: found: 285.2 [M+H]⁺, C₁₃H₁₀N₆Cl⁺ requires M, 285.71. ¹H NMR (400 MHz, DMSO-d₆) δ 8.27 (s, 1H), 7.87-7.78 (m, 4H), 7.42 (d, J=8.3 Hz, 2H), 5.45 (s, 2H). ¹³C NMR (101 MHz, DMSO-d₆) δ 156.83, 153.21, 150.55, 142.23, 141.49, 132.72, 128.13, 118.58, 117.77, 110.59, 45.87.

3) KRJ1-040: 4-((6-amino-2-(2-methoxyethoxy)-9H-purin-9-yl)methyl)benzonitrile

Sodium metal (2.08 g, 90.5 mmol) was added to 2-methoxyethanol (300 mL) at 0° C., and the reaction mixture was left to heat to room temperature while dissolving. KRJ1-024 (8.88 g, 31.21 mmol) dissolved in 2-methoxyethanol (900 mL) was added and the reaction mixture was stirred at 115° C. for 21 hr. The reaction mixture was concentrated in vacuo. The crude product was purified by dry column chromatography (d: 5 cm, fractions: 200 mL, increments: 1%, from 0-5% MeOH in DCM, v/v) to give the product KRJ1-040 as an orange solid (5.96 g, 59%). Impure fractions were not purified further. TLC R_{f}=0.30 (5% MeOH in DCM, v/v). LC-MS: found: 325.3 [M+H]⁺, C₁₆H₁₇H₆O₂⁺ requires M, 325.14. ¹H NMR (400 MHz, DMSO-d₆) 8.07 (s, 1H), 7.82 (d, J=8.4 Hz, 2H), 7.48-7.42 (m, 2H), 7.27 (s, 2H), 5.37 (s, 2H), 4.32-4.26 (m, 2H), 3.61-3.55 (m, 2H), 3.26 (s, 3H). ¹³C NMR (101 MHz, DMSO-d₆) δ 161.35, 156.78, 151.17, 142.67, 139.46, 132.63, 128.38, 118.62, 115.14, 110.45, 70.27, 65.33, 58.08, 45.65.

4) KRJ1-050: 4-((6-amino-8-bromo-2-(2-methoxyethoxy)-9H-purin-9-yl)methyl)benzonitrile

A solution of N-bromosuccinimide in DCM was prepared by addition of N-bromosuccinimide (2.48 g, 13.96 mmol) to DCM (100 mL) at 0° C. This was added dropwise to a solution of KRJ1-040 (3.742 g, 11.54 mmol) in DCM (300 mL) at 0° C. The reaction mixture was stirred at room temperature for 20 hr, changing color from yellow, over green and brown, to red. Aq. Na₂S₂O₃ (0.1 M, 500 mL) was added to the reaction mixture and the phases were separated. The organic phase was washed with sat. NaHCO₃ (500 mL), dried over MgSO₄ and concentrated in vacuo to give the product KRJ1-050 as a bright orange solid (4.545 g, 98%). TLC R_{f}=0.40 (5% MeOH in DCM, v/v). LC-MS: found: 403.3 [M+H]+, C₁₆H₁₆BrN₆O₂⁺ requires M, 403.05. ¹H NMR (400 MHz, DMSO-d₆) δ 7.83 (d, J=8.4 Hz, 2H), 7.55-7.42 (m, 2H), 7.39 (d, J=8.4 Hz, 2H), 5.35 (s, 2H), 4.34-4.26 (m, 2H), 3.63-3.55 (m, 2H), 3.26 (s, 3H). ¹³C NMR (101 MHz, DMSO-d₆) δ 161.39, 155.73, 152.43, 141.55, 132.75, 127.98, 123.71, 118.54, 115.39, 110.60, 70.20, 65.54, 58.09, 46.03.

5) KRJ1-058: 4-((6-amino-8-methoxy-2-(2-methoxyethoxy)-9H-purin-9-yl)methyl)benzonitrile

A solution of NaOMe in MeOH was prepared by addition of NaOMe (5.40 g, 100 mmol) to MeOH (200 mL) at 0° C. This was added dropwise to a solution of KRJ1-050 (4.02 g, 10 mmol) in MeOH (200 mL) at 0° C. The reaction mixture was heated to reflux for 18 hr. The reaction mixture was transferred to a separatory funnel with brine (100 mL) and DCM (250 mL) and the phases were separated. The aqueous phase was extracted with DCM (2×350 mL) and the combined organic phases were dried over MgSO₂ and concentrated in vacuo. This gave the crude product KRJ1-058 as an orange solid (3.9 g), which was deemed to be of sufficient purity to be used in the next step with no further purification. TLC R_{f}=0.30 (5% MeOH in DCM, v/v). LC-MS: found: 355.1 [M+H]⁺, C₁₇H₁₉N₆O₃⁺ requires M, 355.15. HPLC RT: 8.891 min (method A). ¹H NMR (400 MHz, DMSO-d₆) δ 7.81 (d, J=8.3 Hz, 2H), 7.39 (d, J=8.4 Hz, 2H), 6.92 (s, 2H), 5.13 (s, 2H), 4.31-4.23 (m, 2H), 4.03 (s, 3H), 3.61-3.55 (m, 2H), 3.26 (s, 3H). ¹³C NMR (101 MHz, DMSO-d₆) δ 159.99, 154.57, 153.35, 150.89, 142.26, 132.68, 128.05, 118.62, 110.42, 110.07, 70.32, 65.23, 58.09, 56.95, 43.45.

6) KRJ2-110: 6-amino-9-(4-(aminomethyl)benzyl)-2-(2-methoxyethoxy)-9H-purin-8-ol

KRJ2-106: A solution of KRJ1-058 (128 mg, 0.36 mmol) dissolved in anh. THF (5 mL) was added dropwise to a suspension of LiAlH₄ (58 mg, 1.44 mmol) in anh. THF (6 mL) at 0° C. The reaction was allowed to heat to room temperature and stirred for 23 hr. The reaction was cooled to 0° C. and quenched by slowly, dropwise addition of water (5 mL) and aq. NaOH (1M, 1 mL). The aqueous phase was extracted with DCM (2×10 mL) and a mixture of CHCl₃ and EtOH (2:1, 2×10 mL), the combined organic phases were dried over MgSO₄ and concentrated in vacuo. The crude product KRJ2-106 was demethylated without further purification or characterization. LC-MS: found: 359.1 [M+H]⁺, C₁₇H₂₃N₆O₃⁺ requires M, 359.18. ¹H NMR (400 MHz, DMSO-d₆) δ 8.22 (brs, 2H), 7.41 (d, J=8.1 Hz, 2H), 7.27 (d, J=8.1 Hz, 2H), 6.88 (s, 2H), 5.05 (s, 2H), 4.28 (dd, J=5.8, 3.8 Hz, 2H), 4.04 (s, 3H), 4.00 (s, 2H), 3.63-3.56 (m, 2H), 3.28 (s, 3H).

7) KRJ2-110: KRJ2-106 (86 mg, 240 μmol) was dissolved in MeCN (6 mL) and ClSiMe₃ (30 μl, 240 μmol) and Nat (36 mg, 240 μmol) was added. The reaction was stirred for 15 min at room temperature and water (7 mL) was added. The reaction mixture was concentrated in vacuo. The crude product was purified by preparative HPLC giving the product KRJ2-110 as the TFA salt. HPLC RT: 5.404 min (method A). LC-MS: found: 345.1 [M+H]⁺, C₁₆H₂₁N₆O₃⁺ requires M, 345.17. ¹H NMR (400 MHz, DMSO-d₆) δ 10.12 (s, 1H), 8.13 (brs, 3H), 7.40 (d, J=8.3 Hz, 2H), 7.33 (d, J=8.2 Hz, 2H), 6.55 (brs, 2H), 4.87 (s, 2H), 4.28-4.22 (m, 2H), 3.99 (q, J=5.8 Hz, 2H), 3.61-3.56 (m, 2H), 3.26 (s, 3H). ¹³C NMR (101 MHz, DMSO-d₆) δ 159.77, 152.27, 149.17, 147.69, 137.58, 133.11, 129.11, 127.67, 98.36, 70.23, 65.33, 58.08, 42.09, 42.02.

### Example 73: Procedure for preparation of 4-((6-amino-8-hydroxy-2-(2-methoxyethoxy)-9H-purin-9-yl)methyl)-N-(4-(aminomethyl)benzyl)benzamide (KRJ2-024)

KRJ2-024 was synthesized using the following procedure:

1) KRJ1-093 (65 mg, 0.181 mmol) was dissolved in anh. NMP (0.7 mL). PyAOP (95.4 mg, 0.183 mmol) and DIPEA (90 mL, 0.517 mmol) were added and the reaction mixture was stirred for 1 hr at room temperature. tert-Butyl (4-(aminomethyl)benzyl)carbamate (95%, 95.1 mg, 0.382 mmol) was added and the reaction mixture was stirred for 3 hr at room temperature. The mixture was concentrated in vacuo. The crude product was purified by flash column chromatography (3% triethylamine, 7% MeOH in DCM, v/v/v) to give the crude product KRJ2-020 as a yellow solid (141 mg), which was deprotected without further purification. R_{f}=0.15 (3% triethylamine, 7% MeOH in DCM, v/v/v). HPLC RT: 10.097 min (method B). LC-MS: found: 578.4 [M+H]⁺, C₂₉H₃₆N₇O₆⁺ requires M, 578.27.

2) KRJ2-020 (141 mg, 0.244 mmol) was dissolved in a mixture of DCM and DMF (10:1, v/v) (10 mL). A solution of TFA and TIPS [obtained by the addition of TFA (3 mL, 39.2 mmol) and TIPS (30 mL, 0.146 mmol) to DCM (5 mL) at 0° C.] was added dropwise to the reaction mixture, which was stirred for 2 hr at room temperature. Water (10 mL) was added to the reaction mixture and the phases were separated. The aq. phase was extracted with DCM (3×15 mL). The combined organic phases were extracted with water (20 mL). The aq. phase was concentrated in vacuo. The crude product was purified by preparative HPLC giving the product KRJ2-024 as a white solid (14.8 mg, 10%). HPLC RT: 7.134 min (method A). LC-MS: found: 478.2 [M+H]⁺, C₂₄H₂₈N₇O₄⁺ requires M, 478.22. ¹H NMR (400 MHz, DMSO-d₆) δ 10.12 (s, 1H), 9.05 (t, J=6.1 Hz, 1H), 8.13 (brs, 3H), 7.83 (d, J=8.3 Hz, 2H), 7.43-7.26 (m, 6H), 6.56 (brs, 2H), 4.91 (s, 2H), 4.46 (d, J=6.0 Hz, 2H), 4.29-4.21 (m, 2H), 4.00 (d, J=5.8 Hz, 2H), 3.62-3.54 (m, 2H), 3.26 (s, 3H). ¹³C NMR (101 MHz, DMSO-d₆) δ 165.94, 159.78, 152.24, 149.13, 147.72, 140.36, 140.21, 133.44, 132.33, 128.86, 127.49, 127.34, 127.30, 98.38, 70.20, 65.32, 58.06, 42.31, 42.14, 42.06.

### Example 74: Procedure the preparation of 4-((6-amino-8-hydroxy-2-(2-methoxyethoxy)-9H-purin-9-yl)methyl)-N-(2-(4-methylpiperazin-1-yl)ethyl)benzamide (KRJ2-032)

KRJ2-032 was synthesized using the following procedure: KRJ1-093 (25 mg, 0.070 mmol) was dissolved in anh. NMP (0.35 mL). PyAOP (36.9 mg, 0.071 mmol) and DIPEA (40 mL, 0.322 mmol) were added. The reaction mixture was stirred for 2 hr at room temperature. 4-(2-Aminoethyl)morpholine (24.1 mg, 0.176 mmol) was added and the mixture was stirred for 2 hr at room temperature. The mixture was concentrated in vacuo. The crude product was purified by preparative HPLC to give the product KRJ2-032 as a white solid (25.0 mg, 61%). HPLC RT: 6.334 min (method A). LC-MS: found: 472.3 [M+H]⁺, C₂₂H₃₀N₇O₅⁺ requires M, 472.23. ¹H NMR (400 MHz, DMSO-d₆) δ 10.22 (s, 1H), 9.89 (brs, 1H), 8.72 (t, J=5.7 Hz, 1H), 7.81 (d, J=8.3 Hz, 2H), 7.38 (d, J=8.4 Hz, 2H), 6.65 (brs, 2H), 4.92 (s, 2H), 4.28-4.22 (m, 2H), 4.06-3.92 (m, 2H), 3.73-3.49 (m, 8H), 3.31 (t, J=6.2 Hz, 2H), 3.26 (s, 3H), 3.21-3.06 (m, 2H). ¹³C NMR (101 MHz, DMSO-d₆) δ 166.57, 159.64, 152.27, 149.14, 147.61, 140.67, 132.94, 127.59, 127.31, 98.41, 70.20, 65.42, 63.30, 58.07, 55.29, 51.29, 42.17, 33.87. HPLC was recorded on a Shimadzu Nexera X2 UHPLC with a Waters XTerra 5 μm C18 column (4.6×150 mm) with UV detection with PDA from 190-800 nm. MP A: 0.1% TFA, 5% MeCN in H₂O (v/v/v), MP B: 0.1% TFA in MeCN (v/v/v). Flow rate: 1 mL/min.

Method A: Gradient: 0-45% B over 10 min, gradient starting at 2 min.
Method B: Gradient: 0-60% B over 10 min, gradient starting at 2 min.

### Example 75: Crystallization of KRJ Compounds within Liposomes

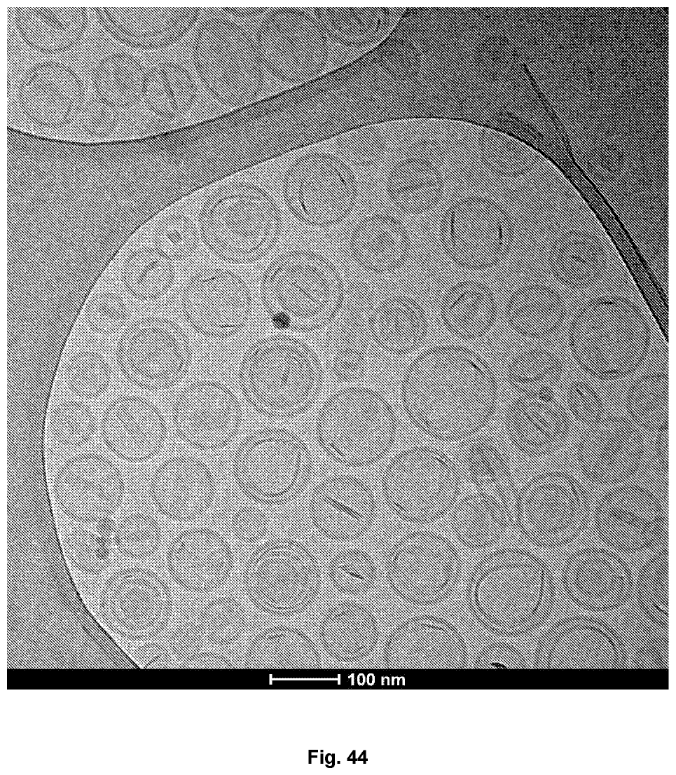

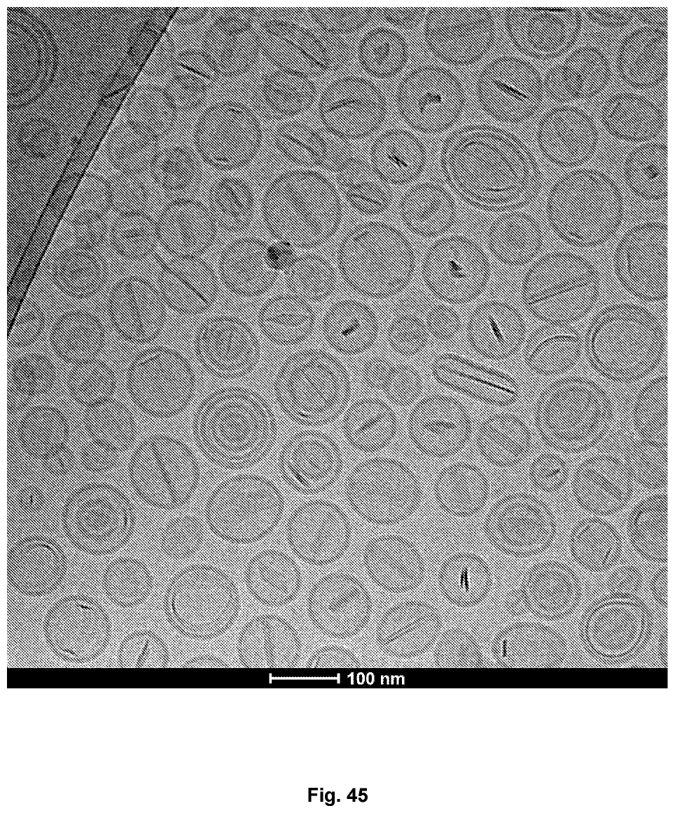

Solutions of liposomal KRJ compounds were examined by Electron microscope (cryo-TEM). Cryo-TEM of the liposomes (FIGS. 44 and 45) established that KRJ2-110 crystallizes inside the liposomes. Notably, the crystals are disc like or rod shaped that are flatter or more elongated in one dimension than another, which can be seen by the difference contrast depending on the angle that they are being observed from.

### Example 76: Procedure for Preparation of Cholesterol-R3

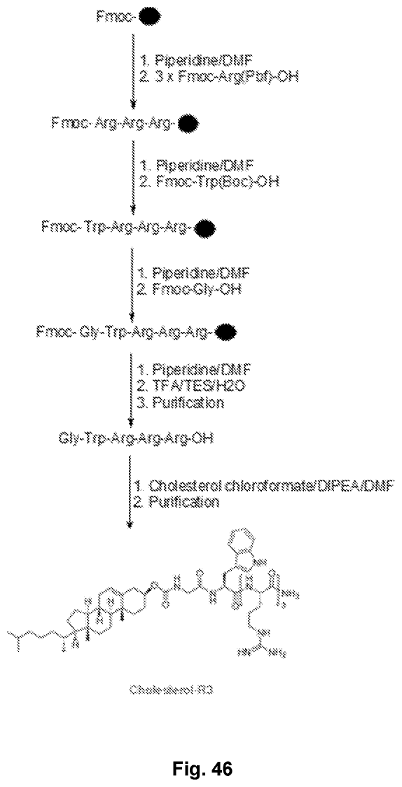

Cholesterol-R3 was prepared as seen in FIG. 46 according to the following procedure:

1) The peptide H-GWR₃-NH₂ was synthesized by standard solid phase peptide synthesis (SPPS) on an automated BioTage Synthesizer with a Tentagel Rink Amide resin (Scale: 0.5 mmol; loading: 0.20 mmol/g) by standard Fmoc methodology. Each coupling was performed using 4.0 equiv. Fmoc protected amino acid, 3.95 equiv. HATU, and 8 equiv. 2,4,6-collidine in DMF for 30 min at room temperature (rt). Deprotection of the Fmoc-protection group was achieved by subjecting the resin to 2×5 min of 20% Piperidine in DMF. The first glycine residue was double-coupled first with 5 min 75° C. microwave (μW) and subsequently 30 min at rt. The resin was cleaved with TFA:Triisopropylsilane (TIPS):H₂O (95:2.5:2.5) for 3 h. The solvent was removed in vacuo and the crude peptide was precipitated in cold diethyl ether. The isolated white peptide powder was purified by semi-preparative HPLC by employing a Knauer Eurosphere 100-5 C18 (20×250 mm) column. Eluent: (A) 5% CH₃CN+0.1% TFA in H₂O, (B) 0.1% TFA in CH₃CN. Gradient profile: 0% B for 10 min and 0% B to 20% B over 20 min. Rf-value=10 min. Flow rate: 17 mL/min. UV detection at 220/280 nm. The solvent was removed in vacuo and the product lyophilized from a mixture of H₂O and CH₃CN to give a white fluffy powder (0.203 mmol, 41%, purity 95%). MALDI-TOF MS (m/z): Calc. mass [M+H]+: 729.86, found mass [M+H]+: 729.81.

2) A flame-dried R^{B} flask was fitted with a magnet, septum and N₂-atmosphere. The lyophilized peptide (0.101 mmol) was dissolved in dry DMF (75 mL) and added to the RB flask followed by adding DIPEA (441 μL, 2.533 mmol). Cholesteryl chloroformate (55 mg, 0.122 mmol) was dissolved in CH₂Cl₂ (25 mL) and added to the RB flask. The reaction was let to react overnight (o/n). The solvent was removed in vacuo and the crude cholesteryl-GWR₃-NH₂ was purified by semi-preparative HPLC by employing a Knauer Eurosphere 100-5 C18 (20×250 mm) column. Eluent: (A) 5% CH₃CN+0.1% TFA in H₂O, (B) 0.1% TFA in CH₃CN. Gradient profile: 50% B to 100% B over 20 min Rf-value=12 min. Flow rate: 17 mL/min. UV detection at 220/280 nm. The solvent was removed in vacuo and the product lyophilized from a mixture of H₂O and CH₃CN to give a white fluffy powder (0.085 mmol, 84%, purity >98%). MALDI-TOF MS (m/z): Calc. mass [M+H]+=1141.77; found mass [M+H]+=1142.19. FT-IR, v (cm-1): 3277.9, 3194.7, 2949.2, 1658.5, 1531.8, 1199.9, 1178.9, 1128.9, 835.4, 800.7, 720.6. This gives an overall isolated yield of 34%.

### Example 77: Formulation of Gardiquimod-Loaded Liposomes

It should be noted that while this example is for Gardiquimod loading, the same general loading method can be applied to other TLR7/8 agonists.

### Overall Method:

a) Mix lipids in tert-butanol:water 9:1
b) Freeze-dry overnight to remove the solvent
c) Re-hydrate in Ammonium sulphate buffer (pH 5.75) and extrude to 120 nm.
d) Dialyze against a HEPES/Sucrose buffer (pH 7.40) to establish a pH gradient over the membrane.
e) Remote-load gardiquimod into the liposomes using the pH gradient.
f) Measure the loading efficiency by separating the liposomes from un-encapsulated drug using PD10 columns and measure the concentration of gardiquimod with HPLC.

Lipid stocks are dissolved in tert-butanol:water (tBut:DI) 9:1 according to tables below.

| Lipid | | | | | | | | Amount |
|---|---|---|---|---|---|---|---|---|
| stocks | | | | | | | | needed |
| Lipid | Cat. No. | Supplier | MW | mg | μL | mg/mL | mM | (μL) |
| POPC | 850457 | Avanti | 760.08 | 40.0 | 1000 | 40 | 52.6 | 1080 |
| Cholesterol | 700000 | Avanti | 386.65 | 4.0 | 1000 | 4 | 10.3 | 3383 |
| DOTAP | 890890 | Avanti | 698.54 | 25.0 | 1000 | 25 | 35.8 | 210 |
| DSPE-PEG | 880120 | Avanti | 2805.5 | 10.0 | 1000 | 10 | 3.6 | 168 |
| TopFluor | 810281 | Avanti | 909.97 | 0.5 | 1000 | 0.5 | 0.5 | 45 |
| PC | | | | | | | | |
| DiO | D4292 | Sigma | 881.7 | 4.49 | 8980 | 0.5 | 0.6 | 44 |
| DiI | 60010 | Biotium | 933.88 | 31.50 | 15750 | 2 | 2.1 | 0 |

Formulation RL11: DOTAP DiO

| | Concentration | |
|---|---|---|
| Volume (mL) | (mM) | |
| 1 | 50 | |
| | Percentage | Add amount |
| Lipid | (mol/mol) | (μL) |
| POPC | 56.85 | 540 |
| Cholesterol | 35 | 1692 |
| DOTAP | 7.5 | 105 |
| DSPE-PEG | 0.6 | 84 |
| TopFluor PC | 0 | 0 |
| DiO | 0.05 | 44 |
| Dil | 0 | 0 |
| | | 2465 |

Formulation RL12: DOTAP TopFluor

| | Concentration | |
|---|---|---|
| Volume (mL) | (mM) | |
| 1 | 50 | |
| | Percentage | Add amount |
| Lipid | (mol/mol) | (μL) |
| POPC | 56.85 | 540 |
| Cholesterol | 35 | 1692 |
| DOTAP | 7.5 | 105 |
| DSPE-PEG | 0.6 | 84 |
| TopFluor PC | 0.05 | 45 |
| DiO | 0 | 0 |
| Dil | 0 | 0 |
| | | 2466 |

Snap-freeze in liquid nitrogen and freeze-dry overnight. Keep pressure below 500 mT.
Ammonium sulfate buffer:
   4.96 g ammonium sulfate (NH4SO4-132.14 g/mol)=150 mM
   250 mL DI water
   Adjust pH to 5.75 using H2SO4 or HCl and NaOH.
Dialysis buffer:
   5.96 g HEPES (238.3012 g/mol)=25 mM
   100 g Sucrose
   1 L DI water
   Adjust pH to 7.4 using NaOH

### Method for Remote Loading:

For a lipid concentration of 40 mM, 0.5 mL contains around 20 μmol.
For drug-lipid ratio of 1:4:
   Make a 2 mg/mL stock of Gdq in DI water.
   Portion out 7834 in an HPLC vial.
   Freeze-dry the sample overnight.
Add the 500 μL liposome sample directly to the powdered gardiquimod.
Put under magnet stirring and 55 C heating for 4 hours.
Allow to equilibrate to room temperature before proceeding.

### Method for Measuring Encapsulating Efficiency:

Add 10 μL liposome stock to 90 μL buffer in a tube. Add this mix to a 0.5 mL Zeba spin filter (7 kDa) in order to separate the liposomes from the free gardiquimod following the protocol below:
   Remove the bottom closure of the column.
   Place column in collection tube and centrifuge 1 minute at 1500 g to remove storage solution.
   Discard flow-through, add 3004 PBS, and centrifuge 1 minute at 1500 g (wash 1)
   Discard flow-through, add 3004 PBS, and centrifuge 1 minute at 1500 g (wash 2)
   Discard flow-through, add 3004 PBS, and centrifuge 1 minute at 1500 g (wash 3)
   Blot the bottom of the column to remove excess liquid. Transfer to a new clean collection tube.
   Apply the 30 μL sample to the top of the column.
   Centrifuge 2 minutes at 1500 g. Retain the flow-through that contains the sample.
Compare the Zeba flowtrough to a similar 1:10 mixture of liposomes.
Transfer to HPLC vials and run HPLC:
   Reverse phase C18 column (XBridgeC18)
   Flow rate 1 mL/min
   Solvent A: HPLC water with 0.1% TFA
   Solvent B: 100% MeCN with 0.1% TFA
   Use the 22 minute program with start ratio 5% A, end ratio 100% B over 15 minutes and equilibration at 100% B followed by at 5% B.
   Loaded sample volume 504.
   Read absorbance at 280 nm and 320 nm.
   Gardiquimod should elute around 9 minutes.
   Also run a 100 μg/mL to 12.5 μg/mL standard curve of Gdq, as well as the liposome diluted 1:20.

### Method for Size and Zeta Measurement:

Add 5 μL of the liposome stock to 1 mL of dialysis buffer and measure the size on the Brookhaven.
Add 7.5 μL of the liposome stock to 1.6 mL of the dialysis buffer and measure the zeta potential on the Brookhaven.

The size, zeta and encapsulation efficiency are shown below. Before loading, the liposomes were relatively polydisperse. After loading, they had both decreased in size and were more monodisperse.

| Liposome | Batch | Size | PDI | Zeta | Encapsulation |
|---|---|---|---|---|---|
| Before loading | RL11 | 115.7 | 0.127 | 19.52 | — |
| Before loading | RL12 | 121.8 | 0.185 | 21.98 | — |
| After loading | RL11 | 109.9 | 0.118 | — | 70.0% |
| After loading | RL12 | 106.2 | 0.027 | — | 77.4% |

### Example 78: Loading of Liposomes onto PMEL Cells

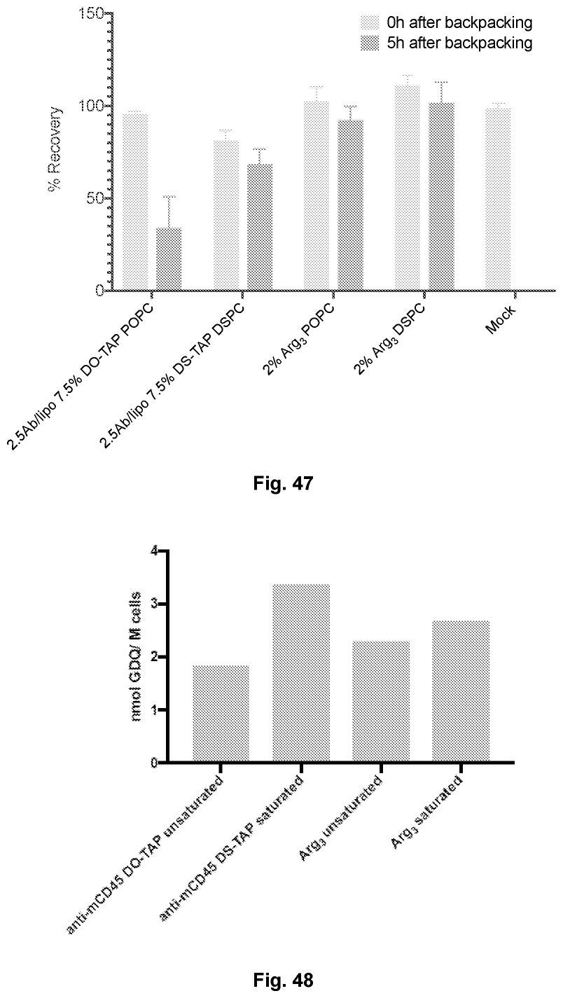

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines and harvested for liposome loading evaluation. Mouse specific anti-CD45 IgG was conjugated to DSPE-PEG via a maleimide reaction with free thiols after reduction with either TCEP or DTT and post inserted into unsaturated (2.5 Ab/liposome, 7.5% DOTAP, 35% cholesterol, 0.5% DSPE-PEG, POPC) and saturated (2.5 Ab/liposome, 7.5% DSTAP, 35% cholesterol, 0.5% DSPE-PEG, DSPC) liposomes at 55 C for 60 min. Antibody density estimation is based on 0.01 nmol of antibody per 1 umol of lipid is equivalent to 1 antibody per liposome. Cholestrol-Arg3 was post inserted into POCP and DSPC liposomes at 55 C for 60 min. PMEL cell at 50×10\hat{ }6/mL were incubated with liposomes at 10 mM lipid concentration for 60 minutes at 37 C, washed twice and analyzed by flow cytometry to measure the % recovery of cells lost due to toxicity relative to a mock loaded cell. As shown in FIG. 47 unsaturated liposome using anti-CD45 antibody were not toxic at 0 hr (light bars) but more toxic after 5 hrs of loading (dark bars) while the saturated liposome with antibody are minimally toxic. Additionally, the Arg3 formulations show minimal toxicity at 0 and 5 hrs, using either unsaturated or saturated liposomes.

### Example 79: Effect of Different Liposome Formulations on GDQ Drug Loading in PMEL Cells

Mouse specific anti-CD45 IgG was conjugated to DSPE-PEG via a maleimide reaction with free thiols after reduction with either TCEP or DTT and 2.5 antibody/liposome post inserted into unsaturated (2.5 Ab/liposome, 7.5% DOTAP, 35% cholesterol, 0.5% DSPE-PEG, POPC, Gardiquimod gradient loaded as previously described) liposomes and saturated (2.5 Ab/liposome, 7.5% DSTAP, 35% cholesterol, 0.5% DSPE-PEG, DSPC, Gardiquimod gradient loaded as previously described) liposomes at 55 C for 60 min. 2% Cholestrol-Arg3 instead of antibody was also post inserted into 7.5% DOTAP POCP liposomes and 7.5% DTAP DSPC liposomes at 55 C for 60 min. Spleens and lymphnodes were harvested from PMEL mice (B6.Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines and harvested for liposome loading evaluation. PMEL cells at 50×10\hat{ }6/mL were incubated with liposomes at 10 mM lipid concentration for 60 minutes at 37 C, washed twice and put into cell culture media with 100 IU/mL mouse IL-2. Samples were taken with time to measure drug loading on the cells and drug release from the cells.

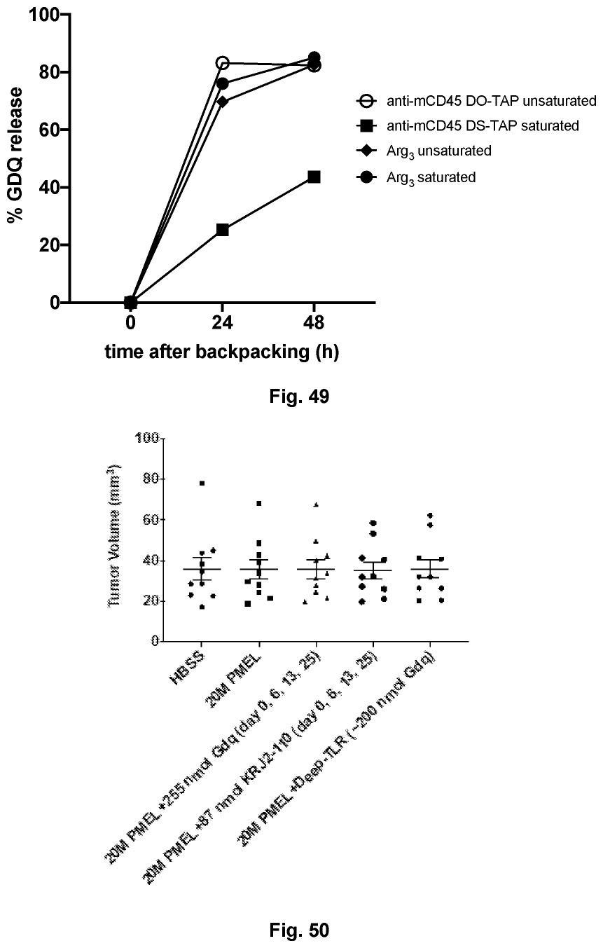

FIG. 48 shows amount of Gardiquimod loaded per 1×10\hat{ }6 PMEL CD8 T cells immediately after 60 minutes incubation and washing of cells. FIG. 49 shows % of Gardiquimod release from PMEL CD8 T cells with time as cells grow in culture with 100 IU/mL IL-2.

### Example 80: In Vivo Efficacy of TLR Agonist Loaded Liposomes

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines, harvested and loaded with liposomes for evaluation in vivo. PMEL cells at 50×10\hat{ }6/mL were incubated with liposomes at 10 mM lipid concentration for 60 minutes at 37 C, washed twice and formulated in HBSS prior to injection into the mice. In vivo study details show below. Briefly, 100 ul of 0.5×10\hat{ }6 B16-F10 cells formulated in HBSS were injected SubQ (SQ) or intra-dermal (ID) and grown for 9 days prior to treatment. 1 day prior to treatment, 4 mg/mouse of cyclophosphamide (CPX) was injected intraperitoneal (IP) with 200 ul at 20 mg/mL in phosphate buffered saline (PBS). There were 10 mice used per group and all treatments were injected intravenous (IV) via tail vein injection, the groups were as follows:
HBSS alone (single dose)
20×10\hat{ }6 PMEL cells/mouse (single dose)
20×10\hat{ }6 PMEL cells/mouse backpacked with 2.5 antiCD45 Ab/liposome, 10% DOTAP, 35% cholersterol, 0.6% DSPE-PEG, POPC, pH gradient loaded as previously described with Gardiquimod (˜200 nmol Gardiquimod/mouse, single dose)
20×10\hat{ }6 PMEL cells/mouse followed by 4 mg/kg Gardiquimod systemically on day 0 (treatment day, day 6, day 13, day 25
20×10\hat{ }6 PMEL cells/mouse followed by 2 mg/kg KRJ2-110 systemically on day 0 (treatment day), day 6, day 13, day 25

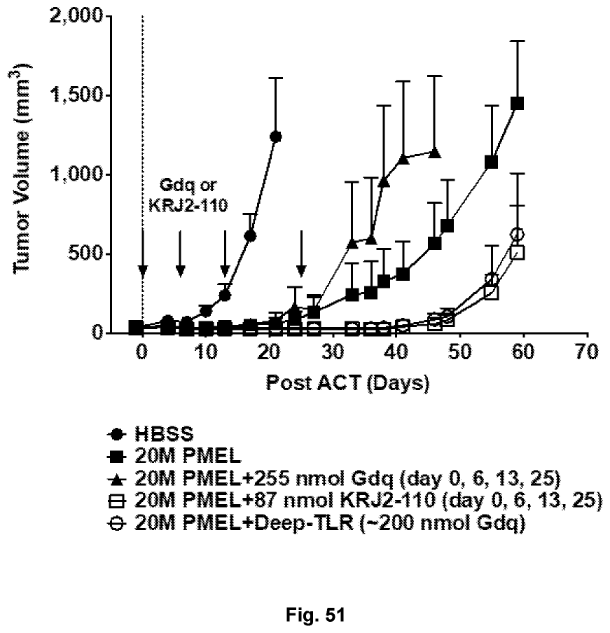

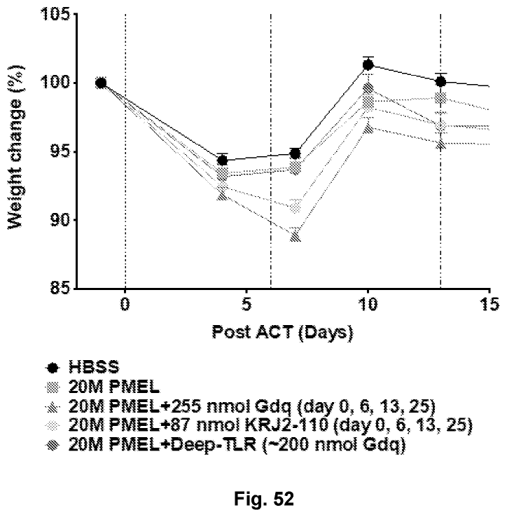

As shown in FIGS. 50-52, KRJ2-110 was dosed at half the mass (3× lower on molar basis) and was more efficacious which is consistent with in vitro potency in a RAW cell assay being ˜10× more potent. 2.5 antiCD45 Ab/liposome, 10% DOTAP, POPC liposomes loaded on PMEL cells were more potent than PMEL cells alone indicating using T cells for small molecule delivery augments cellular therapy efficacy. Additionally, while T cell delivery is more potent than systemic delivery, it also circumvents toxicity indicated by more substantial weight loss in the systemically administered Gardiquimid and KRJ2-110 groups versus HBSS, PMEL alone and PMEL loaded with Gdq liposomes. All of the groups show some weight loss including the untreated HBSS group due to CPX pre-treatment however the weight loss was exacerbated by systemic delivery of TLR7 agonists.

### Example 81: Cytokine/Chemokine Release Kinetics Post Treatment with PMEL Loaded with Liposomes or Systemically Delivered TLR Agonist

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines and harvested for use. The cytokine/chemokine release profile in non-tumor bearing B6D2F1/J mice (Jackson Labs) was evaluated by injecting:
20×10\hat{ }6 PMEL cells alone, 20×10\hat{ }6 PMEL cells loaded with 2.5 ant-CD45 Ab/liposome, 10% DOTAP, 35% cholesterol, 0.6% DSPE-PEG, POPC liposomes Gardiquimod pH gradient loaded as previously described (˜200 nmol gardiquimod, single dose)
4 mg/kg Gdq systemic (no PMEL, single dose)
2 mg/kg KRJ2-110 systemic (no PMEL, single dose)

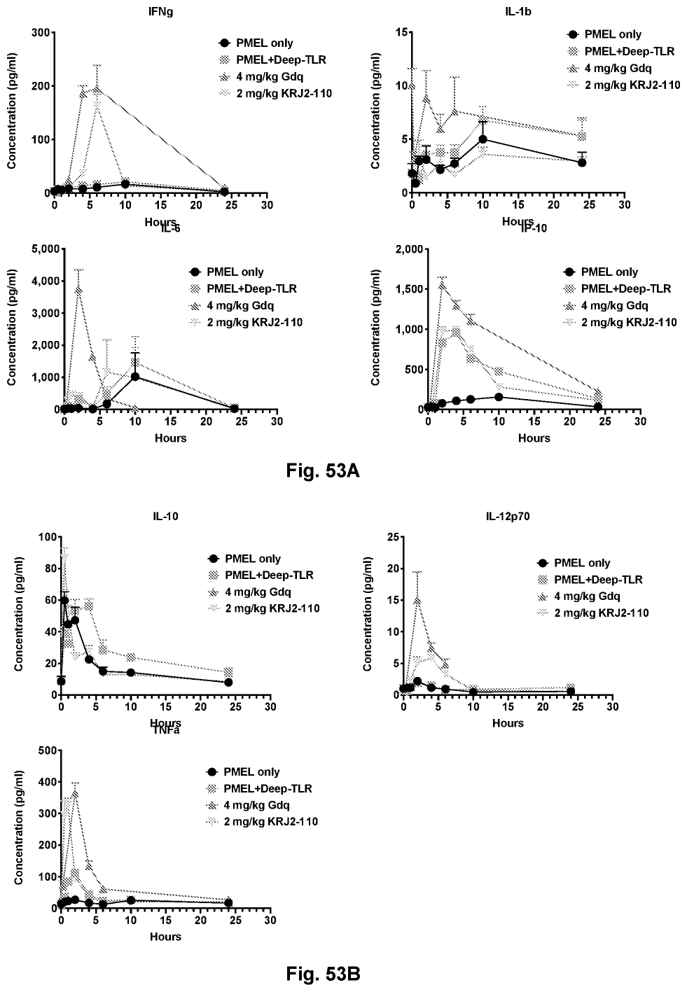

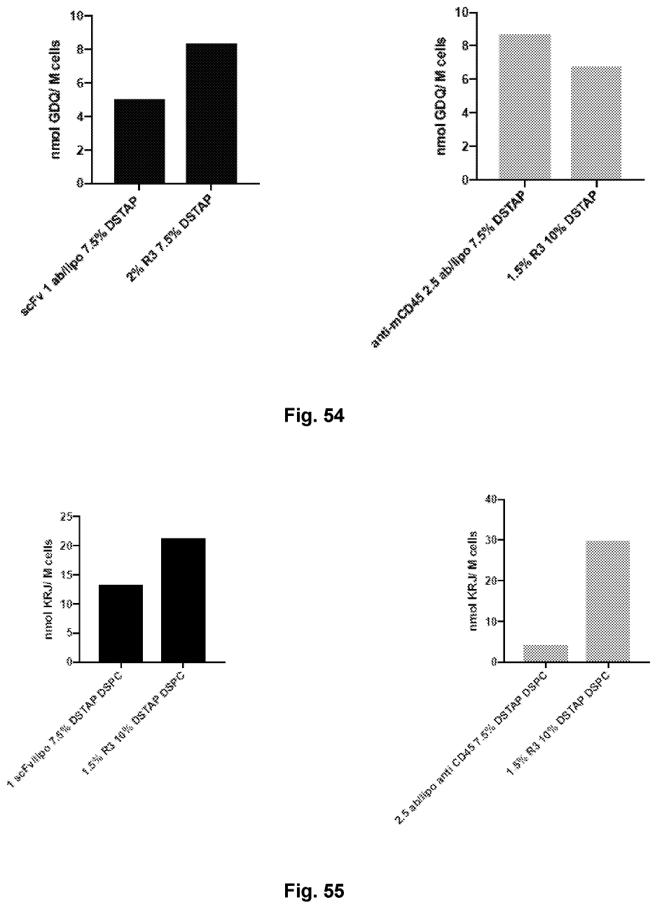

Blood was drawn at 0, 0.25, 0.5, 1, 2, 4, 6, 10 and 24 hr and plasma was recovered for cytokine/chemokine quantitation using a Luminex kit. As shown in FIGS. 53A-53B there was a significant increase in proinflammatory cytokines such as IFNg, IL12 in the groups with systemic delivery of TLR agonists Gardiquimod and KRJ2-110 while the PMEL cells loaded with TLR agonist liposomes did not show a significant increase in these cytokines indicating the systemic exposure of TLR agonist is low for T cells loaded with TLR agonist via liposomes. Interestingly, IP10 and TNFa (nontoxic levels) showed an increase from the PMEL cells loaded with TLR agonist liposomes relative to PMEL cells alone. IP10 and TNFa are commonly used biomarkers for TLR7 agonists in human studies indicating these may be useful biomarkers in clinical studies for T cells loaded with TLR7 agonists.

### Example 82: GDQ and KRJ2-110 Loading onto Human and Mouse T Cells

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines and harvested for liposome loading evaluation. Human CD3 cells were isolated from healthy donor blood by Mylteni bead separation and expanded on anti-CD3/CD28 beads with human IL2 for multiple days prior to use (media is RPMI+10% FBS+1× GlutaMAX+1× Pen/Strep). Mouse specific anti-CD45 IgG or Human specific anti-CD45 ScFv was conjugated to DSPE-PEG via a maleimide reaction with free thiols after reduction with either TCEP or DTT and post inserted into saturated (7.5% DSTAP, 35% cholesterol, 0.5% DSPE-PEG, DSPC, Gardiquimod pH gradient loaded as previously described) liposomes at 55 C for 60 min. Cholestrol-Arg3 was post inserted into DSPC liposomes at 55 C for 60 min. PMEL or human CD3 cells cell at 50×10\hat{ }6/mL were incubated with liposomes at 10 mM lipid concentration for 60 minutes at 37 C, washed twice and cultured in media with time. A C18 column was used to measure the amount of Gardiquimod or KRJ2-110 loaded onto the cells. As shown in FIGS. 54-55, the levels of Gardiquimod loading are within 2-fold of each other across 2 different species with the Arg3 mediated loading being more consistent across species. For KRJ2-110 loading, the level of drug loading is similar on human CD3 cells but much higher for Arg3 mediated loading than antibody mediated loading on mouse PMEL CD8 T cells. Generally the amount of KRJ2-110 loaded on to T cells is higher than Gardiquimod for the various formulations.

### Example 83: Liposomes Containing Antibody and Peptide Conjugated Lipids Increase Drug Loading onto T Cells

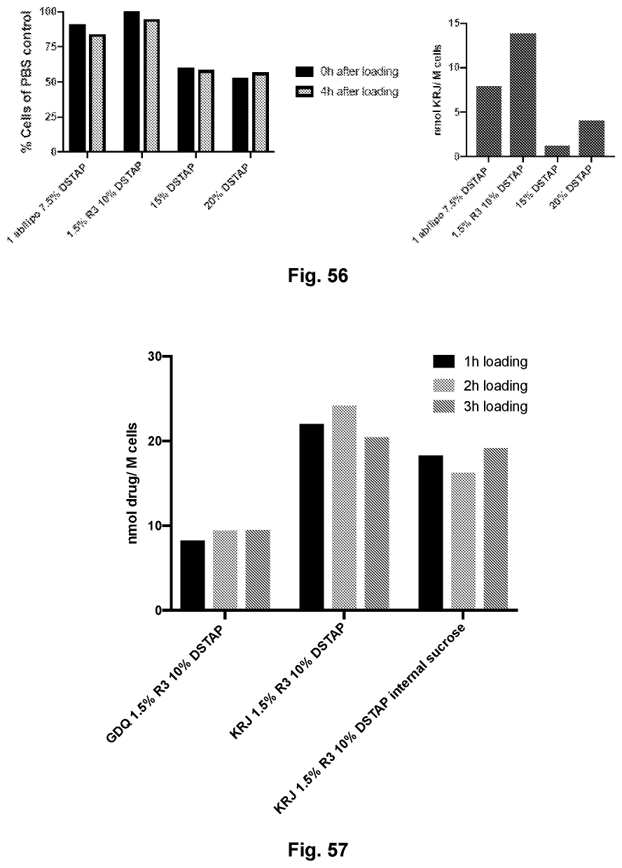

Human CD3 cells were isolated from healthy donor blood by Mylteni bead separation and expanded on anti-CD3/CD28 beads with IL2 for multiple days prior to use (media is RPMI+10% FBS+1× GlutaMAX+1× Pen/Strep). Human specific anti-CD45 ScFv conjugated to DSPE-PEG via a maleimide reaction with free thiols after reduction with either TCEP or DTT and post inserted into saturated (35% cholesterol 0.5% DSPE-PEG, DSPC, KRJ2-110 pH gradient loaded liposomes as previously described) liposomes at 55 C for 60 min. Cholestrol-Arg3 was post inserted into DSPC liposomes at 55 C for 60 min. Human CD3 cells cell at 50×10\hat{ }6/mL were incubated with liposomes at 10 mM lipid concentration for 60 minutes at 37 C, washed twice and cultured in media with time. A C18 column was used to measure the amount of KRJ2-110 loaded onto the cells and flow cytometry was used to measure the toxicity of the liposome loading on live cells relative to mock loaded controls (no liposome). As shown in FIG. 56, the antibody mediated loading and Arg3 mediated loading drove more drug onto human T cells indicating DSTAP alone does not mediate as high of loading of drug onto T cells than DSTAP and Arg3 combined. Additionally, the high amounts of DTAP used to drive some loading onto the cells caused toxicity while the antibody mediated and Arg3 mediated loading displayed minimal toxicity at 0 and 4 hrs after liposomes loading.

### Example 84: Effect of Loading Time on Amount of Drug Loading onto T Cells

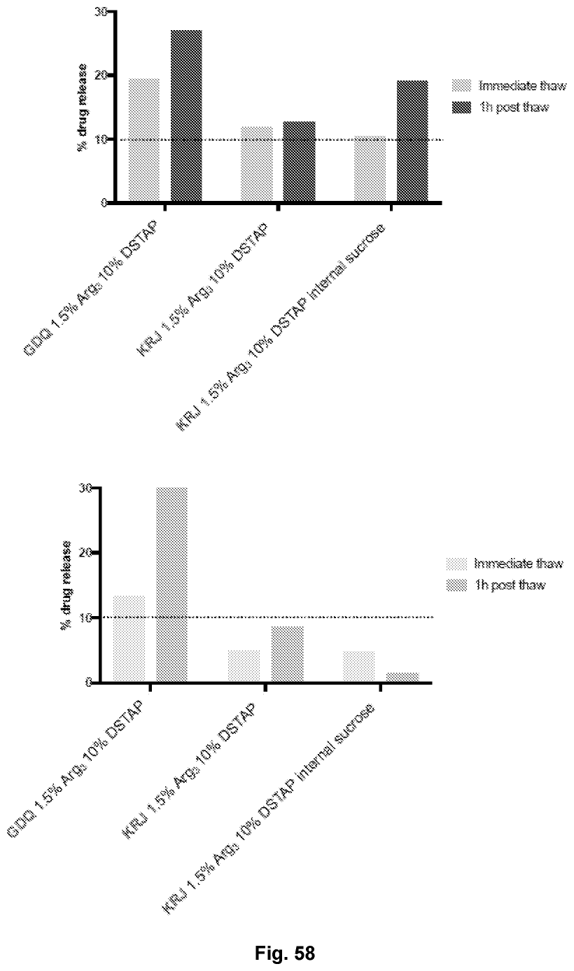

A Leukopak from a healthy donor was processed by an Elutra cell separation system (Terumo BCT) to enrich in human T cells and Monocytes from a mixture of cell types. The T cell rich fraction was frozen while the Monocyte rich fraction was differentiated into Dendritic Cells (DCs) using a protocol similar to fastDC over 3 days. After 3 days the DC rich culture were pulsed with standard CEF peptides (JPT) to load peptide onto DC MHC. T cells were thawed and cultured at 1×10\hat{ }6 cell/mL with CEF-peptide-loaded-DCs for 4 days, washed and resuspended at 1×10\hat{ }6 cells/mL for a total of 7 days prior to harvest and use for loading liposomes. Human specific anti-CD45 ScFv was conjugated to DSPE-PEG via a maleimide reaction with free thiols after reduction with either TCEP or DTT and post inserted into saturated (10% DSTAP, 35% cholesterol, 0.5% DSPE-PEG, DSPC pH gradient loaded with Gardiquimod or KRJ2-110 as previously described) liposomes at 55 C for 60 min. Cholestrol-Arg3 was post inserted into DSPC liposomes at 55 C for 60 min, one formulation contained 6% sucrose inside of the liposome during pH gradient loading of drug. Human CD3 cells cell at 50×10\hat{ }6/mL were incubated with liposomes at 10 mM lipid concentration for 1 hr, 2 hr and 3 hr at 37 C, washed twice and placed in culture or frozen at −80 C in 46% HBSS 46% CSB 8% DMSO using a Corning CoolCell. A C18 column was used to measure the amount of Gardiquimod and KRJ2-110 loaded onto the cells before and after freezing. As shown in FIGS. 57-58, the amount of drug loaded onto the T cells did not vary with 1-3 hrs of load time, however antibody mediated loading for 1 hr had less drug release post thaw than 2 hr loading and Arg3 mediated loading had less drug release at 2 hr loading than 1 hr loading perhaps due to providing more time for internalization. Generally, KRJ2-110 has lower drug release from cells post thaw than Gardiquimod. Sucrose inside the liposome appeared to increase the amount of KRJ2-110 released from R3 liposome loaded cells post thaw for the 1 hr loading condition but reduced the release from cells that were loaded for 2 hrs.

### Example 85: Liposomes Loaded onto PMEL Cells Inhibit Tumor Growth and Prolong Survival

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines, harvested and loaded with liposomes for evaluation in vivo. PMEL cells at 50×10\hat{ }6/mL were incubated with liposomes at 10 mM lipid concentration for 60 minutes at 37 C, washed twice and formulated in HBSS prior to injection into the mice. Briefly, 100 ul of 0.5×10\hat{ }6 B16-F10 cells formulated in HBSS were injected SubQ (SQ) and grown for 9 days prior to treatment. 1 day prior to treatment, 4 mg/mouse of cyclophosphamide (CPX) was injected intraperitoneal (IP) with 200 ul at 20 mg/mL in phosphate buffered saline (PBS). There were 10 mice used per group and all treatments were injected intravenous (IV) via tail vein injection, the groups were as follows:
HBSS alone (single dose)
20×10\hat{ }6 PMEL cells/mouse (single dose)
20×10\hat{ }6 PMEL cells/mouse backpacked with 1.5% Arg3-Chol, 10% DSTAP, 35% cholesterol, 0.5% DSPE-PEG, DSPC, pH gradient loaded as previously described with Gardiquimod (˜200 nmol Gardiquimod/mouse, single dose)
20×10\hat{ }6 PMEL cells/mouse backpacked with 2.5 Ab/liposome, 7.5% DSTAP, 35% cholesterol, 0.5% DSPE-PEG, DSPC, pH gradient loaded as previously described with Gardiquimod (˜200 nmol Gardiquimod/mouse, single dose)
20×10\hat{ }6 PMEL cells/mouse followed by 4 mg/kg Gardiquimod systemically on day 0 (treatment day, day 7, day 14, day 28

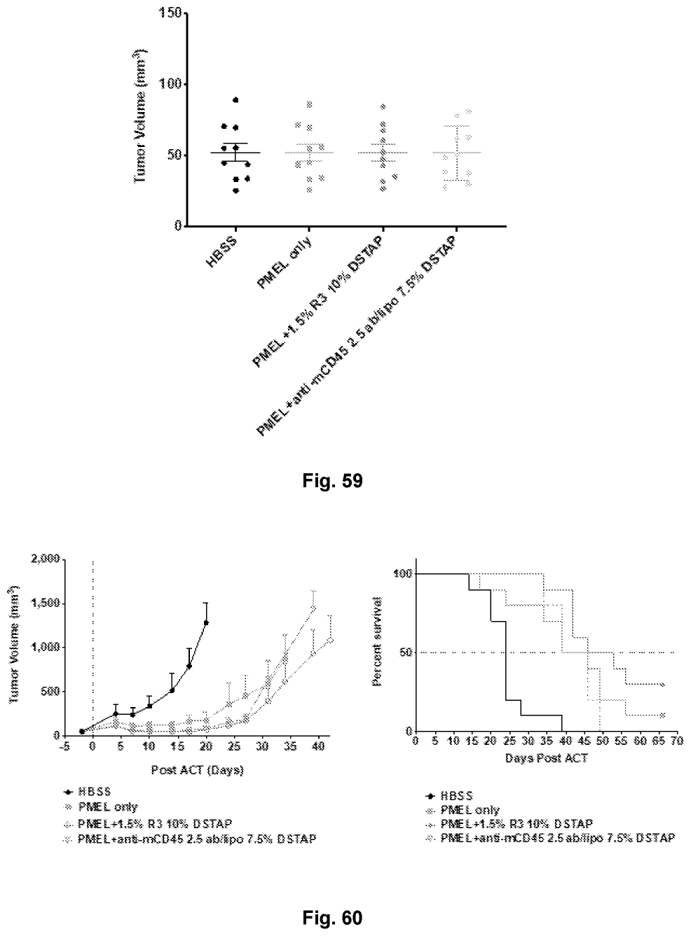

As shown in FIGS. 59-60, the Arg3 and antiCD45 liposomes loaded onto PMEL cells had slightly better tumor growth inhibition than PMEL alone. In terms of survival, the Arg3 liposome loaded PMEL trended towards better survival than PMEL alone and had statistically significantly better survival than antibody mediated loaded liposomes on PMEL.

### Example 86: Peptide Vs. Antibody Mediated Loading and Release

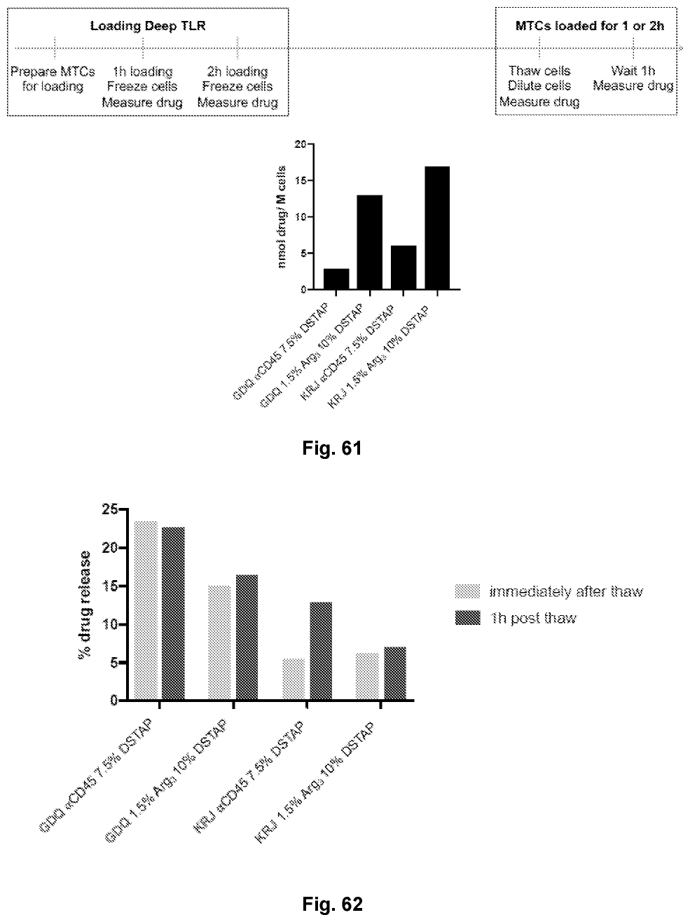

A Leukopak from a healthy donor was processed by an Elutra cell separation system (Terumo BCT) to enrich in human T cells and Monocytes from a mixture of cell types. The T cell rich fraction was frozen while the Monocyte rich fraction was differentiated into Dendritic Cells (DCs) using a protocol similar to fastDC over 3 days. After 3 days the DC rich culture were pulsed with 5 different tumor associated antigen (TAA) peptides (from JPT) to load peptide onto DC MHC. T cells were thawed and cultured at 1×10\hat{ }6 cell/mL with TAA-peptide-loaded-DCs for 4 days, washed and resuspended at 1×10\hat{ }6 cells/mL for a total of 7 days prior to harvest and use for loading liposomes. Human specific anti-CD45 ScFv was conjugated to DSPE-PEG via a maleimide reaction with free thiols after reduction with either TCEP or DTT and post inserted into saturated (1 Ab/liposome, 7.5% DSTAP, 35% cholesterol, 0.5% DSPE-PEG, DSPC, pH gradient loaded with Gardiquimod or KRJ2-110 as previously described) liposomes at 55 C for 60 min. Cholestrol-Arg3 was post inserted into DSPC liposomes at 55 C for 60 min. Human CD3 cells cell at 50×10\hat{ }6/mL were incubated with liposomes at 10 mM lipid concentration for 1 hr or 2 hr at 37 C, washed twice and placed in culture or frozen in a controlled rate freezer (CRF) in 46% HBSS 46% CSB 8% DMSO and stored in liquid nitrogen overnight. A C18 column was used to measure the amount of Gardiquimod and KRJ2-110 loaded onto the cells before and after freezing. It was observed that Arg3 mediated loaded 2-3 more drug than antibody mediated loading (FIG. 61). Consistent with previous studies, KRJ2-110 loaded more than Gardiquimod. Additionally, post thaw, KRJ2-110 released less drug than Gardiquimod (FIG. 62). Generally, antibody mediated loading had a higher release of drug post thaw than Arg3 peptide mediated loading. This could be due to peptide mediated loading driving rapid internalization and more protection for the liposomes to remain intact during the freeze/thaw cycle while during antibody mediated loading, the liposomes are more surface oriented and therefore the freeze/thaw cycle may be more harsh.

### Example 87: Delivering TLR7 Agonist In Vivo Via PMEL Loaded with KRJ2-110 Arg3 Liposomes

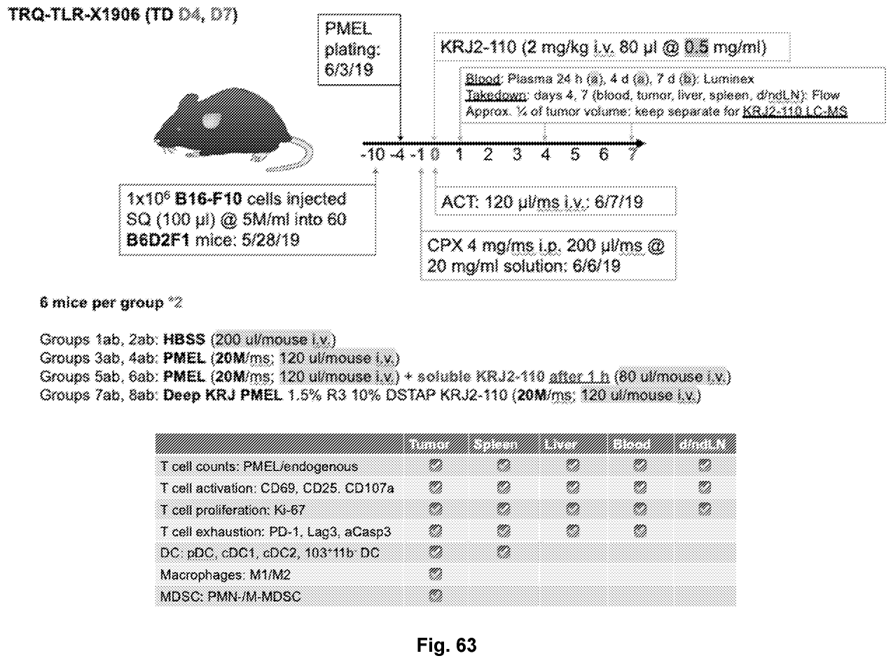

Spleens and lymphnodes were harvested from PMEL mice (B6. Cg-Thy1^{a}/Cy Tg(TcraTcrb)8Rest/J, Jackson Labs) and CD8 T cells purified by Mylteni MACS T cell separation kit. PMEL cells were stimulated and expanded on anti-CD3/CD28 coated plates with mouse IL2/IL7/IL21 cytokines, harvested and loaded with liposomes for evaluation in vivo. PMEL cells at 50×10\hat{ }6/mL were incubated with liposomes at 10 mM lipid concentration for 60 minutes at 37 C, washed twice and formulated in HBSS prior to injection into the mice. Details of the in vivo study are shown in FIG. 63. Briefly, 100 ul of 1×10\hat{ }6 B16-F10 cells formulated in HBSS were injected SubQ (SQ) and grown for 10 days prior to treatment. 1 day prior to treatment, 4 mg/mouse of cyclophosphamide (CPX) was injected intraperitoneal (IP) with 200 ul at 20 mg/mL in phosphate buffered saline (PBS). There were 10 mice used per group and all treatments were injected intravenous (IV) via tail vein injection, the groups were as follows:
HBSS alone (single dose)
20×10\hat{ }6 PMEL cells/mouse (single dose)
20×10\hat{ }6 PMEL cells/mouse backpacked with 1.5% Arg3-Chol, 10% DSTAP, 35% cholesterol, 0.5% DSPE-PEG, DSPC, pH gradient loaded as previously described with KRJ2-110 (single dose)
20×10\hat{ }6 PMEL cells/mouse followed by 2 mg/kg KRJ2-110 systemically on day 0 (small molecule delivered 1 hr after cell dose)

Blood was sampled at 24 hr, 4 days and 7 days for plasma isolation and cytokine/chemokine characterization by Luminex. Tissues and organs were harvested on day 4 and 7 for analysis by flow cytometry and Luminex.

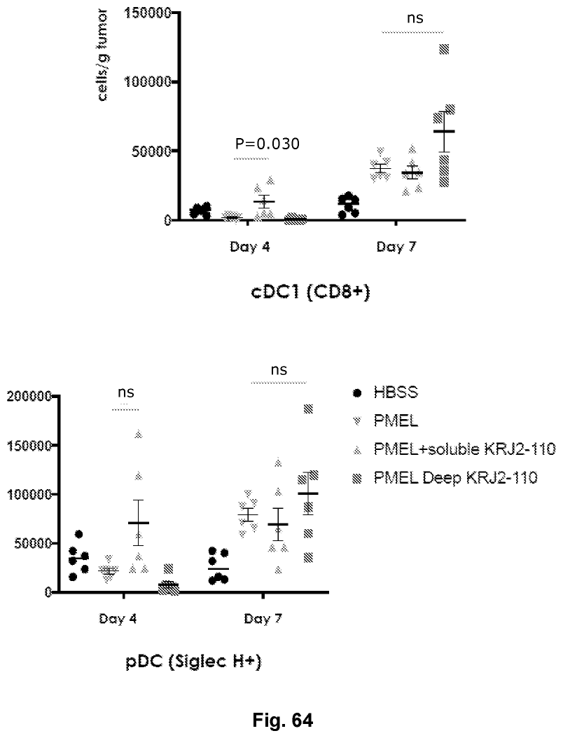

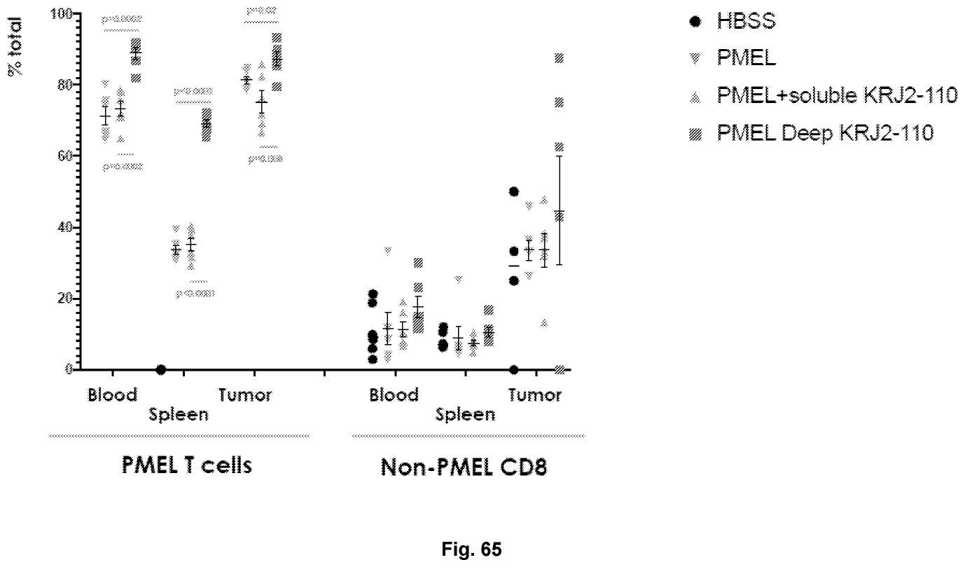

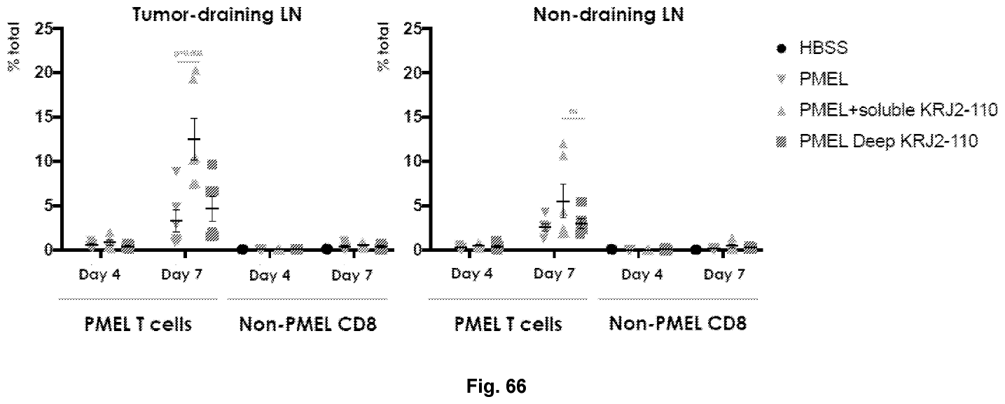

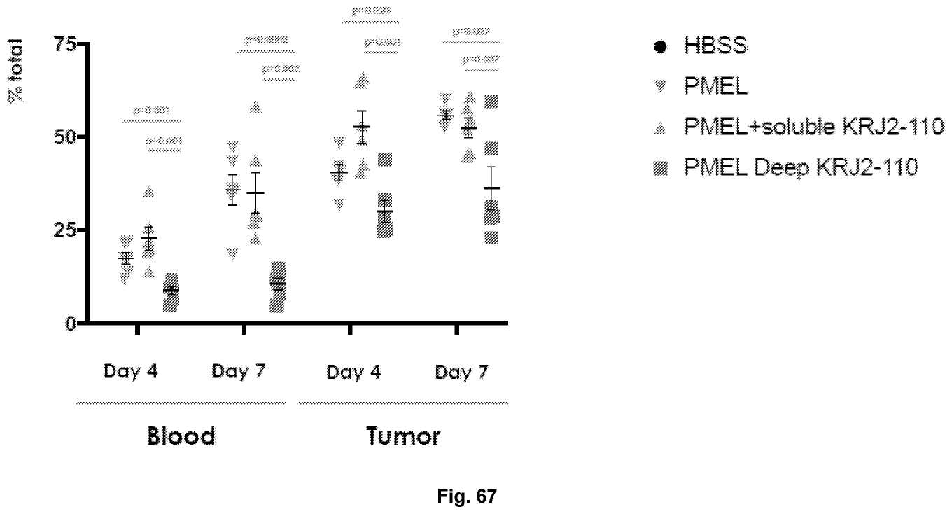

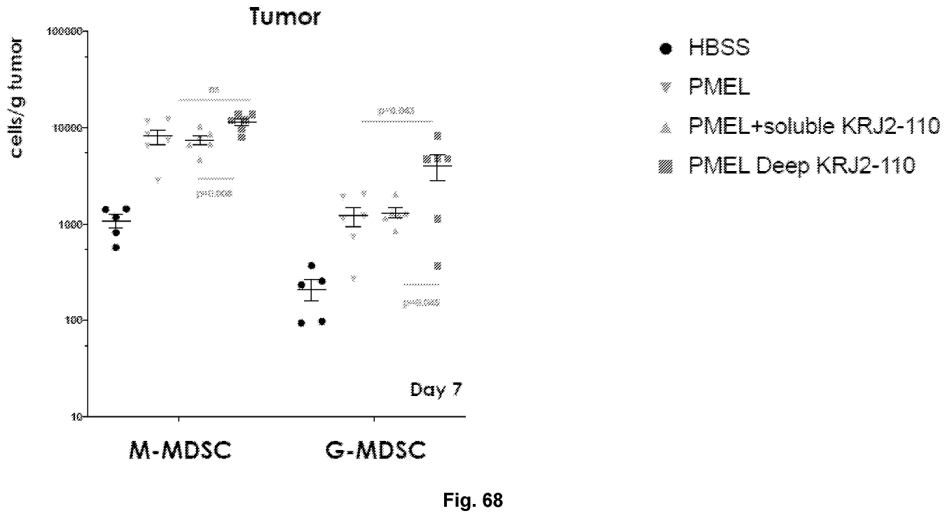

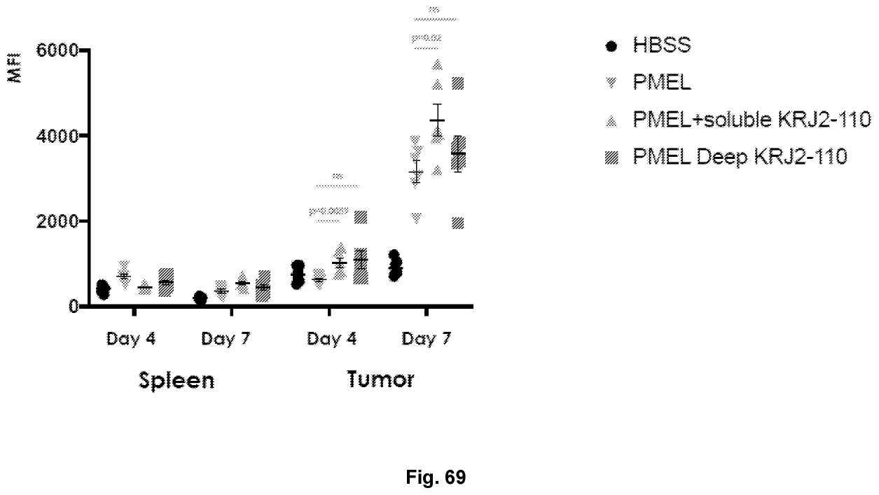

As shown in FIGS. 64-69, delivering TLR7 agonist in vivo via PMEL loaded with KRJ2-110 Arg3 liposomes (“Deep KRJ2-110”):
Increases the abundance of cDC1 and pDC in the tumor
Increases proliferation of PMEL T cells measured by Ki67 in the blood, spleen and tumor, and tends to increase proliferation of endogenous CD8 cells in the tumor
Reduces exhaustion by measurement of PD1+/LAG3+ PMEL cells
Increases Monocytic and Granulocytic Myeloid Derived Suppressor Cells (MDSCs) density in the tumor

While systemically administered KRJ2-110 in conjunction with PMEL administration:
Transiently increases the abundance of cDC1 and pDC
Virtually no effect on PMEL T cell proliferation
Increases activation of PMEL T cells in LN
Does not markedly affect MDSC
Increases MHCII expression in the tumor
Slightly increases exhaustion

### Example 88: Antibody Mediated Loading on PMEL Cells

PMEL CD8 T cells were harvested from PMEL mice and expanded on anti-CD3/CD28 coated plates with cytokines for 4 days and harvested for liposome loading evaluation. Mouse specific anti-CD45 IgG, anti-CD11a Fab, anti-CD8a Fab and anti-PD1 were conjugated to DSPE-PEG via a maleimide reaction with free thiols after reduction with either TCEP or DTT and post inserted into 10% DOTAP POPC liposomes doped with ATTO-488 dye. PMEL cells at 50×10\hat{ }6/mL were incubated with liposomes at 10 mM lipid concentration for 60 minutes, washed twice and analyzed by flow to measure liposome loading on viable cells. The receptor density on CD8 PMEL cells was used measured using the BD Quantibrite kit per the manufacturer's instructions.

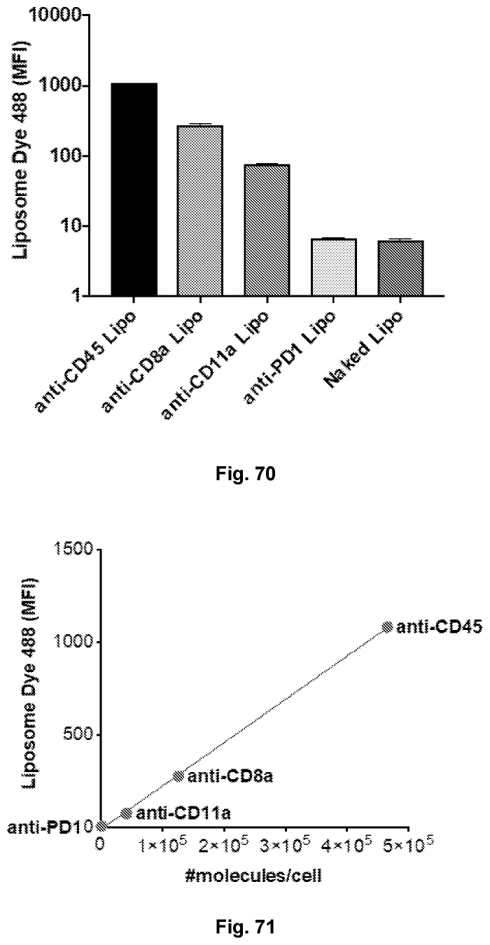

As shown in FIGS. 70-71, CD45 targeting gave the highest loading followed by CD8 and CD11 while PD1 targeting had minimal loading (though above background) onto cells relative to antibody free liposome alone. Additionally, the amount of liposome loaded correlated very strongly with the receptor density on the surface of PMEL cells as measured using the BD Quantibrite kit.

### Example 89: Antibody Mediated Loading on PMEL T Cells and Human CD3s

PMEL CD8 T cells were harvested from PMEL mice and expanded on anti-CD3/CD28 coated plates with cytokines for 4 days and harvested for liposome loading evaluation. Human CD3 cells were bead purified and expanded on CD3/CD28 beads with IL2 for multiple days prior to use. Mouse specific anti-CD45 IgG, anti-CD11a Fab, anti-CD8a Fab and human specific anti-CD45 ScFv and anti-CD11a were conjugated to DSPE-PEG via a maleimide reaction with free thiols after reduction with either TCEP or DTT and post inserted into 10% DOTAP POPC liposomes doped with ATTO-688 dye at approximately 2 and 10 Ab/liposomes (0.01 nmol antibody/1 umol of lipid is ˜1 antibody/liposome. T cells at 50×10\hat{ }6/mL were incubated with liposomes at 10 mM lipid concentration for 60 minutes, washed twice and analyzed by flow to measure liposome loading on viable cells.

As shown in FIG. 72, targeting human CD45 gave the highest loading, then human CD11a then the mouse CD11 and CD8 constructs, respectively. Additionally, it appears the optimal antibody density per liposome is <=10 Ab/liposome as the signal is higher or similar at the lower antibody density of 2 Ab/liposome.

### Example 90: Arg3, Arg8 and Combinations with Anti-CD45 Antibody

Human CD3 cells were bead purified and expanded on CD3/CD28 beads with IL2 for multiple days prior to use. Human specific anti-CD45 ScFv was conjugated to DSPE-PEG via a maleimide reaction with free thiols after reduction with either TCEP or DTT and post inserted into 10% DOTAP POPC liposomes doped with ATTO-488 dye at approximately 2.5, 1.25, 0.625 Ab/liposome (0.01 nmol antibody/1 umol of lipid is ˜1 antibody/liposome) with varying percentages of Arg3-cholesterol and Arg8-cholesterol that was post inserted separately from the antibody. T cells at 50×10\hat{ }6/mL were incubated with liposomes at 10 mM lipid concentration for 60 minutes, washed twice and analyzed by flow to measure liposome loading on viable cells.

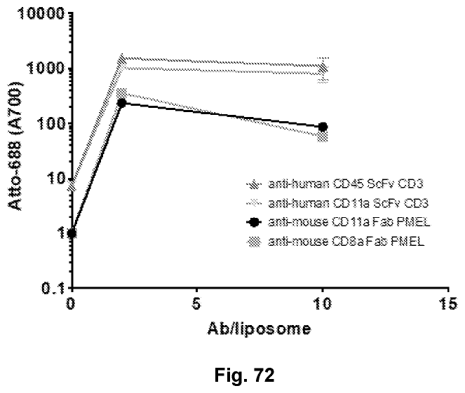

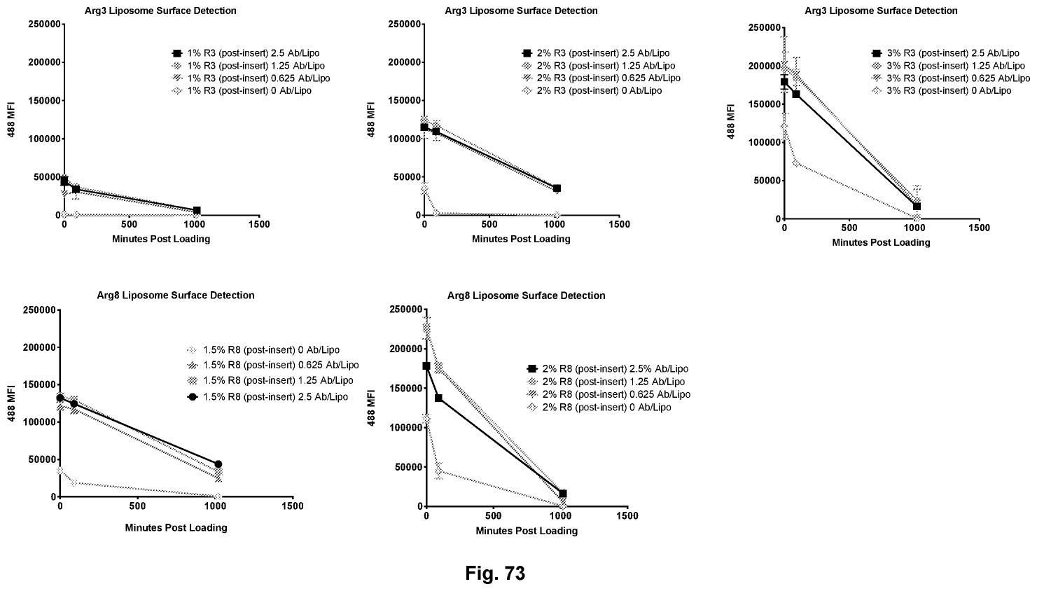

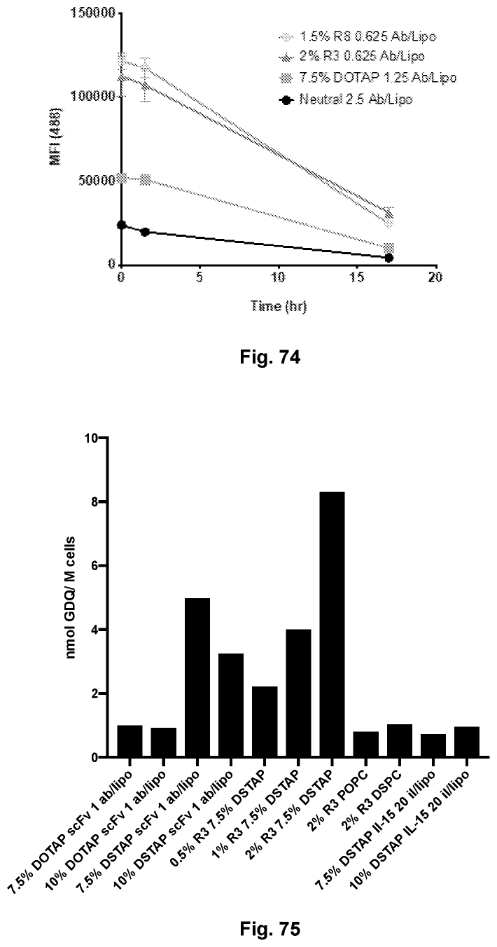

As shown in FIGS. 73-74, Arg3 and Arg8 liposomes show measurable surface levels of liposomes at early time points but the signal degrades with time as liposomes are likely internalized and the dye loses activity. In contrast however, the liposomes with antibody show less degradation of signal with time likely show a slowing of internalization by antibody tethering to the surface. Also shown below is that liposomes with both R3 or R8 and antibody can get higher surface binding than just liposomes with just antibody alone showing a synergistic effect of using both a peptide and antibody to mediate T cell loading of liposomes.

### Example 91: Drug Loading of Various Formulations Containing Gardiquimod onto Human T Cells

Human CD3 cells were isolated from healthy donor blood by Mylteni bead separation and expanded on anti-CD3/CD28 beads with IL2 for multiple days prior to use (media is RPMI+10% FBS+1× GlutaMAX+1× Pen/Strep). Human specific anti-CD45 ScFv or IL-15/sushi-Fc (IgG2-DA) conjugated to DSPE-PEG via a maleimide reaction with free thiols after reduction with either TCEP or DTT and post inserted into saturated or unsaturated liposomes (35% cholesterol, 0.5% DSPE-PEG, DSPC or POPC, Gardiquimod pH gradient loaded liposomes as previously described) at 55 C for 60 min. Cholestrol-Arg3 was post inserted into DSPC liposomes at 55 C for 60 min. Human CD3 cells cell at 50×10\hat{ }6/mL were incubated with liposomes at 10 mM lipid concentration for 60 minutes at 37 C, washed twice and cultured in media with time. A C18 column was used to measure the amount of Gardiquimod loaded onto the cells.

As shown in FIG. 75, Arg3 in 7.5% DSTAP liposomes can load substantially higher amounts of drug relative to POPC or DSPC liposomes that lack charged lipids such as DOTAP or DSTAP. Given we have previously shown that a charged liposome alone, such as DSTAP, does not significantly load drug onto the cells, a synergistic effect is taking place between positively charged liposomes and Arg3-cholesterol. Additionally, IL-15/sushi-Fc decorated liposomes did not drive as much drug into the cell as Arg3/DSTAP liposomes however it does improve the viability of the T cells with time in the absence of growth promoting cytokines such as IL-2 or IL-15 in vitro.

### Example 92: Drug Loading of Various Formulations Containing KRJ2-110 onto Human T Cells

Human CD3 cells were isolated from healthy donor blood by Mylteni bead separation and expanded on anti-CD3/CD28 beads with IL2 for multiple days prior to use (media is RPMI+10% FBS+1× GlutaMAX+1× Pen/Strep). Human specific anti-CD2 IgG was conjugated to DSPE-PEG via a maleimide reaction with free thiols after reduction with either TCEP or DTT and post inserted into saturated or unsaturated liposomes (35% cholesterol 0.5% DSPE-PEG, DSPC or, KRJ2-110 pH gradient loaded liposomes as previously described) at 55 C for 60 min. Cholestrol-Arg3 was post inserted into DSPC liposomes at 55 C for 60 min. Human CD3 cells cell at 50×10\hat{ }6/mL were incubated with liposomes at 10 mM lipid concentration for 60 minutes at 37 C, washed twice and cultured in media with time. A C18 column was used to measure the amount of KRJ2-110 loaded onto the cells.

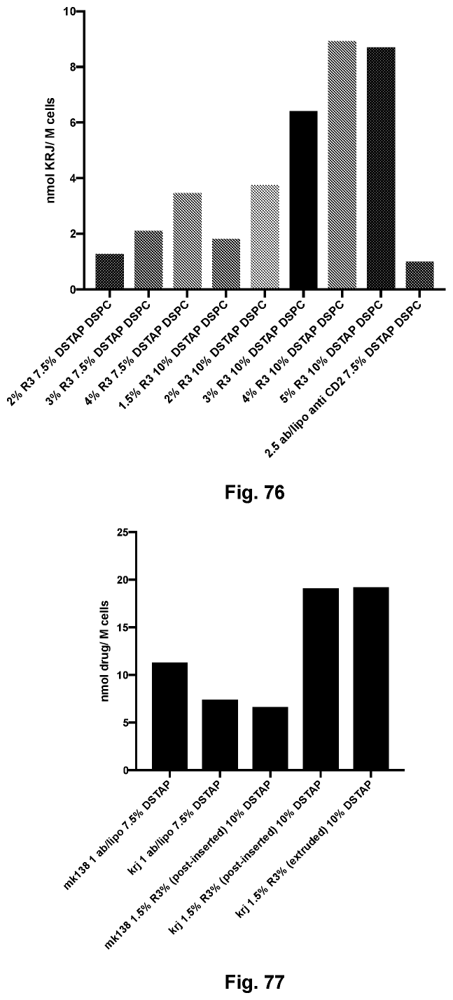

As shown in FIG. 76, the % of Arg3-cholesterol can be increased to improve the drug loading into the liposomes. The benefit of more Arg3 maxes out with 10% DSTAP and 4% Arg3 however higher DSTAP and Arg3 may provide more benefit and toxicity can be optimized by modifying the % of DSPE-PEG. While CD2 is an attractive T cell surface tethering target for liposomes given it has activating properties, it did not load more drug than Arg3-cholesterol mediated loading of liposomes onto human T cells.

### Example 93: Drug Loading of Various Formulations Containing MK-138 or KRJ2-110 onto Human T Cells

Human CD3 cells were isolated from healthy donor blood by Mylteni bead separation and expanded on anti-CD3/CD28 beads with IL2 for multiple days prior to use (media is RPMI+10% FBS+1× GlutaMAX+1× Pen/Strep). Human specific anti-CD45 ScFv was conjugated to DSPE-PEG via a maleimide reaction with free thiols after reduction with either TCEP or DTT and post inserted into saturated or unsaturated liposomes (35% cholesterol 0.5% DSPE-PEG, DSPC, MK-138 a TLR7/8 agonist or KRJ2-110 pH gradient loaded liposomes as previously described) at 55 C for 60 min. Cholesterol-Arg3 was either formulated with other liposome components prior to extrusion (extruded below) or cholestrol-Arg3 was post inserted into DSPC liposomes at 55 C for 60 min (post-inserted below). Human CD3 cells cell at 50×10\hat{ }6/mL were incubated with liposomes at 10 mM lipid concentration for 60 minutes at 37 C, washed twice and cultured in media with time. A C18 column was used to measure the amount of MK-138 or KRJ2-110 loaded onto the cells.

As shown in FIG. 77, MK-138 loads slightly better than KRJ2-110 onto human T cells via antibody mediated liposomes. Additionally, Arg3-cholesterol can be formulated with other liposome components or post inserted after extrusion without an effect on T cell loading which simplifies the manufacturing process.

### REFERENCES

Duong, A. D. et al., Mol. Pharmaceutics 2016, 13, 92-99.
Fox, C. B. et al., J. Nanobiotechnology 2014, 12, 17.
Fujita et al., Amino Acids 2016, 48, 1319-1329.
Gerster et al., J. Med. Chem. 2005, 48, 3481-3491.
Izumi et al., Bioorganic & Medicinal Chemistry 2003, 11, 2541-2550.
Johansen, P. T. et al., Expert Opin. Drug Deliv. 2015, 12, 1045-1058.
Klauber, T. C. B. et al., Acta Biomaterialia, 2017, 53, 367-377.
Pedersen and Rosenbohm Synthesis 2001, 16, 2431-2434.
Shukla et al., J. Med. Chem. 2010, 53, 4450-4465.
Smirnov, D. et al., Vaccine, 2011, 29, 5434.5442.
Viswanadhan, V. N.; Ghose, A. K.; Revankar, G. R. and Robins, R. K. J. Chem. Inf. Comput. Sci., 1989, 29 (3), 163-172.
WO2006/029115.
U.S. Pat. No. 6,908,939B2.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound or a pharmaceutically acceptable salt thereof, wherein the compound or salt thereof is substantially purified and wherein the compound has formula (I): 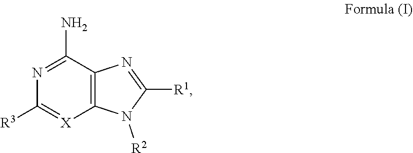
wherein:
X is selected from C or N;
R¹ is selected from the group consisting of OH and R^{A}, wherein R^{A} is optionally substituted with one or more Y;
R² is R^{A} optionally substituted with one or more of: —C₆-C₁₀-aryl (such as phenyl) optionally substituted with one or more R⁴, —C₄-C₁₀-heteroaryl optionally substituted with one or more R⁴, and/or Y;
R³ is (i) R^{A}, or (ii) together with X and the carbon to which it is attached, forms a C₄-C₁₀, preferably —C₅-C₆, aryl or heteroaryl, wherein R³ is optionally substituted with one or more Y;
R⁴ is selected from the group consisting of —C(O)—NH—R^{A}—R⁵, —R^{A}—NH—R^{A}—R⁵, and —R^{A}—NH—R⁵;
R⁵ is selected from the group consisting of H, —C₅-C₁₂-cycloalkyl, —C₄-C₁₂-heterocycloalkyl, —C₆-C₁₀-aryl, and —C₄-C₁₀-heteroaryl, wherein R⁵ is optionally substituted with one or more C₀-C₆-alkyl-Y;
Y is —COOH or —N(R^{B})(R^{C}), wherein (i) R^{B} and R^{C} are each independently selected from the group consisting of H, R^{A}, and (amino acid)ₙ wherein n is an integer selected from 1-6, or (ii) R^{B} and R^{C} together with N form a 3-6-membered ring; and
R^{A}, at each occurrence, is independently selected from the group consisting of —C₁-C₁₂-alkyl, —C₂-C₁₂-alkenyl, —C₂-C₁₂-alkynyl, —C₁-C₁₂-heteroalkyl, —C₂-C₁₂-heteroalkenyl, and —C₂-C₁₂-heteroalkynyl; and
wherein the compound has at least one carboxylic acid group or at least one amine group;
wherein when the compound has at least one carboxylic acid group, the compound has a logD above 0 in the pH range of about 6-10 and/or a logD below 0 in the pH range of about 4-6; and
wherein when the compound has at least one amine group, the compound has a logD above 0 in the pH range of about 2-6 and/or a logD below 0 in the pH range of about 6-9.

2. The compound or salt of claim 1, wherein in formula (I):
R¹ is OH or —C₁-C₄-heteroalkyl;
R² is selected from the group consisting of (a) —C₁-C₆-alkyl-phenyl, wherein the phenyl ring is optionally substituted with R⁴, (b) —C₁-C₆-alkyl-NH₂ and (c) —C₁-C₆-alkyl-NH-amino acid;
R³ is —C₁-C₆-heteroalkyl or when taken together with X (which is C) and the carbon to which it is attached, forms a C₅-C₆ aryl or heteroaryl; and
R⁴ is —C(O)—NH—C₁-C₄-alkyl-R⁵, —CH₂—NH—C₁-C₄-alkyl-R⁵, or —CH₂NHR⁵.

3. The compound or salt of claim 1, wherein in formula (I):
R¹ is selected from the group consisting of OH, —CH₂—O—CH₂—CH₃ and —CH₂—NH—CH₂—CH₃;
R² is selected from the group consisting of benzyl wherein the phenyl ring is substituted with R⁴, —CH₂—CH₂—NH₂ and —CH₂—CH₂—NH—C(O)—C(R⁶R⁷)—NH₂;
R³ is —O—CH₂—CH₂—O—CH₃ or when taken together with X (which is C) and the carbon to which it is attached, forms a benzene ring;
R⁴ is selected from the group consisting of —C(O)—NH—CH₂—CH₂—R⁵, —C(O)—NH—CH₂—R⁵, —CH₂—NH—CH₂—CH₂—R⁵, —CH₂—NH—CH₂—R⁵, and CH₂NHR⁵;
R⁵ is selected from the group consisting of H, N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

4. The compound or salt of claim 1, wherein in formula (I):
X is N;
R¹ is OH;
R² is benzyl, wherein the phenyl ring is substituted with R⁴;
R³ is —O—CH₂—CH₂—O—CH₃;
R⁴ is selected from the group consisting of —C(O)—NH—CH₂—CH₂—R⁵, —C(O)—NH—CH₂—R⁵, —CH₂—NH—CH₂—CH₂—R⁵ or —CH₂—NH—CH₂—R⁵; and
R⁵ is selected from the group consisting of H, N-methyl-N′-piperazinyl, N-pyrrolidinyl, N-methyl-2-pyrolidinyl, 2-amino-4-pyridinyl, p-aminomethyl phenyl, N-pyrrolyl, p-amino phenyl, N-piperazinyl, N-morpholinyl and 2-piperidinyl.

5. The compound or salt of claim 1, wherein in formula (I):
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ is taken together with X, to which it is attached, to form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

6. The compound or salt of claim 1, wherein in formula (I):
X is C;
R¹ is —CH₂—O—CH₂—CH₃ or —CH₂—NH—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is —CH₃ or —CH(CH₃)₂.

7. The compound or salt of claim 1, wherein in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is selected from the group consisting of H, —CH₃, —CH(CH₃)₂, —CH₂—CH(CH₃)₂, —CH₂—CH₂—S—CH₃ and —CH₂—CH₂—CH₂—CH₂—N(CH₃)₂.

8. The compound or salt of claim 1, wherein in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is selected from the group consisting of H, —CH₃, —CH(CH₃)₂ and —CH₂—CH(CH₃)₂.

9. The compound or salt of claim 1, wherein in formula (I):
X is C;
R¹ is —CH₂—O—CH₂—CH₃;
R² is —CH₂—CH₂—NH₂ or —CH₂—CH₂—NH—C(O)—C(R⁶R⁷)—NH₂;
R³ together with X (which is C) and the carbon to which it is attached, form a benzene ring;
R⁶ is H or —CH₃; and
R⁷ is —CH₃ or —CH(CH₃)₂.

10. The compound or salt of claim 1, wherein the compound is

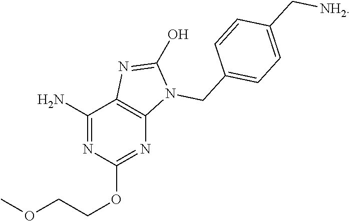 [SMILES: COCCOc1nc(N)c2nc(O)n(Cc3ccc(CN)cc3)c2n1]

11. The compound or salt of any one of claims 1-10, wherein the compound is a TLR7/8 agonist and/or induces expression of one or more cytokines such as IL-6, IL-12p40 and/or IFNα.

12. A liposome composition comprising a liposome and a salt of Gardiquimod and/or one or more of the compounds of any one of claims 1-11, wherein the salt is entrapped inside the liposome, wherein the liposome comprises an interior buffer solution.

13. The liposome composition of claim 12, wherein (1) the compound comprises an aliphatic amine group and the interior buffer solution comprises an acidic component, such that inside the liposome the compound reacts with the acidic component to form the salt; or (2) the compound comprises a carboxylic acid group and the interior buffer solution comprises a basic component, such that inside the liposome the compound reacts with the basic component to form the salt.

14. The liposome composition of claim 12 or 13, wherein the liposome comprises in its membrane one or more of: DSPC (1,2-distearoyl-sn-glycero-3-phosphocholine), HSPC (hydrogenated soybean phosphatidylcholine),CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy(polyethylene glycol)]).

15. The composition of claim 14, wherein the liposome comprises in its membrane about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

16. A method of liposome loading, comprising:
a. providing a liposome in an exterior buffer solution, wherein the liposome comprises an interior buffer solution and wherein a pH gradient exists between the exterior buffer solution and the interior buffer solution across liposome membrane, wherein preferably the pH gradient is at least about 2.0;
b. providing a TLR7/8 agonist comprising a carboxylic acid and/or an aliphatic amine group, wherein optionally the TLR7/8 agonist is Gardiquimod and/or one or more of the compounds of any one of claims 1-11, and
c. combining said liposome in said exterior buffer solution with said compound, thereby loading at least a portion of Gardiquimod and/or said compound into the liposome to form a loaded liposome.

17. The method of claim 16, wherein the method has a loading efficiency of at least 50%, such as at least 70%, at least 80%, or at least 90%.

18. The method of claim 16, wherein the compound comprises an aliphatic amine group and the interior buffer solution comprises an acidic component, such that inside the liposome the compound reacts with the acidic component to form a salt.

19. The method of claim 16, wherein the acidic component is selected from the group consisting of ammonium sulphate, ammonium phosphate, ammonium citrate, ammonium acetate, citric acid, acetic acid, oxalic acid, tartronic acid, dihydroxymalonic acid, fumaric acid, malic acid, tartaric acid, glutaric acid, phosphoric acid, sodium phosphonate, potassium phosphonate, sulfonic acid, sucrose octasulfonic acid, or the basic component is selected from the group consisting of ammonium acetate, potassium acetate, sodium acetate, calcium acetate, ammonium benzoate, potassium benzoate, sodium benzoate and calcium benzoate.

20. The method of claim 16, wherein the compound comprises a carboxylic acid group and the interior buffer solution comprises a basic component, such that inside the liposome the compound reacts with the basic component to form a salt.

21. The method of claim 20, wherein the basic component is selected from the group consisting of ammonium acetate, potassium acetate, sodium acetate, calcium acetate, ammonium benzoate, potassium benzoate, sodium benzoate and calcium benzoate.

22. The method of any one of claims 18-21, wherein the salt is a precipitate.

23. The method of any one of claims 16-22, wherein in the loaded liposome, less than about 20%, preferably less than about 10%, more preferably less than about 5% of the compound is released from the liposome after 1 month or longer at 5° C., such as after 1 week or longer at 5° C.

24. The method of any one of claims 16-23, wherein in the loaded liposome, a drug-to-lipid ratio is at least 0.2, for example at least 0.25, such as at least 0.3.

25. The method of any one of claims 16-24, wherein the compound has a logD above 0 in the exterior buffer solution and/or a logD below 0 in the interior buffer solution.

26. The method of any one of claims 16-19 and 22-25, wherein the compound contains an aliphatic amine group.

27. The method of claim 26, wherein the interior buffer solution has a pH in the range of about 4-6.5, such as in the range of 4-6 or in the range of 5-6.

28. The method of claim 26 or 27, wherein the exterior buffer solution has a pH in the range of 7-9.5, such as in the range of 7-9, in the range of 7-8.5, or in the range of 7-8.

29. The method of any one of claims 26-28, wherein the exterior buffer solution comprises a buffering component selected from the group consisting of HEPES, TAPS, phosphate, histidine, citrate, Bicine, TRIS, TAPSO, TES, Bis-tris, ADA, ACES, PIPES, MOPSO, BES, TES, DIPSO, MOBS, TAPSO, Trizma, HEPPSO, POPSO, TEA, EPPS, Tricine, Gly-Gly, HEPBS, AMPD, TABS, AMPSO, CHES, CAPSO, AMP and MOPS.

30. The method of any one of claims 26-29, wherein a conjugate acid of the compound has a pKa in the range of about 5.5-10, such as in the range of 6-9, for example in the range of 6.5-9, such as in the range of 6.5-8.5, for example in the range of 6.5-8.

31. The method of any one of claims 26-30, wherein the compound has a logD above 0 in the pH range of 6-10 and/or a logD below 0 in the pH range of 4-6.

32. The method of any one of claims 16-17 and 20-25, wherein the compound comprises a carboxylic acid group.

33. The method of claim 32, wherein the interior buffer solution has a pH in the range of 7-9, such as in the range of 7.5-9 or in the range of 8-9.

34. The method of claim 32 or 33, wherein the interior buffer solution comprises a basic component selected from the group consisting of ammonium acetate, potassium acetate, sodium acetate, calcium acetate, ammonium benzoate, potassium benzoate, sodium benzoate and calcium benzoate.

35. The method of any one of claims 32-34, wherein the exterior buffer solution has a pH in the range of 2.5-6, such as in the range of 2.5-5, in the range of 2.5-4, or in the range of 2.5-3.

36. The method of any one of claims 32-35, wherein the exterior buffer solution comprises a buffering component selected from the group consisting of citric acid, acetic acid, phosphate, histidine, MES, Bis-Tris and ADA.

37. The method of any one of claims 32-36, wherein the compound has a pKa in the range of 2-6, for example in the range of 2-5, in the range of 2-4, or in the range of 2-3.

38. The method of any one of claims 32-37, wherein the compound has a logD above 0 in the pH range of 2-6 and/or a logD below 0 in the pH range of 6-9.

39. The method of any one of claims 16-38, wherein the liposome comprises in its membrane one or more of: DSPC (1,2-distearoyl-sn-glycero-3-phosphocholine), HSPC (hydrogenated soybean phosphatidylcholine), CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy(polyethylene glycol)]).

40. The method of claim 39, wherein the liposome comprises in its membrane about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

41. A liposome composition prepared by the method of any one of claims 16-40.

42. A liposome composition comprising a membrane and a TLR7/8 agonist entrapped inside the membrane,
wherein the membrane comprises one or more of: DSPC (1,2-distearoyl-sn-glycero-3-phosphocholine), HSPC (hydrogenated soybean phosphatidylcholine),CHOL (Cholesterol), polyArginine-CHOL (such as Arg3-CHOL and Arg8-CHOL), DSPE-PEG (1,2-distearoyl-sn-glycero-3-phosphoethanolamine-N-[methoxy(polyethyleneglycol)]), antibody (such as anti-CD45 antibody) conjugated DSPE-PEG, POPC (1-palmitoyl-2-oleoyl-sn-glycero-3-phosphocholine), DOTAP (N-[1-(2,3-Dioleoyloxy)propyl]-N,N,N-trimethylammonium), DSTAP (1,2-Distearoyl-3-trimethylammonium-propane) and DOPE-PEG (1,2-Dioleoyl-sn-glycero-3-phosphoethanolamine-N-[methoxy(polyethylene glycol)]);
wherein the TLR7/8 agonist is Gardiquimod and/or one or more of the compounds of any one of claims 1-11.

43. The liposome composition of claim 42, wherein the membrane comprises about 0.1-10 mol % (such as about 1.5-2 mol % or about 1.5 mol %) Arg3-CHOL, about 0.1-10 mol % (such as about 0.5-2 mol % or about 0.5 mol %) DSPE-PEG-2000, about 5-20 mol % (such as about 5-10 mol % or about 10 mol %) DSTAP, about 20-50 mol % (such as about 30-40 mol % or about 35 mol %) cholesterol, and about 40-70 mol % (such as about 50-55 mol % or about 53 mol %) DSPC.

44. A pharmaceutical composition comprising the liposome composition of any one of claims 41-43 and a pharmaceutically acceptable carrier.

45. A cell composition comprising the liposome composition of any one of claims 41-43 and a nucleated cell such as an immune cell.

46. A composition comprising the compound or salt of any one of claims 1-11, and further comprising a pharmaceutically acceptable salt, a liposome, and/or a nucleated cell such as an immune cell.

47. A composition comprising Gardiquimod, and further comprising a pharmaceutically acceptable salt, a liposome, and/or a nucleated cell such as an immune cell.

48. Use of the liposome composition of any one of claims 12-15 and 41-43, the pharmaceutical composition of claim 44, the cell composition of claim 45, or the composition of claim 46 or 47, in the manufacture of a medicament for stimulating an immune response in an individual in need thereof, such as for the treatment of cancer, an infectious disease, an inflammatory condition or disease and autoimmune disease or allergy.
